(19) United States
(12) Patent Application Publication
(10) Pub. No.: US 2005/0014812 A1
(43) Pub. Date: Jan. 20, 2005

(54) Benzimidazole compounds

(75) Inventors: Hisashi Hayashida, Osaka-shi (JP); Keiko Hatanaka, Osaka (JP); Takeshi Kato, Osaka (JP); Yoshiyuki Kido, Osaka (JP); Masaki Tomishima, Osaka (JP); Hiroshi Kayakiri, Osaka (JP); Ichiro Takase, Tsukuba-shi (JP); Takahiro Hiramura, Tsukuba-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignees: Fujisawa Pharmaceutical Co., Ltd., Osaka-shi (JP); Daicel Chemical Industries, Ltd., Sakai-shi (JP)

(21) Appl. No.: 10/860,139

(22) Filed: Jun. 4, 2004

(30) Foreign Application Priority Data
Jun. 6, 2003 (AU) 2003902860

(51) Int. Cl. A61K 31/4184; C 07D 4 3/02; C07D 235/20

(52) U.S. Cl. 514/394; 548/305.4; 548/305.7

(57) ABSTRACT

The present invention provides novel benzimidazole compounds of the formula (I): 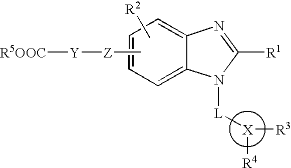 wherein each symbol is as defined in the specification, salts thereof and prodrugs thereof, which are useful in treating, for example, the diseases curable through decrease in blood sugar level.

### TECHNICAL FIELD

The present invention relates to novel benzimidazole compounds. More particularly, the present invention relates to novel benzimidazole compounds, salts thereof and prodrugs thereof having a hypoglycemic activity. The present invention also relates to a method for producing the above-mentioned benzimidazole compounds, salts thereof and prodrugs thereof. Moreover, the present invention relates to pharmaceutical compositions comprising the above-mentioned benzimidazole compound, a salt thereof or a prodrug thereof as an active ingredient.

### BACKGROUND ART

Antidiabetic agents having sulfonylcarbamoyl structure and benzimidazole structure are described in International Publication Nos. WO 97/24334, WO 99/00372 and WO 00/39099.

Antidiabetic agents of the thiazolidinedione (TZD) class, which have been widely used and have proven efficacy in the treatment of patients with type II diabetes via activation of the gamma isoform of the peroxisome proliferator-activated receptor (PPARγ), are known and widely used. However, it has been clarified that the agents of the TZD class have side effects such as edema, weight gain and the like (see Diabetes Metab Res Rev 2002; 18: S23-S29).

There is a demand for development of an antidiabetic agent that has no or decreased side effects.

### DISCLOSURE OF THE INVENTION

The present invention aims at providing novel benzimidazole compounds, pharmaceutically acceptable salts thereof, prodrugs thereof and pharmaceutical preparations comprising the above-mentioned benzimidazole compound, or a pharmaceutically acceptable salt thereof, a prodrug thereof as an active ingredient.

The novel benzimidazole compounds induce improvement in plasma lipid metabolism, improvement in plasma lipoprotein composition, hypoglycemic effect, hypoinsulinemic effect, improvement in insulin resistance, enhancement in insulin sensitivity and the like.

In addition, the novel benzimidazole compounds have a superior ligand activity for peroxisome proliferator-activated receptors (PPARα, PPARγ and PPARδ) and are useful as an agonist, a partial agonist, an antagonist or a partial antagonist for these receptors.

Moreover, the novel benzimidazole compounds have a superior ligand activity for peroxisome proliferator-activated receptor in a heterodimer receptor which is formed by retinoid X receptor and peroxisome proliferator-activated receptor (for example, a heterodimer receptor which is formed by RXRα and PPARδ, a heterodimer receptor which is formed by RXRα and PPARγ, and the like).

The novel benzimidazole compounds are used as an agent for the prophylaxis and treatment of impaired glucose tolerance disorder, diabetes (e.g., type II diabetes), gestational diabetes, diabetic complications (e.g., diabetic gangrene, diabetic arthropathy, diabetic osteopenia, diabetic glomerulosclerosis, diabetic nephropathy, diabetic dermatopathy, diabetic neuropathy, diabetic cataract, diabetic retinopathy and the like), insulin resistance syndrome (e.g., insulin receptor abnormality, Rabson-Mendenhall syndrome, leprechaunism, Kobberling-Dunnigan syndrome, Lawrence-Seip syndrome (lipoatrophy), Cushing syndrome, acromegaly and the like), polycystic ovary syndrome, hyperlipidemia, atherosclerosis, cardiovascular diseases (e.g., stenocardia, cardiac failure and the like), hyperglycemia (e.g., those characterized by abnormal saccharometabolism such as eating disorders), pancreatitis, osteoporosis, hyperuricemia, hypertension, inflammatory bowel diseases, skin disorders related to an anomaly of differentiation of epidermic cells, hypertension, Alzheimer's disease, Parkinson's disease, multiple sclerosis, stroke, traumatic brain and spinal cord injury, and the like. In addition, they, in combination with a retinoid, are useful for treating disease states caused by uncontrolled cell proliferation, including cancer, restenosis and atherosclerosis.

The benzimidazole compound [hereinafter to be also referred to as the objective compound (I)], which is the novel compound of the present invention, has the formula (I): 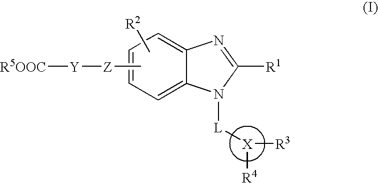
wherein
R¹ is lower alkyl, lower alkoxy, lower alkylthio, mono- or di(lower alkyl)amino or mono-, di- or trihalo(lower)alkyl,
R² is hydrogen or lower alkyl,
R³ is hydrogen, halogen, cyano, optionally esterified carboxy, lower alkoxy, lower alkyl optionally substituted by lower alkoxy, optionally substituted amino, mono-, di- or trihalo(lower)alkyl, optionally substituted aryl or heteroaryl,
R⁴ is halogen, lower alkyl, lower alkoxy, lower alkenyl or mono-, di- or trihalo(lower)alkyl,
R⁵ is hydrogen or carboxy protective group,
L is lower alkylene,
ring X is benzene ring or heteroaryl ring,
Y is lower alkylene, optionally substituted phenylene or bivalent residue derived from optionally substituted heteroaryl, and
Z is bond, —O—, —CH₂O—, —OCH₂—, —N(R⁹)CH₂— or —CH₂N(R⁹)—, wherein R⁹ is hydrogen or lower alkyl,
provided that when Z is a bond, then Y is optionally substituted phenylene or bivalent residue derived from optionally substituted heteroaryl,
or a salt thereof or a prodrug thereof.

The preferable embodiments of the benzimidazole compound of the present invention represented by the general formula (I) are follows.

(1) The compound of general formula (I) wherein ring X is benzene ring, pyridine ring, oxazole ring or thiazole ring, Y is lower alkylene, phenylene optionally substituted by substituent(s) selected from the group consisting of lower alkyl, halogen, amino or nitro or bivalent residue derived from pyridinyl, thiophenyl, imidazolyl or oxazolyl, each of which is optionally mono-substituted by lower alkyl, or a salt thereof or a prodrug thereof.

(2) The compound of (1) above wherein L is methylene, and Z is bond, —O—, —CH₂O— or —OCH₂—, or a salt thereof or a prodrug thereof.

(3) The compound of (2) above wherein Z is —O—, and Y is lower alkylene, or a salt thereof or a prodrug thereof.

(4) The compound of (2) wherein Z is bond, —CH₂O— or —OCH₂— and Y is phenylene optionally substituted by substituent(s) selected from the group consisting of lower alkyl, halogen, amino or nitro, or bivalent residue derived from pyridinyl, thiophenyl, imidazolyl or oxazolyl, each of which is optionally mono-substituted by lower alkyl, or a salt thereof or a prodrug thereof.

(5) The compound of (2), which is selected from the group consisting of

4-{[1-(2,4-dichlorobenzyl)-2,4-dimethyl-1H-benzimidazol-6-yl]oxy}butanoic acid,
4-[(1-{[3-chloro-5-(trifluoromethyl)-2-pyridinyl]methyl}-2,4-dimethyl-1H-denzimidazol-6-yl)oxy]butanoic acid,
4-({1-[(3,5-dichloro-2-pyridinyl)methyl]-2,4-dimethyl-1H-benzimidazol-6-yl}oxy)butanoic acid,
4-{[2-ethoxy-4-methyl-1-({4-methyl-2-[4-(trifluoromethyl)phenyl]-1,3-thiazol-5-yl}methyl)-1H-benzimidazol-6-yl]oxy}butanoic acid,
4-{[1-(2,4-dichlorobenzyl)-2-ethyl-4-methyl-1H-benzimidazol-6-yl]oxy}butanoic acid,
4-{[1-(2,4-dichlorobenzyl)-2-ethoxy-4-methyl-1H-benzimidazol-6-yl]oxy}butanoic acid,
2-[({1-[(3-chloro-1,1′-biphenyl-4-yl)methyl]-2,4-dimethyl-1H-benzimidazol-6-yl}oxy)methyl]benzoic acid,
2-{[(1-{[3-chloro-5-(trifluoromethyl)-2-pyridinyl]methyl}-2,4-dimethyl-1H-benzimidazol-6-yl)oxy]methyl}benzoic acid,
2-[({1-[(2-chloro-6-phenyl-3-pyridinyl)methyl]-2,4-dimethyl-1H-benzimidazol-6-yl}oxy)methyl]benzoic acid,
2-([(1-{[3-chloro-5-(trifluoromethyl)-2-pyridinyl]methyl}-2-methyl-1H-benzimidazol-6-yl)oxy]methyl}benzoic acid,
4-{[1-(2,4-dichlorobenzyl)-2-methyl-1H-benzimidazol-6-yl]oxy}-2,2-dimethylbutanoic acid and
2-[({1-[(3-chloro-1,1′-biphenyl-4-yl)methyl]-2,4-dimethyl-1H-benzimidazol-6-yl}oxy)methyl]-6-methylbenzoic acid,
   or a salt thereof or a prodrug thereof.

Preferable salts of the objective compound (I) are conventional salts that are non-toxic and acceptable for use as pharmaceuticals. Examples thereof include salts with alkali metal such as sodium and potassium, salts with alkaline earth metal such as calcium and magnesium, salts with inorganic base such as ammonium salt, salts with organic amine such as triethylamine, pyridine, picoline, ethanolamine and triethanolamine, salts with inorganic acid such as hydrochloric acid, hydrobromic acid, sulfuric acid and phosphoric acid, salts with organic carboxylic acid such as formic acid, acetic acid, trifluoroacetic acid, maleic acid and tartaric acid, addition salts with sulfonic acid such as methanesulfonic acid, benzenesulfonic acid and p-toluenesulfonic acid, and salts or acid addition salts with base such as basic or acidic amino acid such as arginine, aspartic acid and glutamic acid.

The objective compound (I) or a salt therof may be a prodrug. A prodrug of the objective compound (I) or a salt therof refers to a compound capable of being converted to the objective compound (I) or a salt therof by reactions of an enzyme, gastric juice, and the like, under physiological conditions in vivo, specifically a compound capable of being converted to the objective compound (I) or a salt therof upon enzymatic oxidation, reduction, hydrolysis, and the like, or a compound capable of being converted to the objective compound (I) or a salt therof upon hydrolysis and the like by gastric juice and the like.

The objective compound (I), a salt thereof and a prodrug thereof of the present invention [hereinafter also referred to as the compound of the present invention] can be produced by the method shown by the following reaction formulas. 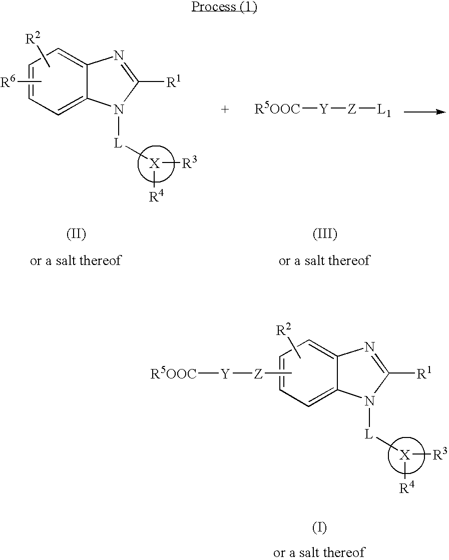 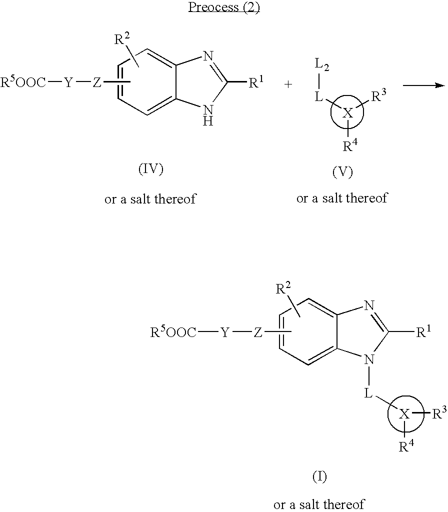 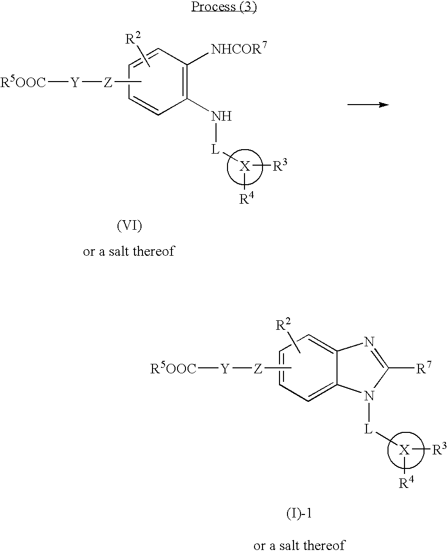 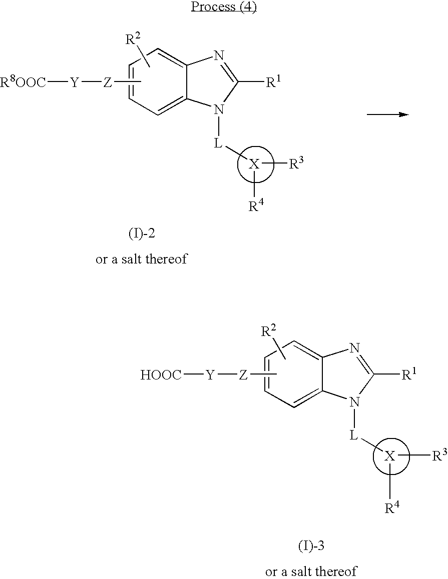
wherein R⁶ is hydroxyl, halogen or carboxy, R⁷is lower alkyl, R⁸ is carboxy protective group, L₁ and L₂ are each leaving group such as halogen (e.g., bromine, iodine etc.), alkylsulfonyloxy, group (e.g., methanesulfonyloxy, ethanesulfonyloxy etc.), optionally substituted arylsulfonyloxy group (e.g., benzenesulfonyloxy, p-toluenesulfonyloxy etc.), and the like and other symbols in the formulas are as defined above.

The starting compounds can be prepared by the method of Preparation Example below or a process known in the art for preparing their structurally analogous compounds.

Various definitions included in the present specification are explained in detail in the following.

Specific examples of “halogen” include fluorine, bromine, chlorine and iodine.

Specific examples of “esterified carboxy” in “optionally esterified carboxy” include alkoxycarbonyl having 2 to 5 carbon atoms (e.g., methoxycarbonyl, ethoxycarbonyl etc.) and the like. Preferable examples of “optionally esterified carboxy” include carboxy and ethoxycarbonyl.

“Lower alkyl” is preferably linear or branched alkyl having up to 6 carbon atoms. Specific examples thereof include methyl, ethyl, 1-propyl, isopropyl, 1-butyl, isobutyl, tert-butyl, sec-butyl, 1-pentyl, isopentyl, tert-pentyl, sec-pentyl, methylbutyl, 1,1-dimethylpropyl, 1-hexyl, 1-methylpentyl, 2-methylpentyl, 3-methylpentyl, 4-methylpentyl, 1-ethylbutyl, 2-ethylbutyl, 3-ethylbutyl, 1,1-dimethylbutyl, 2,2-dimethylbutyl, 3,3-dimethylbutyl, 1-ethyl-1-methylpropyl and the like. Of these, linear alkyl having 1 to 4 carbon atoms is preferable and methyl and ethyl are especially preferable.

“Lower alkoxy” is linear or branched alkyloxy having up to 6 carbon atoms. Specific examples thereof include methoxy, ethoxy, 1-propyloxy, isopropyloxy, 1-butyloxy, isobutyloxy, sec-butyloxy, tert-butyloxy, 1-pentyloxy, isopentyloxy, sec-pentyloxy, tert-pentyloxy, 2-methylbutoxy, 1-hexyloxy, isohexyloxy, tert-hexyloxy, sec-hexyloxy, 2-methylpentyloxy, 3-methylpentyloxy, 1-ethylbutyloxy, 2-ethylbutyloxy, 1,1-dimethylbutyloxy, 2,2-dimethylbutyloxy, 3,3-dimethylbutyloxy, 1-ethyl-1-methylpropyloxy, and the like. Of these, linear alkoxy having 1 to 5 carbon atoms is preferable and methoxy, ethoxy and 1-pentyloxy are especially preferable.

“Lower alkylthio” is linear or branched alkylthio having up to 6 carbon atoms. Specific examples thereof include methylthio, ethylthio, 1-propylthio, isopropylthio, 1-butylthio, isobutylthio, sec-butylthio, tert-butylthio, 1-pentylthio, isopentylthio, sec-pentylthio, tert-pentylthio, 2-methylbutylthio, 1-hexylthio, isohexylthio, tert-hexylthio, sec-hexylthio, 2-methylpentylthio, 3-methylpentylthio, 1-ethylbutylthio, 2-ethylbutylthio, 1,1-dimethylbutylthio, 2,2-dimethylbutylthio, 3,3-dimethylbutylthio, 1-ethyl-1-methylpropyloxy, and the like. Of these, linear alkylthio having 1 to 4 carbon atoms is preferable and ethylthio is especially preferable.

“Mono- or di-(lower alkyl)amino” is amino which is mono- or di-substituted by linear or branched alkyl having up to 6 carbon atoms. Specific examples thereof include methylamino, ethylamino, 1-propylamino, isopropylamino, 1-butylamino, isobutylamino, sec-butylamino, tert-butylamino, 1-pentylamino, isopentylamino, sec-pentylamino, tert-pentylamino, 2-methylbutylamino, 1-hexylamino, isohexylamino, tert-hexylamino, sec-hexylamino, 2-methylpentylamino, 3-methylpentylamino, 1-ethylbutylamino, 2-ethylbutylamino, 1,1-dimethylbutylamino, 2,2-dimethylbutylamino, 3,3-dimethylbutylamino, 1-ethyl-1-methylpropylamino, and the like. Of these, linear alkylamino having 1 to 4 carbon atoms is preferable and methylamino is especially preferable. “Optionally substituted amino” is preferably amino which is optionally substituted by substituent(s) selected from aforementioned lower alkyl and aforementioned optionally esterified carboxy. Preferable examples thereof include dimethylamino and (ethoxycarbonyl)(methyl)amino.

“Mono-, di- or trihalo(lower)alkyl” is linear or branched alkyl having up to 6 carbon atoms, which is substituted by 1 to 3 halogen atom such as fluorine atom, chlorine atom, bromine atom and iodine atom. Specific Examples thereof include fluoromethyl, difluoromethyl, trifluoromethyl, chloromethyl, dichloromethyl, trichloromethyl, bromomethyl, dibromomethyl, tribromomethyl, 1-fluoroethyl, 1-chloroethyl, 1-bromoethyl, 2-fluoroethyl, 2-chloroethyl, 2-bromoethyl, 1,2-difluoroethyl, 1,2-dichloroethyl, 1,2-dibromoethyl, 2,2,2-trifluoroethyl, heptafluoroethyl, 1-fluoropropyl, 1-chloropropyl, 1-bromopropyl, 2-fluoropropyl, 2-chloropropyl, 2-bromopropyl, 3-fluoropropyl, 3-chloropropyl, 3-bromopropyl, 1,2-difluoropropyl, 1,2-dichloropropyl, 1,2-dibromopropyl, 2,3-difluoropropyl, 2,3-dichloropropyl, 2,3-dibromopropyl, 3,3,3-trifluoropropyl, 2,2,3,3,3-pentafluoropropyl, 2-fluorobutyl, 2-chlorobutyl, 2-bromobutyl, 4-fluorobutyl, 4-chlorobutyl, 4-bromobutyl, 4,4,4-trifluorobutyl, 2,2,3,3,4,4,4-heptafluorobutyl, perfluorobutyl, 2-fluoropentyl, 2-chloropentyl, 2-bromopentyl, 5-fluoropentyl, 5-chloropentyl, 5-bromopentyl, perfluoropentyl, 2-fluorohexyl, 2-chlorohexyl, 2-bromohexyl, 6-fluorohexyl, 6-chlorohexyl, 6-bromohexyl, perfluorohexyl and the like. Of these, trifluoromethyl is preferable.

“Optionally substituted aryl” is aryl having 6 to 10 carbon atoms, which is optionally substituted by substituent(s) such as optionally substituted lower alkyl. Suitable examples of aryl include phenyl, tolyl and naphthyl, in which more preferable one is phenyl. Preferable examples thereof include phenyl optionally substituted by aforementioned mono-, di- or trihalo (lower) alkyl, especially trihalo (lower) alkyl. Phenyl and (4-trifluoromethyl)phenyl are especially preferable.

“Lower alkenyl” is linear or branched alkenyl having 2 to 6 carbon atoms. Specific examples thereof include vinyl, 1-propenyl, 2-propenyl, isopropenyl, 1-butenyl, 2-butenyl, 3-butenyl, 1-methyl-1-propenyl, 1-methyl-2-propenyl, 2-methyl-2-propenyl, 1-ethylvinyl, 1-pentenyl, 2-pentenyl, 3-pentenyl, 4-pentenyl, 1,2-dimethyl-1-propenyl, 1,2-dimethyl-2-propenyl, 1-ethyl-1-propenyl, 1-ethyl-2-propenyl, 1-methyl-1-butenyl, 1-methyl-2-butenyl, 2-methyl-1-butenyl, 1-isopropylvinyl, 2,4-pentadienyl, 1-hexenyl, 2-hexenyl, 3-hexenyl, 4-hexenyl, 5-hexenyl, 2,4-hexadienyl, 1-methyl-1-pentenyl and the like. Of these, linear alkenyl having 2 to 4 carbon atoms is preferable and vinyl is especially preferable.

“Lower alkylene” is preferably linear or branched alkylene having up to 6 carbon atoms. Specific examples thereof include methylene, ethylene, trimethylene, tetramethylene, pentamethylene, hexamethylene, methylmethylene, dimethylmethylene, 1-methylethylene, 1,1-dimethylethylene, 1-methyltrimethylene, 1,1-dimethyltrimethylene, and the like. Of these, linear or branched alkylene having 1 to 5 carbon atoms is preferable and methylene, trimethylene, tetramethylene, pentamethylene, dimethylmethylene and 1,1-dimethyltrimethylene are especially preferable.

“Optionally substituted phenylene” is preferably phenylene which is optionally substituted by substituent(s). Examples of the substituent include aforementioned lower alkyl (e.g., methyl etc.), aforementioned halogen (e.g., fluorine, chlorine etc.), amino, nitro and the like.

“Heteroaryl ring” is 5- or 6-membered aromatic heteromonocyclic ring containing 1 to 4 heteroatom(s) selected from sulfur atom, oxygen atom and nitrogen atom. Specific examples thereof include pyridine ring, pyrimidine ring, pyrazine ring, pyridazine ring, pyrrole ring, imidazole ring, pyrazole ring, triazole ring, tetrazole ring, thiazole ring, isothiazole ring, thiadiazole ring, oxazole ring, isoxazole ring, furan ring, thiophene ring and the like. Of these, pyridine ring, oxazole ring and thiazole ring are preferable.

“Heteroaryl” is 5- or 6-membered aromatic heteromonocyclic group containing 1 to 4 heteroatom(s) selected from sulfur atom, oxygen atom and nitrogen atom. Specific examples thereof include pyridinyl, pyrimidinyl, pyrazinyl, pyridazinyl, pyrrolyl, imidazolyl, pyrazolyl, triazolyl, tetrazolyl, thiazolyl, isothiazolyl, thiadiazolyl, oxazolyl, isoxazolyl, furanyl, thiophenyl, and the like. Of these, pyridinyl, pyrazolyl and oxazolyl are preferable.

“Bivalent residue derived from optionally substituted heteroaryl” is bivalent 5- or 6-membered aromatic heteromonocyclic group containing 1 to 4 heteroatom(s) selected from sulfur atom, oxygen atom and nitrogen atom, wherein the group may be substituted. Preferable examples of “heteroaryl” of “bivalent residue derived from optionally substituted heteroaryl” include pyridinyl, thiophenyl, imidazolyl and oxazolyl. “Heteroaryl” of “bivalent residue derived from optionally substituted heteroaryl” is optionally mono-substituted by aforementioned lower alkyl (e.g., methyl etc.).

Suitable examples of “carboxy protective group” include lower alkyl (e.g., methyl, ethyl, tert-butyl etc.), mono(or di or tri)phenyl(lower alkyl) optionally substituted by nitro (e.g., benzyl, 4-nitrobenzyl, benzhydryl, trityl etc.) and lower alkylcarbonyloxy(lower)alkyl) (e.g., pivaloyloxymethyl).

Preferable specific compounds as the objective compound (I) are exemplified by the following:
(1) 4-{[1-(2,4-dichlorobenzyl)-2,4-dimethyl-1H-benzimidazol-6-yl]oxy}butanoic acid,
(2) 4-[(1-{[3-chloro-5-(trifluoromethyl)-2-pyridinyl]methyl}-2,4-dimethyl-1H-dnezimidazol-6-yl)oxy]butanoic acid,
(3) 4-({1-[(3,5-dichloro-2-pyridinyl)methyl]-2,4-dimethyl-1H-benzimidazol-6-yl}oxy)butanoic acid,
(4) 4-{[2-ethoxy-4-methyl-1-({4-methyl-2-[4-(trifluoromethyl)phenyl]-1,3-thiazol-5-yl}methyl)-1H-benzimidazol-6-yl]oxy}butanoic acid,
(5) 4-{[1-(2,4-dichlorobenzyl)-2-ethyl-4-methyl-1H-benzimidazol-6-yl]oxy}butanoic acid,
(6) 4-{[1-(2,4-dichlorobenzyl)-2-ethoxy-4-methyl-1H-benzimidazol-6-yl]oxy}butanoic acid,
(7) 2-[({1-[(3-chloro-1,1′-biphenyl-4-yl)methyl]-2,4-dimethyl-1H-benzimidazol-6-yl}oxy)methyl]benzoic acid,
(8) 2-{[(1-{[3-chloro-5-(trifluoromethyl)-2-pyridinyl]methyl}-2,4-dimethyl-1H-benzimidazol-6-yl)oxy]methyl}benzoic acid,
(9) 2-[({1-[(2-chloro-6-phenyl-3-pyridinyl)methyl]-2,4-dimethyl-1H-benzimidazol-6-yl}oxy)methyl]benzoic acid,
(10) 2-{[(1-{[3-chloro-5-(trifluoromethyl)-2-pyridinyl]methyl}-2-methyl-1H-benzimidazol-6-yl)oxy]methyl}benzoic acid,
(11) 4-{[1-(2,4-dichlorobenzyl)-2-methyl-1H-benzimidazol-6-yl]oxy}-2,2-dimethylbutanoic acid and
(12) 2-[({1-[(3-chloro-1,1′-biphenyl-4-yl)methyl]-2,4-dimethyl-1H-benzimidazol-6-yl}oxy)methyl]-6-methylbenzoic acid,
   or a salt therof or a prodrug thereof.

The production methods of the objective compound (I) are explained in detail in the following.

Process (1)

The objective compound (I) and a salt thereof can be produced by reacting compound (II) or a salt thereof with compound (III) or a salt thereof.

Preferable salts of compound (II) and compound (III) are exemplified by those shown with regard to compound (I).

Preferable leaving group for L₁ is halogen, with more preference to bromine.

The reaction generally proceeds in a conventional solvent such as water, alcohol (e.g., methanol and ethanol), acetone, dioxane, acetonitrile, chloroform, methylene chloride, ethylene chloride, tetrahydrofuran, ethyl acetate, N,N-dimethylformamide, pyridine and a mixture thereof, or in any other solvent which does not adversely affect the reaction. These conventional solvents may be used alone or in combination.

The reaction can be carried out in the presence of an inorganic or organic base such as alkali metal bicarbonate, tri(lower)alkylamine, pyridine, 4-dimethylaminopyridine, N-(lower)alkylmorpholine, N,N-di(lower)alkylaniline (e.g., N,N-dimethylaniline), N,N-di(lower)alkylbenzylamine, and the like.

The reaction temperature is not particularly limited, and the reaction is generally carried out under cooling to heating.

Process (2)

The compound (I) or a salt thereof can be prepared by reacting the compound (IV) or a salt thereof with the compound (V) or salt thereof.

Preferable leaving group for L₂ is halogen, with more preference to bromine.

The reaction is usually carried out in a conventional solvent such as tetrahydrofuran, dioxane, toluene, methylene chloride, ethylene dichloride, N,N-dimethylformamide, N,N-dimethylacetamide, or any other organic solvents which do not adversely affect the reaction, or a mixture thereof.

The reaction temperature is not particularly limited, and the reaction is generally carried out under cooling to heating.

Process (3)

The compound (I)-1 or a salt thereof can be prepared by dehydrating the compound (VI) or a salt thereof.

This dehydration reaction is carried out by a conventional method in the presence of potassium carbonate or sulfuric acid in a conventional solvent such as ethanol, tetrahydrofuran, dioxane, toluene, methylene chloride, ethylene dichloride, N,N-dimethylformamide, N,N-dimethylacetamide, or any other organic solvents which do not adversely affect the reaction, or a mixture thereof.

The reaction temperature is not particularly limited, and the reaction is generally carried out under heating.

Process (4)

The compound (I)-3 or a salt thereof can be prepared by hydrolyzing the compound (I)-2 or a salt thereof.

This hydrolysis reaction is carried out by a conventional method in the presence of an acid or a base in a hydrated solvent.

Examples of the acid include hydrochloric acid, hydrobromic acid, sulfuric acid and acetic acid.

Examples of the base include alkali metal carbonates such as potassium carbonate and sodium carbonate; alkali metal alkoxides such as sodium methoxide; and alkali metal hydroxides such as potassium hydroxide, sodium hydroxide and lithium hydroxide.

Examples of the hydrated solvent include solvent mixtures of water and one or more solvents selected from alcohols such as methanol and ethanol; ethers such as tetrahydrofuran, dioxane and diethyl ether; dimethyl sulfoxide and acetone.

The reaction temperature is not particularly limited, and the reaction is generally carried out under cooling to heating.

The aforementioned compounds can be converted to preferable salts or prodrugs as necessary by a conventional method. All of them can be purified as necessary according to a conventional method for purifying an organic compound (i.e., recrystallization, column chromatography, thin layer chromatography, high performance liquid chromatography and the like). The compound can be identified by NMR spectrum analysis, mass spectrum analysis, IR spectrum analysis, elemental analysis, melting point measurement and the like.

The compound of the present invention may have one or more chiral centers and, therefore, may be presented in enantiomers or diastereomers. The present invention encompasses these isomers and mixtures thereof.

The compound of the present invention may be in the form of a solvate, which is also encompassed in the present invention. The solvate is preferably exemplified by hydrate and ethanol solvate.

The compound of the present invention can be used for therapeutic purposes in the form of a pharmaceutical preparation. This pharmaceutical preparation contains any one of the compounds (I) as an active ingredient in admixture with a pharmaceutically acceptable organic or inorganic excipient which is a solid, semisolid or liquid and which is suitable for oral, parenteral or external (local) administration. Examples of the pharmaceutical preparation include capsules, tablets, sugar coating tablets, granules, suppositories, liquid, lotion, suspension, emulsion, ointment, gel and the like. When desired, these preparations may contain adjuvant, auxiliary substance, stabilizer, moistening agent, emulsifier, buffering agent, and other conventional additives. While the dose of the compound (I) varies depending on the age and symptom of patients, compound (I) may be administered for the therapy of the above-mentioned diseases in an average single dose amount of about 0.1 mg, 1 mg, 10 mg, 50 mg, 100 mg, 250 mg, 500 mg or 1000 mg. In general, its daily dose may be from about 0.1 mg/patient to about 1000 mg/patient.

The compound of the present invention shows a superior hypoglycemic activity. Therefore, it can be used as a medicament for mammals (e.g., human, calf, horse, pig, dog, cat, monkey, mouse, rat etc., particularly human).

The compound of the present invention may be used in combination with other pharmaceutical agents.

As the pharmaceutical agent used in combination with the compound of the present invention, α-glucosidase inhibitor, sulfonylurea, insulin secretagogue, insulin preparation, biguanide, β-hydroxy-β-methylglutaryl CoA (HMG-CoA) reductase inhibitor, calcium antagonist, fibrate, diuretic, angiotensin converting enzyme (ACE) inhibitor, angiotensin II antagonist, cholesterol absorption inhibitor, antioxidant, nicotinic acid derivative, squalene synthesis inhibitor, aldose reductase inhibitor, β3 agonist, peroxisome proliferator-activated receptor (PPAR) modulator, dipeptidylpeptidase 4 (DPP4) inhibitor, glucagon-like peptide-1 (GLP-1) analog, sodium-dependent glucose cotransporter (SGLT) inhibitor, 11β-hydroxysteroiddehydrogenase 1 (11β-HSD1) inhibitor, microsomal triglyceride transfer protein (MTP) inhibitor, acyl-CoA: cholesterol.acyltransferase (ACAT) inhibitor, ileal bile acid transporter (IBAT) inhibitor, ezetimibe, vascular adhesion protein 1 (VAP1) inhibitor, advanced glycation end products (AGE) inhibitor, lipase inhibitor, anorexiant, leptin, adiponectin and the like can be mentioned. Of these, α-glucosidase inhibitor, insulin secretagogue, sulfonylurea and biguanide are preferable.

The α-glucosidase inhibitor is a pharmaceutical agent having an action of delaying digestion of starch or sucrose by inhibiting digestive enzymes such as amylase, maltase, α-dextrinase, sucrase and the like.

As preferable α-glucosidase inhibitor, miglitol; [2R(2α,3β,4α,5β)]-1-(2-hydroxyethyl)-2-(hydroxymethyl)-3,4,5-piperidinetriol), voglibose; 3,4-dideoxy-4-[[2-hydroxy-1-(hydroxymethyl)ethyl]amino]-2-C-(hydroxymethyl)-D-epi-inositol, miglustat; N-butyl-1-deoxynojirimicin, acarbose; O-4,6-dideoxy-4-[[1S-(1α,4α,5β,6α)-4,5,6-trihydroxy-3-(hydroxymethyl)-2-cyclohexen-1-yl]amino]-α-D-glucopyranosyl-(1→4)-O-α-D-glucopyranosyl-(1→4)-D-glucose, celgosivir hydrochloride; [1S-(1α,6β,7α,8β,8αβ)]-octahydro-1,7,8-trihydroxy-6-indolizinyl butanoate hydrochloride and the like can be mentioned.

Insulin secretagogue is a pharmaceutical agent having an action to promote secretion of insulin from pancreatic β cells. As the insulin secretagogue, for example, sulfonylurea (SU) can be mentioned. Said sulfonylurea (SU) is an agonist of cell membrane SU receptors, thereby promoting secretion of insulin from pancreatic β cells.

As preferable insulin secretagogue, nateglinide; N-(trans-4-isopropylcyclohexylcarbonyl)-D-phenylalanine, glimepiride; trans-3-ethyl-2,5-dihydro-4-methyl-N-2-[4-[[[[(4-methylcyclohexyl)amino]carbonyl]amino]sulfonyl]phenyl]ethyl-2-oxo-1H-pyrrole-1-carboxamide, repaglinide; (+)-2-ethoxy-α-[[(S)-α-isobutyl-o-piperidinobenzyl]carbamoyl]-p-toluic acid, glisentide; 1-cyclopentyl-3-p-(2-o-anisamideethyl)benzenesulfonylurea, mitiglinide; (−)-2(S)-benzyl-4-oxo-4-(cis-perhydroisoindol-2-yl)butyric acid calcium salt.dihydrate, glucagon-like peptide-17-36-amide, glucagon-like peptide-1-amylin, CJC1131 and the like can be mentioned.

As other insulin secretagogues, for example, N-[[4-(1-methylethyl)cyclohexyl]carbonyl]-D-phenylalanine (AY-4166), (2S)-2-benzyl-3-(cis-hexahydro-2-isoindolinylcarbonyl)propionic acid calcium.dihydrate (KAD-1229), glimepiride (Hoe490) can be mentioned.

As preferable sulfonylurea, glimepiride; trans-3-ethyl-2,5-dihydro-4-methyl-N-2-[4-[[[[(4-methylcyclohexyl)amino]carbonyl]amino]sulfonyl]phenyl]ethyl-2-oxo-1H-pyrrole-1-carboxamide, glisentide; 1-cyclopentyl-3-p-(2-o-anisamideethyl)benzenesulfonylurea and the like can be mentioned.

As other sulfonylureas, for example, tolbutamide, chlorpropamide, tolazamide, acetohexamide, 4-chloro-N-[(1-pyrrolidinylamino)carbonyl]-benzenesulfonamide (glyclopyramide) and its ammonium salt, glibenclamide (glyburide), gliclazide, 1-butyl-3-metanilylurea, carbutamide, glibornuride, glipizide, gliquidone, glisoxepide, glybuthiazole, glybuzole, glyhexamide, glymidine, glypinamide, phenbutamide, tolycyclamide and the like can be mentioned.

Biguanide is a pharmaceutical agent that acts to enhance anaerobic glycolysis, sensitize insulin in the periphery, inhibit glucose absorption from the intestine, inhibit hepatic gluconeogenesis, increase fatty acid oxidation and the like.

As preferable biguanide, phenformin; 1-phenethyl biguanide, metformin; 1,1-dimethylbiguanide, buformin; 1-butyl biguanide and the like can be mentioned.

Examples of the HMG-CoA reductase inhibitor include rosuvastatine calcium, atorvastatine calcium hydrate, pitavastatine calcium, fluvastatine sodium, simvastatine, lovastatine, pravastatine sodium and the like.

As the calcium antagonist, aranidipine, lacidipine, naftopidil, felodipine, azelnidipine, cilnidipine, lomeridine, diltiazem, gallopamil, efonidipine, nisoldipine, amlodipine, lercanidipine, bevantolol, nicardipine, isradipine, benidipine, verapamil, nitrendipine, barnidipine, propafenone, manidipine, bepridil, nifedipine, nilvadipine, nimodipine, fasudil, pirmenol, carvedilol, trimetazidine, ethosuximide, zonisamide, felodipine, propiverine, manidipine, temiverine, ziconotide and the like can be mentioned.

As the fibrate, gemfibrozil, fenofibrate, bezafibrate, ciprofibrate, clinofibrate, clofibrate and the like can be mentioned.

As the diuretic, cicletanine hydrochloride, torasemide, tripamide, potassium canrenoate, isosorbide, piretanide, azosemide, indapamide, hydrochlorothiazide, trichlormethiazide, benzylhydrochlorothiazide, meticrane, chlortalidone, mefruside, furosemide, spironolactone, triamterene, amiloride and the like can be mentioned.

As the ACE inhibitor, trandolapril, moexipril, perindopril, quinapril hydrochloride, spirapril hydrochloride, temocapril, cilazapril, fosinopril, zofenopril calcium, imidapril hydrochloride, quinaprilate, benazepril hydrochlorde, lisinopril, captopril, ramipril, delapril, alacepril, enalapril malate, omapatrilat and the like can be mentioned.

As the angiotensin II antagonist, candesartan cilexetil, irbesartan, olmesartan medoxomil, telmisartan, valsartan, eprosartan mesilate, losartan potassium and the like can be mentioned.

As the cholesterol absorption inhibitor, colesevelam, ezetimibe, colestilan, colestyramine, ion exchange resin preparation and the like can be mentioned.

As the antioxidant, probucol, vitamin E and the like can be mentioned.

As the nicotinic acid derivative, tocopherol nicotinate, nicomol, niceritrol and the like can be mentioned.

As the squalene synthesis inhibitor, TAK-475, YM-53601 and the like can be mentioned.

As the aldose reductase inhibitor, lindolrestat, epalrestat, zenarestat, IDD-598, NZ-314, AS-3201 and the like can be mentioned.

As the PPAR modulator, thiazolidinedione antidiabetic agents such as rosiglitazone, pioglitazone, troglitazone, EML-16336 and the like, and the like can be mentioned.

As the β3 agonist, GRC-1087, YM-178, SR58611A, L 796568 and the like can be mentioned.

As the ACAT inhibitor, melinamide, eflucimibe, pactimibe and the like can be mentioned.

As the lipase inhibitor, docosanol, orlistat and the like can be mentioned.

As the anorexiant, mazindol and the like can be mentioned.

The present invention is described in more detail by way of the following Preparation Examples and Examples. However, they are not intended to limit the present invention in any way.

Some of the compounds obtained in the following Preparation Examples are encompassed within the scope of the present invention.

### EXPERIMENTAL EXAMPLE 1

Test Animal

Female C57BL/KsJ-db/db (db/db) mice and C57BL/KsJ-+m/+m (lean) mice (5 weeks old) were purchased from Jackson Laboratory, and subjected to the test after 1-2 weeks of acclimating period. Animals were maintained on standard laboratory chow and water ad libitum.

Administration of Test Compound

Test compound was dissolved or suspended in 0.5% methylcellulose solution and administered orally once a day at a volume of 5 mL/kg of body weight.

Test Compound:

4-({1-[(3,5-dichloro-2-pyridinyl)methyl]-2,4-dimethyl-1H-benzimidazol-6-yl}oxy)butanoic acid (Example 21)
4-{[1-(2,4-dichlorobenzyl)-2-ethoxy-4-methyl-1H-benzimidazol-6-yl]oxy}butanoic acid (Example 39)
2-{[(1-{[3-chloro-5-(trifluoromethyl)-2-pyridinyl]methyl}-2-methyl-1H-benzimidazol-6-yl)oxy]methyl}benzoic acid (Example 75)
   Test Schedule

At 6 weeks of age, blood was obtained from the retro-orbital sinus by capillary pipette with heparin under feeding condition. Mice were divided into groups matched for body weight, plasma glucose and triglyceride. From 6-7 weeks of age, test compound was administered orally once daily for 7 days. On the next morning of the last administration day, body weight was measured and blood was obtained under feeding condition, and plasma glucose and triglyceride were measured.

Measurement Method

Plasma glucose and triglyceride were determined by a mutarotase.glucose oxidase method and a glycerol-3-phosphate oxidase.3,5-dimethoxy-N-ethyl-N-(2′-hydroxy-3′-sulfopropyl)aniline natrium method, respectively, using assay kits from Wako Pure Chemical Industries, Ltd. (Osaka, Japan).

Result

Values of plasma glucose and triglyceride for the lean group (+m/+m) were set as 100% inhibition, and for the control group (db/db) as 0% inhibition. The inhibition rate of plasma glucose and triglyceride for the test group was determined. Results are shown in Table 1.

TABLE 1

| | | Inhibition | Inhibition |
|---|---|---|---|
| | | rate of plasma | rate of plasma |
| Test compound | Dose | glucose | triglyceride |
| (Example No.) | (mg/kg) | (%) | (%) |
| 21 | 10 | 41 | 55 |
| 39 | 10 | 45 | 60 |
| 75 | 10 | 72 | 89 |

### PREPARATION EXAMPLE 1

A mixture of 4-(acetylamino)-3-aminophenyl acetate (3.0 g), 1-(bromomethyl)-2-chloro-4-(pentyloxy)benzene (5.0 g), potassium carbonate (2.4 g) and N,N-dimethylformamide (30 mL) was stirred at 80° C. for half an hour. After cooling, the mixture was partitioned between ethyl acetate and water. The organic layer was separated, washed with brine, dried over anhydrous sodium sulfate, and concentrated in vacuo. The residue was purified by column chromatography on silica gel (gradient elution; chloroform to chloroform-methanol 100:1 to 5:1) to give 4-(acetylamino)-3-{[2-chloro-4-(pentyloxy)benzyl]amino}phenyl acetate (4.25 g) as a solid.

NMR(DMSO-d₆,δ): 0.88 (3H, t, J=7 Hz), 1.2-1.8 (6H, m), 2.04 (3H, s), 2.17 (3H, s), 3.95 (2H, t, J=6 Hz), 4.26 (2H, d, J=6 Hz), 5.76 (1H, t, J=6 Hz), 6.10 (1H, d, J=2 Hz), 6.29 (1H, dd, J=2 Hz, 8 Hz), 6.85 (1H, dd, J=2 Hz, 9 Hz), 7.02 (1H, d, J=2 Hz), 7.07 (1H, d, J=8 Hz), 7.28 (1H, d, J=9 Hz), 9.19 (1H, s).

### PREPARATION EXAMPLE 2

A mixture of 4-(acetylamino)-3-{[2-chloro-4-(pentyloxy)benzyl]amino}phenyl acetate (2.8 g), concentrated sulfuric acid (3 mL) and ethanol (30 mL) was stirred at 80° C. for 8 hours. After cooling, the mixture was concentrated in vacuo and the residue was neutralized with 4 N sodium hydroxide with cooling in an ice-bath. The resulting suspension was diluted-with ethyl acetate-and filterd to give 1-[2-chloro-4-(pentyloxy)benzyl]-2-methyl-1H-benzimidazol-6-ol (0.80 g) as a white solid.

NMR(DMSO-d₆,δ): 0.87 (3H, t, J=7 Hz), 1.15 (3H, t, J=7 Hz), 1.2-1.4 (4H, m), 1.6-2.1 (4H, m), 2.39 (3H, s), 2.43 (2H, t, J=7 Hz), 3.92 (2H, t, J=6 Hz), 3.93 (2H, t, J=6 Hz), 4.04 (2H, q, J=7 Hz), 5.39 (2H, s), 6.46 (1H, d, J=8 Hz), 6.7-6.9 (2H, m), 6.94 (1H, d, J=2 Hz), 7.10 (1H, d, J=2 Hz), 7.42 (1H, d, J=9 Hz).

### PREPARATION EXAMPLE 3

A mixture of 1-[2-chloro-4-(pentyloxy)benzyl]-2-methyl-1H-benzimidazol-6-ol (108 mg), ethyl bromoacetate (60 mg), potassium carbonate (62 mg) and N,N-dimethylformamide (2 mL) was stirred at 90° C. for an hour. Additional ethyl bromoacetate (10 mg) was added to the mixture and stirring was continued for 2 hours. After cooling, the mixture was partitioned between ethyl acetate and water. The organic layer was separated, washed with 1 N sodium hydroxide and brine, dried over anhydrous magnesium sulfate, and concentrated in vacuo. The residue was purified by column chromatography on silica gel (elution with 1:1 dichloromethane-ethyl acetate) to give ethyl ({1-[2-chloro-4-(pentyloxy)benzyl]-2-methyl-1H-benzimidazol-6-yl}oxy)acetate (63 mg).

MS(API-ES, Posi): 445.3.

### PREPARATION EXAMPLE 4

Ethyl 2-({1-[2-chloro-4-(pentyloxy)benzyl]-2-methyl-1H-benzimidazol-6-yl}oxy)-2-methylpropanoate (124 mg) was synthesized from 1-[2-chloro-4-(pentyloxy)benzyl]-2-methyl-1H-benzimidazol-6-ol (108 mg) in a manner similar to that described in Preparation Example 3 except that ethyl 2-bromo-2-methylpropanoate (294 mg) was used instead of ethyl bromoacetate.

NMR(DMSO-d₆,δ): 0.87 (3H, t, J=7.0 Hz), 1.09 (3H, t, J=7.1 Hz), 1.2-1.4 (6H, m), 1.42 (6H, s), 1.5-1.8 (2H, m), 2.46 (3H, s), 3.9-4.1 (4H, m), 5.35 (2H, s), 6.54 (1H, d, J=8.6 Hz), 7.6-7.9 (3H, m), 7.10 (1H, d, J=2.5 Hz), 7.42 (1H, dd, J=8.0 Hz, 1.3 Hz).

MS(API-ES, Posi): 473.4.

### PREPARATION EXAMPLE 5

To a suspension of 1-[2-chloro-4-(pentyloxy)benzyl]-2-methyl-1H-benzimidazol-6-ol (0.25 g) in a solvent mixture of tetrahydrofuran (2.5 mL) and N,N-dimethylformamide (2.5 mL) was added sodium hydride (60% dispersion in mineral oil; 31 mg). The mixture was stirred at 80° C. for 15 minutes and allowed to cool to ambient temperature. To the mixture was added ethyl 4-bromobutanoate (0.15 g) in an ice-bath. After stirring overnight at ambient temperature, the mixture was partitioned between ethyl acetate and saturated aqueous ammonium chloride. The organic layer was separated, washed with brine, dried over anhydrous sodium sulfate, and concentrated in vacuo. The residue was purified by column chromatography on silica gel (elution; 50:1 chloroform-methanol) to give ethyl 4-({1-[2-chloro-4-(pentyloxy)benzyl]-2-methyl-1H-benzimidazol-6-yl}oxy)butanoate (0.29 g) as an oil.

NMR(DMSO-d₆,δ): 0.87 (3H, t, J=7 Hz), 1.15 (3H, t, J=7 Hz), 1.2-1.4 (4H, m), 1.6-2.1 (4H, m), 2.39 (3H, s), 2.43 (2H, t, J=7 Hz), 3.92 (2H, t, J=6 Hz), 3.93 (2H, t, J=6 Hz), 4.04 (2H, q, J=7 Hz), 5.39 (2H, s), 6.46 (1H, d, J=8 Hz), 6.7-6.9 (2H, m), 6.94 (1H, d, J=2 Hz), 7.10 (1H, d, J=2 Hz), 7.42 (1H, d, J=9 Hz).

### PREPARATION EXAMPLE 6

Ethyl 5-({1-[2-chloro-4-(pentyloxy)benzyl]-2-methyl-1H-benzimidazol-6-yl}oxy)pentanoate (0.23 g) was synthesized from 1-[2-chloro-4-(pentyloxy)benzyl]-2-methyl-1H-benzimidazol-6-ol (200 mg) in a manner similar to that described in Preparation Example 5 except that ethyl 5-bromopentanoate (128 mg) was used instead of ethyl 4-bromobutanoate.

NMR(DMSO-d₆,δ): 0.87 (3H, t, J=7 Hz), 1.16 (3H, t, J=7 Hz), 1.2-1.7 (10H, m), 2.33 (2H, t, J=7 Hz), 2.39 (3H, s), 3.8-4.0 (4H, m), 4.03 (2H, q, J=7 Hz), 5.39 (2H, s), 6.48 (1H, d, J=8 Hz), 6.7-6.9 (2H, m), 6.94 (1H, d, J=2 Hz), 7.10 (1H, d, J=2 Hz), 7.39 (1H, d, J=9 Hz).

### PREPARATION EXAMPLE 7

Ethyl 6-({1-[2-chloro-4-(pentyloxy)benzyl]-2-methyl-1H-benzimidazol-6-yl}oxy)hexanoate (0.30 g) was synthesized from 1-[2-chloro-4-(pentyloxy)benzyl]-2-methyl-1H-benzimidazol-6-ol (250 mg) in a manner similar to that described in Preparation Example 5 except that ethyl 6-bromohexanoate (171 mg) was used instead of ethyl 4-bromobutanoate.

NMR(DMSO-d₆,δ): 0.87 (3H, t, J=7 Hz), 1.19 (3H, t, J=7 Hz), 1.2-1.7 (12H, m), 2.29 (2H, t, J=7 Hz), 2.39 (3H, s), 3.89 (2H, t, J=6 Hz), 3.93 (2H, t, J=6 Hz), 4.03 (2H, q, J=7 Hz), 5.39 (2H, s), 6.48 (1H, d, J=9 Hz), 6.74 (1H, dd, J=2 Hz, 9 Hz), 6.81 (1H, dd, J=2 Hz, 9 Hz), 6.93 (1H, d, J=2 Hz), 7.10 (1H, d, J=2 Hz), 7.41 (1H, d, J=9 Hz).

### PREPARATION EXAMPLE 8

A mixture of N-(2-hydroxy-6-nitrophenyl)acetamide (3 g), ethyl 4-bromobutanoate (3.43 g), potassium carbonate (2.54 g), potassium iodide (254 mg) and N,N-dimethylformamide (15 mL) was stirred at ambient temperature for 16 hours. The mixture was poured into water, and the suspension was stirred for 15 minutes and filtered to give ethyl 4-[2-(acetylamino)-3-nitrophenoxy]butanoate (4.48 g) as a solid.

NMR(DMSO-d₆,δ): 1.17 (3H, t, J=7.1 Hz), 1.9-2.1 (5H, m), 2.5-2.6 (2H, m), 4.0-4.1 (4H, m), 7.3-7.5 (3H, m), 9.69 (1H, s).

MS(API-ES, Posi): 333.2 (M+Na).

### PREPARATION EXAMPLE 9

A mixture of ethyl 4-[2-(acetylamino)-3-nitrophenoxy]butanoate (333 mg), 1-(bromomethyl)-2-chloro-4-(pentyloxy)benzene (375 mg), potassium carbonate (193 mg) and N,N-dimethylformamide (3 mL) was stirred at 100° C. for 16 hours. After cooling, the mixture was partitioned between ethyl acetate and water. The organic layer was separated, washed successively with 1 N sodium hydroxide, saturated aqueous ammonium chloride, water and brine, dried over anhydrous magnesium sulfate, and concentrated in vacuo. The residue was triturated with diisopropyl ether and filtered to remove the starting material. The filtrate was concentrated in vacuo, and the residue was dissolve in a solvent mixture of acetic acid (3 mL) and ethanol (9 mL). The mixture was heated with iron (300 mg) at 80° C. for 2 hours and at 110° C. for 2 hours. After cooling, the mixture was concentrated in vacuo and the residue was neutralized with sodium bicarbonate. The suspension was diluted with ethyl acetate and stirred for 5 minutes. The insoluble materials were filtered off, and the filtrate was washed with water and brine, dried over anhydrous magnesium sulfate, and concentrated in vacuo. The residue was purified by column chromatography on silica gel (elution; 2% methanol in dichloromethane) to give ethyl 4-({1-[2-chloro-4-(pentyloxy)benzyl]-2-methyl-1H-benzimidazol-7-yl}oxy)butanoate (322 mg).

NMR(DMSO-d₆,δ): 0.87 (3H, t, J=7.1 Hz), 1.15 (3H, t, J=7.1 Hz), 1.3-1.4 (4H, m), 1.6-1.8 (4H, m), 2.14 (2H, t, J=7.8 Hz), 2.40 (3H, s), 3.9-4.1 (6H, m), 5.58 (2H, s), 6.11 (1H, d, J=8.6 Hz), 6.7-6.9 (2H, m), 7.0-7.1 (1H, m), 7.16 (1H, d, J=8.1 Hz).

MS(API-ES, Posi): 473.3.

### PREPARATION EXAMPLE 10

A mixture of ethyl 4-[2-(acetylamino)-3-nitrophenoxy]butanoate (1 g), 10% palladium on carbon (50% wet; 200 mg), tetrahydrofuran (5 mL) and ethanol (10 mL) was stirred under 1 atmosphere of hydrogen at ambient temperature for 3 hours. The catalyst was filtered off and the filtrate was concentrated in vacuo. The residue was triturated with diisopropyl ether to give ethyl 4-[2-(acetylamino)-3-aminophenoxy]butanoate (805 mg) as a white solid.

NMR(DMSO-d₆,δ): 1.18 (3H, t, J=7.1 Hz), 1.8-1.9 (2H, m), 2.00 (3H, s), 2.45 (2H, t, J=7.5 Hz), 3.88 (2H, t, J=6.2 Hz), 4.06 (2H, q, J=7.1 Hz), 4.74 (2H, s), 6.20 (1H, d, J=8.0 Hz), 6.31 (1H, d, J=8.0 Hz), 6.86 (1H, t, J=8.0 Hz), 8.70 (1H, s).

MS(API-ES, Posi): 303 (M+Na).

### PREPARATION EXAMPLE 11

A mixture of ethyl 4-[2-(acetylamino)-3-aminophenoxy]butanoate (400 mg), 1-(bromomethyl)-2-chloro-4-(pentyloxy)benzene (541 mg), potassium carbonate (256 mg) and N,N-dimethylformamide (10 mL) was stirred at ambient temperature for 16 hours. The mixture was partitioned between ethyl acetate and water. The organic layer was separated, washed with water (twice) and brine, dried over anhydrous magnesium sulfate, and concentrated in vacuo to give ethyl 4-(2-(acetylamino)-3-{[2-chloro-4-(pentyloxy)benzyl]amino}phenoxy)butanoate (374 mg).

NMR(DMSO-d₆,δ): 0.88 (3H, t, J=7.1 Hz), 1.86 (3H, t, J=7.1 Hz), 1.3-1.4 (4H, m), 1.6-1.9 (4H, m), 2.03 (3H, s), 2.46 (2H, t, J=7.4 Hz), 3.9-4.0 (4H, m), 4.06 (2H, q, J=7.1 Hz), 4.28 (2H, d, J=6.3 Hz), 5.58 (1H, t, J=6.3 Hz), 5.96 (1H, d, J=8.1 Hz), 6.81 (1H, dd, J=8.6 Hz, 2.5 Hz), 6.89 (1H, t, J=8.3 Hz), 7.00 (1H, d, J=2.5 Hz), 7.22 (1H, d, J=8.6 Hz), 8.75 (1H, s).

MS(API-ES, Posi): 491.3.

### PREPARATION EXAMPLE 12

Ethyl 4-({1-[2-chloro-4-(pentyloxy)benzyl]-2-methyl-1H-benzimidazol-4-yl}oxy)butanoate (192 mg) was synthesized from ethyl 4-(2-(acetylamino)-3-{[2-chloro-4-(pentyloxy)benzyl]amino}phenoxy}butanoate (200 mg) in a manner similar to that described in Preparation Example 27.

NMR(DMSO-d₆,δ): 0.87 (3H, t, J=7.1 Hz), 1.18 (3H, mt, J=7.1 Hz), 1.3-1.4 (4H, m), 1.6-1.7 (2H, m), 2.0-2.1 (2H, m), 2.44 (3H, s), 3.93 (2H, t, J=6.5 Hz), 4.08 (22H, sq, J=7.1 Hz), 4.21 (3H, t, J=6.3 Hz), 5.40 (2H, s), 6.48 (1H, d, J=8.7 Hz), 6.66 (1H, d, J=7.7 Hz), 6.80 (1H, dd, J=8.6, 2.6 Hz), 6.90 (1H, d, J=8.0 Hz), 7.01 (1H, t, J=8.0 Hz), 7.09 (1H, d, J=2.6 Hz).

MS(API-ES, Posi): 473.3.

### PREPARATION EXAMPLE 13

tert-Butyl [2-(acetylamino)-3-nitrophenoxy]acetate (1.41 g) was synthesized from N-(2-hydroxy-6-nitrophenyl)acetamide (895 mg) in a manner similar to that described in Preparation Example 8 except that tert-butyl bromoacetate (1.02 g) was used instead of ethyl 4-bromobutanoate.

MS(API-ES, Posi): 333.3(M+Na).

### PREPARATION EXAMPLE 14

tert-Butyl (2-{acetyl[2-chloro-4-(pentyloxy)benzyl]amino}-3-nitrophenoxy)acetate (239 mg) was synthesized from tert-butyl [2-(acetylamino)-3-nitrophenoxy]acetate (310 mg) in a manner similar to that described in Preparation Example 26.

NMR(DMSO-d₆,δ): 0.87 (3H, t, J=7.0 Hz), 1.3-1.4 (4H, m), 1.43 (9H, s), 1.6-1.7 (2H, m), 1.87 (3H, s), 3.92 (2H, t, J=6.5 Hz), 4.46 (1H, d, J=7.1 Hz), 4.72 (1H, d, J=8.2 Hz), 4.82 (1H, d, J=8.2 Hz), 5.01 (1H, d, J=7.1 Hz), 6.74 (1H, dd, J=4.3 Hz, 1.3 Hz), 6.83 (1H, d, J=1.3 Hz), 7.05 (1H, d, J=4.3 Hz), 7.35 (1H, dd, J=4.3 Hz, 0.6 Hz), 7.45 (1H, dd, J=4.1 Hz, 0.6 Hz), 7.55 (1H, t, J=4.2 Hz).

MS(ESI, Posi): 521.29.

### PREPARATION EXAMPLE 15

tert-Butyl ({1-[2-chloro-4-(pentyloxy)benzyl]-2-methyl-1H-benzimidazol-7-yl}oxy)acetate (215 mg) was synthesized from tert-butyl (2-{acetyl [2-chloro-4-(pentyloxy)benzyl]amino}-3-nitrophenoxy)acetate (222 mg) in a manner similar to that described in Preparation Example 27.

NMR(DMSO-d₆,δ): 0.87 (3H, t, J=7.1 Hz), 1.2-1.4 (13H, m), 1.6-1.7 (2H, m), 2.36 (3H, s), 3.92 (2H, t, J=6.5 Hz), 4.65 (2H, s), 5.71 (2H, s), 6.34 (1H, d, J=8.7 Hz), 6.69 (1H, d, J=7.6 Hz), 6.77 (1H, dd, J=8.7 Hz, 2.6 Hz), 7.0-7.1 (2H, m), 7.19 (1H, d, J=7.5 Hz).

MS(API-ES, Posi): 473.2.

### PREPARATION EXAMPLE 16

Ethyl 6-[2-(acetylamino)-3-nitrophenoxy]hexanoate (9.55 g) was synthesized from N-(2-hydroxy-6-nitrophenyl)acetamide (6.0 g) in a manner similar to that described in Preparation Example 25.

### PREPARATION EXAMPLE 17

Ethyl 6-[2-(acetylamino)-3-aminophenoxy]hexanoate (1.0 g) was synthesized from 6-[2-(acetylamino)-3-nitrophenoxy]hexanoate (1.5 g) in a manner similar to that described in Preparation Example 10.

NMR(DMSO-d₆,δ): 1.17 (3H, t, J=7 Hz), 1.3-1.7 (6H, m), 1.98 (3H, s), 2.29 (2H, t, J=7 Hz), 3.83 (2H, t, J=6 Hz), 4.04 (2H, q, J=7 Hz), 4.72 (2H, br s), 6.20 (1H, d, J=8 Hz), 6.30 (1H, d, J=7 Hz), 6.86 (1H, t-like, J=8 Hz), 8.67 (1H, br s).

### PREPARATION EXAMPLE 18

A mixture of ethyl 6-[2-(acetylamino)-3-aminophenoxy]hexanoate (0.95 g), 1-(bromomethyl)-2-chloro-4-(pentyloxy)benzene (1.0 g), potassium carbonate (0.51 g) and N,N-dimethylformamide (6 mL) was stirred at 80° C. for 2 hours. After cooling, the mixture was partitioned between ethyl acetate and water. The organic layer was separated, washed with brine, dried over anhydrous sodium sulfate, and concentrated in vacuo to give ethyl 6-(2-(acetylamino)-3-{[2-chloro-4-(pentyloxy)benzyl]amino}phenoxy)hexanoate (1.59 g).

### PREPARATION EXAMPLE 19

Ethyl 6-({1-[2-chloro-4-(pentyloxy)benzyl]-2-methyl-1H-benzimidazol-4-yl}oxy)hexanoate (1.16 g) was synthesized from ethyl 6-(2-(acetylamino)-3-{[2-chloro-4-(pentyloxy)benzyl]amino}phenoxy)hexanoate (1.59 g) in a manner similar to that described in Preparation Example 27.

NMR(DMSO-d₆,δ): 0.87 (3H, t, J=7 Hz), 1.17 (3H, t, J=7 Hz), 2.32 (2H, t, J=6 Hz), 1.2-1.8 (12H, m), 2.44 (3H, s), 3.93 (2H, t, J=6 Hz), 4.05 (2H, q, J=7 Hz), 4.17 (2H, t, J=6 Hz), 5.39 (2H, s), 6.47 (1H, d, J=9 Hz), 6.65 (1H, d, J=7 Hz), 6.7-7.1 (3H, m), 7.09 (1H, d, J=2 Hz).

### PREPARATION EXAMPLE 20

Ethyl 6-(2-{acetyl[2-chloro-4-(pentyloxy)benzyl]amino}-3-nitrophenoxy)hexanoate (1.6 g) was synthesized ethyl 6-[2-(acetylamino)-3-nitrophenoxy]hexanoate (1.0 g) in a manner similar to that described in Example 26.

### PREPARATION EXAMPLE 21

Ethyl 6-({1-[2-chloro-4-(pentyloxy)benzyl]-2-methyl-1H-benzimidazol-7-yl}oxy)hexanoate (0.73 g) was synthesized from ethyl 6-(2-{acetyl[2-chloro-4-(pentyloxy)benzyl]amino}-3-nitrophenoxy)hexanoate (1.622 g) in a manner similar to that described in Preparation Example 27.

### PREPARATION EXAMPLE 22

A mixture of 4-(acetylamino)-3-nitrophenyl acetate (1.19 g), 1-(bromomethyl)-2-chloro-4-(pentyloxy)benzene (1.89 g), potassium carbonate (898 mg) and N,N-dimethylformamide (12 mL). was stirred at ambient temperature for 4 days. The mixture was partitioned between ethyl acetate and water. The organic layer was separated, washed successively with aqueous ammonium chloride (twice), water and brine, dried over anhydrous magnesium sulfate, and concentrated in vacuo. The residue was purified by column chromatography on silica gel (elution with 2:1. n-hexane-ethyl acetate) to give 4-{acetyl[2-chloro-4-(pentyloxy)benzyl]amino}-3-nitrophenyl acetate (1.36 g) as a yellow oil.

MS(API-ES, posi): 471.1(M+Na).

### PREPARATION EXAMPLE 23

A mixture of 4-{acetyl[2-chloro-4-(pentyloxy)benzyl]amino}-3-nitrophenyl acetate (1.35 g), iron (672 mg), acetic acid (3 mL) and ethanol (9 mL) was stirred at 80° C. for 2 hours. After cooling, the mixture was concentrated in vacuo. The residue was neutralized with sodium hydrogen carbonate and diluted with ethyl acetate. The suspension was stirred at ambient temperature for 5 minutes and filtered through a celite pad. The filtrate was washed with water and brine, dried over anhydrous magnesium sulfate, and concentrated in vacuo. The residue was dissolved in ethanol (5 mL) and concentrated sulfuric acid (1 mL) was added. The mixture was refluxed for 2 hours and allowed to cool to ambient temperature. The mixture was concentrated in vacuo, and the residue was neutralized with sodium hydroxide and partitioned between ethyl acetate and water. The organic layer was separated, washed with brine, dried over anhydrous sodium sulfate, and concentrated in vacuo. The residue was triturated with ethanol followed by filtration to give 1-[2-chloro-4-(pentyloxy)benzyl]-2-methyl-1H-benzimidazol-5-ol (100 mg) as a white solid. The filtrate was concentrated in vacuo and the residue was triturated with diisopropyl ether to give the further product (92 mg).

NMR(DMSO-d₆,δ); 0.87 (3H, t, J=7.0 Hz), 1.2-1.4 (4H, m), 1.6-1.8 (2H, m), 2.41 (3H, s), 3.93 (2H, t, J=6.4 Hz), 5.34 (2H, s), 6.51 (1H, d, J=8.7 Hz), 6.60 (1H, dd, J=8.6 Hz, 2.3 Hz), 6.81 (1H, dd, J=8.6 Hz, 2.5 Hz), 6.88 (1H, d, J=2.2 Hz), 7.0-7.1 (2H, m), 8.93 (1H, s).

MS(API-ES, Posi): 359.2.

### PREPARATION EXAMPLE 24

Ethyl 4-({1-[2-chloro-4-(pentyloxy)benzyl]-2-methyl-1H-benzimidazol-5-yl}oxy)butanoate (64 mg) was synthesized from 1-[2-chloro-4-(pentyloxy)benzyl]-2-methyl-1H-benzimidazol-5-ol (90 mg) in a manner similar to that described in Preparation Example 5.

NMR(DMSO-d₆,δ); 0.87 (3H, t, J=7.1 Hz), 1.17 (3H, t, J=7.1 Hz), 1.2-1.4 (4H, m), 1.6-1.7 (2H, m), 1.9-2.0 (2H, m), 2.4-2.5 (5H, m), 3.93 (2H, t, J=6.5 Hz), 3.98 (2H, t, J=6.3 Hz), 4.06 (2H, q, J=7.1 Hz), 5.39 (2H, s), 6.51 (1H, d, J=8.6 Hz), 6.74 (1H, dd, J=8.8 Hz, 2.4 Hz), 6.81 (1H, dd, J=8.6 Hz, 2.6 Hz), 7.0-7.1 (2H, m), 7.18 (1H, d, J=8.8 Hz).

MS(API-ES, Posi): 473.3.

### PREPARATION EXAMPLE 25

A mixture of N-(4-hydroxy-2-nitrophenyl)acetamide (1.6 g), ethyl 6-bromohexanoate (2.0 g), potassium carbonate (1.2 g) and N,N-dimethylformamide (30 mL) was stirred at ambient temperature overnight. The mixture was diluted with EtOAc (30 mL) and the insoluble materials were filtered off. The filtrate was washed successively with aq. sodium bicarbonate, water and brine, dried over anhydrous magnesium sulfate, and concentrated in vacuo. The resulting solid was triturate with diisopropyl ether (50 mL) and filtered to give ethyl 6-[4-(acetylamino)-3-nitrophenoxy]hexanoate (1.9 g) as a yellow solid.

NMR(DMSO-d₆,δ): 1.17 (3H, t, J=7 Hz), 1.4-1.8 (6H, m), 2.01 (3H, s), 2.30 (2H, t, J=7 Hz), 4.02 (2H, t, J=6 Hz), 4.04 (2H, q, J=7 Hz), 7.26 (1H, dd, J=3 Hz, 9 Hz), 7.4-7.5 (2H, m), 10.02 (1H, br s).

### PREPARATION EXAMPLE 26

To a solution of ethyl 6-[4-(acetylamino)-3-nitrophenoxy]hexanoate (1.5 g) in a solvent mixture of tetrahydrofuran (15 mL) and N,N-dimethylformamide (15 mL) was added sodium hydride (60% dispersion in mineral oil; 186 mg) in an ice-bath. The ice-bath was removed and the mixture was stirred at ambient temperature for half an hour. To the mixture was added 1-(bromomethyl)-2-chloro-4-(pentyloxy)benzene (1.55 g) and stirring was continued for 3 hours at ambient temperature. The mixture was partitioned between ethyl acetate and saturated aqueous ammonium chloride. The organic layer was separated, washed with water and brine, dried over anhydrous magnesium sulfate, and concentrated in vacuo to give ethyl 6-(4-{acetyl[2-chloro-4-(pentyloxy)benzyl]amino}-3-nitrophenoxy)hexanoate (2.4 g) as a yellow oil.

NMR(DMSO-d₆,δ): 0.88 (3H, t, J=7 Hz), 1.16 (3H, t, J=7 Hz), 1.2-1.8 (12H, m), 1.78 (3H, s), 2.29 (2H, t, J=7 Hz), 3.92 (2H, t, J=6 Hz), 4.03 (2H, t, J=6 Hz), 4.04 (2H, q, J=7 Hz), 5.02, 4.44 (2H AB q, J=14 Hz), 6.81 (1H, dd, J=2 Hz, 8 Hz), 6.89 (1H, d, J=2 Hz), 7.15 (1H, d, J=8 Hz), 7.1-7.3 (2H, m), 7.52 (1H, d, J=3 Hz).

### PREPARATION EXAMPLE 27

A mixture of ethyl 6-(4-{acetyl[2-chloro-4-(pentyloxy)benzyl]amino}-3-nitrophenoxy)hexanoate (2.4 g) and iron (977 mg) in a solvent mixture of ethanol (25 mL) and acetic acid (7.5 mL) was reflux for 5 hours. After cooling, the insoluble materials were filtered off and the residue was partitioned between ethyl acetate and 1 N sodium hydroxide. The organic layer was separated, washed with brine, dried over anhydrous magnesium sulfate, and concentrated in vacuo. The residue was purified by column chromatography on silica gel (gradient elution; n-hexane-ethyl acetate 1:1 to 1:2 to 1:3) to give ethyl 6-({1-[2-chloro-4-(pentyloxy)benzyl]-2-methyl-1H-benzimidazol-5-yl}oxy)hexanoate (1.58 g) as a solid.

NMR(DMSO-d₆,δ): 0.87 (3H, t, J=7 Hz), 1.17 (3H, t, J=7 Hz), 1.2-1.8 (12H, m), 2.30 (2H, t, J=7 Hz), 2.43 (3H, s), 3.93 (2H, t, J=6 Hz), 3.94 (2H, t, J=6 Hz), 4.04 (2H, q, J=7 Hz), 5.38 (2H, s), 6.51 (1H, d, J=9 Hz), 6.73 (1H, dd, J=2 Hz, 9 Hz), 6.80 (1H, dd, J=3 Hz, 9 Hz), 7.0-7.1 (2H, m), 7.17 (1H, d, J=9 Hz).

### PREPARATION EXAMPLE 28

To nitric acid (HNO₃) (fuming) (8 ml) was added 4-(acetylamino)-3-methylphenyl acetate (2.0 g) at −40-−50° C. After stirring for 2 hours, the reaction mixture was added ice-water and neutralized with 20%-sodium hydroxide (NaOH). The precipitates were collected by filtration and washed with water (3×5 ml). The crude crystals were triturated with ethyl acetate (EtOAc) to give 4-(acetylamino)-3-methyl-5-nitrophenyl acetate (0.91 g) as pale yellow crystals.

NMR(DMSO-d₆,δ): 2.04 (3H, s), 2.28 (3H, s), 2.29 (3H, s), 7.44 (1H, d, J=2.5 Hz), 7.62 (1H, d, J=2.5 Hz), 9.85 (1H, br s).

MS: 275 (M+Na).

### PREPARATION EXAMPLE 29

To a suspension of 4-(acetylamino)-3-methyl-5-nitrophenyl acetate (500 mg) in a mixture of ethyl alcohol (EtOH) (5 ml) and tetrahydrofuran (THF) (2.5 ml) was added palladium on carbon (10%, 50% wet, 0.2 g) at ambient temperature, and the resultant mixture was hydrogenated under atmospheric pressure of hydrogen for 3 hours. The catalyst was removed by filtration (washed with EtOH). The filtrate was evaporated and triturated with EtOAc to give 4-(acetylamino)-3-amino-5-methylphenyl acetate (377 mg) as crystals.

NMR(DMSO-d₆,δ): 2.01 (3H, s), 2.02 (3H, s), 2.21 (3H, s), 4.95 (2H, br s), 6.14 (1H, d, J=2.5 Hz), 6.26 (1H, d, J=2.5 Hz), 8.82 (1H, br s).

MS: 245 (M+Na).

### PREPARATION EXAMPLE 30

A mixture of 4-(acetylamino)-3-amino-5-methylphenyl acetate (50 mg), 1-(bromomethyl)-2-chloro-4-(pentyloxy)benzene (66 mg), potassium carbonate (K₂CO₃) (31 mg) and N,N-dimethylformamide (DMF) (1 ml) was heated at 80° C. for 3 hours. After cooling, the reaction mixture was diluted with EtOAc (40 ml) and washed with water (2×30 ml) and brine (20 ml). The organic layer was dried over magnesium sulfate (MgSO₄). filtered, and evaporated to give 4-(acetylamino)-3-{[2-chloro-4-(pentyloxy)benzyl]amino}-5-methylphenyl acetate (109 mg) as a crude oil.

NMR(DMSO-d₆,δ): 0.88 (3H, t, J=3.5 Hz), 1.2-1.5 (4H, m), 1.6-1.8 (2H, m), 2.04 (3H, s), 2.06 (3H, s), 2.15 (3H, s), 3.94 (2H, t, J=3.2 Hz), 4.25 (2H, d, J=3.1 Hz), 5.78 (1H, t, J=3.1 Hz), 5.89 (1H, d, J=1.2 Hz), 6.18 (1H, d, J=1.2 Hz), 6.82 (1H, dd, J=1.3 Hz, 4.3 Hz), 7.01 (1H, d, J=1.3 Hz), 7.23 (1H, d, J=4.3 Hz), 8.92 (1H, br s).

MS: 433 (M+1).

### PREPARATION EXAMPLE 31

A mixture of 4-(acetylamino)-3-{[2-chloro-4-(pentyloxy)benzyl]amino}-5-methylphenyl acetate (600 mg) and 4N-hydrochloric acid (HCl)/dioxane (3.5 ml) was stirred at ambient temperature for 18 hours. The reaction mixture was poured into ice-water, neutralized with saturated aqueous sodium hydrogencarbonate (NaHCO₃), and extracted with EtOAc (2×50 ml). The combined organic layers were dried over MgSO4 and filtered. Evaporation gave a residue (370 mg) which was chromatographed (silica gel, dichloromethane (CH₂Cl₂)—methyl alcohol (MeOH); 1→2%) to give 1-[2-chloro-4-(pentyloxy)benzyl]-2,4-dimethyl-1H-benzimidazol-6-yl acetate (204 mg) as a pale brown oil.

NMR(DMSO-d₆,δ): 0.87 (3H, t, J=6.7 Hz), 1.2-1.4 (4H, m), 1.5-1.8 (2H, m), 2.22 (3H, s), 2.45 (3H, s), 2.49 (3H, s), 3.94 (2H, t, J=6.4 Hz), 5.39 (2H, br s), 6.47 (1H, d, J=8.7 Hz), 6.74 (1H, d, J=2.0 Hz), 6.82 (1H, dd, J=2.5 Hz, 8.7 Hz), 6.99 (1H, d, J=2.0 Hz), 7.10 (1H, d, J=2.5 Hz).

MS: 415 (M+1).

### PREPARATION EXAMPLE 32

To a solution of 1-[2-chloro-4-(pentyloxy)benzyl]-2,4-dimethyl-1H-benzimidazol-6-yl acetate (197 mg) in MeOH (2 ml) was added K₂CO₃ (146 mg) at ambient temperature. After stirring for 2 hours, the reaction mixture was neutralized with 1 N-HCl. The precipitates were collected by filtration and washed with water (3×2 ml) and MeOH (1 ml) to give 1-[2-chloro-4-(pentyloxy)benzyl]-2,4-dimethyl-1H-benzimidazol-6-ol (142 mg) as pale brown crystals.

NMR(DMSO-d₆,δ): 0.87 (3H, t, J=6.8 Hz), 1.2-1.4 (4H, m), 1.5-1.8 (2H, m), 2.41 (3H, s), 2.42 (3H, s), 3.94 (2H, t, J=6.4 Hz), 5.28 (2H, br s), 6.38 (1H, d, J=2.0 Hz), 6.4-6.5 (2H, m), 6.82 (1H, dd, J=2.5 Hz, 8.6 Hz), 7.10 (1H, d, J=2.5 Hz), 8.98 (1H, br s).

MS: 373 (M+1).

### PREPARATION EXAMPLE 33

To a solution of 1-[2-chloro-4-(pentyloxy)benzyl]-2,4-dimethyl-1H-benzimidazol-6-ol (50 mg) in a mixture of DMF (0.5 ml) and THF (0.5 ml) was added sodium hydride (NaH; 60% dispersion in mineral oil) (6.4 mg) at ambient temperature. After stirring for 30 minutes, the reaction mixture was added ethyl 4-bromobutanoate (29 mg) and the stirring was continued for 12 hours. The reaction mixture was added water (30 ml) and extracted with EtOAc (2×20 ml). The combined organic extracts were washed with brine (20 ml), dried over MgSO₄, and filtered. Evaporation gave a residue (63 mg) which was chromatographed (preparative thin-layer chromatography (TLC), EtOAc/n-hexane=2/1) to give ethyl 4-({1-[2-chloro-4-(pentyloxy)benzyl]-2,4-dimethyl-1H-benzimidazol-6-yl}oxy)butanoate (50 mg) as an oil.

NMR(DMSO-d₆,δ): 0.87 (3H, t, J=6.8 Hz), 1.16 (3H, t, J=7.1 Hz), 1.2-1.4 (4H, m), 1.5-1.8 (2H, m), 2.39 (3H, s), 2.3-2.5 (5H, m), 3.8-4.0 (4H, m), 4.05 (2H, q, J=7.1 Hz), 5.37 (2H, br s), 6.42 (1H, d, J=8.6 Hz), 6.59 (1H, d, J=2.0 Hz), 6.76 (1H, d, J=2.0 Hz), 6.81 (1H, dd, J=2.5 Hz, 8.6 Hz), 7.10 (1H, d, J=2.5 Hz).

MS: 487 (M+1).

### PREPARATION EXAMPLE 34

To a suspension of 4-(acetylamino)-3-methyl-5-nitrophenyl acetate (330 mg) in EtOH (6.6 ml) was added sulfuric acid (H₂SO₄) (1.28 g) at ambient temperature. After stirring for 2 hours, the reaction mixture was diluted with ice-water (20 ml), and the pH of the mixture was adjusted to around 3.5 with 20%-NaOH, and then extracted with EtOAc (2×30 ml). The combined organic extracts were washed with brine (20 ml), dried over MgSO₄, and evaporated to give N-(4-hydroxy-2-methyl-6-nitrophenyl)acetamide (274 mg) as dark yellow crystals.

NMR(DMSO-d₆,δ): 1.98 (3H, s), 2.17 (3H, s), 6.98 (1H, d, J=2.8 Hz), 7.06 (1H, d, J=2.8 Hz), 9.52 (1H, br s), 10.20 (1H, br s).

MS: 233 (M+Na).

### PREPARATION EXAMPLE 35

To a suspension of NaH (60% dispersion in mineral oil) (1.37 g) was washed with ether (Et₂O) twice) in a mixture of DMF (60 ml) and THF (60 ml) was added N-(4-hydroxy-2-methyl-6-nitrophenyl)acetamide (6.0 g) under ice cooling. After stirring for 30 minutes at ambient temperature, the reaction mixture was added ethyl 4-bromobutanoate (6.12 g) and the stirring was continued for 5 days. The reaction mixture was added water (100 ml) and extracted with EtOAc (3×100 ml). The combined organic extracts were washed with brine (2×200 ml), dried over MgSO₄, and filtered. Evaporation gave a residue which was triturated with EtOAc (6 ml)—n-hexane (6 ml) to give ethyl 4-[4-(acetylamino)-3-methyl-5-nitrophenoxy]butanoate (4.57 g) as pale yellow crystals.

NMR(DMSO-d₆,δ): 1.18 (3H, t, J=7.1 Hz), 1.8-2.1 (2H, m), 1.99 (3H, s), 2.22 (3H, s), 2.45 (2H, t, J=7.2 Hz), 4.0-4.2 (4H, m), 7.20 (1H, d, J=2.8 Hz), 7.26 (1H, d, J=2.8 Hz), 9.62 (1H, br s).

MS: 347 (M+Na).

### PREPARATION EXAMPLE 36

To a suspension of ethyl 4-[4-(acetylamino)-3-methyl-5-nitrophenoxy]butanoate (4.5 g) in EtOH (45 ml) was added palladium on carbon (10%, 50% wet, 1.35 g) at ambient temperature, and the resultant mixture was hydrogenated under atmospheric pressure of hydrogen for 6 hours. The catalyst was removed by filtration. The filtrate was evaporated to give ethyl 4-[4-(acetylamino)-3-amino-5-methylphenoxy]butanoate (4.33 g) as an oil.

NMR(DMSO-d₆,δ): 1.18 (3H, t, J=7.1 Hz), 1.8-2.1 (2H, m), 1.99 (3H, s), 2.42 (2H, t, J=7.2 Hz), 3.85 (2H, t, J=6.3 Hz), 4.07 (2H, q, J=7.1 Hz), 4.73 (2H, br s), 5.99 (1H, d, J=2.6 Hz), 6.09 (1H, d, J=2.6 Hz), 8.68 (1H, br s).

MS: 317 (M+Na).

### PREPARATION EXAMPLE 37

To a suspension of ethyl 4-[4-(acetylamino)-3-amino-5-methylphenoxy]butanoate (4.33 g) in EtOH (20 ml) was added H₂SO₄ (2 ml) at ambient temperature. After stirring for 23 hours, the reaction mixture was heated at 50° C. for 5 hours. After cooling, the reaction mixture was evaporated, ice was added, and the pH of the mixture was adjusted to around 8.5 with 20%-NaOH. The mixture was extracted with EtOAc (2×50 ml). The combined organic extracts were washed with brine (50 ml), dried over MgSO₄, and filtered. Evaporation gave a residue which was triturated with EtOAc (4 ml)—n-hexane (4 ml) to give ethyl 4-[(2,4-dimethyl-1H-benzimidazol-6-yl)oxy]butanoate (2.9 g) as white crystals.

NMR(DMSO-d₆,δ): 1.18 (3H, t, J=7.1 Hz), 1.8-2.1 (2H, m), 2.3-2.5 (8H, m), 3.94 (2H, t, J=6.3 Hz), 4.07 (2H, q, J=7.1 Hz), 6.53 (1H, br s), 6.74 (1H, br s), 11.93 (1H, br s).

MS: 277 (M+1).

### PREPARATION EXAMPLE 38

To a mixture of ethyl 4-[(2,4-dimethyl-1H-benzimidazol-6-yl)oxy]butanoate (200 mg), K₂CO₃ (130 mg) and DMF (2 ml) was added 2,4-dichloro-1-(chloromethyl)benzene (170 mg) at ambient temperature. After stirring for 24 hours, the reaction mixture was heated at 70° C. for 7 hours. After cooling, the reaction mixture was diluted with EtOAc (50 ml) and washed with 5%-NaHCO₃ (20 ml) and brine (2×30 ml). The organic layer was dried over MgSO₄ and filtered. Evaporation gave a residue (346 mg) which was triturated with n-hexane (4 ml) to give ethyl 4-{[1-(2,4-dichlorobenzyl)-2,4-dimethyl-1H-benzimidazol-6-yl]oxy}butanoate (280 mg) as white crystals.

NMR(DMSO-d₆,δ): 1.15 (3H, t, J=7.1 Hz), 1.8-2.1 (2H, m), 2.3-2.5 (8H, m), 3.90 (2H, t, J=6.3 Hz), 4.04 (2H, q, J=7.1 Hz), 5.44 (2H, br s), 6.39 (1H, d, J=8.4 Hz), 6.61 (1H, d, J=2.0 Hz), 6.79 (1H, d, J=2.0 Hz), 7.32 (1H, dd, J=2.1 Hz, 8.4 Hz), 7.72 (1H, d, J=2.1 Hz).

MS: 435 (M+1).

### PREPARATION EXAMPLE 39

To a mixture of ethyl 4-[(2,4-dimethyl-1H-benzimidazol-6-yl)oxy]butanoate (200 mg), K₂CO₃ (130 mg) and DMF (2 ml) was added 3-chloro-2-(chloromethyl)-5-(trifluoromethyl)pyridine (200 mg) at ambient temperature. After stirring for 24 hours, the reaction mixture was diluted with EtOAc (50 ml) and washed with 5%-NaHCO₃ (30 ml) and brine (2×30 ml). The organic layer was dried over MgSO₄ and filtered. Evaporation gave a residue which was triturated with n-hexane (4 ml) to give ethyl 4-[(1-{[3-chloro-5-(trifluoromethyl)-2-pyridinyl]methyl}-2,4-dimethyl-1H-benzimidazol-6-yl)oxy]butanoate (290 mg) as white crystals.

NMR(DMSO-d₆,δ): 1.15 (3H, t, J=7.1 Hz), 1.8-2.0 (2H, m), 2.3-2.5 (8H, m), 3.89 (2H, t, J=6.3 Hz), 4.04 (2H, q, J=7.1 Hz), 5.70 (2H, br s), 6.56 (!H, d, J=1.8 Hz), 6.77 (1H, d, J=1.8 Hz), 8.55 (1H, d, J=1.2 Hz), 8.77 (1H, d, J=1.2 Hz).

MS: 470 (M+1).

### PREPARATION EXAMPLE 40

To a mixture of ethyl 4-[(2,4-dimethyl-1H-benzimidazol-6-yl)oxy]butanoate (200 mg), K₂CO₃ (130 mg) and DMF (2 ml) was added 4-(chloromethyl)-5-methyl-2-phenyl-1,3-oxazole (180 mg) at ambient temperature. After stirring for 13 hours at 70-90° C., the reaction mixture was diluted with EtOAc (30 ml) and washed with 5%-NaHCO₃ (20 ml) and brine (2×20 ml). The organic layer was dried over MgSO₄ and filtered. Evaporation gave a residue (361 mg) which was triturated with EtOAc (2 ml)—n-hexane (2 ml) to give ethyl 4-({2,4-dimethyl-1-[(5-methyl-2-phenyl-1,3-oxazol-4-yl)methyl]-1H-benzimidazol-6-yl}oxy)butanoate (160 mg) as white crystals.

NMR(DMSOd₆, δ): 1.16 (3H, t, J=7.1 Hz), 1.9-2.1 (2H, m), 2.3-2.5 (8H, m), 2.65 (3H, s), 3.9-4.2 (4H, m), 5.29 (2H, br s), 6.57 (1H, d, J=1.8 Hz), 6.93 (1H, d, J=1.8 Hz), 7.4-7.6 (3H, m), 7.8-7.9 (2H, m).

MS: 448 (M+1).

### PREPARATION EXAMPLE 41

Ethyl 4-{[2,4-dimethyl-1-({4-methyl-2-[4-(trifluoromethyl)phenyl]-1,3-thiazol-5-yl}methyl)-1H -benzimidazol-6-yl]oxy}butanoate (144 mg) was synthesized from 5-(chloromethyl)-4-methyl-2-(4-trifluoromethyl)phenyl-1,3-thiazole (120 mg) in a manner similar to that described in Preparation Example 40.

NMR(DMSO-d₆,δ): 1.15 (3H, t, J=7.1 Hz), 1.8-2.1 (2H, m), 2.4-2.5 (8H, m), 2.58 (3H, s), 3.9-4.2 (4H, m), 5.66 (2H, br s), 6.62 (1H, d, J=1.8 Hz), 6.95 (1H, d, J=1.8 Hz), 7.77 (2H, d, J=8.3 Hz), 8.02 (2H, d, J=8.3 Hz).

MS: 532 (M+1).

### PREPARATION EXAMPLE 42

A mixture of ethyl 4-[(2,4-dimethyl-1H-benzimidazol-6-yl)oxy]butanoate (265 mg), 4-ethoxy-2-methylbenzyl methanesulfonate (351 mg), K₂CO₃ (199 mg), sodium iodide (NaI) (216 mg) and DMF (5 ml) was heated at 80° C. for 2 hours. After cooling, the reaction mixture was diluted with EtOAc (40 ml) and washed with saturated NaHCO₃ (30 ml) and brine (2×30 ml). The organic layer was dried over MgSO4 and filtered. Evaporation gave a residue (487 mg) which was chromatographed (silica gel, EtOAc) to give ethyl 4-{[1-(4-ethoxy-2-methylbenzyl)-2,4-dimethyl-1H-benzimidazol-6-yl]oxy}butanoate (279 mg) as an oil.

NMR(DMSO-d₆,δ): 1.1-1.2 (3H, m), 1.27 (3H, t, J=7.0 Hz), 1.8-2.0 (2H, m), 2.32 (3H, s), 2.35 (3H, s), 2.42 (2H, t, J=7.3 Hz), 2.46 (3H, s), 3.8-4.1 (6H, m), 5.28 (2H, br s), 6.15 (1H, d, J=8.5 Hz), 6.5-6.7 (2H, m), 6.70 (1H, d, J=2.3 Hz), 6.80 (1H, d, J=2.3 Hz).

MS: 425 (M+1).

### PREPARATION EXAMPLE 43

To a mixture of ethyl 4-[(2,4-dimethyl-1H-benzimidazol-6-yl)oxy]butanoate (200 mg), K₂CO₃ (120 mg) and DMF (2 ml) was added 1-(bromomethyl)-2-chlorobenzene (164 mg) at ambient temperature. After stirring at 80° C. for 3 hours, the reaction mixture was diluted with EtOAc (40 ml) and washed with water (30 ml) and brine (2×30 ml). The organic layer was dried over MgSO₄, filtered, and evaporated to give ethyl 4-{[1-(2-chlorobenzyl)-2,4-dimethyl-1H-benzimidazol-6-yl]oxy}butanoate (304 mg) as a crude oil.

### PREPARATION EXAMPLE 44

To a mixture of ethyl 4-[(2,4-dimethyl-1H-benzimidazol-6-yl)oxy]butanoate (200 mg), (3,5-dichloro-2-pyridinyl)methyl methanesulfonate (204 mg) and DMF (2 ml) was added K₂CO₃ (150 mg) at ambient temperature. After stirring at 80° C. for 6 hours, the reaction mixture was diluted with EtOAc (40 ml) and washed with water (30 ml) and brine (2×30 ml). The organic layer was dried over MgSO₄ and filtered. Evaporation gave a residue (369 mg) which was triturated with n-hexane (6 ml)—EtOAc (1 ml) to give ethyl 4-({1-[(3,5-dichloro-2-pyridinyl)methyl]-2,4-dimethyl-1H-benzimidazol-6-yl}oxy)butanoate (236 mg) as white crystals.

NMR(DMSO-d₆,δ): 1.16 (3H, t, J=7.1 Hz), 1.8-2.0 (2H, m), 2.3-2.5 (8H, m), 3.89 (2H, t, J=6.2 Hz), 4.05 (2H, q, J=7.1 Hz), 5.58 (2H, br s), 6.56 (1H, d, J=1.8 Hz), 6.74 (1H, d, J=1.8 Hz), 8.32 (1H, d, J=2.1 Hz), 8.44 (1H, d, J=2.1 Hz).

MS: 436 (M+1).

### PREPARATION EXAMPLE 45

Ethyl 4-({1-[(2,6-dichloro-3-pyridinyl)methyl]-2,4-dimethyl-1H-benzimidazol-6-yl}oxy)butanoate (254 mg) was synthesized from ethyl 4-[(2,4-dimethyl-1H-benzimidazol-6-yl)oxy]butanoate (200 mg) in a manner similar to that described in Preparation Example 44 except that (2,6-dichloro-3-pyridinyl)methyl methanesulfonate (204 mg) was used instead of (3,5-dichloro-2-pyridinyl)methyl methanesulfonate.

NMR(DMSO-d₆,δ): 1.15 (3H, t, J=7.1 Hz), 1.8-2.0 (2H, m), 2.3-2.5 (8H, m), 3.90 (2H, t, J=6.3 Hz), 4.05 (2H, q, J=7.1 Hz), 5.45 (2H, br s), 6.61 (1H, d, J=1.5 Hz), 6.7-6.9 (2H, m), 7.45 (1H, d, J=8.1 Hz).

MS: 436 (M+1).

### PREPARATION EXAMPLE 46

To a mixture of ethyl 4-[(2,4-dimethyl-1H-benzimidazol-6-yl)oxy]butanoate (200 mg), 2,5-dichlorobenzyl methanesulfonate (203 mg) and DMF (2 ml) were added K₂CO₃ (150 mg) and NaI (108 mg) at ambient temperature. After stirring at 80° C. for 2 hours, the reaction mixture was diluted with EtOAc (40 ml) and washed with water (30 ml) and brine (2×30 ml). The organic layer was dried over MgSO₄, filtered, and evaporated to give ethyl 4-{[1-(2,5-dichlorobenzyl)-2,4-dimethyl-1H-benzimidazol-6-yl]oxy}butanoate (350 mg) as a crude oil.

### PREPARATION EXAMPLE 47

Ethyl 4-[(1-{2-chloro-4-[(ethoxycarbonyl)(methyl)amino]benzyl}-2,4-dimethyl-1H-benzimidazol-6-yl)oxy]butanoate (280 mg) was synthesized from ethyl 4-[(2,4-dimethyl-1H-benzimidazol-6-yl)oxy]butanoate (157 mg) in a manner similar to that described in Preparation Example 43 except that ethyl 4-(bromomethyl)-3-chlorophenyl(methyl)carbamate (192 mg) was used instead of 1-(bromomethyl)-2-chlorobenzene.

### PREPARATION EXAMPLE 48

Ethyl 4-{[1-(2,3-dichlorobenzyl)-2,4-dimethyl-1H-benzimidazol-6-yl]oxy}butanoate (199 mg) was synthesized from ethyl 4-[(2,4-dimethyl-1H-benzimidazol-6-yl)oxy]butanoate (200 mg) in a manner similar to that described in Preparation Example 38 except that 2,3-dichloro-1-(chloromethyl)benzene (170 mg) was used instead of 2,4-dichloro-1-(chloromethyl)benzene.

NMR(DMSO-d₆,δ): 1.15 (3H, t, J=7.1 Hz), 1.8-2.1 (2H, m), 2.3-2.5 (8H, m), 3.89 (2H, t, J=6.3 Hz), 4.04 (2H, q, J=7.1 Hz), 5.50 (2H, br s), 6.28 (1H, dd, J=1.2 Hz, 7.9 Hz), 6.61 (1H, d, J=1.8 Hz), 6.81 (1H, d, J=1.8 Hz), 7.24 (1H, t, J=7.9 Hz), 7.59 (1H, dd, J=1.2 Hz, 7.9 Hz).

MS: 435 (M+1).

### PREPARATION EXAMPLE 49

Ethyl 4-{[1-(3,4-dichlorobenzyl)-2,4-dimethyl-1H-benzimidazol-6-yl]oxy}butanoate was synthesized from ethyl 4-[(2,4-dimethyl-1H-benzimidazol-6-yl)oxy]butanoate (200 mg) in a manner similar to that described in Preparation Example 43 except that 3,4-dichloro-1-(chloromethyl)benzene (170 mg) was used instead of 1-(bromomethyl)-2-chlorobenzene.

### PREPARATION EXAMPLE 50

To a mixture of ethyl 4-[(2,4-dimethyl-1H-benzimidazol-6-yl)oxy]butanoate (200 mg), K₂CO₃ (150 mg) and DMF (0.6 ml) was added 4-(bromomethyl)-3-chloro-1,1′-biphenyl (306 mg) at ambient temperature. After stirring for 2 hours, the reaction mixture was diluted with EtOAc (40 ml) and washed with water (30 ml) and brine (2×30 ml). The organic layer was dried over MgSO₄ and filtered. Evaporation gave a residue (395 mg) which was chromatographed (silica gel, EtOAc) to give ethyl 4-({1-[(3-chloro-1,1′-biphenyl-4-yl)methyl]-2,4-dimethyl-1H-benzimidazol-6-yl}oxy)butanoate (266 mg) as a pale yellow oil.

NMR(DMSO-d₆,δ): 1.13 (3H, t, J=7.1 Hz), 1.8-2.0 (2H, m), 2.3-2.5 (8H, m), 3.91 (2H, t, J=6.3 Hz), 4.03 (2H, q, J=7.1 Hz), 5.50 (2H, br s), 6.48 (1H, d, J=8.1 Hz), 6.62 (1H, d, J=1.7 Hz), 6.82 (1H, d, J=1.7 Hz), 7.3-7.7 (6H, m), 7.83 (1H, d, J=1.8 Hz).

MS: 477 (M+1).

### PREPARATION EXAMPLE 51

To a mixture of ethyl 4-[(2,4-dimethyl-1H-benzimidazol-6-yl)oxy]butanoate (400 mg), K₂CO₃ (260 mg) and DMF (4 ml) was added 4-(bromomethyl)-3-chlorophenyl acetate (458 mg) at ambient temperature. After stirring for 24 hours, the reaction mixture was diluted with EtOAc (40 ml) and washed with water (30 ml) and brine (2×30 ml). The organic layer was dried over MgSO₄, filtered and evaporated to give ethyl 4-({1-[4-(acetyloxy)-2-chlorobenzyl]-2,4-dimethyl-1H-benzimidazol-6-yl}oxy)butanoate (772 mg) as a crude oil.

### PREPARATION EXAMPLE 52

To a suspension of ethyl 4-({1-[4-(acetyloxy)-2-chlorobenzyl]-2,4-dimethyl-1H-benzimidazol-6-yl}oxy)butanoate (770 mg: crude) in MeOH (7 ml) was added K₂CO₃ (464 mg) at ambient temperature. After stirring for 5 hours, the pH of the reaction mixture was adjusted to around 4 with 1 N-HCl. The precipitates were collected by filtration and washed with water. The crude crystals were triturated with EtOAc—n-hexane to give methyl 4-{[1-(2-chloro-4-hydroxybenzyl)-2,4-dimethyl-1H-benzimidazol-6-yl]oxy}butanoate (330 mg) as white crystals.

NMR(DMSO-d₆,δ): 1.8-2.1 (2H, m), 2.3-2.5 (8H, m), 3.59 (3H, s), 3.90 (2H, t, J=6.3 Hz), 5.32 (2H, br s), 6.39 (1H, d, J=8.5 Hz), 6.59 (1H, d, J=1.8 Hz), 6.64 (1H, dd, J=2.4 Hz, 8.5 Hz), 6.74 (1H, d, J=1.8 Hz), 6.88 (1H, d, J=2.4 Hz), 9.97 (1H, br s).

MS: 403 (M+1).

### PREPARATION EXAMPLE 53

To a solution of methyl 4-{[1-(2-chloro-4-hydroxybenzyl)-2,4-dimethyl-1H-benzimidazol-6-yl]oxy}butanoate (150 mg) in DMF (1.5 ml) were added K₂CO₃ (67 mg) and iodomethane (MeI) (106 mg) at ambient temperature. The mixture was stirred at 40° C. for 3 hours. The reaction mixture was diluted with EtOAc (40 ml) and washed with brine (2×30 ml). The organic layer was dried over MgSO₄, filtered, and evaporated to give methyl 4-{[1-(2-chloro-4-methoxybenzyl)-2,4-dimethyl-1H-benzimidazol-6-yl]oxy}butanoate (96 mg) as white crystals.

NMR(DMSO-d₆,δ): 1.8-2.1 (2H, m), 2.3-2.5 (8H, m), 3.59 (3H, s), 3.74 (3H, s), 3.90 (2H, t, J=6.3 Hz), 5.37 (2H, br s), 6.46 (1H, d, J=8.7 Hz), 6.59 (1H, d, J=1.8 Hz), 6.75 (1H, d, J=1.8 Hz), 6.82 (1H, dd, J=2.4 Hz, 8.7 Hz), 7.12 (1H, d, J=2.4 Hz).

MS: 417 (M+1).

### PREPARATION EXAMPLE 54

Methyl 4-{[1-(2-chloro-4-ethoxybenzyl)-2,4-dimethyl-1H-benzimidazol-6-yl]oxy}butanoate (158 mg) was synthesized from methyl 4-{[1-(2-chloro-4-hydroxybenzyl)-2,4-dimethyl-1H-benzimidazol-6-yl]oxy}butanoate (150 mg) in a manner similar to that described in Preparation Example 53 except that iodoethane (MeI). (EtI) (116 mg) was used instead of iodomethane (MeI).

NMR(DMSO-d₆,δ): 1.29 (3H, t, J=6.9 Hz), 1.8-2.1 (2H, m), 2.3-2.5 (8H, m), 3.59 (3H, s), 3.90 (2H, t, J=6.4 Hz), 4.00 (2H, q, J=6.9 Hz), 5.37 (2H, br s), 6.43 (1H, d, J=8.6 Hz), 6.59 (1H, d, J=1.8 Hz), 6.75 (1H, d, J=1.8 Hz), 6.81 (1H, dd, J=2.5 Hz, 8.6 Hz), 7.09 (1H, d, J=2.5 Hz).

MS: 431 (M+1).

### PREPARATION EXAMPLE 55

To a mixture of ethyl 4-[4-(acetylamino)-3-aminophenoxy]butanoate hydrochloride (300 mg), K₂CO₃ (288 mg) and DMF (3 ml) was added 2,4-dichloro-1-(chloromethyl)benzene (204 mg) at ambient temperature. The mixture was heated at 80° C. for 2 hours and at 90° C. for 2 hours. After cooling, the. reaction mixture was diluted with EtOAc (50 ml) and washed with water (30 ml) and brine (2×30 ml). The organic layer was dried over MgSO₄, filtered, and evaporated to give ethyl 4-{4-(acetylamino)-3-[(2,4-dichlorobenzyl)amino]phenoxy}butanoate (448 mg) as a crude oil.

### PREPARATION EXAMPLE 56

To a solution of ethyl 4-{4-(acetylamino)-3-[(2,4-dichlorobenzyl)amino]phenoxy}butanoate (448 mg: crude) in EtOH (4.5 ml) was added H₂SO₄ (200 mg) at ambient temperature. The mixture was stirred at room temperature for 3 days and at 80° C. for 1 hour. After cooling, the pH of the reaction mixture was adjusted to around 8 with 1 N-NaOH and extracted with EtOAc (2×20 ml). The combined organic extracts were washed with water (30 ml) and brine (30 ml). The organic layer was dried over MgSO₄ and filtered. Evaporation gave a residue (277 mg) which was chromatographed (silica gel, EtOAc) to give ethyl 4-{[1-(2,4-dichlorobenzyl)-2-methyl-1H-benzimidazol-6-yl]oxy}butanoate (102 mg) as white crystals.

NMR(DMSO-d₆,δ): 1.15 (3H, t, J=7.1 Hz), 1.8-2.1 (2H, m), 2.38 (3H, s), 2.44 (2H, t, J=7.2 Hz), 3.93 (2H, t, J=6.3 Hz), 4.05 (2H, q, J=7.1 Hz), 5.47 (2H, br s), 6.42 (1H, d, J=8.4 Hz), 6.77 (1H, dd, J=2.2 Hz, 8.7 Hz), 6.99 (1H, d, J=2.2 Hz), 7.33 (1H, dd, J=2.1 Hz, 8.4 Hz), 7.44 (1H, d, J=8.7 Hz), 7.73 (1H, d, J=2.1 Hz).

MS: 423 (M+1).

### PREPARATION EXAMPLE 57

Ethyl 4-{4-(acetylamino)-3-[(2-chloro-4-ethoxybenzyl)amino]phenoxy}butanoate (454 mg) was synthesized from ethyl 4-[4-(acetylamino)-3-aminophenoxy]butanoate hydrochloride (300 mg) in a manner similar to that described in Preparation Example 55 except that 1-(bromomethyl)-2-chloro-4-ethoxybenzene (284 mg) was used instead of 2,4-dichloro-1-(chloromethyl)benzene.

### PREPARATION EXAMPLE 58

Ethyl 4-{[1-(2-chloro-4-ethoxybenzyl)-2-methyl-1H-benzimidazol-6-yl]oxy}butanoate (200 mg) was synthesized from ethyl 4-{4-(acetylamino)-3-[(2-chloro-4-ethoxybenzyl)amino]phenoxy}butanoate (454 mg) in a manner similar to that described in Preparation Example 56.

NMR(DMSO-d₆,δ): 1.16 (3H, t, J=7.1 Hz), 1.29 (3H, t, J=7.0 Hz), 1.8-2.1 (2H, m), 2.3-2.5 (5H, m), 3.8-4.2 (6H, m), 5.40 (2H, br s), 6.47 (1H, d, J=8.6 Hz), 6.7-6.9 (2H, m), 6.95 (1H, d, J=2.2 Hz), 7.10 (1H, d, J=2.5 Hz), 7.42 (1H, d, J=8.7 Hz).

MS: 431 (M+1).

### PREPARATION EXAMPLE 59

Ethyl 4-[4-(acetylamino)-3-({2-chloro-4-[(ethoxycarbonyl)(methyl)amino]benzyl}amino)phenoxy]butanoate was synthesized from ethyl 4-[4-(acetylamino)-3-aminophenoxy]butanoate hydrochloride (300 mg) in a manner similar to that described in Preparation Example 55 except that ethyl 4-(bromomethyl)-3-chlorophenyl(methyl)carbamate (348 mg) was used instead of 2,4-dichloro-1-(chloromethyl)benzene.

### PREPARATION EXAMPLE 60

Ethyl 4-[(1-{2-chloro-4-[(ethoxycarbonyl)(methyl)amino]benzyl}-2-methyl-1H-benzimidazol-6-yl)oxy]butanoate (199 mg) was synthesized from ethyl 4-[4-(acetylamino)-3-({2-chloro-4-[(ethoxycarbonyl)(methyl)amino]benzyl]amino}phenoxy]butanoate (509 mg) in a manner similar to that described in Preparation Example 56.

NMR(DMSO-d₆,δ): 1.1-1.2 (6H, m), 1.8-2.0: (2H, m)), 2.40 (3H, s), 2.44 (2H, t, J=7.3 Hz), 3.19 (3H, s), 3.93 (2H, t, J=6.3 Hz), 4.0-4.2 (4H, m), 5.47 (2H, br s), 6.43 (1H, d, J=8.4 Hz), 6.77 (1H, dd, J=2.4 Hz, 8.7 Hz), 7.00 (1H, d, J=2.4 Hz), 7.18 (1H, dd, J=2.2 Hz, 8.4 Hz), 7.44 (1H, d, J=8.7 Hz), 7.56 (1H, d, J=2.2 Hz).

MS: 488 (M+1).

### PREPARATION EXAMPLE 61

To a mixture of ethyl 4-[4-(acetylamino)-3-aminophenoxy]butanoate hydrochloride (300 mg), K₂CO₃ (314 mg) and DMF (3 ml) were added 3-chloro-2-(chloromethyl)-5-(trifluoromethyl)pyridine (261 mg) and NaI (170 mg) at ambient temperature. After stirring for 4 hours, the reaction mixture was diluted with EtOAc (40 ml) and washed with water (30 ml) and brine (2×30 ml). The organic layer was dried over MgSO₄ and filtered. Evaporation gave a residue which was triturated with EtOAc (3 ml)—n-hexane (2 ml) to give ethyl 4-[4-(acetylamino)-3-({[3-chloro-5-(trifluoromethyl)-2-pyridinyl]methyl}amino)phenoxy]butanoate (240 mg) as white crystals.

NMR(DMSO-d₆,δ): 1.17 (3H, t, J=7.1 Hz), 1.8-2.1 (2H, m), 2.03 (3H, s), 2.42 (2H, t, J=7.3 Hz), 3.90 (2H, t, J=6.3 Hz), 4.06 (2H, q, J=7.1 Hz), 4.56 (2H, d, J=5.3 Hz), 5.77 (1H, t, J=5.3 Hz), 6.1-6.3 (2H, m), 6.94 (1H, d, J=8.4 Hz), 8.50 (1H, d, J=1.3 Hz), 8.95 (1H, d, J=1.3 Hz), 9.11 (1H, br s).

MS: 496 (M+Na)

### PREPARATION EXAMPLE 62

To a solution of ethyl 4-[4-(acetylamino)-3-({[3-chloro-5-(trifluoromethyl)-2-pyridinyl]methyl}amino)phenoxy]butanoate (200 mg) in EtOH (2 ml) was added H₂SO₄ (41 mg) at ambient temperature. The mixture was heated at 80° C. for 2 hours. After cooling, the pH of the reaction mixture was adjusted to around 8 with 1 N-NaOH and extracted with EtOAc (2×20 ml). The combined organic extracts were washed with water (30 ml) and brine (30 ml). The organic layer was dried over MgSO₄, filtered, and evaporated to give ethyl 4-[(1-{[3-chloro-5-(trifluoromethyl)-2-pyridinyl]methyl)-2-methyl-1H-benzimidazol-6-yl)oxy]butanoate (188 mg) as pale brown crystals.

NMR(DMSO-d₆,δ): 1.15 (3H, t, J=7.1 Hz), 1.8-2.1 (2H, m), 2.38 (3H, s), 2.43 (2H, t, J=7.4 Hz), 3.92 (2H, t, J=6.3 Hz), 4.04 (2H, q, J=7.1 Hz), 5.73 (2H, br s), 6.73 (1H, dd, J=2.3 Hz, 8.6 Hz), 6.98 (1H, d, J=2.3 Hz), 7.39 (1H, d, J=8.6 Hz), 8.56 (1H, d, J=1.2 Hz), 8.77 (1H, d, J=1.2 Hz).

MS: 456 (M+1).

### PREPARATION EXAMPLE 63

To a suspension of ethyl 4-[4-(acetylamino)-3-methyl-5-nitrophenoxy]butanoate (18.2 g) in EtOH (182 ml) was added 1 N-NaOH (112 ml) at ambient temperature. The mixture was heated at 80-90° C. for 2 days. After cooling, the pH of the reaction mixture was adjusted to around 3 with 1 N-HCl. The precipitates were collected by filtration and washed with water to give 4-(4-amino-3-methyl-5-nitrophenoxy)butanoic acid (13.7 g) as orange crystals.

NMR(DMSO-d₆,δ): 1.8-2.0 (2H, m), 2.20 (3H, s), 2.37 (2H, t, J=7.4 Hz), 3.92 (2H, t, J=6.4 Hz), 6.99 (2H, br s), 7.14 (1H, d, J=2.6 Hz), 7.30 (1H, d, J=2.6 Hz), 12.09 (1H, br s).

MS: 253 (M−1).

### PREPARATION EXAMPLE 64

To a suspension of 4-(4-amino-3-methyl-5-nitrophenoxy)butanoic acid (13 g) in EtOH (130 ml) was added H₂SO₄ (10 g) at ambient temperature. The mixture was heated at reflux. for 13 hours. After cooling, the reaction mixture was poured into ice-water (200 ml) and EtOAc (200 ml). The aqueous layer was neutralized with 20%-NaOH and the organic layer was separated. The aqueous layer was extracted with EtOAc (2×200 ml). The combined organic extracts were washed with saturated NaHCO₃ (3×200ml) and brine (300 ml). The organic layer was dried over MgSO₄, filtered, and evaporated to give ethyl 4-(4-amino-3-methyl-5-nitrophenoxy)butanoate (12.8 g) as a red oil.

NMR(DMSO-d₆,δ): 1.18 (3H, t, J=7.1 Hz), 1.8-2.1 (2H, m), 2.20 (3H, s), 2.44 (2H, t, J=7.3 Hz), 3.93 (2H, t, J=6.4 Hz), 4.07 (2H, q, J=7.1 Hz), 7.00 (2H, br s), 7.13 (1H, d, J=2.6 Hz), 7.30 (1H, d, J=2.6 Hz).

MS: 305 (M+Na).

### PREPARATION EXAMPLE 65

To a solution of ethyl 4-(4-amino-3-methyl-5-nitrophenoxy)butanoate (5.0 g) in EtOH (50 ml) was added palladium on carbon (10%, 50% wet, 2 g) at ambient temperature, and the resultant mixture was hydrogenated under atmospheric pressure of hydrogen for 2 hours. The catalyst was removed by filtration. The filtrate was evaporated to give ethyl 4-(3,4-diamino-5-methylphenoxy)butanoate (4.12 g) as a brown oil.

NMR(DMSO-d₆,δ): 1.18 (3H, t, J=7.2 Hz), 1.7-2.0 (2H, m), 2.00 (3H, s), 2.40 (2H, t, J=7.2 Hz), 3.76 (2H, t, J=6.3 Hz), 4.06 (2H, q, J=7.2 Hz), 4.45 (2H, br s), 5.91 (1H, d, J=2.7 Hz), 6.05 (1H, d, J=2.7 Hz).

MS: 275 (M+Na).

### PREPARATION EXAMPLE 66

A mixture of ethyl 4-(3,4-diamino-5-methylphenoxy)butanoate (4.1 g), tetraethyl orthocarbonate (17 ml) and AcOH (1.27 g) was heated at 80° C. for 1 hour. After cooling, the reaction mixture was evaporated. The residue was dissolved in EtOAc (100 ml) and washed with saturated NaHCO₃ (2×50 ml) and brine (50 ml). The organic layer was dried over MgSO₄ and filtered. Evaporation gave a residue which was triturated with n-hexane (20 ml) to give ethyl 4-[(2-ethoxy-4-methyl-1H-benzimidazol-6-yl)oxy]butanoate (4.35 g) as pale brown crystals.

NMR(DMSO-d₆,δ): 1.18 (3H, t, J=7.1 Hz), 1.36 (3H, t, J=7.0 Hz), 1.8-2.1 (2H, m), 2.33 (3H, s), 2.45 (2H, t, J=7.3 Hz), 3.92 (2H, t, J=6.3 Hz), 4.07 (2H, q, J=7.1 Hz), 4.44 (2H, q, J=7.0 Hz), 6.46 (1H, d, J=1.7 Hz), 6.66 (1H, br s), 11.58 (1H, br s).

MS: 307 (M+1).

### PREPARATION EXAMPLE 67

Ethyl 4-{[1-(2,4-dichlorobenzyl)-2-ethoxy-4-methyl-1H-benzimidazol-6-yl]oxy}butanoate (151 mg) was synthesized from ethyl 4-[(2-ethoxy-4-methyl-1H-benzimidazol-6-yl)oxy]butanoate (200 mg) in a manner similar to that described in Preparation Example 38.

NMR(DMSO-d₆,δ): 1.16 (3H, t, J=7.1 Hz), 1.31 (3H, t, J=7.0 Hz), 1.8-2.0 (2H, m), 2.3-2.5 (5H, m), 3.89 (2H, t, J=6.3 Hz), 4.05 (2H, q, J=7.1 Hz), 4.48 (2H, q, J=7.0 Hz), 5.23 (2H, br s), 6.55 (1H, d, J=1.9 Hz), 6.71 (1H, d, J=1.9 Hz), 6.78 (1H, d, J=8.3 Hz), 7.36 (1H, dd, J=2.1 Hz, 8.3 Hz), 7.68 (1H, d, J=2.1 Hz).

MS: 465 (M+1).

### PREPARATION EXAMPLE 68

To a mixture of ethyl 4-[(2-ethoxy-4-methyl-1H-benzimidazol-6-yl)oxy]butanoate (200 mg), K₂CO₃ (117 mg) and DMF (2 ml) was added 1-(bromomethyl)-2-chloro-4-ethoxybenzene (195 mg) at ambient temperature. The mixture was heated at 80° C. for 3 hours. After cooling, the reaction mixture was diluted with EtOAc (50 ml) and washed with water (30 ml) and brine. (2×30 ml). The organic layer was dried over MgSO₄ and filtered. Evaporation gave a residue which was chromatographed (silica gel, EtOAc/n-hexane=1/4) to give ethyl 4-{[1-(2-chloro-4-ethoxybenzyl)-2-ethoxy-4-methyl-1H-benzimidazol-6-yl]oxy}butanoate (215 mg) as an oil.

NMR(DMSO-d₆,δ): 1.1-1.4 (9H, m), 1.8-2.0 (2H, m), 2.3-2.5 (5H, m), 3.89 (2H, t, J=6.3 Hz), 3.9-4.2 (4H, m), 4.48 (2H, q, J=7.0 Hz), 5.15 (2H, br s), 6.53 (1H, d, J=2.0 Hz), 6.65 (1H, d, J=2.0 Hz), 6.7-6.9 (2H, m), 7.05 (1H, d, J=1.9 Hz).

MS: 475 (M+1).

### PREPARATION EXAMPLE 69

Ethyl 4-({1-[2-chloro-4-(pentyloxy)benzyl]-2-ethoxy-4-methyl-1H-benzimidazol-6-yl}oxy)butanoate (209 mg) was synthesized from ethyl 4-[(2-ethoxy-4-methyl-1H-benzimidazol-6-yl)oxy]butanoate (200 mg) in a manner similar to that described in Preparation Example 68 except that 1-(bromomethyl)-2-chloro-4-(pentyloxy)benzene (228 mg) was used instead of 1-(bromomethyl)-2-chloro-4-ethoxybenzene.

NMR(CDCl₃,δ): 0.8-1.0-(3H, m), 1.1-1.5 (10H, m), 1.6-1.9 (2H, m), 1.9-2.2 (2H, m), 2.49 (2H, t, J=7.3 Hz), 2.54 (3H, s), 3.8-4.0 (4H, m), 4.13 (2H, q, J=7.2 Hz), 4.60 (2H, q, J=7.1 Hz), 5.12 (2H, br s), 6.42 (1H, d, J=2.3 Hz), 6.58 (1H, d, J=2.3 Hz), 6.64 (1H, dd, J=2.4 Hz, 8.6 Hz), 6.73 (1H, d, J=8.6 Hz), 6.94 (1H, d, J=2.4 Hz).

MS: 517 (M+1).

### PREPARATION EXAMPLE 70

To a mixture of ethyl 4-[(2-ethoxy-4-methyl-1H-benzimidazol-6-yl)oxy]butanoate (200 mg), K₂CO₃ (108 mg), NaI (98 mg) and DMF (2 ml) was added 5-(chloromethyl)-4-methyl-2-[4-(trifluoromethyl)phenyl]-1,3-thiazole (229 mg) at ambient temperature. The mixture was heated at 80° C. for 4 hours. After cooling, the reaction mixture was diluted with EtOAc (50 ml) and washed with water (30 ml) and brine (2×30 ml). The organic layer was dried over MgSO₄ and filtered. Evaporation gave a residue (410 mg) which was triturated with n-hexane (2 ml)—EtOAc (2 drops) to give ethyl 4-{[2-ethoxy-4-methyl-1-({4-methyl-2-[4-(trifluoromethyl)phenyl]-1,3-thiazol-5-yl}methyl)-1H-benzimidazol-6-yl]oxy}butanoate (250 mg) as crude crystals.

MS: 562 (M+1).

### PREPARATION EXAMPLE 71

To a solution of ethyl 4-(4-amino-3-methyl-5-nitrophenoxy)butanoate (350 mg) in DMF (3.5 ml) were added 4-(dimethylamino)pyridine (DMAP) (30 mg) and propanoyl chloride (138 mg) at ambient temperature. After stirring for 14 hours, the reaction mixture was diluted with EtOAc (50 ml), and washed with 1 N-HCl (2×30 ml), saturated NaHCO₃ (2×30 ml), and brine (20 ml). The organic layer was dried over MgSO₄ and evaporated to give ethyl 4-[3-methyl-5-nitro-4-(propionylamino)phenoxy]butanoate (411 mg) as pale yellow crystals.

NMR(DMSO-d₆,δ): 1.06 (3H, t, J=7.5 Hz), 1.18 (3H, t, J=7.2 Hz), 1.8-2.1 (2H, m), 2.22 (3H, s), 2.28 (2H, q, J=7.5 Hz), 2.45 (2H, t, J=7.2 Hz), 4.0-4.2 (4H, m), 7.20 (1H, d, J=2.7 Hz), 7.26 (1H, d, J=2.7 Hz), 9.54 (1H, br s).

MS: 361 (M+Na).

### PREPARATION EXAMPLE 72

To a solution of ethyl 4-[3-methyl-5-nitro-4-(propionylamino)phenoxy]butanoate (410 mg) in a mixture of EtOH (4.1 ml) and ACOH (4.1 ml) was added iron powder (338 mg) at ambient temperature. The mixture was heated at 110° C. for 1 hour. The reaction mixture was evaporated, added saturated NaHCO₃ (40 ml), and extracted with EtOAc (2×30 ml). The combined organic extracts were washed with saturated NaHCO₃ (30 ml) and brine (30 ml). The organic layer was dried over MgSO₄, decolored (activated carbon), and evaporated to give ethyl 4-[(2-ethyl-4-methyl-1H-benzimidazol-6-yl)oxy]butanoate (341 mg) as an oil.

NMR(DMSO-d₆,δ): 1.18 (3H, t, J=7.1 Hz), 1.29 (3H, t, J=7.5 Hz), 1.8-2.1 (2H, m), 2.3-2.5 (5H, m), 2.77 (2H, q, J=7.5 Hz), 3.95 (2H, q, J=6.2 Hz), 4.07 (2H, q, J=7.1 Hz), 6.54 (1H, br s), 6.77 (1H, br s), 11.90 (1H, br s).

MS: 291 (M+1).

### PREPARATION EXAMPLE 73

Ethyl 4-{[1-(2,4-dichlorobenzyl)-2-ethyl-4-methyl-1H-benzimidazol-6-yl]oxy}butanoate (181 mg) was synthesized from ethyl 4-[(2-ethyl-4-methyl-1H-benzimidazol-6-yl)oxy]butanoate (165 mg) in a manner similar to that described in Preparation Example 38.

NMR(DMSO-d₆,δ): 1.0-1.3 (6H, m), 1.8-2.0 (2H, m), 2.3-2.5 (5H, m), 2.70 (2H, q, J=7.4 Hz), 3.90 (2H, t, J=6.3 Hz), 4.04 (2H, q, J=7.1 Hz), 5.45 (2H, br s), 6.36 (1H, d, J=8.4 Hz), 6.61 (1H, d, J=1.7 Hz), 6.79 (1H, d, J=1.7 Hz), 7.31 (1H, dd, J=2.1 Hz, 8.4 Hz), 7.72 (1H, d, J=2.1 Hz).

MS: 449 (M+1).

### PREPARATION EXAMPLE 74

Ethyl 4-{3-methyl-5-nitro-4-[(trifluoroacetyl)amino]phenoxy}butanoate (548 mg) was synthesized from ethyl 4-(4-amino-3-methyl-5-nitrophenoxy)butanoate (400 mg) in a manner similar to that described in Preparation Example 71 except that trifluoroacetic anhydride (357 mg) was used instead of propanoyl chloride.

NMR(DMSO-d₆,δ): 1.18 (3H, t, J=7.1 Hz), 1.8-2.1 (2H, m), 2.24 (3H, s), 2.47 (2H, t, J=7.2 Hz), 4.0-4.2 (4H, m), 7.32 (1H, d, J=2.8 Hz), 7.43 (1H, d, J=2.8 Hz), 11.23 (1H, br s).

MS: 401 (M+Na).

### PREPARATION EXAMPLE 75

Ethyl 4-{[4-methyl-2-(trifluoromethyl)-1H-benzimidazol-6-yl]oxy}butanoate (461 mg) was synthesized from ethyl 4-{3-methyl-5-nitro-4-[(trifluoroacetyl)amino]phenoxy}butanoate (533 mg) in a manner similar to that described in Preparation Example 72.

NMR(DMSO-d₆,δ): 1.18 (3H, t, J=7.1 Hz), 1.9-2.1 (2H, m), 2.3-2.6 (5H, m), 3.9-4.2 (4H, m), 6.80 (1H, br s), 6.90 (1H, br s), 13.63 (1H, br s).

MS: 353 (M+Na).

### PREPARATION EXAMPLE 76

Ethyl 4-{[1-(2,4-dichlorobenzyl)-4-methyl-2-(trifluoromethyl)-1H-benzimidazol-6-yl]oxy}butanoate (237 mg) was synthesized from ethyl 4-{[4-methyl-2-(trifluoromethyl)-1H-benzimidazol-6-yl]oxy}butanoate (225 mg) in a manner similar to that described in Preparation Example 38.

NMR(DMSO-d₆,δ): 1.16 (3H, t, J=7.1 Hz), 1.8-2.1 (2H, m), 2.44 (2H, t, J=7.1 Hz), 2.55 (3H, s), 3.9-4.2 (4H, m), 5.68 (2H, br s), 6.36 (1H, d, J=8.4 Hz), 6.86 (1H, d, J=1.7 Hz), 7.08 (1H, d, J=1.7 Hz), 7.32 (1H, dd, J=2.1 Hz, 8.4 Hz), 7.75 (1H, d, J=2.1 Hz).

MS: 489 (M+1).

### PREPARATION EXAMPLE 77

To a solution of ethyl 4-(3,4-diamino-5-methylphenoxy)butanoate (442 mg) in DMF (2.2 ml) was added 1-(1H-imidazol-1-ylcarbothioyl)-1H-imidazole (375 mg) at ambient temperature. After stirring for 19 hours, to the reaction mixture was added water (4 ml) dropwise and the mixture was stirred for 1 hour. The precipitates were collected by filtration and washed with water and MeOH (1 ml) to give ethyl 4-[(7-methyl-2-thioxo-2,3-dihydro-1H-benzimidazol-5-yl)oxy]butanoate (447 mg) as pale brown crystals.

NMR(DMSO-d₆,δ): 1.18 (3H, t, J=7.1 Hz), 1.8-2.1 (2H, m), 2.32 (3H, s), 2.44 (2H, t, J=7.2 Hz), 3.93 (2H, t, J=6.3 Hz), 4.07 (2H, q, J=7.1 Hz), 6.47 (1H, d, J=1.8 Hz), 6.54 (1H, d, J=1.8 Hz), 12.35 (1H, br s), 12.46 (1H, br s).

MS: 317 (M+Na).

### PREPARATION EXAMPLE 78

To a mixture of N-(4-hydroxy-2-methyl-6-nitrophenyl)acetamide (215 mg), ethyl 4-bromo-2,2-dimethylbutanoate (251 mg) and DMF (1 ml) was added K₂CO₃ (170 mg) at ambient temperature. After stirring for 6 hours at 60° C., the reaction mixture was diluted with EtOAc (30 ml) and washed with water (20 ml), 1 N-NaOH (2×20 ml), and brine (20 ml). The organic layer was dried over MgSO₄ and filtered. Evaporation gave a residue (260 mg) which was triturated with EtOAc (0.5 ml)—n-hexane (1 ml) to give ethyl 4-[4-(acetylamino)-3-methyl-5-nitrophenoxy]-2,2-dimethylbutanoate (171 mg) as pale yellow crystals.

NMR(DMSO-d₆,δ): 1.16 (3H, t, J=7.1 Hz), 1.19 (6H, s), 1.9-2.1 (5H, m), 2.22 (3H, s), 3.9-4.2 (4H, m), 7.15 (1H, d, J=2.8 Hz), 7.23 (1H, d, J=2.8 Hz), 9.62 (1H, br s).

MS: 375 (M+Na).

### PREPARATION EXAMPLE 79

To a solution of ethyl 4-[4-(acetylamino)-3-methyl-5-nitrophenoxy]-2,2-dimethylbutanoate (2.51 g) in a mixture of EtOH (25 ml) and AcOH (25 ml) was added iron powder (1.19 g) at ambient temperature. The mixture was heated at 110° C. for 1 hour. After cooling, the reaction mixture was evaporated, saturated NaHCO₃ (150 ml) added thereto, and the mixture was extracted with EtOAc (3×100 ml). The combined organic extracts were washed with saturated NaHCO₃ (100 ml) and brine (100 ml). The organic layer was dried over MgSO₄, decolored (activated carbon), and evaporated to give ethyl 4-[(2,4-dimethyl-1H-benzimidazol-6-yl)oxy]-2,2-dimethylbutanoate (1.46 g) as a yellow oil.

NMR(DMSO-d₆,δ): 1.17 (3H, t, J=7.0 Hz), 1.19 (6H, s), 1.97 (2H, t, J=6.7 Hz), 2.3-2.5 (6H, m), 3.94 (2H, t, J=6.7 Hz), 4.07 (2H, q, J=7.0 Hz), 6.48 (1H, br s), 6.73 (1H, br s), 11.92 (1H, br s).

MS: 305 (M+1).

### PREPARATION EXAMPLE 80

To a mixture of ethyl 4-[(2,4-dimethyl-1H-benzimidazol-6-yl)oxy]-2,2-dimethylbutanoate (250 mg), K₂CO₃ (159 mg) and DMF (1.2 ml) was added 2,4-dichloro-1-(chloromethyl)benzene (209 mg) at ambient temperature. The mixture was heated at 90° C. for 3 hours. After cooling, the reaction mixture was diluted with EtOAc (50 ml) and washed with water (20 ml) and brine (2×50 ml). The organic layer was dried over MgSO₄, decolored (activated carbon), and evaporated-to give ethyl 4-{[1-(2,4-dichlorobenzyl)-2,4-dimethyl-1H-benzimidazol-6-yl]oxy}-2,2-dimethylbutanoate (430 mg) as a crude oil.

### PREPARATION EXAMPLE 81

Ethyl 4-({1-[2-chloro-4-(pentyloxy)benzyl]-2,4-dimethyl-1H-benzimidazol-6-yl}oxy)-2,2-dimethylbutanoate was synthesized from ethyl 4-[(2,4-dimethyl-1H-benzimidazol-6-yl)oxy]-2,2-dimethylbutanoate (250 mg) in a manner similar to that described in Preparation Example 80 except that 1-(bromomethyl)-2-chloro-4-(pentyloxy)benzene (335 mg) was used instead of 2,4-dichloro-1-(chloromethyl)benzene.

### PREPARATION EXAMPLE 82

To a mixture of ethyl 4-[(2,4-dimethyl-1H-benzimidazol-6-yl)oxy]-2,2-dimethylbutanoate (200 mg), 3-chloro-2-(chloromethyl)-5-(trifluoromethyl)pyridine (212 mg), K₂CO₃ (136 mg) and DMF (1 ml) was added NaI (49 mg) at ambient temperature. The mixture was heated at 90° C. for 2 hours. After cooling, the reaction mixture was diluted with EtOAc (50 ml) and washed with water (20 ml) and brine (2×50 ml). The organic layer was dried over MgSO₄, decolored (activated carbon), and filtered. Evaporation gave a residue (348 mg) which was triturated with EtOAc (1 ml)—n-hexane (5 ml) to give ethyl 4-[(1-{[3-chloro-5-(trifluoromethyl)-2-pyridinyl]methyl}-2,4-dimethyl-1H-benzimidazol-6-yl)oxy]-2,2-dimethylbutanoate (140 mg) as white crystals.

NMR(DMSO-d₆,δ): 1.10 (3H, t, J=7.1 Hz), 1.15 (6H, s), 1.92 (2H, t, J=6.6 Hz), 2.39 (3H, s), 2.44 (3H, s), 3.88 (2H, t, J=6.6 Hz), 4.02 (2H, q, J=7.1 Hz), 5.70 (2H, br s), 6.51 (1H, d, J=1.8 Hz), 6.71 (1H, d, J=1.8 Hz), 8.56 (1H, br s), 8.77 (1H, br s).

MS: 498 (M+1).

### PREPARATION EXAMPLE 83

2,5-Dichlorobenzyl methanesulfonate was synthesized from (2,5-dichlorophenyl)methanol (354 mg) in a manner similar to that described in Preparation Example 204.

### PREPARATION EXAMPLE 84

A mixture of ethyl 4-[(2-ethoxy-4-methyl-1H-benzimidazol-6-yl)oxy]butanoate (200 mg), 3-chloro-2-(chloromethyl)-5-(trifluoromethyl)pyridine (165 mg), NaI (98 mg) and K₂CO₃ (108 mg) in N,N-dimethylformamide (DMF) (2 mL) was stirred for 4 hours at room temperature. The mixture was then diluted with EtOAc (10 mL) and poured into water (20 mL). The layers were separated and the aqueous layer was extracted with EtOAc (2×10 mL), the combined organic layers were washed with brine (20 mL), dried (Na₂SO₄), and concentrated. Purification of the residue by column chromatography (silica gel 20g, EtOAc/n-hexane=1/4) afforded 178 mg of ethyl 4-[(1-{[3-chloro-5-(trifluoromethyl)-2-pyridinyl]methyl}-2-ethoxy-4-methyl-1H-benzimidazol-6-yl)oxy]butanoate as a yellow oil.

NMR(200 MHz,DMSO-d₆,δ): 1.15 (3H, t, J=7.1 Hz), 1.25 (3H, t, J=7.0 Hz), 1.87-1.99 (2H, m), 2.40 (3H, s), 2.42 (2H, t, J=7.4 Hz), 3.88 (2H, t, J=6.3 Hz), 4.08 (2H, q, J=7.1 Hz), 4.44 (2H, q, J=7.0 Hz), 5.48 (2H, s), 6.51 (1H, d, J=2.0 Hz), 6.73 (1H, d, J=2.0 Hz), 8.53 (1H, s), 8.90 (1H, s).

MS: 522 (M+Na).

### PREPARATION EXAMPLE 85

To a 0° C. suspension of NaH (5.82 g, 60% oil dispersion), previously washed with n-hexane, in DMF (500 mL) was added a solution of ethyl 4-(4-amino-3-nitrophenoxy)butanoate (15 g) in DMF (50 mL). After 30 minutes at room temperature, the solution was treated with a solution of di-tert-butyl dicarbonate (14.1 ml) in DMF (30 mL). The resultant mixture was stirred for 3 hours at room temperature before the reaction was quenched by addition of water (200 mL). The mixture was extracted with EtOAc (3×100 mL). The combined organic extracts were washed with brine (200 mL), dried (Na₂SO₄), and filtered, and the filtrates were concentrated under reduced pressure. Purification by chromatography on silica gel (EtOAc/n-hexane 1/1) gave ethyl 4-{4-[(tert-butoxycarbonyl)amino]-3-nitrophenoxy}butanoate (8.4 g) as a gray oil which solidified upon standing.

NMR(200 MHz,DMSO-d₆,δ): 1.18 (3H, t, J=7.2 Hz), 1.42 (9H, s), 1.93-2.05 (2H, m), 2.45 (2H, t, J=7.2 Hz), 4.03 (2H, t, J=7.0 Hz), 4.06 (2H, q, J=7.2 Hz), 7.25 (1H, dd, J=2.7 Hz, 8.8 Hz), 7.43 (1H, d, J=2.7 Hz), 7.45 (1H, d, J=8.8 Hz), 9.30 (1H, s).

MS: 391 (M+Na).

### PREPARATION EXAMPLE 86

To a solution of ethyl 4-{4-[(tert-butoxycarbonyl)amino]-3-nitrophenoxy}butanoate (2.95 g) in EtOH (60 mL) was added palladium on carbon (10%, 50% wet, 1.7 g) at room temperature, and the resultant mixture was hydrogenated under atmospheric pressure of hydrogen for 3 hours. The catalyst was removed by filtration. The filtrate was evaporated to give ethyl 4-{3-amino-4-[(tert-butoxycarbonyl)amino]phenoxy}butanoate (2.66 g).

NMR(200 MHz,DMSO-d₆,δ): 1.14 (3H, t, J=7.1 Hz), 1.46 (9H, s), 1.88-1.99 (2H, m), 2.42 (2H, t, J=7.2 Hz), 3.85 (2H, t, J=6.2 Hz), 4.06 (2H, q, J=7.1 Hz), 5.24 (2H, s), 6.08 (1H, dd, J=2.8 Hz, 8.6 Hz), 6.24 (1H, d, J=2.8 Hz), 6.94 (1H, d, J=8.6 Hz), 8.08 (1H, s).

MS: 361 (M+Na).

### PREPARATION EXAMPLE 87

A mixture of ethyl 4-{3-amino-4-[(tert-butoxycarbonyl)amino]phenoxy}butanoate (658 mg), 2,4-dichlorobenzylchloride (0.3 mL) and K₂CO₃ (198 mg) in DMF (7 mL) was stirred for 1.5 hours at room temperature. The mixture was heated at 80° C. for 1 hour and 90° C. for 2 hours. NaI (20 mg), K₂CO₃ (198 mg) and 2,4-dichlorobenzylchloride (0.3 mL) were added at room temperature, and the resulting suspension was heated at 80° C. for 3 hours. After cooling to room temperature, the reaction mixture was poured into a saturated NH₄Cl solution (30 mL). The phases were separated and the aqueous layer was extracted with EtOAc (2×15 mL). The combined extracts were washed with brine (20 mL), dried over Na₂SO₄, and concentrated to provide the crude ester (840 mg) Purification of the product by column chromatography (EtOAc/n-hexane 1/8) provided ethyl 4-{4-[(tert-butoxycarbonyl)amino]-3-[(2,4-dichlorobenzyl)amino]phenoxy}butanoate (502 mg).

NMR(200 MHz,DMSO-d₆,δ): 1.13 (3H, t, J=7.1 Hz), 1.44 (9H, s), 1.86-1.99 (2H, m), 2.36 (2H, t, J=7.3 Hz), 3.82 (2H, t, J=6.3 Hz), 4.02 (2H, q, J=7.1 Hz), 4.34 (2H, d, J=5.9 Hz), 5.64 (1H, t, J=5.9 Hz), 5.82 (1H, d, J=2.5 Hz), 6.12 (1H, dd, J=2.5 Hz, 8.6 Hz), 6.96 (1H, d, J=8.6 Hz), 7.38 (1H, s), 7.39 (1H, s), 7.62 (1H, s), 8.15 (1H, s).

MS: 497 (M+1).

### PREPARATION EXAMPLE 88

To a solution of ethyl 4-{4-[(tert-butoxycarbonyl)amino]-3-[(2,4-dichlorobenzyl)amino]phenoxy}butanoate (502 mg) in EtOH (3.3 mL) was added hydrogen chloride, 4M solution in 1,4-dioxane (4N HCl in dioxane) (2.6 mL) at room temperature. The mixture was stirred at room temperature for 14 hours, and then concentrated in vacuo without external heating. The residue was dissolved in 5 mL of EtOAc, and neutralized with 1N NaOH. The phases were separated and the aqueous layer was extracted with EtOAc (2×10 mL). The combined organic extracts were washed with brine (10 mL), dried (Na₂SO₄), and concentrated. The residue was purified by column chromatography on silica gel (EtOAc/hexane 1/4) to afford ethyl 4-{4-amino-3-[(2,4-dichlorobenzyl)amino]phenoxy}butanoate (240 mg) as a pale brown solid.

NMR(200 MHz,DMSO-d₆,δ): 1.15 (3H, t, J=7.2 Hz), 1.78-1.85 (2H, m), 2.34 (2H, t, J=7.3 Hz), 3.72 (2H, t, J=6.4 Hz), 4.02 (2H, q, J=7.2 Hz), 4.16 (2H, br), 4.31 (2H, d, J=5.8 Hz), 5.35 (1H, t, J=5.8 Hz), 5.76 (1H, d, J=2.6 Hz), 6.00 (1H, dd, J=2.6 Hz, 8.2 Hz), 6.48 (1H, d, J=8.2 Hz), 7.37 (1H, s), 7.38 (1H, d, J=1.9 Hz), 7.61 (1H, d, J=1.9 Hz).

MS: 397 (M+1).

### PREPARATION EXAMPLE 89

A mixture of ethyl 4-{4-amino-3-[(2,4-dichlorobenzyl)amino]phenoxy}butanoate (236 mg), tetraethyl orthocarbonate (C(OEt)₄) (0.62 ml) and acetic acid (AcOH) (0.044 mL) was heated at 80° C. for 1 hour. After cooling to room temperature, the mixture was evaporated. The residue was dissolved in EtOAc (5 mL) and washed with saturated aqueous sodium hydrogencarbonate (NaHCO₃) solution (2×20 mL) and brine (20 mL). The organic layer was dried (Na₂SO₄), filtered and concentrated. The residue was triturated with hexane to give ethyl 4-{[1-(2,4-dichlorobenzyl)-2-ethoxy-1H-benzimidazol-6-yl]oxy}butanoate (170 mg) as pale red crystals.

NMR(200 MHz,DMSO-d₆,δ): 1.16 (3H, t, J=7.2 Hz), 1.31 (3H, t, J=7.0 Hz), 1.89-1.99 (2H, m), 2.43 (2H, t, J=7.2 Hz), 3.91 (2H, t, J=6.3 Hz), 4.04 (2H, q, J=7.2 Hz), 4.46 (2H, q, J=7.0 Hz), 5.25 (2H, s), 6.70 (1H, dd, J=2.4 Hz, 8.6 Hz), 6.80 (1H, d, J=8.4 Hz), 6.91 (1H, d, J=2.4 Hz), 7.30 (1H, d, J=8.6 Hz), 7.37 (1H, dd, J=2.2 Hz, 8.4 Hz), 7.69 (1H, d, J=2.2 Hz).

MS: 451 (M+1).

### PREPARATION EXAMPLE 90

To a 0° C. suspension of NaH (184 mg, 60% oil dispersion) in DMF (10 mL) was added a solution of ethyl 4-(4-amino-3-methyl-5-nitrophenoxy)butanoate (0.5 g) in DMF (10 mL). After 30 minutes at room temperature, the solution was treated with di-tert-butyl dicarbonate (0.9 ml). The resultant mixture was stirred for 3 hours at room temperature before the reaction was quenched by addition of water (20 mL). The mixture was extracted with EtOAc (3×20 mL). The combined extracts were washed with brine (20 mL), dried over Na₂SO₄ and concentrated under reduced pressure. Purification of the residue by filtration through a pad of silica gel (EtOAc/hexane 1/2) afforded ethyl 4-{4-[bis(tert-butoxycarbonyl)amino]-3-methyl-5-nitrophenoxy}butanoate (1.2 g) as a dark yellow oil.

NMR(200 MHz,DMSO-d₆,δ): 1.14 (3H, t, J=7.2 Hz), 1.47 (18H, s), 1.95-2.02 (2H, m), 2.18 (3H, s), 2.46 (2H, t, J=7.2 Hz), 4.03 (2H, t, J=7.1 Hz), 4.09 (2H, q, J=7.2 Hz), 7.29 (1H, d, J=2.6 Hz), 7.43 (1H, d, J=2.6 Hz).

MS: 505 (M+Na)

### PREPARATION EXAMPLE 91

To a solution of ethyl 4-{4-[bis(tert-butoxycarbonyl)amino]-3-methyl-5-nitrophenoxy}butanoate (834 mg) in EtOH (16 mL) was added palladium on carbon (10%, 50% wet, 300 mg) at room temperature, and the resultant mixture was hydrogenated under atmospheric pressure of hydrogen for 3 hours. The catalyst was removed by filtration. The filtrate was evaporated to give ethyl 4-{3-amino-4-[bis(tert-butoxycarbonyl)amino]-5-methylphenoxy}butanoate (534 mg).

NMR(200 MHz,DMSO-d₆,δ): 1.18 (3H, t, J=7.2 Hz), 1.36 (18H, s), 1.85-1.99 (5H, m), 2.43 (2H, t, J=7.2 Hz), 3.86 (2H, t, J=6.3 Hz), 4.03 (2H, q, J=7.2 Hz), 4.73 (2H, s), 6.00 (1H, d, J=2.6 Hz), 6.13 (1H, d, J=2.6 Hz).

MS: 475 (M+1).

### PREPARATION EXAMPLE 92

A mixture of ethyl 4-{3-amino-4-[bis(tert-butoxycarbonyl)amino]-5-methylphenoxy}butanoate (528 mg), 2,4-dichlorobenzylchloride (251 mg), NaI (17.5 mg) and K₂CO₃ (194 mg) in DMF (5 mL) was heated at 80° C. for 5 hours. After cooling to room temperature, the reaction mixture was poured into a saturated NH₄Cl solution (30 mL). The phases were separated and the aqueous layer was extracted with 2×15 mL of EtOAc. The combined extracts were washed with brine (20 mL), dried over Na₂SO₄, and concentrated to provide the crude ester. Purification of the product by column chromatography (EtOAc/n-hexane 1/4) provided ethyl 4-{4-[bis(tert-butoxycarbonyl)amino]-3-[(2,4-dichlorobenzyl)amino]-5-methylphenoxy}butanoate (480 mg), which was used in the next step without further purification.

### PREPARATION EXAMPLE 93

To a solution of ethyl 4-{4-[bis(tert-butoxycarbonyl)amino]-3-[(2,4-dichlorobenzyl)amino]-5-methylphenoxy}butanoate (560 mg) in EtOH (4 mL) was added 4N HCl in dioxane (2.3 mL) at room temperature. The mixture was stirred at room temperature for 14 hours, and then concentrated in vacuo without external heating. The residue was dissolved in 5 mL of EtOAc, and neutralized with 1N NaOH. The phases were separated and the aqueous layer was extracted with EtOAc (2×10 mL). The combined organic extracts were washed with brine (10 mL), dried (Na₂SO₄), and concentrated. The residue was purified by column chromatography on silica gel (EtOAc/hexane 1/4) to afford ethyl 4-{4-amino-3-[(2,4-dichlorobenzyl)amino]-5-methylphenoxy}butanoate (206 mg) as a pale brown solid.

NMR(200 MHz,DMSO-d₆,δ): 4.30(2H, d, J=5.8 Hz), 5.43 (1H, t, J=5.8 Hz), 5.67 (1H, d, J=2.5 Hz), 5.96 (1H, d, J=2.5 Hz), 7.33 (1H, d, J=8.4 Hz), 7.38 (1H, dd, J=1.8 Hz, 8.4 Hz), 7.61 (1H, d, J=1.8 Hz).

MS: 411 (M+1).

### PREPARATION EXAMPLE 94

To a solution of ethyl 4-{4-amino-3-[(2,4-dichlorobenzyl)amino]-5-methylphenoxy}butanoate (200 mg) in EtOH (2 mL) was added methyl isothiocyanate (46 mg) at room temperature. The resulting solution was heated at 50° C. for 2 hours. After cooling to room temperature, methyl isothiocyanate (80 mg) was added. The resulting solution was heated at 60° C. for 2 hours. After cooling, to the reaction mixture was added dropwise water (10 mL). The mixture was extracted with EtOAc (2×10 mL). The combined extracts were washed with brine (10 mL), dried over Na₂SO₄. Removal of the solvent proceeded the crude ethyl 4-(3-[(2,4-dichlorobenzyl)amino]-5-methyl-4-{[(methylamino)carbonothioyl]amino}phenoxy)butanoate (252 mg), which was used in the next step without further purification.

### PREPARATION EXAMPLE 95

To a solution of ethyl 4-(3-[(2,4-dichlorobenzyl)amino]-5-methyl-4-{[(methylamino)carbonothioyl]amino}phenoxy)butanoate (252 mg) in toluene (5 ml) was added 1-(3-dimethylaminopropyl)-3-ethylcarbodiimide hydrochloride (WSCD.HCl) (150 mg) at ambient temperature. The mixture was heated at 110° C. for 1 hour. After cooling to room temperature, the reaction mixture was diluted with EtOAc (20 ml), and washed with saturated aq NaHCO₃ (2×20 ml), saturated aqueous NH₄Cl (2×20 ml), and brine (20 ml). The organic layer was dried (Na₂SO₄) and filtered. Evaporation gave a residue (606 mg) which was triturated with EtOAc (1 ml)—n-hexane (3 ml) to give ethyl 4-{[1-(2,4-dichlorobenzyl)-4-methyl-2-(methylamino)-1H-benzimidazol-6-yl]oxy}butanoate (156 mg) as white crystals.

NMR(200 MHz,DMSO-d₆,δ): 1.14 (3H, t, J=7.1 Hz), 1.85-1.95 (2H, m), 2.38 (3H, s), 2.40 (2H, t, J=7.0 Hz), 2.86 (3H, d, J=4.6 Hz), 3.84 (2H, t, J=6.3 Hz), 6.03 (2H, q, J=7.1 Hz), 5.21 (2H, s), 6.35 (1H, d, J=8.4 Hz), 6.42-6.52 (3H, m), 7.31 (1H, dd, J=2.1 Hz, 8.3 Hz), 7.69 (1H, d, J=2.1 Hz).

MS: 450 (M+1).

### PREPARATION EXAMPLE 96

A mixture of ethyl 4-[(7-methyl-2-thioxo-2,3-dihydro-1H-benzimidazol-5-yl)oxy]butanoate (350 mg), iodoethane (EtI) (0.191 mL), diisopropylethylamine (0.414 mL) and DMF (3.5 mL) was stirred overnight at room temperature. The mixture was then diluted with EtOAc (15 mL) and poured into water (20 mL). The phases were separated and the aqueous layer was extracted with EtOAc (2×10 mL). The combined extracts were washed with brine (20 mL), dried over Na₂SO₄, and concentrated to provide the crude ester. Purification by chromatography on silica gel (EtOAc/n-hexane 1/1) gave ethyl 4-{[2-(ethylthio)-4-methyl-1H-benzimidazol-6-yl]oxy}butanoate (434 mg) as a dark brown oil.

NMR(200 MHz,DMSO-d₆,δ): 1.18 (3H, t, J=7.1 Hz), 1.36 (3H, t, J=7.3 Hz), 1.94-1.99 (2H, m), 2.46 (2H, t, J=7.3 Hz), 2.51 (3H, S), 3.22 (2H, q, J=7.3 Hz), 3.94 (2H, t, J=6.3 Hz), 4.07 (2H, q, J=7.1 Hz), 6.55 (1H, s), 6.7 (1H, br), 12.2 (1H, br).

MS: 323 (M+1).

### PREPARATION EXAMPLE 97

A mixture of ethyl 4-{[2-(ethylthio)-4-methyl-1H-benzimidazol-6-yl]oxy}butanoate (384 mg), 2,4-dichlorobenzyl chloride (0.18 mL) and K₂CO₃ (198 mg) in DMF (4 mL) was stirred overnight at room temperature. The mixture was then diluted with EtOAc (10 mL) and poured into water (25 mL). The phases were separated and the aqueous layer was extracted with EtOAc (2×15 mL). The combined organic extracts were washed with brine (20 mL), dried over Na₂SO₄, and concentrated to provide the crude ester (840 mg). Purification of the product by column chromatography (EtOAc/n-hexane 1/6) provided ethyl 4-{[1-(2,4-dichlorobenzyl)-2-(ethylthio)-4-methyl-1H-benzimidazol-6-yl]oxy}butanoate (477 mg).

NMR(200 MHz,DMSO-d₆,δ): 1.22 (3H, t, J=7.1 Hz), 1.39 (3H, t, J=7.3 Hz), 2.00-2.13 (2H, m), 2.46 (2H, t, J=7.2 Hz), 2.62 (3H, s), 3.29 (2H, q, J=7.3 Hz), 3.94 (2H, t, J=6.1 Hz), 4.12 (2H, q, J=7.1 Hz), 5.32 (2H, s), 6.40 (1H, d, J=2.2 Hz), 6.46 (1H, d, J=8.4 Hz), 6.67 (1H, d, J=2.2 Hz), 7.06 (1H, dd, J=2.0 Hz, 8.4 Hz), 7.45 (1H, d, J=2.0 Hz).

MS: 481 (M+1).

### PREPARATION EXAMPLE 98

Ethyl 2-[4-(acetylamino)-3-methyl-5-nitrophenoxy]-2-methylpropanoate (1.49 g) was synthesized from N-(4-hydroxy-2-methyl-6-nitrophenyl)acetamide (1.0 g) in a manner similar to that described in Preparation Example 35 except that ethyl 2-bromo-2-methylpropanoate (1.39 g) was used instead of ethyl 4-bromobutanoate.

NMR(CDCl₃,δ): 1.28 (3H, t, J=6.0 Hz), 1.62 (6H, s), 2.20 (3H, s), 2.28 (3H, s), 4.26 (2H, q, J=6.0 Hz), 7.02 (1H, d, J=1.0 Hz), 7.29 (1H, d, J=1.0 Hz), 8.02 (1H, br s).

MS: 347 (M+Na).

### PREPARATION EXAMPLE 99

Ethyl 2-[(2,4-dimethyl-1H-benzimidazol-6-yl)oxy]-2-methylpropanoate (1.43 g) was synthesized from ethyl 2-[4-(acetylamino)-3-methyl-5-nitrophenoxy]-2-methylpropanoate (1.49 g) in a manner similar to that described in Preparation Example 72.

NMR(CDCl₃,δ): 1.28 (3H, t, J=6.0 Hz), 1.56 (6H, s), 2.48 (3H, s), 2.57 (3H, s), 4.24 (2H, q, J=6.0 Hz), 6.66 (1H, br s), 6.84 (1H, br s).

MS: 276 (M+).

### PREPARATION EXAMPLE 100

Ethyl 2-({1-[(3-chloro-1,1′-biphenyl-4-yl)methyl]-2,4-dimethyl-1H-benzimidazol-6-yl)oxy)-2-methylpropanoate (155 mg) was synthesized from ethyl 2-[(2,4-dimethyl-1H-benzimidazol-6-yl)oxy]-2-methylpropanoate (100 mg) in a manner similar to that described in Preparation Example 105 except that (3-chloro-1,1′-biphenyl-4-yl)methyl bromide (122 mg) was used instead of (3-methoxy-1,1′-biphenyl-4-yl)methyl chloride.

NMR(CDCl₃,δ): 1.26 (3H, t, J=6.0 Hz), 1.52 (6H, s), 2.57 (3H, s), 2.62 (3H, s), 4.12 (2H, q, J=6.0 Hz), 5.33 (2H, s), 6.45-6.55 (2H, m), 6.68 (1H, br s), 7.22-7.57 (6H, m), 7.68 (1H, br s).

MS: 477 (M+).

### PREPARATION EXAMPLE 101

Ethyl 2-({1-[2-chloro-4-(pentyloxy)benzyl]-2,4-dimethyl-1H-benzimidazol-6-yl}oxy)-2-methylpropanoate (142 mg) was synthesized from ethyl 2-[(2,4-dimethyl-1H-benzimidazol-6-yl)oxy]-2-methylpropanoate (100 mg) in a manner similar to that described in Preparation Example 105 except that 1-bromomethyl-2-chloro-4-(pentyloxy)benzene (158 mg) was used instead of (3-methoxy-1,1′-biphenyl-4-yl)methyl chloride.

NMR(CDCl₃,δ): 0.90 (3H, t, J=6.0 Hz), 1.20 (3H, t, J=6.0 Hz), 1.26-1.48 (4H, m), 1.52 (6H, s), 1.65-1.87 (2H, m), 2.53 (3H, s), 2.61 (3H, s), 3.88 (2H, t, J=6.0 Hz), 4.12 (2H, q, J=6.0 Hz), 5.22 (2H, s), 6.37 (1H, d, J=8.0 Hz), 6.51 (1H, d, J=2.0 Hz), 6.60 (1H, dd, J=8.0 Hz, 2.0 Hz), 6.67 (1H, d, J=2.0 Hz), 6.97 (1H, d, J=2.0 Hz).

MS: 487 (M+).

### PREPARATION EXAMPLE 102

Ethyl 4-({1-[(3-chloro-5-ethoxy-2-pyridinyl)methyl]-2,4-dimethyl-1H-benzimidazol-6-yl}oxy)butanoate (188 mg) was synthesized from ethyl 2-[(2,4-dimethyl-1H-benzimidazol-6-yl)oxy]butanoate (146 mg) in a manner similar to that described in Preparation Example 105 except that (3-chloro-5-ethoxy-2-pyridinyl)methyl methanesulfonate (140 mg) was used instead of (3-methoxy-1,1′-biphenyl-4-yl)methyl chloride.

NMR(CDCl₃,δ): 1.23 (3H, t, J=6.0 Hz), 1.40 (3H, t, J=6.0 Hz), 2.00-2.18 (2H, m), 2.52 (2H, t, J=6.0 Hz), 2.58 (3H, s), 2.65 (3H, s), 3.90-4.20 (6H, m), 5.36 (2H, s), 6.63 (2H, s), 7.20 (1H, d, J=2.0 Hz), 8.08 (1H, d, J=2.0 Hz).

MS: 446 (M+).

### PREPARATION EXAMPLE 103

Ethyl 4-({1-[2-chloro-4-(1,3-oxazol-2-yl)benzyl]-2,4-dimethyl-1H-benzimidazol-6-yl}oxy)butanoate (153 mg) was synthesized from ethyl 2-[(2,4-dimethyl-1H-benzimidazol-6-yl)oxy]butanoate (100 mg) in a manner similar to that described in Preparation Example 105 except that 1-bromomethyl-2-chloro-4-(1,3-oxazol-2-yl)benzene (108 mg) was used instead of (3-methoxy-1,1′-biphenyl-4-yl)methyl chloride.

NMR(DMSO-d₆,δ): 1.13 (3H, t, J=6.0 Hz), 1.84-2.00 (2H, m), 2.40 (2H, t, J=6.0 Hz), 2.41 (3H, s), 2.51 (3H, s), 3.90 (2H, t, J=6.0 Hz), 4.02 (2H, q, J=6.0 Hz), 5.52 (2H, s), 6.50-6.62 (2H, d), 6.80 (1H, d, J=1.0 Hz), 7.40 (1H, s), 7.92 (1H, dd, J=8.0 Hz, 1.0 Hz), 8.05 (1H, d, J=1.0 Hz), 8.24 (1H, s).

MS: 468 (M+).

### PREPARATION EXAMPLE 104

Ethyl 4-({1-[(2-chloro-6-phenyl-3-pyridinyl)methyl]-2,4-dimethyl-1H-benzimidazol-6-yl}oxy)butanoate (365 mg) was synthesized from ethyl 2-[(2,4-dimethyl-1H-benzimidazol-6-yl)oxy]butanoate (236 mg) in a manner similar to that described in Preparation Example 105 except that (2-chloro-6-phenyl-3-pyridinyl)methyl methanesulfonate (280 mg) was used instead of (3-methoxy-1,1′-biphenyl-4-yl)methyl chloride.

NMR(CDCl₃,δ): 1.22 (3H, t, J=6.0 Hz), 2.00-2.18 (2H, m), 2.50 (2H, t, J=6.0 Hz), 2.56 (3H, s), 2.64 (3H, s), 3.97 (2H, t, J=6.0 Hz), 4.11 (2H, q, J=6.0 Hz), 5.34 (2H, s), 6.49 (1H, d, J=1.0 Hz), 6.72 (1H, d, J=1.0 Hz), 6.78 (1H, d, J=8.0 Hz), 7.39-7.52 (4H, m), 7.90-8.00 (2H, m).

MS: 478 (M+)

### PREPARATION EXAMPLE 105

A mixture of ethyl 4-[(2,4-dimethyl-1H-benzimidazol-6-yl)oxy]butanoate (148 mg), (3-methoxy-1,1′-biphenyl-4-yl)methyl chloride (125 mg), sodium carbonate (222 mg) and N,N-dimethylformamide (DMF, 2 mL) was stirred at 80° C. for 7 hours. The mixture was partitoned between ethyl acetate and brine. The organic layer was washed with brine, dried over magnesium sulfate (MgSO₄) and evaporated in vacuo. The residue was purified by preparative thin layer chromatography (p-TLC, ethyl acetate) to give ethyl 4-({1-[(3-methoxy-1,1′-biphenyl-4-yl)methyl]-2,4-dimethyl-1H-benzimidazol-6-yl}oxy)butanoate as a pale yellow oil (181 mg).

NMR(CDCl₃,δ): 1.22 (3H, t, J=6.0 Hz), 2.00-2.18 (2H, m), 2.50 (2H, t, J=6.0 Hz), 2.57 (3H, s), 2.63 (3H, s), 3.97 (2H, t, J=6.0 Hz), 3.98 (3H, s), 4.12 (2H, q, J=6.0 Hz), 5.27 (2H, s), 6.52-6.72 (3H, m), 7.02 (1H, dd, J=8.0 Hz, 1.0 Hz), 7.10 (1H, d, J=1.0 Hz), 7.28-7.58 (5H, m).

MS: 473 (M+).

### PREPARATION EXAMPLE 106

Ethyl 4-[(1-{4-[bis(tert-butoxycarbonyl)amino]-2-chlorobenzyl}-2,4-dimethyl-1H-benzimidazol-6-yl)oxy]butanoate (1.67 g) was synthesized from ethyl 2-[(2,4-dimethyl-1H-benzimidazol-6-yl)oxy]butanoate (10 g) in a manner similar to that described in Preparation Example 134.

NMR(DMSO-d₆,δ): 1.15 (3H, t, J=7 Hz), 1.36 (18H, s), 1.8-2.0 (2H, m), 2.40 (3H, s), 2.4-2.6 (total 5H: 3H, s and 2H, t), 3.89 (2H, t, J=6 Hz), 4.04 (2H, q, J=7 Hz), 5.48 (2H, s), 6.45 (1H, d, J=8 Hz), 6.60 (1H, d, J=1 Hz), 6.78 (1H, d, J=2 Hz), 7.08 (1H, dd, J=2 Hz, 8 Hz), 7.47 (1H, d, J=2 Hz).

MS: 616 (M+H).

### PREPARATION EXAMPLE 107

Ethyl 4-{[1-(4-amino-2-chlorobenzyl)-2,4-dimethyl-1H-benzimidazol-6-yl]oxy}butanoate (1.0 g) was synthesized from ethyl 4-[(1-(4-[bis(tert-butoxycarbonyl)amino]-2-chlorobenzyl}-2,4-dimethyl-1H-benzimidazol-6-yl)oxy]butanoate (1.67 g) in a manner similar to that described in Preparation Example 135.

NMR(DMSO-d₆,δ): 1.16 (3H, t, J=7 Hz), 1.8-2.0 (2H, m), 2.40 (3H, s), 2.44 (3H, s), 2.4-2.6 (2H, t), 3.90 (2H, t, J=6 Hz), 4.05 (2H, q, J=7 Hz), 5.23 (2H, s), 5.39 (2H, br s), 6.3-6.5 (2H, m), 6.57 (1H, d, J=1 Hz), 6.65 (1H, d, J=2 Hz), 6.72 (1H, d, J=2 Hz).

MS: 415 (M+H).

### PREPARATION EXAMPLE 108

Ethyl 4-({1-[2-chloro-4-(dimethylamino)benzyl]-2,4-dimethyl-1H-benzimidazol-6-yl}oxy)butanoate (0.18 g) was synthesized from ethyl 4-{[1-(4-amino-2-chlorobenzyl)-2,4-dimethyl-1H-benzimidazol-6-yl]oxy}butanoate (600 mg) in a manner similar to that described in Preparation Example 136.

NMR(DMSO-d₆,δ): 1.15 (3H, t, J=7 Hz), 1.8-2.0 (2H, m), 2.40 (3H, s), 2.42 (2H, t, J=7 Hz), 2.44 (3H, s), 2.85 (6H, s), 3.90 (2H, t, J=6 Hz), 4.04 (2H, q, J=7 Hz), 5.30 (2H, s), 6.43 (1H, d, J=8 Hz), 6.5-6.6 (2H, m), 6.72 (1H, d, J=2 Hz), 6.75 (1H, d , J=2 Hz).

MS: 444 (M+H).

### PREPARATION EXAMPLE 109

A mixture of N-(4-hydroxy-2-nitrophenyl)acetamide (5.0 g), ethyl 4-bromobutanoate (5.47 g), potassium carbonate (3.88 g) and N,N-dimethylformamide (50 mL) was stirred at ambient temperature for 2 days. The mixture was poured into water and extracted twice with ethyl acetate. The extracts were combined, washed with 1-N sodium hydroxide and brine, dried over anhydrous magnesium sulfate, and concentrated in vacuo. The resulting solid was suspended in diisopropyl ether (150 mL). The suspension was stirred at ambient temperature for an hour and filtered to give ethyl 4-[4-(acetylamino)-3-nitrophenoxy]butanoate (6.8 g) as a yellow solid.

NMR(DMSO-d₆,δ): 1.17 (3H, t, J=7 Hz), 1.9-2.0 (2H, m), 2.01 (3H, s), 2.45 (2H, t, J=6 Hz), 4.06 (2H, t, J=6 Hz), 4.06 (2H, q, J=7 Hz), 7.26 (1H, dd, J=3 Hz, 9 Hz), 7.42 (1H, d J=3 Hz), 7.44 (1H, d , J=9 Hz), 10.04 (1H, br s).

MS: 333 (M+Na)

### PREPARATION EXAMPLE 110

A mixture of ethyl 4-[4-(acetylamino)-3-nitrophenoxy]butanoate (8.0 g), 10% palladium on carbon (50% wet; 0.80 g), tetrahydrofuran (180 mL) and ethanol (180 mL) was stirred under 1 atmosphere of hydrogen at ambient temperature for 3 hours. The catalyst was filtered off and washed with ethanol. The filtrate and washings were concentrated in vacuo. The residue was dissolved in ethanol (20 mL) and treated with 4 N hydrogen chloride/1,4-dioxane (8 mL). The resulting suspension was stirred at ambient temperature for half an hour and diluted with diisopropyl ether (80 mL). After stirring for additional half an hour, the suspension was filtered to give ethyl 4-[4-(acetylamino)-3-aminophenoxy]butanoate hydrochloride (7.2 g) as a white solid.

### PREPARATION EXAMPLE 111

To an ice-cooled solution of 3-methoxy-1,1′-biphenyl-4-carbaldehyde (940 mg) in methanol (9.4 mL) and tetrahydrofuran (THF, 9.4 mL) was added sodium borohydride (168 mg). The mixture was stirred at the same temperature for 40 minutes. The reaction mixture was partitioned between ethyl acetate and brine. The organic layer was separated, dried over magnesium sulfate (MgSO₄) and evaporated in vacuo to give (3-methoxy-1,1′-biphenyl-4-yl)methanol (985 mg) as a colorless oil.

NMR(CDCl₃,δ): 2.30 (1H, t, J=6.4 Hz), 3.93 (3H, s), 4.73 (2H, d, J=6.4 Hz), 7.09 (1H, d, J=1.4 Hz), 7.19 (1H, dd, J=8.0 Hz, 1.4 Hz), 7.22-7.61 (6H, m).

MS: 237 (M+Na).

### PREPARATION EXAMPLE 112

To a solution of (3-methoxy-1,1′-biphenyl-4-yl)methanol (500 mg) in toluene (5 mL) was added methanesulfonyl chloride (294 mg), then to the mixture was added triethylamine (260 mg) dropwise with cooling in an ice bath. And the mixture was stirred at ambient temperature for 1 hour. The reaction mixture was partitioned between ethyl acetate and water, and the organic layer was separated, washed with saturated NaHCO₃ and brine, dried over MgSO₄ and concentrated in vacuo to give 4-(chloromethyl)-1,1′-biphenyl-3-yl methyl ether (694 mg) as a pale yellow oil, which was used without further purification.

¹H-NMR(CDCl₃,δ): 3.95 (3H, s), 4.71 (2H, s), 7.09 (1H, d, J=2 Hz), 7.17 (1H, dd, J=2.8 Hz), 7.34-7.48 (4H, m), 7.55-7.61 (2H, m).

### PREPARATION EXAMPLE 113

To a solution of oxazole (800 mg) in tetrahydrofuran (60 mL) was added n-butyllithium (1.59 M solution in hexanes, 8.01 mL) at −70° C. After stirring for 30 minutes, the mixture was added zinc chloride (1.0 M solution in diethyl ether, 34.7 mL). The reaction mixture was warmed to 0° C., and added a solution of 2-chloro-4-iodotoluene (2.92 g) in tetrahydrofuran (40 mL) and tetrakis (triphenylphosphine) palladium(0) (1.34 g). The resulting mixture was heated to reflux for 7 hours. After cooling, the final reaction mixture was evaporated in vacuo. The residue was partitioned between ethyl acetate with H₂O. The organic layer was separated, washed with brine, dried over MgSO₄, filtered, and evaporated in vacuo. The crude product was purified by column chromatography on silica gel (eluted with hexane-ethyl acetate=15-1 to 10-1) to give 2-(3-chloro-4-methylphenyl)-1,3-oxazole (843 mg).

¹H-NMR(CDCl₃,δ): 2.43 (3H, s), 7.23 (1H, s), 7.32 (1H, d, J=8 Hz), 7.70 (1H, s), 7.83 (1H, dd, J=2 Hz, 8 Hz), 8.03 (1H, d, J=2 Hz).

MS (ESI): m/z 194 (M+1)

### PREPARATION EXAMPLE 114

To a mixture of 2-(3-chloro-4-methylphenyl)-1,3-oxazole (200 mg) in carbon tetrachloride (3.1 mL) were added N-bromosuccinimide (184 mg) and V-70 (15.9 mg). The mixture was stirred at room temperature for 1 hour, at 60° C. for 2 hours and at 80° C. for 2 hours. To the reaction mixture were added N-bromosuccinimide (92 mg) and V-70 (15.9 mg). The resulting mixture was stirred at 80° C. for 1 hour. After cooling, to the final reaction mixture was added hexane. A resulting precipitate was filtered out, and the filtrate was evaporated in vacuo to give 2-[4-(bromomethyl)-3-chlorophenyl]-1,3-oxazole (346 mg).

¹H-NMR(CDCl₃,δ): 4.63 (2H, s), 7.27 (1H, s), 7.55 (1H, d, J=8 Hz), 7.75 (1H, s), 7.94 (1H, dd, J=2.8 Hz), 8.10 (1H, d, J=2 Hz).

MS (ESI): m/z 272 (M+1).

### PREPARATION EXAMPLE 115

Methyl 3-[({1-[2-chloro-4-(pentyloxy)benzyl]-2,4-dimethyl-1H-benzimidazol-6-yl}oxy)methyl]benzoate (50 mg) was synthesized from 1-[2-chloro-4-(pentyloxy)benzyl]-2,4-dimethyl-1H-benzimidazol-6-ol (50 mg) in a manner similar to that described in Preparation Example 33 except that methyl 3-(bromomethyl)benzoate (30.7 mg) was used instead of 4-bromobutanoate.

NMR(DMSO-d₆,δ): 0.8-1.0 (3H, m), 1.2-1.5 (4H, m), 1.5-1.8 (2H, m), 2.40 (3H, s), 2.47 (3H, s), 3.85 (3H, s), 3.93 (2H, t, J=6.4 Hz), 5.12 (2H, br s), 5.36 (2H, br s), 6.41 (1H, d, J=8.7 Hz), 6.72 (1H, d, J=2.0 Hz), 6.78 (1H, dd, J=2.5 Hz, 8.6 Hz), 6.87 (1H, d, J=2.0 Hz), 7.08 (1H, d, J=2.5 Hz), 7.51 (1H, t, J=7.7 Hz), 7.68 (1H, d, J=7.7 Hz), 7.89 (1H, d, J=7.7 Hz), 8.01 (1H, br s).

MS: 521 (M+1).

### PREPARATION EXAMPLE 116

A two-phase mixture of 4-amino-3-nitrophenol (1.0 g), methyl 2-(bromomethyl)benzoate (1.78 g), tetra-n-butylammonium hydrogen sulfate (0.22 g), 1 N sodium hydroxide (13 mL) and dichloromethane (26 mL) was stirred vigorously at ambient temperature for 6 hours. The pH of the mixture was adjusted to 3 with 10N hydrochloric acid. The precipitate formed was collected and washed successively with water and ethyl acetate to give methyl 2-[(4-amino-3-nitrophenoxy)methyl]benzoate (0.76 g) as an orange solid. The filtrate was diluted with tetrahydrofuran, and the organic layer was separated, washed with 1N sodium hydroxide, water and brine, dried over anhydrous magnesium sulfate, concentrated in vacuo. The resulting solid was suspended in ethanol (20 mL) and filtered to afford the further product (0.45 g).

NMR(DMSO-d₆,δ): 3.82 (3H, s), 5.35 (2H, s), 7.02 (1H, d, J=9 Hz), 7.24 (1H, dd, J=3 Hz, 9 Hz), 7.29 (2H, br s), 7.43 (1H, d, J=3 Hz), 7.4-8.0 (4H, m).

MS: 325 (M+Na).

### PREPARATION EXAMPLE 117

To a solution of methyl 2-[(4-amino-3-nitrophenoxy)methyl]benzoate (0.50 g) in a solvent mixture of tetrahydrofuran (5 mL) and N,N-dimethylformamide (5 mL) was added sodium hydride (60% dispersion in mineral oil; 72 mg), and the mixture was stirred at ambient temperature for half an hour. To the mixture was added di-tert-butyl dicarbonate (0.39 g) and stirring was continued overnight at ambient temperature. The mixture was partitioned between ethyl acetate and saturated aqueous ammonium chloride. The organic layer was separated, washed with brine, dried over anhydrous sodium sulfate, and concentrated in vacuo. The residue was purified by column chromatography on silica gel (100:1 chloroform-ethyl acetate) to give methyl 2-({4-[(tert-butoxycarbonyl)amino]-3-nitrophenoxy}methyl)benzoate (0.19 g) as a yellow solid.

NMR(DMSO-d₆,δ): 1.42 (9H, s), 3.81 (3H, s), 5.47 (2H, s), 7.32 (1H, dd; J=3 Hz, 9 Hz), 7.48 (1H, d, J=9 Hz), 7.51 (1H, d, J=3 Hz), 7.4-8.0 (4H, m), 9.34 (1H, s).

MS: 425 (M+Na).

### PREPARATION EXAMPLE 118

A mixture of methyl 2-({4-[(tert-butoxycarbonyl)amino]-3-nitrophenoxy}methyl)benzoate (0.15 g), iron (0.28 g) and ammonium chloride (0.40 g) in a solvent mixture of methanol (10 mL) and water (4 mL) was refluxed for an hour. After cooling, the mixture was filtered and the insoluble material was washed with methanol. The filtrate and the washings were combined and concentrated in vacuo. The residue was partitioned between ethyl acetate and 1 N sodium hydroxide. The organic layer was separated, washed with brine, dried over anhydrous sodium sulfate, and concentrated in vacuo. The residue was purified by column chromatography on silica gel (elution; 1:10 chloroform-ethyl acetate) followed by trituration with a mixture of n-hexane and ethyl acetate to give methyl 2-({3-amino-4-[(tert-butoxycarbonyl)amino]phenoxy}methyl)benzoate (0.10 g).

NMR(DMSO-d₆,δ): 1.43 (9H, s), 3.82 (3H, s), 4.84 (2H, br s), 5.30 (2H, s), 6.15 (1H, dd, J=3 Hz, 9 Hz), 6.32 (1H, d, J=3 Hz), 6.96 (1H, d, J=9 Hz), 7.4-8.0 (4H, m), 8.10 (1H, br s).

MS: 395 (M+Na).

### PREPARATION EXAMPLE 119

A mixture of methyl 2-({3-amino-4-[(tert-butoxycarbonyl)amino]phenoxy}methyl)benzoate (0.20 g), 4-(bromomethyl)-3-chloro-1,1′-biphenyl (0.18 g), potassium carbonate (89 mg) and N,N-dimethylformamide (4 mL) was stirred at 80° C. for 2 hours. After cooling, the mixture was partitioned between ethyl acetate and water. The organic layer was separated, washed with brine, dried over anhydrous sodium sulfate, and-concentrated in vacuo. The residue was purified by column chromatography on silica gel (elution; 4:1 n-hexane-acetone) to give methyl 2-[(4-[(tert-butoxycarbonyl)amino]-3-{[(3-chloro-1,1′-biphenyl-4-yl)methyl]amino}phenoxy)methyl]benzoate (0.27 g) as an amorphous form.

NMR(DMSO-d₆,δ): 1.45 (9H, s), 3.73 (3H, s), 4.37 (2H, br d, J=6 Hz), 5.26 (2H, s), 5.69 (1H, t, J=6 Hz), 5.93 (1H, d, J=2 Hz), 6.16 (1H, dd, J=2 Hz, 8 Hz), 6.98 (1H, d, J=8 Hz), 7.3-7.9 (12H, m), 8.1-8.3 (1H, br m).

MS: 573 (M+H).

### PREPARATION EXAMPLE 120

A solution of methyl 2-[(4-[(tert-butoxycarbonyl)amino]-3-{[(3-chloro-1,1′-biphenyl-4-yl)methyl]amino}phenoxy)methyl]benzoate (0.25 g) in methanol (4 mL) was treated with 4 N hydrochloric acid/1,4-dioxane (4 mL), and the mixture was stirred at 50° C. for an hour. After cooling, the mixture was partitioned between ethyl acetate and 1 N sodium hydroxide. The organic layer was separated in vacuo, washed with brine, dried over anhydrous sodium sulfate, and concentrated in vacuo to give the crude product which was used in the next reaction without further purification.

A mixture of the crude product, acetic acid (0.50 mL) and tetraethoxymethane (4.2 mL) was stirred at 80° C. for half an hour. After cooling, the mixture was partitioned between ethyl acetate and 1 N sodium hydroxide. The organic layer was separated, washed with brine, dried over anhydrous sodium sulfate, and concentrated in vacuo. The residue was purified by column chromatography on silica gel (gradient elution; chloroform-ethyl acetate 50:1 to 10:1) to afford the product which was triturated with methanol to give methyl 2-[({1-[(3-chloro-1,1′-biphenyl-4-yl)methyl]-2-ethoxy-1H-benzimidazol-6-yl}oxy)methyl]benzoate (0.12 g).

NMR(DMSO-d₆,δ): 1.35 (3H, t, J=7 Hz), 3.76 (3H, s), 4.51 (2H, q, J=7 Hz), 5.30 (2H, s), 5.35 (2H, s), 6.8-7.9 (15H, m).

MS: 527 (M+H).

### PREPARATION EXAMPLE 121

A mixture of N-(4-hydroxy-2-methyl-6-nitrophenyl)acetamide (10 g), methyl 2-(bromomethyl)benzoate (14.2 g), potassium carbonate (7.23 g) and N,N-dimethylformamide (100 mL) was stirred at ambient temperature overnight. To the mixture was added water (400 mL) to afford the precipitate and the suspension was stirred at ambient temperature for an hour. The precipitate was collected by filtration, washed with water, and dried in vacuo to give methyl 2-{[4-(acetylamino)-3-methyl-5-nitrophenoxy]methyl}benzoate (16.5 g) as a solid.

NMR(DMSO-d₆,δ): 2.00 (3H, s), 2.24 (3H, s), 3.82 (3H, s), 5.47 (2H, s), 7.27 (1H, d, J=3 Hz), 7.34 (1H, d, J=3 Hz), 7.4-7.9 (4H, m), 9.64 (1H, s).

MS: 381 (M+Na).

### PREPARATION EXAMPLE 122

A mixture of methyl 2-{[4-(acetylamino)-3-methyl-5-nitrophenoxy]methyl}benzoate (15.5 g) and iron (12.1 g) in a solvent mixture of tetrahydrofuran (80 mL), methanol (40 mL) and acetic acid (40 mL) was reflux for 8 hours. After cooling, the mixture was evaporated in vacuo. The residue was partitioned between ethyl acetate and 1 N sodium hydroxide/brine to afford the white precipitate which was collected to give methyl 2-{[(2,4-dimethyl-1H-benzimidazol-6-yl)oxy]methyl}benzoate (4.1 g). The organic layer was separated, washed with brine, dried over anhydrous sodium sulfate, and concentrated in vacuo. The residue was suspended in ethyl acetate (50 mL) and the suspension was stirred at ambient temperature for half an hour, filtered to give the further product (4.1 g) as a white solid.

NMR(DMSO-d₆,δ): 2.52 (3H, s), 2.74 (3H, s), 3.81 (3H, s), 5.48 (2H, s), 7.05 (1H, s), 7.07 (1H, d, J=2 Hz), 7.4-7.9 (4H, m), 14.33 (1H, br s).

MS: 311 (M+H).

### PREPARATION EXAMPLE 123

A mixture of methyl 2-{[(2,4-dimethyl-1H-benzimidazol-6-yl)oxy]methyl}benzoate (0.20 g), 4-(bromomethyl)-3-chloro-1,1′-biphenyl (0.29 g), potassium carbonate (0.10 g) and N,N-dimethylformamide (4 mL) was stirred at 80° C. for 5 hours. After cooling, the mixture was partitioned between ethyl acetate and water. The organic layer was separated, washed with brine, dried over anhydrous sodium sulfate, and concentrated in vacuo. The residue was purified by column chromatography on silica gel (gradient elution; chloroform-methanol 100:1 to 50:1) to afford the solid which was recrystallized from a mixture of diisopropyl ether (15 mL) and ethyl acetate (3 mL) to give methyl 2-[({1-[(3-chloro-1,1′-biphenyl-4-yl)methyl]-2,4-dimethyl-1H-benzimidazol-6-yl}oxy)methyl]benzoate (0.17 g).

NMR(DMSO-d₆,δ): 2.45 (3H, s), 2.49 (3H, s), 3.75 (3H, s), 5.34 (2H, s), 5.48 (2H, s), 6.54 (1H, d, J=8 Hz), 6.69 (1H, d, J=1 Hz), 6.83 (1H, d, J=2 Hz), 7.3-7.9 (11H, m).

MS: 511 (M+H).

### PREPARATION EXAMPLE 124

Methyl 2-({[1-(2-chloro-4-ethoxybenzyl)-2,4-dimethyl-1H-benzimidazol-6-yl]oxy}methyl)benzoate (0.27 g) was synthesized from methyl 2-{[(2,4-dimethyl-1H-benzimidazol-6-yl)oxy]methyl}benzoate (300 mg) in a manner similar to that described in Preparation Example 123 except that 2-chloro-4-ethoxybenzyl bromide (289 mg) was used instead of 4-(bromomethyl)-3-chloro-1,1′-biphenyl.

NMR(DMSO-d₆,δ): 1.29 (3H, t, J=7 Hz), 2.42 (3H, s), 2.47 (3H, s), 3.78 (3H, s), 3.99 (2H, q, J=7 Hz), 5.34 (4H, s), 6.4-6.6 (1H, m), 6.67 (1H, d, J=1 Hz), 6.76 (1H, d, J=1 Hz), 6.7-6.9 (1H, m), 7.05 (1H, d, J=3 Hz), 7.4-7.7 (4H, m).

MS: 479 (M+H).

### PREPARATION EXAMPLE 125

Methyl 2-{[(1-{[3-chloro-5-(trifluoromethyl)-2-pyridinyl]methyl}-2,4-dimethyl-1H-benzimidazol-6-yl)oxy]methyl}benzoate (0.30 g) was synthesized from methyl 2-{[(2,4-dimethyl-1H-benzimidazol-6-yl)oxy]methyl}benzoate (300 mg) in a manner similar to that described in Preparation Example 123 except that 3-chloro-2-(chloromethyl)-5-(trifluoromethyl)pyridine (311 mg) was used instead of 4-(bromomethyl)-3-chloro-1,1′-biphenyl.

NMR(DMSO-d₆,δ): 2.42 (3H, s), 2.46 (3H, s), 3.77 (3H, s), 5.33 (2H, s), 5.67 (2H, s), 6.64 (1H, d, J=1 Hz), 6.76 (1H, d, J=2 Hz), 7.3-8.0 (4H, m), 8.52 (1H, d , J=1 Hz), 8.74 (1H, s).

MS: 504 (M+H).

### PREPARATION EXAMPLE 126

Methyl 2-{[(1-{2-chloro-4-[(ethoxycarbonyl)(methyl)amino]benzyl}-2,4-dimethyl-1H-benzimidazol-6-yl)oxy]methyl}benzoate (1.10 g) was synthesized from methyl 2-{[(2,4-dimethyl-1H-benzimidazol-6-yl)oxy]methyl}benzoate (200 mg) in a manner similar to that described in Preparation Example 123 except that {1-bromomethyl-2-chloro-4-[(ethoxycarbonyl)(methyl)amino]}benzene (257 mg) was used instead of 4-(bromomethyl)-3-chloro-1,1′-biphenyl.

NMR(DMSO-d₆,δ): 1.14 (3H, t, J=7 Hz), 2.43 (3H, s), 2.48 (3H, s), 3.19 (3H, s), 3.76 (3H, s), 4.06 (2H, q, J=Hz), 5.33 (2H, S), 5.42 (2H, s), 6.47 (1H, d, J=8 Hz), 6.69 (1H, d, J=1 Hz), 6.83 (1H, d, J=2 Hz), 7.16 (1H, dd, J=2 Hz, 8 Hz), 7.3-7.7 (4H, m), 7.8-8.0 (1H, m).

MS: 536 (M+H).

### PREPARATION EXAMPLE 127

Methyl 2-[({1-[(3,5-dichloro-2-pyridinyl)methyl]-2,4-dimethyl-1H-benzimidazol-6-yl}oxy)methyl]benzoate (0.48 g) was synthesized from methyl 2-{[(2,4-dimethyl-1H-benzimidazol-6-yl)oxy]methyl}benzoate (560 mg) in a manner similar to that described in Preparation Example 123 except that (3,5-dichloro-2-pyridinyl)methyl methanesulfonate (508 mg) was used instead of 4-(bromomethyl)-3-chloro-1,1′-biphenyl.

NMR(DMSO-d₆,δ): 2.43 (3H, s), 2.45 (3H, s), 3.79 (3H, s), 5.33 (2H, s), 5.55 (2H, s), 6.64 (1H, d, J=1 Hz), 6.71 (1H, d, J=2 Hz), 7.3-8.0 (4H, m), 8.27 (1H, d, J=2 Hz), 8.40 (1H, d, J=2 Hz).

MS: 470 (M+H).

### PREPARATION EXAMPLE 128

Methyl 2-({[1-(2,4-dichlorobenzyl)-2,4-dimethyl-1H-benzimidazol-6-yl]oxy}methyl)benzoate (0.60 g) was synthesized from methyl 2-{[(2,4-dimethyl-1H-benzimidazol-6-yl)oxy]methyl}benzoate (500 mg) in a manner similar to that described in Preparation Example 123 except that 1-chloromethyl-2,4-dichlorobenzene (346 mg) was used instead of 4-(bromomethyl)-3-chloro-1,1′-biphenyl.

NMR(DMSO-d₆,δ): 2.41 (3H, s), 2.47 (3H, s), 3.77 (3H, s), 5.33 (2H, s), 5.42 (2H, s), 6.46 (1H, d, J=8 Hz), 6.68 (1H, d, J=1 Hz), 6.76 (1H, d, J=2 Hz), 7.28 (1H, dd, J=2 Hz, 8 Hz), 7.4-7.6 (3H, m), 7.68 (1H, d, J=2 Hz), 7.8-7.9 (1H, m).

MS: 469 (M+H).

### PREPARATION EXAMPLE 129

Methyl 2-[({1-[(2,6-dichloro-3-pyridinyl)methyl]-2,4-dimethyl-1H-benzimidazol-6-yl}oxy)methyl]benzoate (0.54 g) was synthesized from methyl 2-{[(2,4-dimethyl-1H-benzimidazol-6-yl)oxy]methyl}benzoate (560 mg) in a manner similar to that described in Preparation Example 123 except that (2,6-dichloro-3-pyridinyl)methyl methanesulfonate (508 mg) was used instead of 4-(bromomethyl)-3-chloro-1,1′-biphenyl.

NMR(DMSO-d₆,δ): 2.43 (3H, s), 2.48 (3H, s), 3.77 (3H, s), 5.34 (2H, s), 5.43 (2H, s), 6.69 (1H, d, J=1 Hz), 6.8-6.9 (2H, m), 7.41 (1H, d, J=8 Hz), 7.4-8.0 (4H, m).

MS: 472, 470 (M+H).

### PREPARATION EXAMPLE 130

Methyl 2-[({1-[(2-chloro-6-phenyl-3-pyridinyl)methyl]-2,4-dimethyl-1H-benzimidazol-6-yl}oxy)methyl]benzoate (0.18 g) was synthesized from methyl 2-{[(2,4-dimethyl-1H-benzimidazol-6-yl)oxy]methyl}benzoate (200 mg) in a manner similar to that described in Preparation Example 123 except that (2-chloro-6-phenyl-3-pyridinyl)methyl methanesulfonate (230 mg) was used instead of 4-(bromomethyl)-3-chloro-1,1′-biphenyl.

### PREPARATION EXAMPLE 131

Methyl 2-[({1-[(3-methoxy-1,1′-biphenyl-4-yl)methyl]-2,4-dimethyl-1H-benzimidazol-6-yl}oxy)methyl]benzoate (0.12 g) was synthesized from methyl 2-{[(2,4-dimethyl-1H-benzimidazol-6-yl)oxy]methyl}benzoate (180 mg) in a manner similar to that described in Preparation Example 123 except that (3-methoxy-1,1′-biphenyl-4-yl)methyl methanesulfonate (162 mg) was used instead of 4-(bromomethyl)-3-chloro-1,1′-biphenyl.

### PREPARATION EXAMPLE 132

Methyl 2-[({1-[2-chloro-4-(1,3-oxazol-2-yl)benzyl]-2,4-dimethyl-1H-benzimidazol-6-yl}oxy)methyl]benzoate (87 mg) was synthesized from methyl 2-{[(2,4-dimethyl-1H-benzimidazol-6-yl)oxy]methyl}benzoate (200 mg) in a manner similar to that described in Preparation Example 123 except that 1-bromomethyl-2-chloro-4-(1,3-oxazol-2-yl)benzene (132 mg) was used instead of 4-(bromomethyl)-3-chloro-1,1′-biphenyl.

NMR(DMSO-d₆,δ): 2.44 (3H, s), 2.49 (3H, s), 3.77 (3H, s), 5.33 (2H, s), 5.55 (2H, s), 6.63 (1H, d, J=8 Hz), 6.69 (1H, d, J=1 Hz), 6.77 (1H, d, J=2 Hz), 7.3-7.9 (6H, m), 8.02 (1H, d, J=1 Hz), 8.26 (1H, s).

MS: 502 (M+H).

### PREPARATION EXAMPLE 133

Methyl 2-[({1-[(3-chloro-5-ethoxy-2-pyridinyl)methyl]-2,4-dimethyl-1H-benzimidazol-6-yl}oxy)methyl]benzoate (0.21 g) was synthesized from methyl 2-{[(2,4-dimethyl-1H-benzimidazol-6-yl)oxy]methyl}benzoate (170 mg) in a manner similar to that described in Preparation Example 123 except that (3-chloro-5-ethoxy-2-pyridinyl)methyl methanesulfonate (146 mg) was used instead of 4-(bromomethyl)-3-chloro-1,1′-biphenyl.

NMR(DMSO-d₆,δ): 1.30 (3H, t, J=7 Hz), 2.44 (3H, s), 2.46 (3H, s), 3.80 (3H, s), 4.09 (2H, q, J=7 Hz), 5.33 (2H, s), 5.44 (2H, s), 6.63 (1H, d, J=1 Hz), 6.71 (1H, d, J=2 Hz), 7.4-7.7 (4H, m), 7.8-8.0 (1H, m), 8.08 (1H, d , J=2 Hz).

MS: 480 (M+H).

### PREPARATION EXAMPLE 134

A mixture of methyl 2-{[(2,4-dimethyl-1H-benzimidazol-6-yl)oxy]methyl}benzoate (1.0 g), 1-(bromomethyl)-4-bis(tert-butoxycarbonyl)amino-3-chlorobenzene (1.76 g), potassium carbonate (579 mg) and sodium iodide (483 mg) in N,N-dimethylformamide (10 mL) was stirred at ambient temperature for 3 days. The mixture was partitioned between ethyl acetate and water, and the organic layer was separated, washed with brine, dried over anhydrous sodium sulfate, and concentrated in vacuo. The residue was purified by column chromatography on silica gel (elution; 4:1 chloroform-ethyl acetate) to give methyl 2-{[(1-{4-[bis(tert-butoxycarbonyl) amino]-2-chlorobenzyl}-2,4-dimethyl-1H-benzimidazol-6-yl)oxy]methyl}benzoate (1.02 g) as a white solid.

NMR(DMSO-d₆,δ): 1.35 (18H, s), 2.40 (3H, s), 2.48-2.52 (3H, s), 3.77 (3H, s), 5.31 (2H, s), 5.48 (2H, s), 6.52 (1H, d, J=8 Hz), 6.67 (1H, d, J=1 Hz), 6.85 (1H, d, J=2 Hz), 7.09 (1H, dd, J=2 Hz, 8 Hz), 7.4-8.0 (5H, m).

MS: 650 (M+H).

### PREPARATION EXAMPLE 135

A mixture of methyl 2-{[(1-(4-[bis(tert-butoxycarbonyl)amino]-2-chlorobenzyl}-2,4-dimethyl-1H-benzimidazol-6-yl)oxy]methyl}benzoate (1.0 g), 4 N hydrogen chloride/1,4-dioxane (8 mL) and methanol (40 mL) was stand at ambient temperature overnight. The mixture was concentrated in vacuo and the residue was partitioned between ethyl acetate and 0.1N sodium hydroxide. The organic layer was separated, washed with brine, dried over anhydrous sodium sulfate, and concentrated in vacuo to give methyl 2-({[1-(4-amino-2-chlorobenzyl)-2,4-dimethyl-1H-benzimidazol-6-yl]oxy}methyl)benzoate (0.647 g) as a yellow solid.

NMR (DMSO-d₆,δ): 2.43 (3H, s), 2.45 (3H, s), 3.79 (3H, s), 5.22 (2H, s), 5.34 (2H, s), 5.40 (2H, br s), 6.37 (2H, s), 6.5-6.7 (2H, m), 6.77 (1H, d, J=2 Hz), 7.4-8.0 (4H, m).

MS: 450 (M+H).

### PREPARATION EXAMPLE 136

To a mixture of methyl 2-({[1-(4-amino-2-chlorobenzyl)-2,4-dimethyl-1H-benzimidazol-6-yl]oxy}methyl) benzoate (0.53 g) and iodomethane (836 mg) in a solvent mixture of tetrahydrofuran (3 ml) and N,N-dimethylformamide (3 mL) was added sodium hydride (60% dispersion in mineral oil; 118 mg). The mixture was stirred at ambient temperature for 4 days and partitioned between ethyl acetate and water. The organic layer was separated, washed with brine, dried over anhydrous magnesium sulfate and concentrated in vacuo. The residue was purified by column chromatography on silica gel (gradient elution; chloroform-ethyl acetate 10:1 to 8:1 to 4:1) to give methyl 2-[({1-[2-chloro-4-(dimethylamino)benzyl]-2,4-dimethyl-1H-benzimidazol-6-yl}oxy)methyl]benzoate (0.12 g) as an oil.

NMR(DMSO-d₆,δ): 2.43 (3H, s), 2.46 (3H, s), 2.86 (6H, s), 3.78 (3H, s), 5.28 (2H, s), 5.34 (2H, s), 6.4-6.8 (5H, m), 7.4-8.0 (4H, m).

MS: 478 (M+H).

### PREPARATION EXAMPLE 137

To a suspension of methyl 2-{[4-(acetylamino)-3-methyl-5-nitrophenoxy]methyl}benzoate (12 g) in methanol (240 mL) was added dropwise conc. sulfuric acid (5 mL), and the resulting solution was refluxed for 36 hours. After cooling, the mixture was concentrated in vacuo and the residue was suspended in water (100 mL). The pH of the suspension was adjusted to 4 with 50% sodium hydroxide with cooling in an ice-bath. The suspension was diluted with ethyl acetate (300 mL), stirred in an ice-bath for half an hour, and filtered to give (3.48 g) as a orange solid. The second crop (0.79 g) was obtained from the mother liquor by filtration. The filtrate was partitioned between ethyl acetate and brine. The organic layer was separated, dried over anhydrous sodium sulfate, and concentrated in vacuo. The residue was suspended in ethyl acetate (30 mL) and filtered to give the product (5.0 g). The filtrate was concentrated in vacuo and the residue was purified by column chromatography on silica gel (gradient elution; chloroform to 50:1 chloroform-ethyl acetate) followed by trituration with ethyl acetate to give the further product methyl 2-[(4-amino-3-methyl-5-nitrophenoxy)methyl]benzoate (0.36 g).

NMR(DMSO-d₆,δ): 2.21 (3H, s), 3.82 (3H, s), 5.35 (2H, s), 7.03 (2H, br s), 7.2-7.7 (5H, m), 7.91 (1H, d, J=7 Hz).

MS: 316 (M+Na).

### PREPARATION EXAMPLE 138

To a solution of methyl 2-[(4-amino-3-methyl-5-nitrophenoxy)methyl]benzoate (0.50 g) in tetrahydrofuran (10 mL) was added trifluoroacetic anhydride (1 mL). The mixture was stirred at ambient temperature for 2 hours and concentrated in vacuo. The residue was suspended in n-hexane (15 mL). The suspension was stirred at ambient temperature for half an hour and filtered to give methyl 2-({3-methyl-5-nitro-4-[(trifluoroacetyl)amino]phenoxy}methyl)benzoate (0.62 g) as a white solid.

NMR(DMSO-d₆,δ): 2.25 (3H, s), 3.82 (3H, s), 5.51 (2H, s), 7.39 (1H, d, J=2 Hz), 7.4-7.7 (4H, m), 7.94 (1H, d, J=8 Hz).

MS: 411 (M−H).

### PREPARATION EXAMPLE 139

A mixture of methyl 2-({3-methyl-5-nitro-4-[(trifluoroacetyl)amino]phenoxy}methyl)benzoate (0.60 g), iron (0.40 g), acetic acid (6 mL) and methanol (30 mL) was refluxed overnight. After cooling, the insoluble materials were filtered off and the filtrate was concentrated in vacuo. The residue was partitioned between ethyl acetate and 1 N sodium hydroxide. The organic layer was separated, washed with brine, dried over anhydrous sodium sulfate, and concentrated in vacuo. The residue was purified by column chromatography on silica gel (10:1 chloroform-ethyl acetate) to give methyl 2-({[4-methyl-2-(trifluoromethyl)-1H-benzimidazol-6-yl]oxy}methyl)benzoate (0.46 g) as a white amorphous form.

NMR(DMSO-d₆,δ): 2.48-2.52 (3H, s), 3.82 (3H, s), 5.45 (2H, s), 6.89 (2H, br s), 7.4-8.0 (4H, m), 13.62 (1H, br s).

MS: 365 (M+H).

### PREPARATION EXAMPLE 140

Methyl 2-({[1-[(3-chloro-1,1′-biphenyl-4-yl)methyl]-4-methyl-2-(trifluoromethyl)-1H-benzimidazol-6-yl]oxy}methyl)benzoate (0.10 g) was synthesized from methyl 2-({[4-methyl-2-(trifluoromethyl)-1H-benzimidazol-6-yl]oxy}methyl)benzoate (110 mg) in a manner similar to that described in Preparation Example 123.

NMR(DMSO-d₆,δ): 2.58 (3H, s), 3.75 (3H, s), 5.39 (2H, s), 5.72 (2H, s), 6.47 (1H, d, J=8 Hz), 6.94 (1H, d, J=1 Hz), 7.09 (1H, d, J=2 Hz), 7.3-7.7 (11H, m).

MS: 565 (M+H).

### PREPARATION EXAMPLE 141

To a suspension of methyl 2-[(4-amino-3-methyl-5-nitrophenoxy)methyl]benzoate (0.50 g) in 1,4-dioxane (10 mL) was added 4 N hydrogen chloride/1,4-dioxane (5 mL) followed by addition of propionic anhydride (2.2 mL). The mixture was stirred at ambient temperature for 2 hours and concentrated in vacuo. The residue was suspended in methanol (10 mL) and filtered to give methyl 2-{[3-methyl-5-nitro-4-(propionylamino)phenoxy]methyl}benzoate (0.49 g) as a white solid. The filtrate was concentrated in vacuo and the residue was triturated with n-hexane (15 mL) to give the second crop of the further product (95 mg).

NMR(DMSO-d₆,δ): 1.06 (3H, t, J=7 Hz), 2.21 (3H, s), 2.28 (2H, q, J=7 Hz), 3.82 (3H, s), 5.47 (2H, s), 7.27 (1H, d, J=1 Hz), 7.34 (1H, d, J=1 Hz), 7.4-7.7 (3H, m), 7.92 (1H, d, J=8 Hz), 9.56 (1H, s).

MS: 373 (M+H).

### PREPARATION EXAMPLE 142

A mixture of methyl 2-{[3-methyl-5-nitro-4-(propionylamino)phenoxy]methyl}benzoate (0.56 g), iron (0.42 g), acetic acid (6 mL) and methanol (30 mL) was refluxed overnight. After cooling, the insoluble materials were filtered off and the filtrate was concentrated in vacuo. The residue was partitioned between ethyl acetate and 1 N sodium hydroxide. The organic layer was separated, washed with brine, dried over anhydrous sodium sulfate and concentrated in vacuo. The residue was purified by column chromatography on silica gel (gradient elution; 10:1 chloroform-ethyl acetate) to give methyl 2-{[(2-ethyl-4-methyl-1H-benzimidazol-6-yl)oxy]methyl}benzoate (0.22 g) which was solidified on standing overnight.

NMR(DMSO-d₆,δ): 1.28, 1.29 (total 3H, each t, J=7 Hz), 2.43 (3H, d, J=6 Hz), 2.44 (3H, s), 2.76 (2H, q, J=7 Hz), 3.82 (3H, s), 5.39 (2H, s), 6.68, 6.85 (total 2H, δ6.68: dd, J=2 Hz, 16 Hz, δ6.85: d, J=2 Hz), 7.4-7.8 (4H, m), 7.90 (1H, d, J=8 Hz), 11.91 (1H, br d, J=16 Hz).

MS: 325 (M+H).

### PREPARATION EXAMPLE 143

Methyl 2-[({1-[(3-chloro-1,1′-biphenyl-4-yl)methyl]-2-ethyl-4-methyl-1H-benzimidazol-6-yl}oxy)methyl]benzoate (0.13 g) was synthesized from methyl 2-{[(2-ethyl-4-methyl-1H-benzimidazol-6-yl)oxy]methyl}benzoate (210 mg) in a manner similar to that described in Preparation Example 123.

NMR(DMSO-d₆,δ): 1.26 (3H, t, J=7 Hz), 2.48-2.51 (3H, s), 2.78 (2H, q, J=7 Hz), 3.75 (3H, s), 5.34 (2H, s), 5.49 (2H, s), 6.50 (1H, d, J=8 Hz), 6.70 (1H, d, J=1 Hz), 6.83 (1H, d, J=2 Hz), 7.3-7.9 (11H, m).

MS: 325 (M+H).

### PREPARATION EXAMPLE 144

A mixture of methyl 2-[(4-amino-3-methyl-5-nitrophenoxy)methyl]benzoate (1.0 g), iron (1.77 g) and ammonium chloride (3.38 g) in a solvent mixture of methanol (40 mL) and water (16 mL) was refluxed for an hour. After cooling, the mixture was filtered and the insoluble material was washed with methanol. The filtrate and the washings were combined and concentrated in vacuo. The residue was partitioned between ethyl acetate and water. The organic layer was separated, washed with brine, dried over anhydrous sodium sulfate, and concentrated in vacuo. The residue was dissolved in methanol (20 mL) and treated with 4 N hydrogen chloride/1,4-dioxane (5 mL). The mixture was concentrated in vacuo and the residue was suspended in ethyl acetate (30 mL) and filtered to give methyl 2-[(3,4-diamino-5-methylphenoxy)methyl]benzoate dihydrochloride (1.1 g) as a grayish red solid.

NMR(DMSO-d₆,δ): 2.22 (3H, s), 3.82 (3H, s), 5.32 (2H, s), 6.27 (1H, d, J=2 Hz), 6.35 (1H, d, J=2 Hz), 6.5-8.0 (6H, br m), 7.3-7.7 (3H, m), 7.91 (1H, d, J=7 Hz).

MS: 309 (M+Na)

### PREPARATION EXAMPLE 145

A mixture of methyl 2-[(3,4-diamino-5-methylphenoxy)methyl]benzoate dihydrochloride (0.50 g), acetic acid (0.10 mL) and tetraethoxymethane (2.9 mL) was stirred at 80° C. for 2 hours. After cooling, the mixture was partitioned between ethyl acetate and 1 N sodium hydroxide. The organic layer was separated, washed with brine, dried over anhydrous sodium sulfate, and concentrated in vacuo. The residue was purified by column chromatography on silica gel (gradient elution; chloroform-ethyl acetate 6:1 to 3:1) to afford the product which was solidified on standing overnight. The white solid was triturated with diisopropyl ether (5 mL) and filtered to give methyl 2-{[(2-ethoxy-4-methyl-1H-benzimidazol-6-yl)oxy]methyl}benzoate (0.42 g).

NMR(DMSO-d₆,δ): 1.36 (3H, t, J=7 Hz), 2.32, 2.37 (total 3H, each s), 3.82 (3H, s), 4.43 (2H, q, J=7 Hz), 5.36 (2H, s), 6.5-6.6, 6.75 (total 2H, δ6.5-6.6: m, δ6.75: d, J=2 Hz), 7.4-8.0 (3H, m), 7.89 (1H, d, J=8 Hz), 11.58 (1H, br d, J=17 Hz).

MS: 341 (M+H).

### PREPARATION EXAMPLE 146

Methyl 2-[({1-[(3-chloro-1,1′-biphenyl-4-yl)methyl]-2-ethoxy-4-methyl-1H-benzimidazol-6-yl}oxy)methyl]benzoate (0.17 g) was synthesized from methyl 2-{[(2-ethoxy-4-methyl-1H-benzimidazol-6-yl)oxy]methyl}benzoate (210 mg) in a manner similar to that described in Preparation Example 123.

NMR(DMSO-d₆,δ): 1.35 (3H, t, 7 Hz), 2.42 (3H, s), 3.76 (3H, s), 4.52 (2H, q, J=7 Hz), 5.27 (2H, s), 5.33 (2H, s), 6.63 (1H, d, J=2 Hz), 6.76 (1H, d, J=2 Hz), 6.89 (1H, d, J=8 Hz), 7.3-7.9 (11H, m).

MS: 541 (M+H).

### PREPARATION EXAMPLE 147

Methyl 2-{[(1-{[3-chloro-5-(trifluoromethyl)-2-pyridinyl]methyl}-2-ethoxy-4-methyl-1H-benzimidazol-6-yl)oxy]methyl}benzoate (0.20 g) was synthesized from methyl 2-{[(2-ethoxy-4-methyl-1H-benzimidazol-6-yl)oxy]methyl}benzoate (190 mg) in a manner similar to that described in Preparation Example 123 except that 3-chloro-2-(chloromethyl)-5-(trifluoromethyl)pyridine (167 mg) was used instead of 4-(bromomethyl)-3-chloro-1,1′-biphenyl.

NMR(DMSO-d₆,δ): 1.26 (3H, t, 7 Hz), 2.48 (3H, s), 3.77 (3H, s), 4.45 (2H, q, J=7 Hz), 5.31 (2H, s), 5.47 (2H, s), 6.59 (1H, d, J=2 Hz), 6.74 (1H, d, J=2 Hz), 7.3-7.9 (4H, m), 8.51 (1H, d, J=1 Hz), 8.76 (1H, d, J=1 Hz).

MS: 534 (M+H).

### PREPARATION EXAMPLE 148

To a solution-of methyl 2-[(3,4-diamino-5-methylphenoxy)methyl]benzoate dihydrochloride (0.50 g) in N,N-dimethylformamide (5 mL) was added 1,1′-thiocarbonyldiimidazole (0.32 g). The mixture was stirred at ambient temperature for 10 minutes, and partitioned between ethyl acetate/tetrahydrofuran and water. The organic layer was separated, washed with brine, dried over anhydrous sodium sulfate, and concentrated in vacuo. The resulting solid was suspended in ethanol (5 mL). The suspension was stirred at ambient temperature for half an hour and filtered to give methyl 2-{[(7-methyl-2-thioxo-2,3-dihydro-1H-benzimidazol-5-yl)oxy]methyl}benzoate (0.39 g) as a yellow ocher solid.

NMR(DMSO-d₆,δ): 2.34 (3H, s), 3.81 (3H, s), 5.37 (2H, s), 6.54 (1H, d, J=2 Hz), 6.63 (1H, d, J=2 Hz), 7.4-7.9 (3H, m), 7.91 (1H, d, J=8 Hz).

MS: 351 (M+Na).

### PREPARATION EXAMPLE 149

To a mixture of methyl 2-{[(7-methyl-2-thioxo-2,3-dihydro-1H-benzimidazol-5-yl)oxy]methyl}benzoate (0.35 g), N,N-diisopropylethylamine (0.22 mL) and N,N-dimethylformamide (7 mL) was added iodoethane (0.10 mL). The mixture was stirred at ambient temperature overnight and partitioned between ethyl acetate and water. The organic layer was separated, washed with brine, dried over anhydrous sodium sulfate, and concentrated in vacuo. The residue was purified by column chromatography on silica gel (elution; 10:1 chloroform-ethyl acetate) to give methyl 2-({[2-(ethylthio)-4-methyl-1H-benzimidazol-6-yl]oxy}methyl)benzoate (0.34 g) as an oil.

NMR(DMSO-d₆,δ): 1.36 (3H, t, J=7 Hz), 2.42 (3H, s), 3.22 (2H, q, J=7 Hz), 3.82 (3H, s), 5.39 (2H, s), 6.65 (1H, d, J=2 Hz), 6.78 (1H, br s), 7.4-8.0 (4H, m), 12.29 (1H, br s).

MS: 355 (M−H).

### PREPARATION EXAMPLE 150

Methyl 2-({[1-[(3-chloro-1,1′-biphenyl-4-yl)methyl]-2-(ethylthio)-4-methyl-1H-benzimidazol-6-yl]oxy}methyl)benzoate (0.37 g) was synthesized from methyl 2-({[2-(ethylthio)-4-methyl-1H-benzimidazol-6-yl]oxy}methyl)benzoate (240 mg) in a manner similar to that described in Preparation Example 123.

NMR(DMSO-d₆,δ): 1.33 (3H, t, J=7 Hz), 2.48-2.51 (3H, s), 3.26 (2H, q, J=7 Hz), 3.76 (3H, s), 5.35 (2H, s), 5.45 (2H, s), 6.58 (1H, d, J=8 Hz), 6.72 (1H, d, J=1 Hz), 6.91 (1H, d, J=2 Hz), 7.3-7.9 (11H, m).

MS: 557 (M+H).

### PREPARATION EXAMPLE 151

Methyl 2-{[4-(acetylamino)-3-nitrophenoxy]methyl}benzoate (2.9 g) was synthesized from N-(4-hydroxy-2-nitrophenyl)acetamide (1.6 g) in a manner similar to that described in Preparation Example 121.

NMR(DMSO-d₆,δ): 2.02 (3H, s), 3.81 (3H, s), 5.48 (2H, s), 7.33 (1H, dd, J=3 Hz, 9 Hz), 7.4-8.0 (6H, m), 9.6-10.5 (1H, br m).

MS: 424 (M+Na).

### PREPARATION EXAMPLE 152

A mixture of methyl 2-{[4-(acetylamino)-3-nitrophenoxy]methyl}benzoate (2.3 g), 10% palladium on carbon (50% wet; 0.23 g), methanol (5 mL) and tetrahydrofuran (50 mL) was stirred for 8 hours under 1 atmosphere of hydrogen at ambient temperature. The catalyst was filtered off and the filtrate was concentrated in vacuo. The residue was purified by column chromatography on silica gel (10:1 chloroform-methanol) to afford the product which was suspended in ethyl acetate (5 mL). The suspension was diluted with diisopropyl ether (5 mL), stirred at ambient temperature for half an hour, and filtered to give methyl 2-{[4-(acetylamino)-3-aminophenoxy]methyl}benzoate (0.43 g) as a solid.

NMR(DMSO-d₆,δ): 1.99 (3H, s), 3.82 (3H, s), 4.90 (2H, br s), 5.31 (2H, s), 6.16 (1H, dd, J=3 Hz, 9 Hz), 6.33 (1H, d, J=3 Hz), 6.96 (1H, d, J=8 Hz), 7.4-8.0 (4H, m), 8.97 (1H, s).

MS: 337 (M+Na).

### PREPARATION EXAMPLE 153

Methyl 2-({4-(acetylamino)-3-[(2,4-dichlorobenzyl)amino]phenoxy}methyl)benzoate was synthesized from methyl 2-{[4-(acetylamino)-3-aminophenoxy]methyl}benzoate (100 mg) in a manner similar to that described in Preparation Example 155 except that 1-chloromethyl-2,4-dichlorobenzene (68.4 mg) was used instead of 4-(bromomethyl)-3-chloro-1,1′-biphenyl.

### PREPARATION EXAMPLE 154

Methyl 2-({[1-(2,4-dichlorobenzyl)-2-methyl-1H-benzimidazol-6-yl]oxy}methyl)benzoate (0.14 g) was synthesized from methyl 2-({4-(acetylamino)-3-[(2,4-dichlorobenzyl)amino]phenoxy}methyl)benzoate (150 mg) in a manner similar to that described in Preparation Example 156.

NMR(DMSO-d₆,δ): 2.42 (3H, s), 3.77 (3H, s), 5.36 (2H, s), 5.46 (2H, s), 6.49 (1H, d, J=8 Hz), 6.84 (1H, dd, J=2 Hz, 9 Hz), 6.99 (1H, d, J=2 Hz), 7.2-7.7 (5H, m), 7.69 (1H, d, J=2 Hz), 7.8-8.0 (1H, m).

### PREPARATION EXAMPLE 155

A mixture of 4-(acetylamino)-3-aminophenyl acetate (5.1 g), 4-(bromomethyl)-3-chloro-1,1′-biphenyl (6.9 g), potassium carbonate (3.5 g) and N,N-dimethylformamide (49 mL) was stirred at 80° C. for 1.5 hours. After cooling, the mixture was partitioned between ethyl acetate/tetrahydrofuran and water. The organic layer was separated, washed with brine, dried over anhydrous sodium sulfate, and concentrated in vacuo. The resulting solid was suspended in toluene (100 mL). The suspension was stirred at ambient temperature for half an hour and filtered to give 4-(acetylamino)-3-{[(3-chloro-1,1′-biphenyl-4-yl)methyl]amino}phenyl acetate (7.0 g) as a white solid. The filtrate was concentrated in vacuo and the residue was purified by column chromatography on silica gel (1:1 chloroform-ethyl acetate) followed by trituration with ethyl acetate (20 mL) to give the further product (1.0 g).

NMR(DMSO-d₆,δ): 2.02 (3H, s), 2.16 (3H, s), 4.39 (2H, d, J=6 Hz), 5.95 (1H, t, J=6 Hz), 6.13 (1H, d, J=2 Hz), 6.30 (1H, dd, J=2 Hz, 8 Hz), 7.10 (1H, d, J=8 Hz), 7.3-7.7 (8H, m), 9.28 (1H, s).

MS: 409 (M+H).

### PREPARATION EXAMPLE 156

To a suspension of 4-(acetylamino)-3-{[(3-chloro-1,1′-biphenyl-4-yl)methyl]amino}phenyl acetate (7.0 g) in ethanol (30 mL) was added dropwise concentrated sulfuric acid (7 mL) and the mixture was stirred at 80° C. overnight. After cooling, the mixture was concentrated in vacuo and the residue was neutralized with sodium hydroxide with cooling in an ice-bath. The resulting suspension was diluted with ethanol (10 mL), stirred at ambient temperature for half an hour, and filtered to give 1-[(3-chloro-1,1′-biphenyl-4-yl)methyl]-2-methyl-1H-benzimidazol-6-ol (5.4 g) as a pale purple solid.

NMR(DMSO-d₆,δ): 2.45 (3H, s), 5.45 (2H, s), 6.5-6.7 (3H, m), 7.3-7.7 (7H, m), 8.84 (1H, d, J=2 Hz), 9.14 (1H, s).

MS: 349 (M+H).

### PREPARATION EXAMPLE 157

To a suspension of 1-[(3-chloro-1,1′-biphenyl-4-yl)methyl]-2-methyl-1H-benzimidazol-6-ol (0.93 g) in a solvent mixture of tetrahydrofuran (5 mL) and N,N-dimethylformamide (2.5 mL) was added sodium hydride (60% dispersion in mineral oil; 117 mg). The mixture was stirred at 80° C. for 15 minutes and allowed to cool to ambient temperature. To the mixture was added methyl 2-(bromomethyl)benzoate. After stirring for 3 days, the mixture was partitioned between ethyl acetate and water. The organic layer was separated, washed with brine, dried over anhydrous magnesium sulfate, and concentrated in vacuo. The residue was purified by column chromatography on silica gel (elution; 50:1 chloroform-methanol) followed by trituration with ethyl acetate (5 mL) to give methyl 2-[({1-[(3-chloro-1,1′-biphenyl-4-yl)methyl]-2-methyl-1H-benzimidazol-6-yl}oxy)methyl]benzoate (0.97 g) as a white solid. The filtrate was concentrated in vacuo and the residue was triturated with n-hexane (5 mL) to afford the further product (0.35 g).

NMR(DMSO-d₆,δ): 2.45 (3H, s), 3.75 (3H, s), 5.36 (2H, s), 5.52 (2H, s), 6.57 (1H, d, J=8 Hz), 6.85 (1H, dd, J=2 Hz, 8 Hz), 7.04 (1H, d, J=2 Hz), 7.3-7.9 (12H, m).

MS: 497 (M+H).

### PREPARATION EXAMPLE 158

4-(Acetylamino)-3-({[3-chloro-5-(trifluoromethyl)-2-pyridinyl]methyl}amino)phenyl acetate (0.34 g) was synthesized from 4-(acetylamino)-3-aminophenyl acetate (200 mg) in a manner similar to that described in Preparation Example 155 except that 3-chloro-2-(chloromethyl)-5-(trifluoromethyl)pyridine (265 mg) was used instead of 4-(bromomethyl)-3-chloro-1,1′-biphenyl.

NMR(DMSO-d₆,δ): 2.06 (3H, s), 2.21 (3H, s), 4.54 (2H, br d, J=6 Hz), 5.90 (1H, t, J=6 Hz), 6.34 (1H, dd, J=2 Hz, 8 Hz), 6.44 (1H, d, J=2 Hz), 7.10 (1H, d, J=8 Hz), 8.50 (1H, d, J=2 Hz), 8.95 (1H, d, J=1 Hz), 9.27 (1H, br s).

MS: 424 (M+Na).

### PREPARATION EXAMPLE 159

1-{[3-Chloro-5-(trifluoromethyl)-2-pyridinyl]methyl}-2-methyl-1H-benzimidazol-6-ol (0.18 g) was synthesized from 4-(acetylamino)-3-({[3-chloro-5-(trifluoromethyl)-2-pyridinyl]methyl}amino)phenyl acetate (320 mg) in a manner similar to that described in Preparation Example 156.

NMR(DMSO-d₆,δ): 2.41 (3H, s), 5.64 (2H, s), 6.5-6.7 (2H, m), 7.2-7.4 (1H, m), 8.55 (1H, d, J=2 Hz), 8.79 (1H, d, J=1 Hz), 9.08 (1H, br s).

MS: 342 (M+H).

### PREPARATION EXAMPLE 160

Methyl 2-{[(1-{[3-chloro-5-(trifluoromethyl)-2-pyridinyl]methyl}-2-methyl-1H-benzimidazol-6-yl)oxy]methyl}benzoate (0.11 g) was synthesized from 1-{[3-chloro-5-(trifluoromethyl)-2-pyridinyl]methyl}-2-methyl-1H-benzimidazol-6-ol (270 mg) in a manner similar to that described in Preparation Example 157.

### PREPARATION EXAMPLE 161

[1-(2,4-Dichlorobenzyl)-2,4-dimethyl-1H-benzimidazol-6-yl]methanol (1.96 g) was synthesized from ethyl 1-(2,4-dichlorobenzyl)-2,4-dimethyl-1H-benzimidazole-6-carboxylate (2.50 g) in a manner similar to that described in Preparation Example 180.

MS (ES+): 335 (M⁺+1).

### PREPARATION EXAMPLE 162

Manganese dioxide (1.56 g) was added to a solution of [1-(2,4-dichlorobenzyl)-2,4-dimethyl-1H-benzimidazol-6-yl]methanol (200 mg) in acetone (6 mL), and the mixture was stirred at room temperature for 2 hours. Manganese salts were removed by celite pad filtration, and the solution was concentrated in vacuo to give 1-(2,4-dichlorobenzyl)-2,4-dimethyl-1H-benzimidazole-6-carbaldehyde. The crude product was used for the next step without purification.

### PREPARATION EXAMPLE 163

Ethyl 3-({[1-(2,4-dichlorobenzyl)-2,4-dimethyl-1H-benzimidazol-6-yl]methyl}amino)benzoate (224 mg) was synthesized from 1-(2,4-dichlorobenzyl)-2,4-dimethyl-1H-benzimidazole-6-carbaldehyde (200 mg) and ethyl 3-aminobenzoate (149 mg) in a manner similar to that described in Preparation Example 165.

MS (ES+): 482 (M⁺+1).

### PREPARATION EXAMPLE 164

Sodium triacetoxyborohydride (132 mg) and aqueous solution of formaldehyde (37%, 51 μL) were added to a solution of ethyl 3-({[1-(2,4-dichlorobenzyl)-2,4-dimethyl-1H-benzimidazol-6-yl]methyl}amino)benzoate (150 mg) in 1,2-dichloroethane (3 mL). The mixture was stirred at room temperature for 4 days. The reaction was quenched by addition of water, and the organic materials were extracted with chloroform. The organic layer was washed with water, dried over sodium sulfate, and concentrated in vacuo. The crude product was purified over preparative thin layer chromatography (chloroform:methanol=20:1) to give ethyl 3-[{[1-(2,4-dichlorobenzyl)-2,4-dimethyl-1H-benzimidazol-6-yl]methyl}(methyl)amino]benzoate (153 mg) as a powder.

MS (ES+): 496 (M⁺+1).

### PREPARATION EXAMPLE 165

Sodium triacetoxyborohydride (191 mg) and acetic acid (52 μL) were added to a solution of 1-(2,4-dichlorobenzyl)-2,4-dimethyl-1H-benzimidazole-6-carbaldehyde (crude, 150 mg) and 2-aminobenzoic acid (92.6 mg) in tetrahydrofuran (3 mL). Triethylamine (94 μL) was added to the mixture, and it was stirred at room temperature for 15 hours. The reaction was quenched by addition of water, and the organic materials were extracted with chloroform. The organic layer was washed with water, dried over sodium sulfate, and concentrated in vacuo. The crude product was purified over preparative thin layer chromatography (chloroform:methanol=10:1) to give 2-({[1-(2,4-dichlorobenzyl)-2,4-dimethyl-1H-benzimidazol-6-yl]methyl)amino)benzoic acid (155 mg) as a powder.

¹H NMR (200 MHz, DMSO-d₆, δ/ppm): 2.46 (3H, s), 2.50 (3H, s), 4.41 (2H, s), 5.45 (2H, s), 6.48 (1H, d, J=8.3 Hz), 6.53 (1H, t, J=7.5 Hz), 6.63 (1H, d, J=8.3 Hz), 6.99 (1H, s), 7.15 (1H, s), 7.2-7.4 (2H, m), 7.66 (1H, d, J=2.1 Hz), 7.78 (1H, dd, J=7.9 Hz, 1.5 Hz), 12.1-13.0 (1H, br).

### PREPARATION EXAMPLE 166

A solution of diethyl azodicarboxylate in toluene (40%, 0.14 mL) was added dropwise to a suspension of [1-(2,4-dichlorobenzyl)-2,4-dimethyl-1H-benzimidazol-6-yl]methanol (150 mg), methyl salicylate (68 mg) and triphenylphosphine (159 mg) in tetrahydrofuran at room temperature. The mixture was stirred at room temperature for 3 hours. The solution was treated with aqueous solution of sodium bicarbonate and brine and dried over sodium sulfate. The crude product was purified over silica gel chromatography (chloroform:methanol=50:1) to give methyl 2-([1-(2,4-dichlorobenzyl)-2,4-dimethyl-1H-benzimidazol-6-yl]methoxy}benzoate (99 mg) as a powder.

MS (ES+): 469 (M⁺+1).

### PREPARATION EXAMPLE 167

Methyl 3-{[1-(2,4-dichlorobenzyl)-2,4-dimethyl-1H-benzimidazol-6-yl]methoxy}benzoate (131 mg) was synthesized from [1-(2,4-dichlorobenzyl)-2,4-dimethyl-1H-benzimidazol-6-yl]methanol (150 mg) and methyl 3-hydroxybenzoate (68 mg) in a manner similar to that described in Preparation Example 166.

MS (ES+): 469 (M⁺+1).

### PREPARATION EXAMPLE 168

Ethyl 2-{[1-(2,4-dichlorobenzyl)-2,4-dimethyl-1H-benzimidazol-6-yl]methoxy}isonicotinate (53 mg) was synthesized from [1-(2,4-dichlorobenzyl)-2,4-dimethyl-1H-benzimidazol-6-yl]methanol (100 mg) and ethyl 2-hydroxyisonicotinate (60 mg) in a manner similar to that described in Preparation Example 171.

¹H NMR (200 MHz, DMSO-d₆, δ/ppm): 1.32 (3H, t, J=7.1 Hz), 2.47 (3H, s), 2.52 (3H, s), 4.33 (2H, q, J=7.2 Hz), 5.39 (2H, s), 5.49 (2H, s), 6.47 (1H, d, J=8.4 Hz), 7.09 (1H, s), 7.2-7.4 (3H, m), 7.39 (1H, dd, J=5.1 Hz, 1.3 Hz), 7.69 (1H, d, J=2.1 Hz), 8.32 (1H, d, J=5.3 Hz).

### PREPARATION EXAMPLE 169

Methyl 6-{[1-(2,4-dichlorobenzyl)-2,4-dimethyl-1H-benzimidazol-6-yl]methoxy}-2-pyridinecarboxylate (37 mg) was synthesized from [1-(2,4-dichlorobenzyl)-2,4-dimethyl-1H-benzimidazol-6-yl]methanol (100 mg) and methyl 6-hydroxy-2-pyridinecarboxylate (55 mg) in a manner similar to that described in Preparation Example 171.

¹H NMR (200 MHz, DMSO-d₆, δ/ppm): 2.46 (3H, s), 2.53 (3H, s), 3.86 (3H, s), 5.39 (2H, s), 5.48 (2H, s), 6.40 (1H, d, J=8.4 Hz), 7.06 (1H, d, J=8.6 Hz), 7.16 (1H, s), 7.27 (1H, dd, J=8.3 Hz, 2.2 Hz), 7.39 (1H, s), 7.66 (1H, d, J=7.5 Hz), 7.71 (1H, d, J=2.2 Hz), 7.85 (1H, d, J=8.3 Hz).

### PREPARATION EXAMPLE 170

{1-[(3-Chloro-1,1′-biphenyl-4-yl)methyl]-2,4-dimethyl-1H-benzimidazol-6-yl}methanol (340 mg) was synthesized from ethyl 1-[(3-chloro-1,1′-biphenyl-4-yl)methyl]-2,4-dimethyl-1H-benzimidazole-6-carboxylate (392 mg) in a manner similar to that described in Preparation Example 180.

MS (ES+): 377 (M⁺+1).

### PREPARATION EXAMPLE 171

A solution of diethyl azodicarboxylate in toluene (40%, 0.14 mL) was added dropwise to a suspension of {1-[(3-chloro-1,1′-biphenyl-4-yl)methyl]-2,4-dimethyl-1H-benzimidazol-6-yl}methanol (100 mg), methyl salicylate (48 mg) and polystyrene-supported triphenylphosphine (1.12 mmol/g, 284 mg) in tetrahydrofuran at room temperature. The mixture was stirred at room temperature for 3 days, and resin was removed by filtration. The solution was treated with aqueous solution of sodium bicarbonate and brine and dried over sodium sulfate. The crude product was purified over preparative thin layer chromatography (ethyl acetate) to give methyl 2-({1-[(3-chloro-1,1′-biphenyl-4-yl)methyl]-2,4-dimethyl-1H-benzimidazol-6-yl}methoxy)benzoate (102 mg) as a powder.

MS (ES+) 511 (M⁺+1).

### PREPARATION EXAMPLE 172

Methyl 3-({1-[(3-chloro-1,1′-biphenyl-4-yl)methyl]-2,4-dimethyl-1H-benzimidazol-6-yl}methoxy)benzoate (83 mg) was synthesized from {1-[(3-chloro-1,1′-biphenyl-4-yl)methyl]-2,4-dimethyl-1H-benzimidazol-6-yl}methanol (100 mg) and methyl 3-hydroxybenzoate (49 mg) in a manner similar to that described in Preparation Example 171.

MS (ES+): 511 (M⁺+1).

### PREPARATION EXAMPLE 173

Manganese dioxide (2.31 g) was added to a solution of {1-[(3-chloro-1,1′-biphenyl-4-yl)methyl]-2,4-dimethyl-1H-benzimidazol-6-yl)methanol (200 mg) in acetone (5 mL), and the mixture was stirred at room temperature for 2 hours. Manganese salts were removed by celite pad filtration, and the solution was concentrated in vacuo. The residue was dissolved in tetrahydrofuran (2 mL), methyl 2-aminoisonicotinate (161 mg) and titanium tetraisopropoxide (0.31 mL) were added to the solution, and the mixture was stirred at room temperature for 15 hours. Sodium borohydride (80 mg in two portions) and methanol (1 mL) were added to it, and the mixture was stirred for further 30 minutes. The reaction was quenched by addition of water, and the product was extracted with ethyl acetate. The organic layer was washed with water and brine and dried over sodium sulfate. The crude product was purified over silica gel chromatography (hexane:ethyl acetate 1:1 to ethyl acetate only) to give isopropyl 2-[({1-[(3-chloro-1,1′-biphenyl-4-yl)methyl]-2,4-dimethyl-1H-benzimidazol-6-yl}methyl)amino]isonicotinate (189 mg).

MS (ES+): 539 (M⁺+1).

### PREPARATION EXAMPLE 174

Isopropyl 6-[({1-[(3-chloro-1,1′-biphenyl-4-yl)methyl]-2,4-dimethyl-1H-benzimidazol-6-yl}methyl)amino]-2-pyridinecarboxylate (136 mg) was synthesized from {1-[(3-chloro-1,1′-biphenyl-4-yl)methyl]-2,4-dimethyl-1H-benzimidazol-6-yl}methanol (200 mg) and methyl 6-amino-2-pyridinecarboxylate (161 mg) in a manner similar to that described in Preparation Example 173.

MS (ES+): 539 (M⁺+1).

### PREPARATION EXAMPLE 175

Ethyl 1-(2-chloro-4-ethoxybenzyl)-2,4-dimethyl-1H-benzimidazole-6-carboxylate (2.20 g) was synthesized from ethyl 2,4-dimethyl-1H-benzimidazole-6-carboxylate (1.50 g) and 1-(bromomethyl)-2-chloro-4-ethoxybenzene (2.06 g) in a manner similar to that described in Preparation Example 179.

MS (ES+): 387 (M⁺+1).

### PREPARATION EXAMPLE 176

[1-(2-Chloro-4-ethoxybenzyl)-2,4-dimethyl-1H-benzimidazol-6-yl]methanol (1.86 g) was synthesized from ethyl 1-(2-chloro-4-ethoxybenzyl)-2,4-dimethyl-1H-benzimidazole-6-carboxylate (2.20 g) in a manner similar to that described in preparation example 180.

MS (ES+): 345 (M⁺+1).

### PREPARATION EXAMPLE 177

Methyl 2-{[1-(2-chloro-4-ethoxybenzyl)-2,4-dimethyl-1H-benzimidazol-6-yl]methoxy}benzoate (102 mg) was synthesized from [1-(2-chloro-4-ethoxybenzyl)-2,4-dimethyl-1H-benzimidazol-6-yl]methanol (100 mg) and methyl salicylate (53 mg) in a manner similar to that described in Preparation Example 171.

MS (ES+): 479 (M⁺+1).

### PREPARATION EXAMPLE 178

Methyl 3-{[1-(2-chloro-4-ethoxybenzyl)-2,4-dimethyl-1H-benzimidazol-6-yl]methoxy}benzoate (82 mg) was synthesized from [1-(2-chloro-4-ethoxybenzyl)-2,4-dimethyl-1H-benzimidazol-6-yl]methanol (100 mg) and methyl 3-hydroxybenzoate (53 mg) in a manner similar to that described in Preparation Example 171.

MS (ES+): 479 (M⁺+1).

### PREPARATION EXAMPLE 179

Potassium carbonate (633 mg) and 3-chloro-2-(chloromethyl)-5-(trifluoromethyl)pyridine (1.05 g) were added to a solution of ethyl 2,4-dimethyl-1H-benzimidazole-6-carboxylate (800 mg) in N,N-dimethylformamide (16 mL) at room temperature, and the mixture was stirred at 80° C. for 2 hours. The reaction was quenched by addition of water, and the organic materials were extracted with ethyl acetate. The organic layer was washed with brine and dried over sodium sulfate. The crude product was purified over preparative thin layer chromatography (chloroform methanol=20:1) to give ethyl 1-{[3-chloro-5-(trifluoromethyl)-2-pyridinyl]methyl}-2,4-dimethyl-1H-benzimidazole-6-carboxylate (940 mg) as a powder.

¹H NMR (200 MHz, DMSO-d₆, δ/ppm): 1.30 (3H, t, J=7.1 Hz), 2.49 (3H, s), 2.56 (3H, s), 4.27 (2H, q, J=7.1 Hz), 5.87 (2H, s), 7.61 (1H, s), 7.87 (1H, s), 8.57 (1H, d, J=1.5 Hz), 8.75 (1H, d, J=0.9 Hz).

### PREPARATION EXAMPLE 180

A solution of diisobutylalminum hydride in toluene (1.0 M, 3.76 mL) was added dropwise to a cooled solution of ethyl 1-{[3-chloro-5-(trifluoromethyl)-2-pyridinyl]methyl}-2,4-dimethyl-1H-benzimidazole-6-carboxylate (516 mg) in tetrahydrofuran (12 mL) at −78° C. The mixture was stirred for 3 hours with warming slowly to 0° C. The reaction was quenched by careful addition of methanol, followed by addition of water. It was diluted with ethyl acetate, and inorganic salts were removed by celite pad filtration. Organic solvents were removed in vacuo, and the product was extracted with chloroform. The organic layer was washed with brine, dried over sodium sulfate, and concentrated in vacuo to give (1-{[3-chloro-5-(trifluoromethyl)-2-pyridinyl]methyl}-2,4-dimethyl-1H-benzimidazol-6-yl)methanol (456 mg) as a powder.

MS (ES+): 370 (M⁺+1).

### PREPARATION EXAMPLE 181

Ethyl 2-[(1-{[3-chloro-5-(trifluoromethyl)-2-pyridinyl]methyl}-2,4-dimethyl-1H-benzimidazol-6-yl)methoxy]isonicotinate (75 mg) was synthesized from (1-{[3-chloro-5-(trifluoromethyl)-2-pyridinyl]methyl}-2,4-dimethyl-1H-benzimidazol-6-yl)methanol (150 mg) and ethyl 2-hydroxyisonicotinate (102 mg) in a manner similar to that described in Preparation Example 171.

MS (ES+): 519 (M⁺+1).

### PREPARATION EXAMPLE 182

Methyl 6-[(1-{[3-chloro-5-(trifluoromethyl)-2-pyridinyl]methyl}-2,4-dimethyl-1H-benzimidazol-6-yl)methoxy]-2-pyridinecarboxylate (63 mg) was synthesized from (1-{[3-chloro-5-(trifluoromethyl)-2-pyridinyl]methyl}-2,4-dimethyl-1H-benzimidazol-6-yl)methanol (150 mg) and methyl 6-hydroxy-2-pyridinecarboxylate (93 mg) in a manner similar to that described in Preparation Example 171.

MS (ES+): 505 (M⁺+1).

### PREPARATION EXAMPLE 183

To a mixture of 6-bromo-2,4-dimethyl-1H-benzimidazole (711 mg), K₂CO₃ (480 mg) and DMF (7 ml) was added 1-(bromomethyl)-2-chloro-4-(pentyloxy)benzene (1.01 g) and the mixture was heated at 50° C. for 6 hours. After cooling, the reaction mixture was diluted with EtOAc (40 ml) and washed with water (30 ml) and brine (2×30 ml). The organic layer was dried over MgSO₄ and filtered. Evaporation gave a residue (1.52 g) which was chromatographed (silica gel, EtOAc/n-hexane=2/1→EtOAc) to give 6-bromo-1-[2-chloro-4-(pentyloxy)benzyl]-2,4-dimethyl-1H-benzimidazole (0.70 g) as a pale yellow oil.

NMR(DMSO-d₆,δ): 0.87 (3H, t, J=6.8 Hz), 1.2-1.5 (4H, m), 1.5-1.8 (2H, m), 2.45 (3H, s), 2.50 (3H, s), 3.94 (2H, t, J=6.4 Hz), 5.43 (2H, br s), 6.49 (1H, d, J=8.6 Hz.), 6.83 (1H, dd, J=2.5 Hz, 8.6 Hz), 7.11 (1H, d, J=2.5 Hz), 7.14 (1H, br s), 7.46 (1H, br s).

MS: 435, 437 (1:1 ratio, Br isotopes, M+1).

### PREPARATION EXAMPLE 184

To a suspension of 1-[2-chloro-4-(pentyloxy)benzyl]-2-methyl-1H-benzimidazole-6-carboxylic acid (100 mg) in DMF (2 ml) was added NaH (NaH; 60% dispersion in mineral oil) (12 mg) at 5° C. After stirring for 30 minutes at ambient temperature, ethyl 2-chloro-3-oxobutanoate (51 mg) was added and stirred for 4 hours. The reaction mixture was poured into water (30 ml) and extracted with EtOAc (2×30 ml). The combined organic extracts were washed with brine (2×30 ml), dried over MgSO₄, and evaporated to give 1-(ethoxycarbonyl)-2-oxopropyl 1-[2-chloro-4-(pentyloxy)benzyl]-2-methyl-1H-benzimidazole-6-carboxylate (148 mg) as a crude oil.

NMR(DMSO-d₆,δ): 0.8-1.0 (3H, m), 1.1-1.5 (7H, m), 1.5-1.8 (2H, m), 2.39 (3H, s), 2.57 (3H, s), 3.94 (2H, t, J=6.4 Hz), 4.22 (2H, q, J=7.1 Hz), 5.57 (2H, br s), 5.84 (1H, s), 6.72 (1H, d, J=8.7 Hz), 6.84 (1H, dd, J=2.4 Hz, 8.7 Hz), 7.10 (1H, d, J=2.4 Hz), 7.71 (1H, d, J=8.4 Hz), 7.88 (1H, dd, J=1.5 Hz, 8.4 Hz), 8.05 (1H, d, J=1.5 Hz).

MS: 515 (M+1).

### PREPARATION EXAMPLE 185

To a solution of 1-(ethoxycarbonyl)-2-oxopropyl 1-[2-chloro-4-(pentyloxy)benzyl]-2-methyl-1H-benzimidazole-6-carboxylate (610 mg) in AcOH (3 ml) was added ammonium acetate (548 mg) at ambient temperature. The mixture was heated at 120° C. for 1 hour. After cooling, the reaction mixture was added saturated NaHCO₃ (40 ml) and extracted with EtOAc (2×30 ml). The combined organic extracts were washed with saturated NaHCO₃ (10 ml) and brine (30 ml). The organic layer was dried MgSO₄ and filtered. Evaporation gave a residue (549 mg) which was chromatographed (silica gel, CH₂Cl₂/MeOH=100/1→100/2) to give ethyl 2-{1-[2-chloro-4-(pentyloxy)benzyl]-2-methyl-1H-benzimidazol-6-yl)-4-methyl-1,3-oxazole-5-carboxylate (132 mg) and ethyl 2-{1-[2-chloro-4-(pentyloxy)benzyl]-2-methyl-1H-benzimidazol-6-yl}-4-methyl-1H-imidazole-5-carboxylate (183 mg). Ethyl 2-{1-[2-chloro-4-(pentyloxy)benzyl]-2-methyl-1H-benzimidazol-6-yl}-4-methyl-1,3-oxazole-5-carboxylate

NMR(DMSO-d₆,δ): 0.8-1.0 (3H, m), 1.2-1.5 (7H, m), 1.5-1.8 (2H, m), 2.44 (3H, s), 2.53 (3H, s), 3.94 (2H, t, J=6.4 Hz), 4.33 (2H, q, J=7.0 Hz), 5.58 (2H, br s), 6.56 (1H, d, J=8.6 Hz), 6.83 (1H, dd, J=2.3 Hz, 8.6 Hz), 7.13 (1H, d, J=2.3 Hz), 7.73 (1H, d, J=8.4 Hz), 7.87 (1H, dd, J=1.4 Hz, 8.4 Hz), 8.03 (1H, br s).

MS: 496 (M+1).

Ethyl 2-{1-[2-chloro-4-(pentyloxy)benzyl]-2-methyl-1H-benzimidazol-6-yl}-4-methyl-1H-imidazole-5-carboxylate

NMR(DMSO-d₆,δ): 0.8-1.0 (3H, m), 1.2-1.4 (7H, m), 1.5-1.8 (2H, m), 2.46 (3H, s), 3.93 (2H, t, J=6.4 Hz), 4.24 (2H, q, J=7.0 Hz), 5.48 (2H, br s), 6.35 (1H, d, J=8.6 Hz), 6.80 (1H, dd, J=2.5 Hz, 8.6 Hz), 7.14 (1H, d, J=2.5 Hz), 7.64 (1H, d, J=8.4 Hz), 7.7-8.2 (2H, m), 12.74 (1H, br s).

MS: 493 (M−1).

### PREPARATION EXAMPLE 186

To a solution of ethyl 4-amino-3-methylbenzoate (7.43 g) in DMF (35 ml) were added DMAP (506 mg) and acetic anhydride (4.66 g) at ambient temperature. After stirring for 4 hours, the reaction mixture was diluted with water (40 ml) and extracted with EtOAc (4×60 ml). The combined organic extracts were washed with saturated NaHCO₃ (2×30 ml) and brine (3×100 ml). The organic layer was dried over MgSO₄ and filtered. Evaporation gave a residue which was triturated with EtOAc—n-hexane to give ethyl 4-(acetylamino)-3-methylbenzoate (4.324 g) as white crystals. The filtrate was evaporated and triturated with EtOAc—n-hexane to give second crop (1.64 g).

NMR(DMSO-d₆,δ): 1.31 (3H, t, J=7.1 Hz), 2.11 (3H, s), 2.28 (3H, s), 4.29 (2H, q, J=7.1 Hz), 7.7-7.8 (3H, m), 9.37 (1H, br s).

MS: 244 (M+Na).

### PREPARATION EXAMPLE 187

To a mixture of H₂SO₄ (3 ml) and HNO₃ (70%, d=1.42, 2.5 ml) was added ethyl 4-(acetylamino)-3-methylbenzoate (1.0 g) at 5° C. After stirring for 30 minutes, the reaction mixture was poured into ice and the precipitates were collected by filtration. The precipitates were dissolved in EtOAc (50 ml) and washed with brine (2×30 ml) and small portion of saturated NaHCO₃. The organic layer was dried over MgSO₄ and filtered. Evaporation gave a residue which was triturated with EtOAc (10 ml)—n-hexane (10 ml) to give ethyl 4-(acetylamino)-3-methyl-5-nitrobenzoate (963 mg) as white crystals.

NMR(DMSO-d₆,δ): 1.34 (3H, t, J=7.1 Hz), 2.08 (3H, s), 2.38 (3H, s), 4.35 (2H, q, J=7.1 Hz), 8.1-8.2 (2H, m), 10.10 (1H, br s).

MS: 289 (M+Na).

### PREPARATION EXAMPLE 188

To a solution of ethyl 4-(acetylamino)-3-methyl-5-nitrobenzoate (900 mg) in EtOH (20 ml) was added palladium on carbon (10%, 50% wet, 270 mg) at ambient temperature, and the resultant mixture was hydrogenated under atmospheric pressure of hydrogen for 9 hours. The catalyst was removed by filtration. Evaporation gave a residue (868 mg) which was triturated with EtOAc (5 ml)—n-hexane (5 ml) to give ethyl 4-(acetylamino)-3-amino-5-methylbenzoate (717 mg) as white crystals.

NMR(DMSO-d₆,δ): 1.29 (3H, t, J=7.1 Hz), 2.05 (3H, s), 2.09 (3H, s), 4.26 (2H, q, J=7.1 Hz), 5.06 (2H, br s), 7.02 (1H, d, J=1.6 Hz), 7.20 (1H, d, J=1.6 Hz), 9.04 (1H, br s).

MS: 259 (M+Na).

### PREPARATION EXAMPLE 189

To a suspension of ethyl 4-(acetylamino)-3-amino-5-methylbenzoate (23.9 g) in EtOH (239 ml) was added H₂SO₄ (10.8 ml) at ambient temperature. The mixture was heated at reflux for 12 hours. After cooling, the reaction mixture was evaporated, and added ice (300 g) and EtOAc (250 ml). The aqueous layer was neutralized with 20%-NaOH and the organic layer was separated. The aqueous layer was extracted with EtOAc (2×100 ml) again. The combined organic layers were washed with brine (150 ml), dried over MgSO₄, and filtered. Evaporation gave a residue which was triturated with EtOAc—n-hexane to give ethyl 2,4-dimethyl-1H-benzimidazole-6-carboxylate (20.47 g) as white crystals. The filtrate was evaporated and triturated with EtOAc—n-hexane to give second crop (685 mg).

NMR(DMSO-d₆,δ): 1.33 (3H, t, J=7.1 Hz), 2.4-2.6 (6H), 4.30 (2H, q, J=7.1 Hz), 7.58 (1H, br s), 7.89 (1H, br s), 12.50 (1H, br s).

MS: 219 (M+1).

### PREPARATION EXAMPLE 190

Ethyl 1-(2,4-dichlorobenzyl)-2,4-dimethyl-1H-benzimidazole-6-carboxylate (714 mg) was synthesized from ethyl 2,4-dimethyl-1H-benzimidazole-6-carboxylate (500 mg) in a manner similar to that described in Preparation Example 38.

NMR(DMSO-d₆,δ): 1.30 (3H, t, J=7.1 Hz), 2.52 (3H, s), 2.57 (3H, s), 4.28 (2H, q, J=7.1 Hz), 5.60 (2H, br s), 6.53 (1H, d, J=8.4 Hz), 7.33 (1H, dd, J=2.1 Hz, 8.4 Hz), 7.64 (1H, br s), 7.74 (1H, d, J=2.1 Hz), 7.83 (1H, br s).

MS: 377 (M+1).

### PREPARATION EXAMPLE 191

1-(2,4-Dichlorobenzyl)-2,4-dimethyl-1H-benzimidazole-6-carboxylic acid (588 mg) was synthesized from ethyl 1-(2,4-dichlorobenzyl)-2,4-dimethyl-1H-benzimidazole-6-carboxylate (690 mg) in a manner similar to that described in Example 14.

NMR(DMSO-d₆,δ): 2.52 (3H, s), 2.56 (3H, s), 5.59 (2H, br s), 6.50 (1H, d, J=8.4 Hz), 7.33 (1H, dd, J=2.2 Hz, 8.4 Hz), 7.63 (1H, br s), 7.74 (1H, d, J=2.2 Hz), 7.79 (1H, br s), 12.62 (1H, br s).

MS: 347 (M−1).

### PREPARATION EXAMPLE 192

1-(Ethoxycarbonyl)-2-oxopropyl 1-(2,4-dichlorobenzyl)-2,4-dimethyl-1H-benzimidazole-6-carboxylate (1.0 g) was synthesized from 1-(2,4-dichlorobenzyl)-2,4-dimethyl-1H-benzimidazole-6-carboxylic acid (550 mg) in a manner similar to that described in Preparation Example 184.

### PREPARATION EXAMPLE 193

Ethyl 2-[1-(2,4-dichlorobenzyl)-2,4-dimethyl-1H-benzimidazol-6-yl]-4-methyl-1,3-oxazole-5-carboxylate (251 mg) as white crystals, and ethyl 2-[1-(2,4-dichlorobenzyl)-2,4-dimethyl-1H-benzimidazol-6-yl]-4-methyl-1H-imidazole-5-carboxylate (174 mg) were synthesized from 1-(ethoxycarbonyl)-2-oxopropyl 1-(2,4-dichlorobenzyl)-2,4-dimethyl-1H-benzimidazole-6-carboxylate (750 mg) in a manner similar to that described in Preparation Example 185.

Ethyl 2-[1-(2,4-dichlorobenzyl)-2,4-dimethyl-1H-benzimidazol-6-yl]-4-methyl-1,3-oxazole-5-carboxylate

NMR(DMSO-d₆,δ): 1.33 (3H, m), 2.44 (3H, s), 2.49 (3H, s), 2.61 (3H, s), 4.33 (2H, q, J=7.0 Hz), 5.64 (2H, br s), 6.49 (1H, d, J=8.3 Hz), 7.32 (1H, dd, J=2.1 Hz, 8.3 Hz), 7.71 (1H, br s), 7.75 (1H, d, J=2.1 Hz), 7.89 (1H, br s).

MS: 458 (M+1).

Ethyl 2-[1-(2,4-dichlorobenzyl)-2,4-dimethyl-1H-benzimidazol-6-yl]-4-methyl-1H-imidazole-5-carboxylate

NMR(DMSO-d₆,δ): 1.29 (3H, t, J=7.0 Hz), 2.47 (3H, s), 2.4-2.5 (3H, s), 2.58 (3H, s), 4.23 (2H, q, J=7.0 Hz), 5.52 (2H, br s), 6.35 (1H, d, J=8.4 Hz), 7.32 (1H, dd, J=2.2 Hz, 8.4 Hz), 7.5-8.1 (2H, m), 7.76 (1H, d, J=2.2 Hz), 12.66 (1H, br s).

MS: 457 (M+1).

### PREPARATION EXAMPLE 194

To a mixture of ethyl 2,4-dimethyl-1H-benzimidazole-6-carboxylate (500 mg), K₂CO₃ (412 mg) and DMF (5 ml) was added 4-(bromomethyl)-3-chloro-1,1′-biphenyl (774 mg) at ambient temperature. The mixture was heated at 80° C. for 4 hours. After cooling, the reaction mixture was diluted with EtOAc (150 ml) and washed with water (50 ml) and brine (2×50 ml). The organic layer was dried over MgSO₄ and evaporated to give ethyl 1-[(3-chloro-1,1′-biphenyl-4-yl)methyl]-2,4-dimethyl-1H-benzimidazole-6-carboxylate (1.181 g) as a crude oil.

### PREPARATION EXAMPLE 195

1-[(3-Chloro-1,1′-biphenyl-4-yl)methyl]-2,4-dimethyl-1H-benzimidazole-6-carboxylic acid (830 mg) was synthesized from ethyl 1-[(3-chloro-1,1′-biphenyl-4-yl)methyl]-2,4-dimethyl-1H-benzimidazole-6-carboxylate (959 mg) in a manner similar to that described in Example 14.

NMR(DMSO-d₆,δ): 2.56 (3H, s), 2.57 (3H, s), 5.64 (2H, br s), 6.55 (1H, d, J=8.1 Hz), 7.3-7.6 (4H, m), 7.6-7.7 (3H, m), 7.8-7.9 (2H, m), 12.66 (1H, br s).

### PREPARATION EXAMPLE 196

1-(Ethoxycarbonyl)-2-oxopropyl 1-[(3-chloro-1,1′-biphenyl-4-yl)methyl]-2,4-dimethyl-1H-benzimidazole-6-carboxylate (1.38 g) was synthesized from 1-[(3-chloro-1,1′-biphenyl-4-yl)methyl]-2,4-dimethyl-1H-benzimidazole-6-carboxylic acid (800 mg) in a manner similar to that described in Preparation Example 184.

### PREPARATION EXAMPLE 197

Ethyl 2-{1-[(3-chloro-1,1′-biphenyl-4-yl)methyl]-2,4-dimethyl-1H-benzimidazol-6-yl}-4-methyl-1,3-oxazole-5-carboxylate (377 mg) as white crystals, and ethyl 2-{1-[(3-chloro-1,1′-biphenyl-4-yl)methyl]-2,4-dimethyl-1H-benzimidazol-6-yl}-4-methyl-1H-imidazole-5-carboxylate (236 mg) were synthesized from 1-(ethoxycarbonyl)-2-oxopropyl 1-[(3-chloro-1,1′-biphenyl-4-yl)methyl]-2,4-dimethyl-1H-benzimidazole-6-carboxylate (1.06 g) in a manner similar to that described in Preparation Example 185.

Ethyl 2-{1-[(3-chloro-1,1′-biphenyl-4-yl)methyl]-2,4-dimethyl-1H-benzimidazol-6-yl}-4-methyl-1,3-oxazole-5-carboxylate

NMR(DMSO-d₆,δ): 1.32 (3H, t, J=7.0 Hz), 2.43 (3H, s), 2.56 (3H, s), 2.62 (3H, s), 4.32 (2H, q, J=7.0 Hz), 5.70 (2H, br s), 6.56 (1H, d, J=8.1 Hz), 7.3-7.8 (7H, m), 7.86 (1H, br s), 7.92 (1H, br s).

MS: 500 (M+1).

Ethyl 2-{1-[(3-chloro-1,1′-biphenyl-4-yl)methyl]-2,4-dimethyl-1H-benzimidazol-6-yl}-4-methyl-1H-imidazole-5-carboxylate

NMR(DMSO-d₆,δ): 1.2-1.4 (3H, m), 2.3-2.5 (6H), 2.60 (3H, s), 4.1-4.4 (2H, m), 5.58 (2H, br s), 6.40 (1H, d, J=8.1 Hz), 7.3-8.1 (9H, m), 12.67 (1H, br s).

MS: 499 (M+1).

### PREPARATION EXAMPLE 198

Ethyl 1-[2-chloro-4-(pentyloxy)benzyl]-2,4-dimethyl-1H-benzimidazole-6-carboxylate (1.28 g) was synthesized from ethyl 2,4-dimethyl-1H-benzimidazole-6-carboxylate (500 mg) in a manner similar to that described in Preparation Example 194 except that 1-(bromomethyl)-2-chloro-4-(pentyloxy)benzene (935 mg) was used instead-of 4-(bromomethyl)-3-chloro-1,1′-biphenyl.

### PREPARATION EXAMPLE 199

1-[2-Chloro-4-(pentyloxy)benzyl]-2,4-dimethyl-1H-benzimidazole-6-carboxylic acid (716 mg) was synthesized from ethyl 1-[2-chloro-4-(pentyloxy)benzyl]-2,4-dimethyl-1H-benzimidazole-6-carboxylate (984 mg) in a manner similar to that described in Example 14.

NMR(DMSO-d₆,δ): 0.87 (3H, t, J=7.0 Hz), 1.2-1.3 (4H, m), 1.6-1.8 (2H,m), 2.53 (3H,s), 2.55 (3H,s), 3.94 (2H, t, J=6.5 Hz), 5.50 (2H, br s), 6.52 (1H, d, J=8.7 Hz), 6.82 (1H, dd, J=2.6 Hz, 8.7 Hz), 7.11 (1H, d, J=2.6 Hz), 7.61 (1H, br s), 7.77 (1H, br s), 12.63 (1H, br s).

### PREPARATION EXAMPLE 200

1-(Ethoxycarbonyl)-2-oxopropyl 1-[2-chloro-4-(pentyloxy)benzyl]-2,4-dimethyl-1H-benzimidazole-6-carboxylate (1.15 g) was synthesized from 1-[2-chloro-4-(pentyloxy)benzyl]-2,4-dimethyl-1H-benzimidazole-6-carboxylic acid (680 mg) in a manner similar to that described in Preparation Example 184.

### PREPARATION EXAMPLE 201

Ethyl 2-{1-[2-chloro-4-(pentyloxy)benzyl]-2,4-dimethyl-1H-benzimidazol-6-yl}-4-methyl-1,3-oxazole-5-carboxylate (318 mg) as white crystals, and ethyl 2-{1-[2-chloro-4-(pentyloxy)benzyl]-2,4-dimethyl-1H-benzimidazol-6-yl}-4-methyl-1H-imidazole-5-carboxylate (201 mg) were synthesized from 1-(ethoxycarbonyl)-2-oxopropyl 1-[2-chloro-4-(pentyloxy)benzyl]-2,4-dimethyl-1H-benzimidazole-6-carboxylate (900 mg) in a manner similar to that described in Preparation Example 185. Ethyl 2-{1-[2-chloro-4-(pentyloxy)benzyl]-2,4-dimethyl-1H-benzimidazol-6-yl)-4-methyl-1,3-oxazole-5-carboxylate

NMR(DMSO-d₆,δ): 0.7-1.0 (3H, m), 1.1-1.5 (7H, m), 1.5-1.8 (2H, m), 2.43 (3H, s), 2.53 (3H, s), 2.60 (3H, s), 3.94 (2H, t, J=6.4 Hz), 4.33 (2H, q, J=7.1 Hz), 5.54 (2H, br s), 6.52 (1H, d, J=8.6 Hz), 6.82 (1H, dd, J=2.5 Hz, 8.6 Hz), 7.12 (1H, d, J=2.5 Hz), 7.69 (1H, br s), 7.84 (1H, br s).

MS: 510 (M+1).

Ethyl 2-{1-[2-chloro-4-(pentyloxy)benzyl]-2,4-dimethyl-1H-benzimidazol-6-yl}-4-methyl-1H-imidazole-5-carboxylate

NMR(DMSO-d₆,δ): 0.8-1.0 (3H, m), 1.2-1.4 (7H, m), 1.5-1.8 (2H, m), 2.46 (3H, s), 2.4-2.6 (3H, s), 2.58 (3H, s), 3.93 (2H, t, J=6.4 Hz), 4.1-4.4 (2H, m), 5.45 (2H, br s), 6.31 (1H, d, J=8.6 Hz), 6.79 (1H, dd, J=2.5 Hz, 8.6 Hz), 7.14 (1H, d, J=2.5 Hz), 7.5-8.1 (2H, m), 12.70 (1H, br s).

MS: 509 (M+1).

### PREPARATION EXAMPLE 202

A mixture of 5-bromo-3-methyl-1,2-benzenediamine (1.79 g), tetraethyl orthocarbonate (9.3 ml) and acetic acid (AcOH) (695 mg) was heated at 80° C. for 1 hour. After cooling, the reaction mixture was evaporated. The residue was dissolved in EtOAc (50 ml) and washed with saturated NaHCO₃ (2×30 ml) and brine (30 ml). The organic layer was dried over MgSO₄ and filtered. Evaporation gave a residue which was chromatographed (silica gel, EtOAc/n-hexane=1/2→1/1) to give 6-bromo-2-ethoxy-4-methyl-1H-benzimidazole (1.91 g) as white crystals.

NMR(DMSO-d₆,δ): 1.37 (3H, t, J=7.0 Hz), 2.37 (3H, s), 4.48 (2H, q, J=7.0 Hz), 7.02 (1H, br s), 7.28 (1H, br s), 12.01 (1H, br s).

MS: 255, 257 (1:1 ratio, Br isotopes, M+1).

### PREPARATION EXAMPLE 203

To a mixture of 6-bromo-2-ethoxy-4-methyl-1H-benzimidazole (1.0 g), K₂CO₃ (704 mg) and DMF (10 ml) was added 1-(bromomethyl)-2-chloro-4-(pentyloxy)benzene (1.49 g) at ambient temperature. After stirring for 2 hours, the reaction mixture was diluted with EtOAc (150 ml) and washed with 5%-NaHCO₃ (50 ml) and brine (2×50 ml). The organic layer was dried over MgSO₄ and filtered. Evaporation gave a residue (2.25 g) which was triturated with EtOAc (2 ml)—n-hexane (2 ml) to give 6-bromo-1-[2-chloro-4-(pentyloxy)benzyl]-2-ethoxy-4-methyl-1H-benzimidazole (548 mg) as white crystals. The filtrate was evaporated and triturated with n-hexane to give second crop (589 mg).

NMR(DMSO-d₆,δ): 0.8-1.0 (3H, m), 1.2-1.5 (7H, m), 1.5-1.8 (2H, m), 2.43 (3H, s), 3.95 (2H, t, J=6.5 Hz), 4.53 (2H, q, J=7.0 Hz), 5.20 (2H, br s), 6.8-6.9 (2H, m), 7.0-7.2 (2H, m), 7.29 (1H, d, J=1.6 Hz).

MS: 465, 467 (1:1 ratio, Br isotopes, M+1).

### PREPARATION EXAMPLE 204

To a solution of (4-ethoxy-2-methoxyphenyl)methanol (200 mg) in CH₂Cl₂ (2 ml) were added triethyl amine (Et₃N) (167 mg) and then methanesulfonyl chloride (MsCl) (189 mg) dropwise at 5° C. The reaction mixture was stirred for 2 hours at room temperature. The reaction mixture was diluted with EtOAc (30 ml) and washed with saturated NaHCO₃ (2×20 ml) and brine (20 ml). The organic layer was dried over MgSO₄, filtered, and evaporated to give 4-ethoxy-2-methoxybenzyl methanesulfonate (146 mg) as a crude oil.

### PREPARATION EXAMPLE 205

Celite powder (38.0 g) and iron powder (27.6 g) were added to a mixture of 4-bromo-2-methyl-6-nitroaniline (38.0 g) and ammonium chloride (4.4 g) in a mixed solvent of ethanol (800 mL), tetrahydrofuran (400 mL) and water (400 mL). The mixture was heated at reflux for 2 hours and then cooled to room temperature. Inorganic salts were removed by celite pad filtration and washed with water and ethyl acetate. The organic layer was separated, washed with brine and dried over sodium sulfate. The crude product of 5-bromo-3-methyl-1,2-benzenediamine was used for the next step without purification.

MS (ES+): 201 (M⁺+1), 242 (M⁺+42).

### PREPARATION EXAMPLE 206

Acetic anhydride (15.5 mL) was added to a solution of 5-bromo-3-methyl-1,2-benzenediamine (33.1 g) in acetic acid (330 mL) at room temperature, and the mixture was heated at reflux (bath temperature: 150° C.) for 3 hours. Cooled to room temperature, and acetic acid was removed (about a half) in vacuo. Neutralized with aqueous solution of sodium carbonate, and the organic materials were extracted with ethyl acetate. The organic layer was washed with aqueous solution of sodium bicarbonate and brine and dried over sodium sulfate. It was concentrated in vacuo, and the residue was treated with activated carbon in ethyl acetate. The resulted crude product was dissolved in ethanol (360 mL), sulfuric acid was added to this solution, and the mixture was heated at reflux (bath temperature: 120° C.) for 2 hours. After cooled to room temperature, the mixture was poured into ice-cooled aqueous solution of sodium carbonate. Ethanol was removed in vacuo, and the organic materials were extracted with ethyl acetate. The organic layer was washed with aqueous solution of sodium bicarbonate and brine and dried over sodium sulfate. It was concentrated in vacuo to give 6-bromo-2,4-dimethyl-1H-benzimidazole (33.3 g) as a powder.

¹H NMR (200 MHz, DMSO-d₆, δ/ppm): 2.45 (3H, s), 2.48 (3H, s), 7.07 (1H, br s), 7.44 (1H, br s), 12.3 (1H, br).

### PREPARATION EXAMPLE 207

6-Bromo-1-(2,4-dichlorobenzyl)-2,4-dimethyl-1H-benzimidazole (3.27 g) was synthesized from 6-bromo-2,4-dimethyl-1H-benzimidazole (2.00 g) and 2,4-dichloro-1-(chloromethyl)benzene (1.91 g) in a manner similar to that described in Preparation Example 221.

¹H NMR (200 MHz, DMSO-d₆, δ/ppm): 2.44 (3H, s), 2.51 (3H, s), 5.51 (2H, s), 6.44 (1H, d, J=8.4 Hz), 7.16 (1H, s), 7.33 (1H, dd, J=8.4 Hz, 2.1 Hz), 7.52 (1H, d, J=1.6 Hz), 7.73 (1H, d, J=2.1 Hz).

### PREPARATION EXAMPLE 208

6-Bromo-1-{[3-chloro-5-(trifluoromethyl)-2-pyridinyl]methyl}-2,4-dimethyl-1H-benzimidazole (203 mg) was synthesized from 6-bromo-2,4-dimethyl-1H-benzimidazole (180 mg) and 3-chloro-2-(chloromethyl)-5-(trifluoromethyl)pyridine (230 mg) in a manner similar to that described in Preparation Example 221.

¹H NMR (200 MHz, DMSO-d₆, δ/ppm): 2.41 (3H, s), 2.52 (3H, s), 5.77 (2H, s), 7.11 (1H, s), 7.53 (1H, d, J=1.5 Hz), 8.56 (1H, d, J=1.5 Hz), 8.75 (1H, s).

### PREPARATION EXAMPLE 209

6-Bromo-1-(4-ethoxy-2-methylbenzyl)-2,4-dimethyl-1H-benzimidazole (269 mg) was synthesized from 6-bromo-2,4-dimethyl-1H-benzimidazole (200 mg) and 4-ethoxy-2-methylbenzyl methanesulfonate (267 mg) in a manner similar to that described in Preparation Example 221.

¹H NMR (200 MHz, DMSO-d₆, δ/ppm): 1.28 (3H, t, J=6.9 Hz), 2.32 (3H, s), 2.41 (3H, s), 2.51 (3H, s), 3.94 (2H, q, J=6.9 Hz), 5.35 (2H, s), 6.10 (1H, d, J=8.4 Hz), 6.60 (1H, dd, J=8.4 Hz, 2.6 Hz), 6.82 (1H, d, J=2.4 Hz), 7.13 (1H, s), 7.44 (1H, d, J=1.4 Hz).

### PREPARATION EXAMPLE 210

6-Bromo-1-(4-ethoxy-2-methoxybenzyl)-2,4-dimethyl-1H-benzimidazole (129 mg) was synthesized from 6-bromo-2,4-dimethyl-1H-benzimidazole (200 mg) and 4-ethoxy-2-methoxybenzyl methanesulfonate (285 mg) in a manner similar to that described in Preparation Example 221.

¹H NMR (200 MHz, DMSO-d₆, δ/ppm): 1.29 (3H, t, J=6.9 Hz), 2.47 (3H, s), 2.50 (3H, s), 3.77 (3H, s), 3.99 (2H, q, J=6.9 Hz), 5.24 (2H, s), 6.44 (1H, dd, J=8.0 Hz, 2.5 Hz), 6.57 (1H, d, J=2.6 Hz), 6.77 (1H, d, J=8.0 Hz), 7.09 (1H, s), 7.45 (1H, d, J=1.5 Hz).

### PREPARATION EXAMPLE 211

6-Bromo-1-(2-chloro-4-ethoxybenzyl)-2,4-dimethyl-1H-benzimidazole (292 mg) was synthesized from 6-bromo-2,4-dimethyl-1H-benzimidazole (200 mg) and 1-(bromomethyl)-2-chloro-4-ethoxybenzene (333 mg) in a manner similar to that described in Preparation Example 221.

¹H NMR (200 MHz, DMSO-d₆, δ/ppm): 1.29 (3H, t, J=6.9 Hz), 2.45 (3H, s), 2.50 (3H, s), 4.01 (2H, q, J=6.9 Hz), 5.43 (2H, s), 6.50 (1H, d, J=8.6 Hz), 6.83 (1H, dd, J=8.6 Hz, 2.5 Hz), 7.10 (1H, d, J=2.5 Hz), 7.13 (1H, m), 7.46 (1H, d, J=1.5 Hz).

### PREPARATION EXAMPLE 212

6-Bromo-1-[(3,5-dichloro-2-pyridinyl)methyl]-2,4-dimethyl-1H-benzimidazole (260 mg) was synthesized from 6-bromo-2,4-dimethyl-1H-benzimidazole (200 mg) and (3,5-dichloro-2-pyridinyl)methyl methanesulfonate (284 mg) in a manner similar to that described in Preparation Example 221.

¹H NMR (200 MHz, DMSO-d₆, δ/ppm): 2.41 (3H, s), 2.49 (3H, s), 5.65 (2H, s), 7.11 (1H, t, J=0.9 Hz), 7.50 (1H, d, J=0.9 Hz), 8.33 (1H, d, J=2.3 Hz), 8.42 (1H, d, J=2.2 Hz).

### PREPARATION EXAMPLE 213

6-Bromo-1-(2-chlorobenzyl)-2,4-dimethyl-1H-benzimidazole (270 mg) was synthesized from 6-bromo-2,4-dimethyl-1H-benzimidazole (200 mg) and 1-chloro-2-(chloromethyl)benzene (179 mg) in a manner similar to that described in Preparation Example 221.

MS (ES+): 349 (M⁺+1).

### PREPARATION EXAMPLE 214

Ethyl 4-[(6-bromo-2,4-dimethyl-1H-benzimidazol-1-yl)methyl]-3-chlorophenyl(methyl)carbamate (352 mg) was synthesized from 6-bromo-2,4-dimethyl-1H-benzimidazole (225 mg) and ethyl 4-(bromomethyl)-3-chlorophenyl(methyl)carbamate (460 mg) in a manner similar to that described in Preparation Example 221.

MS (ES+): 450 (M⁺+1).

### PREPARATION EXAMPLE 215

Acetic anhydride (8.58 mL) and sulfuric acid (46 μL) were, added to a solution of 4-bromo-2-methyl-6-nitroaniline (20.0 g) in acetic acid (200 mL) at room temperature, and the mixture was stirred at that temperature for 15 hours. Acetic acid was removed (about a half) in vacuo. Neutralized with aqueous solution of sodium carbonate, and the organic materials were extracted with ethyl acetate. The organic layer was washed with aqueous solution of sodium bicarbonate and brine and dried over sodium sulfate. It was concentrated in vacuo, and the residue was treated with activated carbon in ethyl acetate. The solution was concentrated in vacuo to give N-(4-bromo-2-methyl-6-nitrophenyl)acetamide (9.46 g) as a powder.

¹H NMR (200 MHz, DMSO-d₆, δ/ppm): 2.03 (3H, s), 2.28 (3H, s), 7.87 (1H, d, J=2.0 Hz), 7.95 (1H, d, J=2.1 Hz).

MS (ES+): 314 (M⁺+42).

### PREPARATION EXAMPLE 216

Ethyl 4′-(acetylamino)-3′-methyl-5′-nitro-1,1′-biphenyl-3-carboxylate (975 mg) was synthesized from N-(4-bromo-2-methyl-6-nitrophenyl)acetamide (1.0 g) and 3-(ethoxycarbonyl)phenylboronic acid (923 mg) in a manner similar to that described in Preparation Example 242.

¹H NMR (200 MHz, DMSO-d₆, δ/ppm): 1.35 (3H, t, J=7.1 Hz), 2.07 (3H, s), 2.38 (3H, s), 4.37 (2H, q, J=7.1 Hz), 7.66 (1H, t, J=7.8 Hz), 7.9-8.1 (3H, m), 8.24 (1H, s), 9.94 (1H, s).

### PREPARATION EXAMPLE 217

Iron (powder) was added to a suspension of ethyl 4′-(acetylamino)-3′-methyl-5′-nitro-1,1′-biphenyl-3-carboxylate (1.56 g) in a mixed solvent of ethanol (10 mL) and acetic acid (3.2 mL), and the mixture was heated at 110° C. for 3 hours. It was diluted with ethyl acetate and neutralized by addition of aoueous solution of sodium bicarbonate. Inorganic salts were removed by celite pad filtration. The organic layer was separated, washed with brine and dried over sodium sulfate. The crude product of ethyl 3-(2,4-dimethyl-1H-benzimidazol-6-yl)benzoate (1.01 g) was purified over silica gel chromatography (chloroform:methanol=10:1).

MS (ES+): 295 (M⁺+1).

### PREPARATION EXAMPLE 218

Ethyl 3-{1-[(2,6-dichloro-3-pyridinyl)methyl]-2,4-dimethyl-1H-benzimidazol-6-yl}benzoate (210 mg) was synthesized from ethyl 3-(2,4-dimethyl-1H-benzimidazol-6-yl)benzoate (182 mg) and (2,6-dichloro-3-pyridinyl)methyl methanesulfonate (198 mg) in a manner similar to that described in Preparation Example 221.

MS (ES+): 454 (M⁺+1).

### PREPARATION EXAMPLE 219

Ethyl 3-{2,4-dimethyl-1-[(5-methyl-2-phenyl-1,3-oxazol-4-yl)methyl]-1H-benzimidazol-6-yl}benzoate (226 mg) was synthesized from ethyl 3-(2,4-dimethyl-1H-benzimidazol-6-yl)benzoate (150 mg) and 4-(chloromethyl)-5-methyl-2-phenyl-1,3-oxazole (116 mg) in a manner similar to that described in Preparation Example 221.

¹H NMR (200 MHz, DMSO-d₆, δ/ppm): 2.54 (3H, s), 2.56 (3H, s), 2.75 (3H, s), 4.37 (2H, q, J=7.1 Hz), 5.45 (2H, s), 7.32 (1H, s), 7.4-7.6 (4H, m), 7.64 (1H, d, J=7.9 Hz), 7.76 (1H, s), 5 7.8-8.1 (4H, m), 8.27 (1H, s).

### PREPARATION EXAMPLE 220

Ethyl 3-{1-[(3-chloro-1,1′-biphenyl-4-yl)methyl]-2,4-dimethyl-1H-benzimidazol-6-yl}benzoate (128 mg) was synthesized from ethyl 3-(2,4-dimethyl-1H-benzimidazol-6-yl)benzoate (150 mg) and 4-(bromomethyl)-3-chloro-1,1′-biphenyl (215 mg) in a manner similar to that described in Preparation Example 221.

¹H NMR (400 MHz, DMSO-d₆, δ/ppm): 1.32 (3H, t, J=7.1 Hz), 2.52 (3H, s), 2.62 (3H, s), 4.33 (2H, q, J=7.1 Hz), 5.67 (2H, s), 6.57 (1H, d, J=8.1 Hz), 7.3-7.5 (4H, m), 7.5-7.6 (2H, m), 7.6-7.7 (2H, m), 7.84 (1H, d, J=1.8 Hz), 7.89 (1H, d, J=7.9 Hz), 7.94 (1H, d, J=7.9 Hz), 8.18 (1H, t, J=1.6 Hz).

### PREPARATION EXAMPLE 221

Potassium carbonate (59 mg) and 5-(chloromethyl)-4-methyl-2-[4-(trifluoromethyl)phenyl]-1,3-thiazole (109 mg) were added to a solution of ethyl 3-(2,4-dimethyl-1H-benzimidazol-6-yl)benzoate (100 mg) in N,N-dimethylformamide (2 mL) at room temperature, and the mixture was stirred at 80° C. for 3 hours. The reaction was quenched by addition of water, and the organic materials were extracted with ethyl acetate. The organic layer was washed with brine and dried over sodium sulfate. The crude product was purified over preparative thin layer chromatography (chloroform:methanol=20:1) to give ethyl 3-[2,4-dimethyl-1-({4-methyl-2-[4-(trifluoromethyl)phenyl]-1,3-thiazol-5-yl}methyl)-1H-benzimidazol-6-yl]benzoate (149 mg) as a powder.

MS (ES+): 550 (M⁺+1).

### PREPARATION EXAMPLE 222

Ethyl 4′-(acetylamino)-3′-nitro-1,1′-biphenyl-3-carboxylate (5.57 g) was synthesized from N-(4-bromo-2-nitrophenyl)acetamide (5.0 g) and 3-(ethoxycarbonyl)phenylboronic acid (4.31 g) in a manner similar to that described in Preparation Example 242.

¹H NMR (200 MHz, DMSO-d₆, δ/ppm): 1.35 (3H, t, J=7.1 Hz), 2.10 (3H, s), 4.37 (2H, q, J=7.1 Hz), 7.66 (1H, t, J=7.8 Hz), 7.74 (1H, d, J=8.5 Hz), 7.9-8.1 (3H, m), 8.2-8.3 (2H, m), 10.37 (1H, s).

MS (ES+): 370 (M⁺+42).

### PREPARATION EXAMPLE 223

Palladium on activated carbon (10%, dry, 155 mg) was added to a solution of ethyl 4′-(acetylamino)-3′-nitro-1,1′-biphenyl-3-carboxylate (956 mg) in a mixed solvent of tetrahydrofuran (15 mL) and ethanol (15 mL). The reaction bottle was purged by hydrogen gas, and the mixture was stirred at room temperature under hydrogen atmosphere (3 atm) for 8 hours. The bottle was purged by nitrogen gas, and the catalyst was filtered off. The solution was concentrated in vacuo to give ethyl 4′-(acetylamino)-3′-amino-1,1′-biphenyl-3-carboxylate (515 mg) as a powder.

MS (ES+): 299 (M⁺+1), 340 (M⁺+42).

### PREPARATION EXAMPLE 224

Ethyl 3-[1-(2-chloro-4-ethoxybenzyl)-2-methyl-1H-benzimidazol-6-yl}benzoate (169 mg) was synthesized from ethyl 4′-(acetylamino)-3′-amino-1,1′-biphenyl-3-carboxylate (180 mg) and 1-(bromomethyl)-2-chloro-4-ethoxybenzene (196 mg) in a manner similar to that described in Preparation Example 230.

MS (ES+): 449 (M⁺+1).

### PREPARATION EXAMPLE 225

Ethyl 3-[1-(2,4-dichlorobenzyl)-2-methyl-1H-benzimidazol-6-yl]benzoate (73 mg) was synthesized from ethyl 4′-(acetylamino)-3′-amino-1,1′-biphenyl-3-carboxylate (180 mg) and 2,4-dichloro-1-(chloromethyl)benzene (130 mg) in a manner similar to that described in Preparation Example 230.

¹H NMR (200 MHz, DMSO-d₆, δ/ppm): 1.34 (3H, t, J=7.2 Hz), 2.47 (3H, s), 4.35 (2H, q, J=7.1 Hz), 5.64 (2H, s), 6.51 (1H, d, J=8.4 Hz), 7.33 (1H, dd, J=8.3 Hz, 2.3 Hz), 7.48 (1H, s), 7.5-8.0 (7H, m), 8.19 (1H, s).

### PREPARATION EXAMPLE 226

Ethyl 3-(1-{[3-chloro-5-(trifluoromethyl)-2-pyridinyl]methyl}-2-methyl-1H-benzimidazol-6-yl)benzoate (160 mg) was synthesized from ethyl 4′-(acetylamino)-3′-amino-1,1′-biphenyl-3-carboxylate (185 mg) and 3-chloro-2-(chloromethyl)-5-(trifluoromethyl)pyridine (157 mg) in a manner similar to that described in Preparation Example 230.

¹H NMR (200 MHz, DMSO-d₆, δ/ppm): 1.33 (3H, t, J=7.1 Hz), 2.46 (3H, s), 4.34 (2H, q, J=7.2 Hz), 5.90 (2H, s), 7.48 (1H, dd, J=8.4 Hz, 1.5 Hz), 7.58 (1H, t, J=7.8 Hz), 7.65 (1H, d, J=8.4 Hz), 7.82 (1H, d, J=1.1 Hz), 7.92 (2H, m), 8.18 (1H, s), 8.56 (1H, d, J=1.7 Hz), 8.76 (1H, s).

### PREPARATION EXAMPLE 227

Ethyl 3-[1-(4-ethoxy-2-methylbenzyl)-2-methyl-1H-benzimidazol-6-yl]benzoate (106 mg) was synthesized from ethyl 4′-(acetylamino)-3′-amino-1,1′-biphenyl-3-carboxylate (180 mg) and 4-ethoxy-2-methylbenzyl methanesulfonate (160 mg) in a manner similar to that described in Preparation Example 230.

¹H NMR (200 MHz, DMSO-d₆, δ/ppm): 1.23 (3H, t, J=7.0 Hz), 1.34 (3H, t, J=7.1 Hz), 2.35 (3H, s), 2.45 (3H, s), 3.94 (2H, q, J=6.9 Hz), 4.34 (2H, q, J=7.1 Hz), 5.47 (2H, s), 6.23 (1H, d, J=8.5 Hz), 6.61 (1H, dd, J=8.4 Hz, 2.6 Hz), 6.82 (1H, d, J=2.4 Hz), 7.49 (1H, dd, J=8.3 Hz, 1.5 Hz), 7.58 (1H, t, J=7.7 Hz), 7.67 (1H, d, J=8.4 Hz), 7.71 (1H, d, J=0.9 Hz), 7.8-8.0 (2H, m), 8.16 (1H, s).

### PREPARATION EXAMPLE 228

Ethyl 3-{1-[(2,6-dichloro-3-pyridinyl)methyl]-2-methyl-1H-benzimidazol-6-yl}benzoate (234 mg) was synthesized from ethyl 4′-(acetylamino)-3′-amino-1,1′-biphenyl-3-carboxylate (180 mg) and (2,6-dichloro-3-pyridinyl)methyl methanesulfonate (170 mg) in a manner similar to that described in Preparation Example 230.

¹H NMR (200 MHz, DMSO-d₆, δ/ppm): 1.34 (3H, t, J=7.1 Hz), 2.49 (3H, s), 4.35 (2H, q, J=7.1 Hz), 5.64 (2H, s), 6.92 (1H, d, J=8.1 Hz), 7.45 (1H, d, J=8.1 Hz), 7.53 (1H, dd, J=8.2 Hz, 1.6 Hz), 7.61 (1H, d, J=7.7 Hz), 7.70 (1H, d, J=8.4 Hz), 7.8-8.0 (3H, m), 8.19 (1H, s).

### PREPARATION EXAMPLE 229

Ethyl 3-(1-{2-chloro-4-[(ethoxycarbonyl)(methyl)amino]benzyl}-2-methyl-1H-benzimidazol-6-yl)benzoate (177 mg) was synthesized from ethyl 4′-(acetylamino)-3′-amino-1,1′-biphenyl-3-carboxylate (180 mg) and ethyl 4-(bromomethyl)-3-chlorophenyl(methyl)carbamate (222 mg) in a manner similar to that described in Preparation Example 230.

¹H NMR (200 MHz, DMSO-d₆, δ/ppm): 1.13 (3H, t, J=7.1 Hz), 1.34 (3H, t, J=7.1 Hz), 2.50 (3H, s), 3.18 (3H, s), 4.05 (2H, q, J=7.1 Hz), 4.35 (2H, q, J=7.1 Hz), 5.63 (2H, s); 1H, d, J=8.4 Hz), 7.19 (1H, dd, J=8.4 Hz, 2.2 Hz), 7.5-7.7 (3H, m), 7.82 (1H, d, J=0.9 Hz), 7.9-8.0 (2H, m), 8.19 (1H, s).

### PREPARATION EXAMPLE 230

Potassium carbonate (92 mg) and 4-(bromomethyl)-3-chloro-1,1′-biphenyl (255 mg) were added to a solution of ethyl 4′-(acetylamino)-3′-amino-1,1′-biphenyl-3-carboxylate (180 mg) in N,N-dimethylformamide (4 mL) at room temperature, and the mixture was stirred at 80° C. for 2 hours. The reaction was quenched by addition of water, and the organic materials were extracted with ethyl acetate. The organic layer was washed with brine and dried over sodium sulfate.

The crude product was dissolved in ethanol (4 mL), and sulfuric acid (0.80 mL) was added to it at room temperature. The mixture was heated at reflux for 2 hours and cooled to room temperature. The mixture was neutralized by addition of an aqueous solution of sodium carbonate, and the organic materials were extracted with ethyl acetate. The organic layer was washed with brine and dried over sodium sulfate. The crude product was purified over preparative thin layer chromatography (chloroform:methanol 20:1) to give ethyl 3-{1-[(3-chloro-1,1′-biphenyl-4-yl)methyl]-2-methyl-1H-benzimidazol-6-yl}benzoate (204 mg) as a powder.

MS (ES+): 481 (M⁺+1).

### PREPARATION EXAMPLE 231

1-(2,4-Dichlorobenzyl)-2,4-dimethyl-6-(4,4,5,5-tetramethyl-1,3,2-dioxaborolan-2-yl)-1H-benzimidazole was synthesized from 6-bromo-1-(2,4-dichlorobenzyl)-2,4-dimethyl-1H-benzimidazole (100 mg) and 4,4,4′,4′,5,5,5′,5′-octamethyl-2,2′-bi-1,3,2-dioxaborolane (65 mg) in a manner similar to that described in Preparation Example 238. The crude solution was used for the next step without further treatment.

### PREPARATION EXAMPLE 232

Methyl 2-[1-(2,4-dichlorobenzyl)-2,4-dimethyl-1H-benzimidazol-6-yl]isonicotinate was synthesized from 1-(2,4-dichlorobenzyl)-2,4-dimethyl-6-(4,4,5,5-tetramethyl-1,3,2-dioxaborolan-2-yl)-1H-benzimidazole (crude solution, Preparation Example 231) and methyl 2-chloroisonicotinate (98 mg) in a manner similar to that described in Preparation Example 242. The crude solution was used for the next step without further treatment.

### PREPARATION EXAMPLE 233

1-(2-Chloro-4-ethoxybenzyl)-2,4-dimethyl-6-(4,4,5,5-tetramethyl-1,3,2-dioxaborolan-2-yl)-1H-benzimidazole was synthesized from 6-bromo-1-(2-chloro-4-ethoxybenzyl)-2,4-dimethyl-1H-benzimidazole (100 mg) and 4,4,4′,4′,5,5,5′,5′-octamethyl-2,2′-bi-1,3,2-dioxaborolane (65 mg) in a manner similar to that described in Preparation Example 238. The crude solution was used for the next step without further treatment.

### PREPARATION EXAMPLE 234

Ethyl 2-[1-(2-chloro-4-ethoxybenzyl)-2,4-dimethyl-1H-benzimidazol-6-yl]isonicotinate (86 mg) was synthesized from 1-(2-chloro-4-ethoxybenzyl)-2,4-dimethyl-6-(4,4,5,5-tetramethyl-1,3,2-dioxaborolan-2-yl)-1H-benzimidazole (crude solution, Preparation Example 233) and ethyl 2-chloroisonicotinate (107 mg) in a manner similar to that described in Preparation Example 242.

¹H NMR (200 MHz, DMSO-d₆, δ/ppm): 1.27 (3H, t, J=6.9 Hz), 1.37 (3H, t, J=7.1 Hz), 2.5 (3H, s), 2.61 (3H, s), 4.00 (2H, q, J=7.0 Hz), 4.40 (2H, q, J=7.1 Hz), 5.55 (2H, s), 6.50 (1H, d, J=8.7 Hz), 6.82 (1H, dd, J=8.7 Hz, 2.6 Hz), 7.12 (1H, d, J=2.5 Hz), 7.71 (1H, dd, J=5.0 Hz, 1.2 Hz), 7.83 (1H, s), 8.00 (1H, s), 8.30 (1H, s), 8.81 (1H, d, J=5.0 Hz).

### PREPARATION EXAMPLE 235

Ethyl 6-[1-(2-chloro-4-ethoxybenzyl)-2,4-dimethyl-1H-benzimidazol-6-yl]-2-pyridinecarboxylate (94 mg) was synthesized from 1-(2-chloro-4-ethoxybenzyl)-2,4-dimethyl-6-(4,4,5,5-tetramethyl-1,3,2-dioxaborolan-2-yl)-1H-benzimidazole (crude solution, Preparation Example 233) and ethyl 6-chloro-2-pyridinecarboxylate (54 mg) in a manner similar to that described in Preparation Example 242.

MS (ES+): 464 (M⁺+1).

### PREPARATION EXAMPLE 236

Methyl 6-[1-(2-chloro-4-ethoxybenzyl)-2,4-dimethyl-1H-benzimidazol-6-yl]nicotinate was synthesized from 1-(2-chloro-4-ethoxybenzyl)-2,4-dimethyl-6-(4,4,5,5-tetramethyl-1,3,2-dioxaborolan-2-yl)-1H-benzimidazole (crude solution, Preparation Example 233) and methyl 6-chloroisonicotinate (50 mg) in a manner similar to that described in Preparation Example 242. The crude solution was used for the next step without purification.

### PREPARATION EXAMPLE 237

Ethyl 5-[1-(2-chloro-4-ethoxybenzyl)-2,4-dimethyl-1H-benzimidazol-6-yl]-2-thiophenecarboxylate was synthesized from 1-(2-chloro-4-ethoxybenzyl)-2,4-dimethyl-6-(4,4,5,5-tetramethyl-1,3,2-dioxaborolan-2-yl)-1H-benzimidazole (crude solution, Preparation Example 233) and ethyl 5-bromo-2-thiophenecarboxylate (67 mg) in a manner similar to that described-in Preparation Example 242. The crude product was used for the next step without purification.

MS (ES+): 495 (M⁺+1).

### PREPARATION EXAMPLE 238

Dichlorobis(triphenylphosphine)palladium (17 mg) and potassium acetate (94 mg) were added to a solution of 6-bromo-1-{[3-chloro-5-(trifluoromethyl)-2-pyridinyl]methyl}-2,4-dimethyl-1H-benzimidazole (100 mg) and 4,4,4′,4′,5,5,5′,5′-octamethyl-2,2′-bi-1,3,2-dioxaborolane (61 mg) in 1,4-dioxane (1 mL), and the mixture was heated at 80° C. with stirring for 15 hours. The crude solution of 1-{[3-chloro-5-(trifluoromethyl)-2-pyridinyl]methyl}-2,4-dimethyl-6-(4,4,5,5-tetramethyl-1,3,2-dioxaborolan-2-yl)-1H-benzimidazole was used for the next step without further treatment.

### PREPARATION EXAMPLE 239

Methyl 2-(1-{[3-chloro-5-(trifluoromethyl)-2-pyridinyl]methyl}-2,4-dimethyl-1H-benzimidazol-6-yl)isonicotinate (82 mg) was synthesized from 1-{[3-chloro-5-(trifluoromethyl)-2-pyridinyl]methyl}-2,4-dimethyl-6-(4,4,5,5-tetramethyl-1,3,2-dioxaborolan-2-yl)-1H-benzimidazole (crude solution, Preparation Example 238) and ethyl 2-chloroisonicotinate (106 mg) in a manner similar to that described in Preparation Example 242.

¹H NMR (200 MHz, DMSO-d₆, δ/ppm): 1.36 (3H, t, J=7.1 Hz), 2.47 (3H, s), 2.61 (3H, s), 4.39 (2H, q, J=7.1 Hz), 5.90 (2H, s), 7.70 (1H, dd, J=4.9 Hz, 1.3 Hz), 7.80 (1H, s), 8.02 (1H, s) 8.29 (1H, s), 8.57 (1H, d, J=1.8 Hz), 8.76 (1H, s), 8.80 (1H, d, J=5.0 Hz).

### PREPARATION EXAMPLE 240

Ethyl 6-(1-{[3-chloro-5-(trifluoromethyl)-2-pyridinyl]methyl}-2,4-dimethyl-1H-benzimidazol-6-yl)-2-pyridinecarboxylate (67 mg) was synthesized from 1-{[3-chloro-5-(trifluoromethyl)-2-pyridinyl]methyl}-2,4-dimethyl-6-(4,4,5,5-tetramethyl-1,3,2-dioxaborolan-2-yl)-1H-benzimidazole (crude solution, Preparation Example 238) and ethyl 6-chloro-2-pyridinecarboxylate (53 mg) in a manner similar to that described in Preparation Example 242.

MS (ES+): 489 (M⁺+1).

### PREPARATION EXAMPLE 241

Ethyl 4-[1-(2-chloro-4-ethoxybenzyl)-2,4-dimethyl-1-H-benzimidazol-6-yl]benzoate (95 mg) was synthesized from 6-bromo-1-(2-chloro-4-ethoxybenzyl)-2,4-dimethyl-1H-benzimidazole (100 mg) and 4-(ethoxycarbonyl)phenylboronic acid (64 mg) in a manner similar to that described in Preparation Example 242.

MS (ES+): 463 (M⁺+1).

### PREPARATION EXAMPLE 242

4-(Ethoxycarbonyl)phenylboronic acid (60 mg), tetrakis(triphenylphosphine)palladium (14 mg), and lithium chloride (30 mg) were added to a solution of 6-bromo-1-{[3-chloro-5-(trifluoromethyl)-2-pyridinyl]methyl}-2,4-dimethyl-1H-benzimidazole (100 mg) in 1,2-dimethoxyethane (2 mL). An aqueous solution of sodium carbonate (2 N, 0.36 mL) was added to it, and the mixture was heated at reflux for 5 hours. It was cooled to room temperature, diluted with chloroform, and neutralized by addition of 1 N hydrochloric acid. The organic layer was separated, washed with water, and dried over sodium sulfate. The crude product was purified over preparative thin-layer chromatography (ethyl acetate) to give ethyl 4-(1-{[3-chloro-5-(trifluoromethyl)-2-pyridinyl]methyl}-2,4-dimethyl-1H-benzimidazol-6-yl)benzoate (92 mg) as a powder.

MS (ES+): 488 (M⁺+1).

### PREPARATION EXAMPLE 243

1-(2,4-Dichlorobenzyl)-6-(3-methoxyphenyl)-2,4-dimethyl-1H-benzimidazole (1.75 g) was synthesized from 6-bromo-1-(2,4-dichlorobenzyl)-2,4-dimethyl-1H-benzimidazole (2.0 g) and 3-methoxyphenylboronic acid (989 mg) in a manner similar to that described in Preparation Example 242.

MS (ES+): 411 (M⁺+1).

### PREPARATION EXAMPLE 244

Boron tribromide (0.75 mL) was added dropwise to an ice-cooled solution of 1-(2,4-dichlorobenzyl)-6-(3-methoxyphenyl)-2,4-dimethyl-1H-benzimidazole (1.55 g) in dichloromethane (30 mL), and the mixture was stirred at that temperature for 1 hour. The reaction was quenched by addition of water, and pH was adjusted to 6. The organic layer was separated, washed with water and brine, and dried over sodium sulfate. It was concentrated in vacuo to give 3-[1-(2,4-dichlorobenzyl)-2,4-dimethyl-1H-benzimidazol-6-yl]phenol (1.35 g) as a powder.

MS (ES+): 397 (M⁺+1).

### PREPARATION EXAMPLE 245

Potassium carbonate (115 mg) and ethyl bromoacetate (46 μL) were added to a solution of 3-[1-(2,4-dichlorobenzyl)-2,4-dimethyl-1H-benzimidazol-6-yl]phenol (150 mg) in N,N-dimethylformamide (3 mL) at room temperature, and the mixture was stirred at 60° C. for 2 hours. The reaction was quenched by addition of water, and the organic materials were extracted with ethyl acetate. The organic layer was washed with brine and dried over sodium sulfate. The crude product was purified over preparative thin layer chromatography (chloroform:methanol=20:1) to give ethyl {3-[1-(2,4-dichlorobenzyl)-2,4-dimethyl-1H-benzimidazol-6-yl]phenoxy}acetate (142 mg) as a powder.

MS (ES+): 483 (M⁺+1).

### PREPARATION EXAMPLE 246

Ethyl 3-{1-[2-chloro-4-(pentyloxy)benzyl]-2-ethoxy-4-methyl-1H-benzimidazol-6-yl}benzoate (169 mg) was synthesized from 6-bromo-1-[2-chloro-4-(pentyloxy)benzyl]-2-ethoxy-4-methyl-1H-benzimidazole (200 mg) and 3-(ethoxycarbonyl)phenylboronic acid (108 mg) in a manner similar to that described in Preparation Example 242.

MS (ES+): 535 (M⁺+1).

### EXAMPLE 1

({1-[2-Chloro-4-(pentyloxy)benzyl]-2-methyl-1H-benzimidazol-6-yl}oxy)acetic acid (15 mg) was synthesized from ethyl 4-({1-[2-chloro-4-(pentyloxy)benzyl]-2-methyl-1H-benzimidazol-6-yl}oxy)acetate (61 mg) in a manner similar to that described in Example 4.

NMR(DMSO-d₆,δ): 0.87 (3H, t, J=7.1 Hz), 1.3-1.4 (4H, m), 1.6-1.7 (2H, m), 2.39 (3H, s), 3.94 (2H, t, J=6.5 Hz), 4.60 (2H, s),5.39 (2H, s), 6.50 (1H, d, J=8.6 Hz), 6.7-6.9 (2H, m), 6.98 (1H, d, J=2.3 Hz), 7.10 (1H, d, J=2.5 Hz), 7.43 (1H, d, J=8.7 Hz).

MS(API-ES, Posi): 417.2.

### EXAMPLE 2

2-({1-[2-Chloro-4-(pentyloxy)benzyl]-2-methyl-1H-benzimidazol-6-yl}oxy)-2-methylpropanoic acid (87 mg) was synthesized from ethyl 2-({1-[2-chloro-4-(pentyloxy)benzyl]-2-methyl-1H-benzimidazol-6-yl}oxy)-2-methylpropanoate (120 mg) in a manner similar to that described in Example 4.

NMR(DMSO-d₆,δ): 0.87 (3H, t, J=7.0 Hz), 1.2-1.4 (4H, m), 1.40 (6H, s), 2.45 (3H, s), 3.94 (2H, t, J=6.4 Hz), 5.34 (2H, s), 6.61 (1H, d, J=8.6 Hz), 6.73 (1H, dd, J=8.6 Hz, 2.3 Hz), 7.8-7.9 (2H, m), 7.09 (1H, d, J=2.5 Hz), 7.41 (1H, d, J=8.6 Hz), 13.0 (1H, broad s).

MS(API-ES; Posi): 445.2.

### EXAMPLE 3

To a suspension of 1-[2-chloro-4-(pentyloxy)benzyl]-2-methyl-1H-benzimidazol-6-ol (1.0 g) in a solvent mixture of tetrahydrofuran (5 mL) and N,N-dimethylformamide (5 mL) was added sodium hydride (60% dispersion in mineral oil; 123 mg). The mixture was stirred at 80° C. for 15 minutes and allowed to cool to ambient temperature. To the mixture was added β-propiolactone (1.0 g) in an ice-bath. After stirring overnight at ambient temperature, the mixture was partitioned between ethyl acetate and saturated aqueous ammonium chloride. The organic layer was separated, washed with brine, dried over anhydrous sodium sulfate, and concentrated in vacuo. The residue was purified by column chromatography on silica gel (elution; 10:1 chloroform-methanol) to give 3-({1-[2-chloro-4-(pentyloxy)benzyl]-2-methyl-1H-benzimidazol-6-yl}oxy)propanoic acid (17 mg) as a white solid.

NMR(DMSO-d₆,δ): 0.87 (3H, t, J=7 Hz), 1.2-2.0 (6H, m), 2.39 (3H, s), 2.66 (2H, t, J=6 Hz), 3.93 (2H, t, J=6 Hz), 4.11 (2H, t, J=6 Hz), 5.40 (2H, s), 6.47 (1H, d, J=8 Hz), 6.7-6.9 (2H, m), 6.99 (1H, d, J=2 Hz), 7.10 (1H, d, J=2 Hz), 7.42 (1H, d, J=9 Hz).

MS: 431 (M+1).

### EXAMPLE 4

A solution of ethyl 4-({1-[2-chloro-4-(pentyloxy)benzyl]-2-methyl-1H-benzimidazol-6-yl}oxy)butanoate (0.26 g) in ethanol (4 mL) was treated with 4 N sodium hydroxide (2 mL). After stirring for an hour at ambient temperature, the mixture was acidified with 3 N hydrochloric acid and concentrated in vacuo. The residue was partitioned between ethyl acetate and water. The organic layer was separated, washed with brine, dried over anhydrous sodium sulfate, and concentrated in vacuo. The residue was recrystallized from ethyl acetate to give 4-({1-[2-chloro-4-(pentyloxy)benzyl]-2-methyl-1H-benzimidazol-6-yl}oxy)butanoic acid (0.17 g) as a white solid.

NMR(DMSO-d₆,δ): 0.87 (3H, t, J=7 Hz), 1.2-2.0 (8H, m), 2.37 (2H, t, J=7 Hz), 2.40 (3H, s), 3.92 (2H, t, J=6 Hz), 3.93 (2H, t, J=6 Hz), 5.40 (2H, s), 6.48 (1H, d, J=8 Hz), 6.7-6.9 (2H, m), 6.98 (1H, d, J=2 Hz), 7.10 (1H, d, J=2 Hz), 7.44 (1H, d, J=9 Hz), 12.13 (1H, br s).

MS: 443 (M−H).

### EXAMPLE 5

5-({1-[2-Chloro-4-(pentyloxy)benzyl]-2-methyl-1H-benzimidazol-6-yl}oxy)pentanoic acid (0.15 g) was synthesized from ethyl 5-({1-[2-chloro-4-(pentyloxy)benzyl]-2-methyl-1H-benzimidazol-6-yl}oxy)pentanoate (200 mg) in a manner similar to that described in Example 4.

NMR(DMSO-d₆,δ): 0.87 (3H, t, J=7 Hz), 1.2-1.8 (10H, m), 2.27 (2H, t, J=7 Hz), 2.75 (3H, s), 3.97 (4H, t, J=6 Hz), 5.66 (2H, s), 6.89 (1H, dd, J=3 Hz, 9 Hz), 7.04 (1H, d, J=9 Hz), 7.1-7.2 (2H, m), 7.29 (1H, d, J=2 Hz), 7.70 (1H, d, J=9 Hz), 11.5-12.7 (1H, br m).

MS: 457 (M−H).

### EXAMPLE 6

6-({1-[2-Chloro-4-(pentyloxy)benzyl]-2-methyl-1H-benzimidazol-6-yl}oxy)hexanoic acid (0.14 g) was synthesized from ethyl 6-({1-[2-chloro-4-(pentyloxy)benzyl]-2-methyl-1H-benzimidazol-6-yl}oxy)hexanoate (270 mg) in a manner similar to that described in Example 4.

NMR(DMSO-d₆,δ): 0.87 (3H, t, J=7 Hz), 1.2-1.7 (12H, m), 2.22 (2H, t, J=7 Hz), 2.74 (3H, s), 3.97 (4H, t, J=6 Hz), 5.65 (2H, s), 6.89 (1H, dd, J=2 Hz, 9 Hz), 7.03 (1H, d, J=9 Hz), 7.0-7.2 (2H, m), 7.26 (1H, d, J=9 Hz), 7.68 (1H, d, J=9 Hz), 11.5-12.7 (1H, br m).

MS: 473 (M+1).

### EXAMPLE 7

4-({1-[2-Chloro-4-(pentyloxy)benzyl]-2-methyl-1H-benzimidazol-7-yl}oxy)butanoic acid (80 mg) was synthesized from ethyl 4-({1-[2-chloro-4-(pentyloxy)benzyl]-2-methyl-1H-benzimidazol-7-yl}oxy)butanoate (180 mg) in a manner similar to that described in Example 4.

NMR(DMSO-d₆,δ): 0.86 (3H, t, J=7.0 Hz), 1.2-1.4 (4H, m), 1.6-1.8 (4H, m), 2.11 (2H, t, J=7.4 Hz), 2.41 (3H, s), 3.9-4.0 (4H, m), 5.59 (2H, s), 6.13 (1H, d, J=8.7 Hz), 6.67 (1H, d, J=8.0 Hz), 6.75 (1H, d, J=2.5 Hz), 6.77 (1H, d, J=2.6 Hz), 7.0-7.1 (2H, m), 7.16 (1H, d, J=7.9 Hz).

MS(API-ES Nega): 443.2.

### EXAMPLE 8

4-({1-[2-Chloro-4-(pentyloxy)benzyl]-2-methyl-1H-benzimidazol-4-yl}oxy)butanoic acid (151 mg) was synthesized from ethyl 4-({1-[2-chloro-4-(pentyloxy)benzyl]-2-methyl-1H-benzimidazol-4-yl}oxy)butanoate (209 mg) in a manner similar to that described in Example 4.

NMR(DMSO-d₆,δ): 0.87 (3H, t, J=7.1 Hz), 1.3-1.4 (4H, m), 1.6-1.7 (2H, m), 1.9-2.0 (2H, m), 2.4-2.5 (5H, m), 3.93 (2H, t, J=6.5 Hz), 4.20 (2H, t, J=6.4 Hz), 5.39 (2H, s), 6.48 (1H, d, J=8.6 Hz), 6.67 (1H, d, J=6.6 Hz), 6.80 (1H, dd, J=9.0 Hz, 2.5 Hz), 6.89 (1H, d, J=7.6 Hz), 7.01 (1H, t, J=8.0 Hz), 7.09 (1H, d, J=2.5 Hz), 12.2 (1H, broad s).

MS(API-ES, Posi): 445.2.

### EXAMPLE 9

A mixture of tert-butyl ({1-[2-chloro-4-(pentyloxy)benzyl]-2-methyl-1H-benzimidazol-7-yl}oxy)acetate (210 mg), trifluoroacetic acid (2 mL) and methanol (2 mL) was stirred at ambient temperature for 16 hours to form the corresponding methyl ester. The mixture was concentrated in vacuo and the residue was dissolved in ethanol (2 mL) and treated with 1 N sodium hydroxide (1 mL). After stirring at 60° C. for an hour, the mixture was cooled in an ice-bath and treated with 1 N hydrochloric acid (1 mL). The resulting suspension was filtered to give ({1-[2-chloro-4-(pentyloxy)benzyl]-2-methyl-1H-benzimidazol-7-yl}oxy)acetic acid (180 mg).

NMR(DMSO-d₆,δ): 0.87 (3H, t, J=7.1 Hz), 1.2-1.4 (4H, m), 1.6-1.8 (2H, m), 2.36 (3H, s), 3.91 (2H, t, J=6.5 Hz), 4.66 (2H, s), 5.73 (2H, s), 6.31 (1H, d,J=8.7 Hz), 6.69 (1H, d, J=7.8 Hz), 6.76 (1H, dd, J=8.7 Hz, 2.6 Hz), 7.0-7.1 (2H, m), 7.20 (1H, d, J=7.8 Hz).

MS(API-ES, Nega): 415.2.

### EXAMPLE 10

6-({1-[2-Chloro-4-(pentyloxy)benzyl]-2-methyl-1H -benzimidazol-4-yl}oxy)hexanoic acid (0.35 g) was synthesized from ethyl 6-({1-[2-chloro-4-(pentyloxy)benzyl]-2-methyl-1H -benzimidazol-4-yl}oxy)hexanoate (1.16 g) in a manner similar to that described in Example 4.

NMR(DMSO-d₆,δ): 0.87 (3H, t, J=7 Hz), 0.9-1.8 (12H, m), 2.25 (2H, t, J=7 Hz), 2.44 (3H, s), 3.93 (2H, t, J=6 Hz), 4.17 (2H, t, J=6 Hz), 5.39 (2H, s), 6.47 (1H, d, J=9 Hz), 6.65 (1H, d, J=7 Hz), 6.7-7.1 (3H, m), 7.09 (1H, d, J=2 Hz), 12.03 (1H, br s).

MS: 471 (M−1).

### EXAMPLE 11

6-({1-[2-Chloro-4-(pentyloxy)benzyl]-2-methyl-1H -benzimidazol-7-yl}oxy)hexanoic acid (0.45 g) was synthesized from ethyl 6-({1-[2-chloro-4-(pentyloxy)benzyl]-2-methyl-1H -benzimidazol-7-yl}oxy)hexanoate (730 mg) in a manner similar to that described in Example 4.

NMR(DMSO-d₆,δ): 0.87 (3H, t, J=7 Hz), 1.0-1.8 (12H, m), 2.08 (2H, t, J=7 Hz), 2.76 (3H, s), 3.95 (2H, t, J=6 Hz), 3.99 (2H, t, J=6 Hz), 5.72 (2H, s), 6.57 (1H, d, J=9 Hz), 6.78 (1H, dd, J=2 Hz, 9 Hz), 7.05 (1H, d, J=7 Hz), 7.16 (1H, d, J=2 Hz), 7.3-7.5 (2H, m).

MS: 471 (M−1).

### EXAMPLE 12

4-({1-[2-Chloro-4-(pentyloxy)benzyl]-2-methyl-1H -benzimidazol-5-yl}oxy)butanoic acid (44 mg) was synthesized from ethyl 4-({1-[2-chloro-4-(pentyloxy)benzyl]-2-methyl-1H -benzimidazol-5-yl}oxy)butanoate (62 mg) in a manner similar to that described in Example 4.

NMR(DMSO-d₆,δ): 0.87 (3H, t, J=7.1 Hz), 1.3-1.4 (4H, m), 1.6-1.7 (2H, m), 1.9-2.0 (2H, m), 2.38 (2H, t, J=7.7 Hz), 2.43 (3H, s), 3.9-4.0 (4H, m), 5.38 (2H, s), 6.51 (2H, d, J=4.3 Hz), 6.7-6.9 (2H, m), 7.0-7.1 (2H, m), 7.18 (1H, d, J=4.3 Hz), 12.1 (1H, broad s).

MS(API-ES Posi): 445.3.

### EXAMPLE 13

6-({1-[2-Chloro-4-(pentyloxy)benzyl]-2-methyl-1H -benzimidazol-5-yl}oxy)hexanoic acid (0.76 g) was synthesized from ethyl 6-({1-[2-chloro-4-(pentyloxy)benzyl]-2-methyl-1H -benzimidazol-5-yl)oxy}hexanoate (1.5 g) in a manner similar to that described in Example 4.

NMR(DMSO-d₆,δ): 0.87 (3H, t, J=7 Hz), 1.3-1.8 (12H, m), 2.23 (2H, t, J=7 Hz), 2.81 (3H, s), 3.97 (2H, t, J=6 Hz), 4.03 (2H, t, J=6 Hz), 5.65 (2H, s), 6.89 (1H, dd, J=2 Hz, 9 Hz), 7.0-7.2 (3H, m), 7.24 (1H, d, J=2 Hz), 7.50 (1H, d, J=9 Hz), 11.5-12.7 (1H, br m).

### EXAMPLE 14

To a solution of ethyl 4-({1-[2-chloro-4-(pentyloxy)benzyl]-2,4-dimethyl-1H-benzimidazol-6-yl}oxy)butanoate (46 mg) in dioxane (0.4 ml) was added 1N-NaOH (0.19 ml) at ambient temperature. The mixture was heated at 90° C. for 3 hours. After cooling, the pH of the reaction mixture was adjusted to around 3.5 with 1N-HCl. The precipitates were collected by filtration and washed with water and MeOH to give 4-({1-[2-chloro-4-(pentyloxy)benzyl]-2,4-dimethyl-1H-benzimidazol-6-yl}oxy)butanoic acid (36 mg) as white crystals.

NMR(DMSO-d₆,δ): 0.8-1.0 (3H, m), 1.2-1.5 (4H, m), 1.5-1.8 (2H, m), 1.8-2.0 (2H, m), 2.35 (2H, t, J=7.1 Hz), 2.39 (3H, s), 2.46 (3H, s), 3.8-4.0 (4H, m), 5.37 (2H, br s), 6.42 (1H, d, J=8.6 Hz), 6.60 (1H, d, J=1.5 Hz), 6.7-6.9 (2H, m), 7.10 (1H, d, J=2.5 Hz), 12.15 (1H, br s).

MS: 459 (M+1).

### EXAMPLE 15

4-{[1-(2,4-Dichlorobenzyl)-2,4-dimethyl-1H-benzimidazol -6-yl]oxy}butanoic acid (95 mg) was synthesized from ethyl 4-{[1-(2,4-dichlorobenzyl)-2,4-dimethyl-1H-benzimidazol-6-yl]oxy}butanoate (200 mg) in a manner similar to that described in Example 14.

NMR(DMSO-d₆,δ): 1.8-2.0 (2H, m), 2.36 (2H, t, J=7.2 Hz), 2.38 (3H, s), 2.47 (3H, s), 3.90 (2H, t, J=6.4 Hz), 5.45 (2H, br s), 6.40 (1H, d, J=8.4 Hz), 6.62 (1H, d, J=1.8 Hz), 6.81 (1H, d, J=1.8 Hz), 7.32 (1H, dd, J=2.2 Hz, 8.4 Hz), 7.72 (1H, d, J=2.2 Hz), 12.12 (1H, br s).

MS: 405 (M−1).

### EXAMPLE 16

To a solution of ethyl 4-[(1-{[3-chloro-5-(trifluoromethyl)-2-pyridinyl]methyl}-2,4-dimethyl-1H -benzimidazol-6-yl)oxy]butanoate (200 mg) in dioxane (2 ml) was added 1N-NaOH (0.85 ml) at ambient temperature. The mixture was heated at 80° C. for 3 hours. After cooling, the reaction mixture was neutralized with 1N-HCl. The precipitates were collected by filtration and washed with water to give 4-[(1-{[3-chloro-5-(trifluoromethyl)-2-pyridinyl]methyl}-2,4-dimethyl-1H-benzimidazol-6-yl)oxy]butanoic acid (85 mg) as white crystals.

NMR(DMSO-d₆,δ): 1.8-2.0 (2H, m), 2.35 (2H, t, J=7.2 Hz), 2.38 (3H, s), 2.45 (3H, s), 3.89 (2H, t, J=6.3 Hz), 5.70 (2H, br s), 6.58 (1H, d, J=1.8 Hz), 6.79 (1H, d, J=1.8 Hz), 8.55 (1H, br s), 8.77 (1H, br s), 12.11 (1H, br s).

MS: 440 (M−1).

### EXAMPLE 17

4-({2,4-Dimethyl-1-[(5-methyl-2-phenyl-1,3-oxazol-4-yl)methyl]-1H-benzimidazol-6-yl}oxy)butanoic acid (100 mg) was synthesized from ethyl 4-({2,4-dimethyl-1-[(5-methyl-2-phenyl-1,3-oxazol-4-yl)methyl]-1H-benzimidazol-6-yl}oxy)butanoate (130 mg) in a manner similar to that described in Example 14.

NMR(DMSO-d₆,δ): 1.8-2.1 (2H, m), 2.3-2.5 (8H, m), 2.66 (3H, s), 4.00 (2H, t, J=6.3 Hz), 5.30 (2H, br s), 6.59 (1H, d, J=1.8 Hz), 6.95 (1H, d, J=1.8 Hz), 7.4-7.6 (3H, m), 7.8-7.9 (2H, m), 12.15 (1H, br s).

MS: 418 (M−1).

### EXAMPLE 18

4-{[2,4-Dimethyl-1-({4-methyl-2-[4-(trifluoromethyl)phenyl]-1,3-thiazol-5-yl}methyl)-1H-benzimidazol-6-yl]oxy}butanoic acid (126 mg) was synthesized from ethyl 4-}[2,4-dimethyl-1-({4-methyl-2-[4-(trifluoromethyl)phenyl]-1,3-thiazol-5-yl}methyl)-1H-benzimidazol-6-yl]oxy}butanoate (140 mg) in a manner similar to that described in Example 14.

NMR(DMSO-d₆,δ): 1.8-2.1 (2H, m), 2.3-2.6 (8H, m), 2.58 (3H, s), 3.99 (2H, t, J=6.4 Hz), 5.66 (2H, br s), 6.63 (1H, d, J=1.8 Hz), 6.98 (1H, d, J=1.8 Hz), 7.77 (2H, d, J=8.3 Hz), 8.02 (2H, d, J=8.3 Hz), 12.15 (1H, br s).

MS: 502 (M−1).

### EXAMPLE 19

4-{[1-(4-Ethoxy-2-methylbenzyl)-2,4-dimethyl-1H-benzimidazol-6-yl]oxy}butanoic acid (192 mg) was synthesized from ethyl 4-{[1-(4-ethoxy-2-methylbenzyl)-2,4-dimethyl-1H-benzimidazol-6-yl]oxy}butanoate (275 mg) in a manner similar to that described in Example 14.

NMR(DMSO-d₆,δ): 1.27 (3H, t, J=7.0 Hz), 1.8-2.0 (2H, m), 2.2-2.4 (8H, m), 2.46 (3H, s), 3.8-4.0 (4H, m), 5.28 (2H, br s), 6.14 (1H, d, J=8.5 Hz), 6.5-6.7 (2H, m), 6.73 (1H, d, J=2.2 Hz), 6.80 (1H, d, J=2.2 Hz), 12.15 (1H, br s).

MS: 395 (M+1).

### EXAMPLE 20

4-{[1-(2-Chlorobenzyl)-2,4-dimethyl-1H-benzimidazol-6-yl]oxy}butanoic acid (128 mg) was synthesized from ethyl 4-{[1-(2-chlorobenzyl)-2,4-dimethyl-1H-benzimidazol-6-yl]oxy}butanoate (290 mg) in a manner similar to that described in Example 14.

NMR(DMSO-d₆,δ): 1.7-2.0 (2H, m), 2.35 (2H, t, J=7.2 Hz), 2.38 (3H, s), 2.47 (3H, s), 3.89 (2H, t, J=6.4 Hz), 5.46 (2H, br s), 6.43 (1H, dd, J=1.5 Hz, 7.6 Hz), 6.61 (1H, d, J=1.8 Hz), 6.79 (1H, d, J=1.8 Hz), 7.1-7.4 (2H, m), 7.54 (1H, dd, J=1.3 Hz, 7.7 Hz), 12.11 (1H, br s).

MS: 371 (M−1).

### EXAMPLE 21

4-({1-[(3,5-Dichloro-2-pyridinyl)methyl]-2,4-dimethyl-1H-benzimidazol-6-yl}oxy)butanoic acid (173 mg) was synthesized from ethyl 4-({1-[(3,5-dichloro-2-pyridinyl)methyl]-2,4-dimethyl-1H-benzimidazol-6-yl}oxy)butanoate (200 mg) in a manner similar to that described in Example 14.

NMR(DMSO-d₆,δ): 1.8-2.0 (2H, m), 2.2-2.5 (8H, m), 3.89 (2H, t, J=6.3 Hz), 5.58 (2H, br s), 6.57 (1H, d, J=1.7 Hz), 6.76 (1H, d, J=1.7 Hz), 8.31 (1H, d, J=2.1 Hz), 8.44 (1H, d, J=2.1 Hz), 12.14 (1H, br s).

MS: 408 (M+1).

### EXAMPLE 22

4-({1-[(2,6-Dichloro-3-pyridinyl)methyl]-2,4-dimethyl-1H-benzimidazol-6-yl}oxy)butanoic acid (197 mg) was synthesized from ethyl 4-({1-[(2,6-dichloro-3-pyridinyl)methyl]-2,4-dimethyl-1H-benzimidazol-6-yl}oxy)butanoate (200 mg) in a manner similar to that described in Example 14.

NMR(DMSO-d₆,δ): 1.8-2.0 (2H, m), 2.3-2.5 (8H, m), 3.90 (2H, t, J=6.3 Hz), 5.45 (2H, br s), 6.62 (1H, d, J=1.8 Hz), 6.80 (1H, d, J=8.1 Hz), 6.85 (1H, d, J=1.8 Hz), 7.45 (1H, d, J=8.1 Hz), 12.13 (1H, br s).

MS: 408 (M+1).

### EXAMPLE 23

4-{[1-(2,5-Dichlorobenzyl)-2,4-dimethyl-1H-benzimidazol-6-yl]oxy}butanoic acid (257 mg) was synthesized from ethyl 4-{[1-(2,5-dichlorobenzyl)-2,4-dimethyl-1H-benzimidazol-6-yl]oxy}butanoate (350 mg) in a manner similar to that described in Example 14.

NMR(DMSO-d₆,δ): 1.8-2.0 (2H, m), 2.36 (2H, t, J=7.2 Hz), 2.40 (3H, s), 2.47 (3H, s), 3.91 (2H, t, J=6.4 Hz), 5.47 (2H, br s), 6.41 (1H, d, J=2.4 Hz), 6.63 (1H, d, J=1.7 Hz), 6.83 (1H, d, J=1.7 Hz), 7.42 (1H, dd, J=2.4 Hz, 8.6 Hz), 7.60 (1H, d, J=8.6 Hz), 12.16 (1H, br s).

MS: 405 (M−1).

### EXAMPLE 24

4-[(1-{2-Chloro-4-[(ethoxycarbonyl)(methyl)amino]benzyl}-2,4-dimethyl-1H-benzimidazol-6-yl)oxy]butanoic acid (114 mg) was synthesized from ethyl 4-[(1-{2-chloro-4-[(ethoxycarbonyl)(methyl)amino]benzyl}-2,4-dimethyl-1H-benzimidazol-6-yl)oxy]butanoate (280 mg) in a manner similar to that described in Example 14.

NMR(DMSO-d₆,δ): 1.15 (3H, t, J=7.1 Hz), 1.8-2.0 (2H, m), 2.36 (2H, t, J=7.2 Hz), 2.40 (3H, s), 2.47 (3H, s), 3.19 (3H, s), 3.90 (2H, t, J=6.4 Hz), 4.07 (2H, q, J=7.1 Hz), 5.44 (2H, br s), 6.41 (1H, d, J=8.4 Hz), 6.62 (1H, br s), 6.81 (1H, br s), 7.18 (1H, dd, J=2.1 Hz, 8.4 Hz), 7.56 (1H, d, J=2.1 Hz), 12.11 (1H, br s).

MS: 472 (M−1).

### EXAMPLE 25

4-{[1-(2,3-Dichlorobenzyl)-2,4-dimethyl-1H-benzimidazol-6-yl]oxy}butanoic acid (104 mg) was synthesized from ethyl 4-{[1-(2,3-dichlorobenzyl)-2,4-dimethyl-1H-benzimidazol-6-yl]oxy}butanoate (140 mg) in a manner similar to that described in Example 14.

NMR(DMSO-d₆,δ): 1.7-2.0 (2H, m), 2.35 (2H, t, J=7.2 Hz), 2.38 (3H, s), 2.47 (3H, s), 3.89 (2H, t, J=6.4 Hz), 5.50 (2H, br s), 6.28 (1H, dd, J=1.1 Hz, 7.9 Hz), 6.62 (1H, d, J=1.8 Hz), 6.82 (1H, d, J=1.8 Hz), 7.25 (1H, t, J=7.9 Hz), 7.59 (1H, dd, J=1.1 Hz, 7.9 Hz), 12.12 (1H, br s).

MS: 405 (M−1).

### EXAMPLE 26

4-{[1-(3,4-Dichlorobenzyl)-2,4-dimethyl-1H-benzimidazol-6-yl]oxy}butanoic acid (211 mg) was synthesized from ethyl 4-{λ1-(3,4-dichlorobenzyl)-2,4-dimethyl-1H-benzimidazol-6-yl]oxy}butanoate (348 mg) in a manner similar to that described in Example 14.

NMR(DMSO-d₆,δ): 1.8-2.0 (2H, m), 2.37 (2H, t, J=7.2 Hz), 2.43 (3H, s), 2.45 (3H, s), 3.93 (2H, t, J=6.4 Hz), 5.43 (2H, br s), 6.61 (1H, d, J=1.8 Hz), 6.88 (1H, d, J=1.8 Hz), 6.97 (1H, dd, J=2.0 Hz, 8.3 Hz), 7.41 (1H, d, J=2.0 Hz), 7.58 (1H, d, J=8.3 Hz), 12.14 (1H, br s).

MS: 405 (M−1).

### EXAMPLE 27

4-({1-[(3-Chloro-1,1′-biphenyl-4-yl)methyl]-2,4-dimethyl-1H-benzimidazol-6-yl}oxy)butanoic acid (216 mg) was synthesized from ethyl 4-({1-[(3-chloro-1,1′-biphenyl-4-yl)methyl]-2,4-dimethyl-1H-benzimidazol-6-yl}oxy)butanoate (261 mg) in a manner similar to that described in Example 14.

NMR(DMSO-d₆,δ): 1.8-2.0 (2H, m), 2.2-2.5 (8H, m), 3.91 (2H, t, J=6.3 Hz), 5.51 (2H, br s), 6.48 (1H, d, J=8.1 Hz), 6.63 (1H, d, J=1.8 Hz), 6.84 (1H, d, J=1.8 Hz), 7.3-7.7 (6H, m), 7.83 (1H, d, J=1.7 Hz), 12.13 (1H, br s).

MS: 449 (M+1).

### EXAMPLE 28

4-{[1-(2-Chloro-4-methoxybenzyl)-2,4-dimethyl-1H-benzimidazol-6-yl]oxy}butanoic acid (49 mg) was synthesized from ethyl 4-{[1-(2-chloro-4-methoxybenzyl)-2,4-dimethyl-1H-benzimidazol-6-yl]oxy}butanoate (90 mg) in a manner similar to that described in Example 14.

NMR(DMSO-d₆,δ): 1.8-2.1 (2H, m), 2.2-2.5 (8H, m), 3.74 (3H, s), 3.90 (2H, t, J=6.3 Hz), 5.38 (2H, br s), 6.47 (1H, d, J=8.6 Hz), 6.61 (1H, br s), 6.78 (1H, br s), 6.83 (1H, dd, J=2.4 Hz, 8.6 Hz), 7.12 (1H, d, J=2.4 Hz), 12.12 (1H, br s).

MS: 401 (M−1).

### EXAMPLE 29

4-{[1-(2-Chloro-4-ethoxybenzyl)-2,4-dimethyl-1H-benzimidazol-6-yl]oxy}butanoic acid (132 mg) was synthesized from ethyl 4-{[1-(2-chloro-4-ethoxybenzyl)-2,4-dimethyl-1H-benzimidazol-6-yl]oxy}butanoate (152 mg) in a manner similar to that described in Example 14.

NMR(DMSO-d₆,δ): 1.29 (3H, t, J=6.9 Hz), 1.8-2.0 (2H, m), 2.36 (2H, t, J=7.3 Hz), 3.92 (2H, t, J=6.6 Hz), 4.01 (2H, q, J=6.9 Hz), 5.44 (2H, br s), 6.58 (1H, d, J=8.6 Hz), 6.71 (1H, br s), 6.8-6.9 (2H, m), 7.10 (1H, d, J=2.5 Hz), 12.14 (1H, br s).

MS: 415 (M−1).

### EXAMPLE 30

4-{[1-(2,4-Dichlorobenzyl)-2-methyl-1H-benzimidazol-6-yl]oxy}butanoic acid (83 mg) was synthesized from ethyl 4-{[1-(2,4-dichlorobenzyl)-2-methyl-1H-benzimidazol-6-yl]oxy}butanoate (98 mg) in a manner similar to that described in Example 14.

NMR(DMSO-d₆,δ): 1.8-2.0 (2H, m), 2.3-2.5 (5H, m), 3.93 (2H, t, J=6.3 Hz), 5.49 (2H, br s), 6.45 (1H, d, J=8.4 Hz), 6.80 (1H, dd, J=2.3 Hz, 8.7 Hz), 7.04 (1H, d, J=2.2 Hz), 7.33 (1H, dd, J=2.2 Hz, 8.3 Hz), 7.46 (1H, d, J=8.7 Hz), 7.73 (1H, d, J=2.2 Hz), 12.13 (1H, br s).

MS: 391 (M−1).

### EXAMPLE 31

4-{[1-(2-Chloro-4-ethoxybenzyl)-2-methyl-1H-benzimidazol-6-yl]oxy}butanoic acid (138 mg) was synthesized from ethyl 4-{[1-(2-chloro-4-ethoxybenzyl)-2-methyl-1H-benzimidazol-6-yl]oxy}butanoate (197 mg) in a manner similar to that described in Example 14.

NMR(DMSO-d₆,δ): 1.29 (3H, t, J=6.9 Hz), 1.8-2.0 (2H, m), 2.3-2.5 (5H, m), 3.8-4.1 (4H, m), 5.40 (2H, br s), 6.47 (1H, d, J=8.7 Hz), 6.7-6.9 (2H, m), 6.98 (1H, d, J=2.2 Hz), 7.10 (1H, d, J=2.5 Hz), 7.42 (1H, d, J=8.7 Hz), 12.11 (1H, br s).

MS: 401 (M−1).

### EXAMPLE 32

To a solution of ethyl 4-[(1-{2-chloro-4-[(ethoxycarbonyl)(methyl)amino]benzyl}-2-methyl-1H-benzimidazol-6-yl)oxy]butanoate (195 mg) in dioxane (2 ml) was added 1N-NaOH (0.8 ml) at ambient temperature. The mixture was heated at 80° C. for 1 hour. After cooling, the pH of the reaction mixture was adjusted to around 4 with 1N-HCl. The precipitates were collected by filtration and washed with water to give crude 4-[(1-{2-chloro-4-[(ethoxycarbonyl)(methyl)amino]benzyl}-2-methyl-1H-benzimidazol-6-yl)oxy]butanoic acid (144 mg). The crude crystals were chromatographed (preparative TLC, CH₂Cl₂/MeOH=10/1) and triturated with EtOAc (1 ml)—n-hexane (1 ml) to give pure product (83 mg) as white crystals.

NMR(DMSO-d₆,δ): 1.15 (3H, t, J=7.0 Hz), 1.8-2.0 (2H, m), 2.3-2.5 (5H, m), 3.19 (3H, s), 3.93 (2H, t, J=6.2 Hz), 4.07 (2H, q, J=7.0 Hz), 5.47 (2H, br s), 6.43 (1H, d, J=8.3 Hz), 6.78 (1H, dd, J=2.1 Hz, 8.7 Hz), 7.01 (1H, br s), 7.16 (1H, br s), 7.20 (1H, br s), 7.44 (1H, d, J=8.7 Hz), 7.56 (1H, d, J=2.1 Hz), 12.15 (1H, br s).

MS: 460 (M+1).

### EXAMPLE 33

4-[(1-{[3-Chloro-5-(trifluoromethyl)-2-pyridinyl]methyl}-2-methyl-1H-benzimidazol-6-yl)oxy]butanoic acid (121 mg) was synthesized from ethyl 4-[(1-{[3-chloro-5-(trifluoromethyl)-2-pyridinyl]methyl}-2-methyl-1H-benzimidazol-6-yl)oxy]butanoate (183 mg) in a manner similar to that described in Example 14.

NMR(DMSO-d₆,δ): 1.8-2.0 (2H, m), 2.2-2.5 (5H, m), 3.91 (2H, t, J=6.3 Hz), 5.73 (2H, br s), 6.74 (1H, dd, J=2.2 Hz, 8.7 Hz), 7.00 (1H, d, J=2.2 Hz), 7.39 (1H, d, J=8.7 Hz), 8.56 (1H, d, J=1.1 Hz), 8.77 (1H, d, J=1.1 Hz), 12.15 (1H, br s).

MS: 426 (M−1).

### EXAMPLE 34

To a suspension of ethyl 4-{[2-ethoxy-4-methyl-1-({4-methyl-2-[4-(trifluoromethyl)phenyl]-1,3-thiazol-5-yl}methyl)-1H-benzimidazol-6-yl]oxy}butanoate (220 mg) in EtOH (2.2 ml) was added 1N-NaOH (0.78 ml) at ambient temperature. The mixture was heated at 80° C. for 1 hour. After cooling, the pH of the reaction mixture was adjusted to around 4 with 1N-HCl. The precipitates were collected by filtration and the crude crystals were purified by chromatography (preparative TLC, EtOAc) to give pure 4-{[2-ethoxy-4-methyl-1-({4-methyl-2-[4-(trifluoromethyl)phenyl]-1,3-thiazol-5-yl}methyl)-1H-benzimidazol-6-yl]oxy}butanoic acid (96 mg) as white crystals.

NMR(DMSO-d₆,δ): 1.41 (3H, t, J=7.0 Hz), 1.8-2.1 (2H, m), 2.37 (3H, s), 2.41 (2H, t, J=7.4 Hz), 2.57 (3H, s), 3.99 (2H, t, J=6.3 Hz), 4.52 (2H, q, J=7.0 Hz), 5.42 (2H, br s), 6.56 (1H, d, J=2.0 Hz), 6.96 (1H, d, J=2.0 Hz), 7.79 (2H, d, J=8.2 Hz), 8.05 (2H, d, J=8.2 Hz), 12.11 (1H, br s).

MS: 532 (M−1).

### EXAMPLE 35

4-{[1-(2,4-Dichlorobenzyl)-2-ethyl-4-methyl-1H-benzimidazol-6-yl]oxy}butanoic acid (108 mg) was synthesized from ethyl 4-{[1-(2,4-dichlorobenzyl)-2-ethyl-4-methyl-1H-benzimidazol-6-yl]oxy}butanoate (150 mg) in a manner similar to that described in Example 14.

NMR(DMSO-d₆,δ): 1.21 (3H, t, J=7.4 Hz), 1.8-2.0 (2H, m), 2.36 (2H, t, J=7.4 Hz), 2.48 (3H, s), 2.70 (2H, q, J=7.4 Hz), 3.90 (2H, t, J=6.4 Hz), 5.45 (2H, br s), 6.37 (1H, d, J=8.4 Hz), 6.63 (1H, d, J=1.8 Hz), 6.81 (1H, d, J=1.8 Hz), 7.31 (1H, dd, J=2.1 Hz, 8.4 Hz), 7.72 (1H, d, J=2.1 Hz), 12.11 (1H, br s).

MS: 419 (M−1).

### EXAMPLE 36

4-{[1-(2,4-Dichlorobenzyl)-4-methyl-2-(trifluoromethyl)-1H-benzimidazol-6-yl]oxy}butanoic acid (134 mg) was synthesized from ethyl 4-{[1-(2,4-dichlorobenzyl)-4-methyl-2-(trifluoromethyl)-1H-benzimidazol-6-yl]oxy}butanoate (150 mg) in a manner similar to that described in Example 14.

NMR(DMSO-d₆,δ): 1.8-2.1 (2H, m), 2.37 (2H, t, J=7.4 Hz), 2.55 (3H, s), 3.96 (2H, t, J=6.3 Hz), 5.68 (2H, br s), 6.37 (1H, d, J=8.4 Hz), 6.87 (1H, d, J=1.6 Hz), 7.10 (1H, d, J=1.6 Hz), 7.32 (1H, dd, J=2.1 Hz, 8.4 Hz), 7.75 (1H, d, J=2.1 Hz), 12.15 (1H, br s).

MS: 459 (M−1).

### EXAMPLE 37

To a solution of ethyl 4-{[1-(2,4-dichlorobenzyl)-2,4-dimethyl-1H-benzimidazol-6-yl]oxy}-2,2-dimethylbutanoate (430 mg: crude) in EtOH (4.3 ml) was added 2N-NaOH (1.86 ml) at ambient temperature. The mixture was heated at 90° C. for 4 hours. After cooling, the pH of the reaction mixture was adjusted to around 3.5 with 1N-HCl. The precipitates were collected by filtration and washed with water and MeOH (small portion) to give 4-{[1-(2,4-dichlorobenzyl)-2,4-dimethyl-1H-benzimidazol-6-yl]oxy}-2,2-dimethylbutanoic acid (233 mg) as pale yellow crystals.

NMR(DMSO-d₆,δ): 1.14 (6H, s), 1.90 (2H, t, J=6.9 Hz), 2.37 (3H, s), 2.46 (3H, s), 3.90 (2H, t, J=6.9 Hz), 5.45 (2H, br s), 6.43 (1H, d, J=8.4 Hz), 6.59 (1H, d, J=1.7 Hz), 6.79 (1H, d, J=1.7 Hz), 7.33 (1H, dd, J=2.2 Hz, 8.4 Hz), 7.72 (1H, d, J=2.2 Hz), 12.28 (1H, br s).

MS: 433 (M−1).

### EXAMPLE 38

4-({1-[2-Chloro-4-(pentyloxy)benzyl]-2,4-dimethyl-1H-benzimidazol-6-yl}oxy)-2,2-dimethylbutanoic acid (326 mg) was synthesized from ethyl 4-({1-[2-chloro-4-(pentyloxy)benzyl]-2,4-dimethyl-1H-benzimidazol-6-yl}oxy)-2,2-dimethylbutanoate (457 mg) in a manner similar to that described in Example 37.

NMR(DMSO-d₆,δ): 0.8-1.0 (3H, m), 1.14 (6H, s), 1.2-1.5 (4H, m), 1.5-1.8 (2H, m), 1.91 (2H, t, J=6.6 Hz), 2.39 (3H, s), 2.45 (3H, s), 3.8-4.0 (4H, m), 5.37 (2H, br s), 6.47 (1H, d, J=8.6 Hz), 6.58 (1H, br s), 6.75 (1H, br s), 6.81 (1H, dd, J=2.5 Hz, 8.6 Hz), 7.09 (1H, d, J=2.5 Hz), 12.19 (1H, br s).

MS: 485 (M−1).

### EXAMPLE 39

To a suspension of ethyl 4-{[1-(2,4-dichlorobenzyl)-2-ethoxy-4-methyl-1H-benzimidazol-6-yl]oxy}butanoate (125 mg) in ethyl alcohol (EtOH) (1.2 ml) was added sodium hydroxide, 1N solution in water (1N NaOH) (0.54 mL) at room temperature. The mixture was heated at 70° C. for 0.5 hour. After cooling, the reaction mixture was neutralized with hydrochloric acid, 1N solution in water (1N-HCl) (pH 7). The resulting precipitates were collected by filtration and washed with water to give 4-{[1-(2,4-dichlorobenzyl)-2-ethoxy-4-methyl-1H-benzimidazol-6-yl]oxy}butanoic acid (87 mg) as white crystals.

NMR(200 MHz,DMSO-d₆,δ): 1.30 (3H, t, J=7.0 Hz), 1.88 (2H, tt, J=6.5 Hz, 7.0 Hz), 2.21 (2H, t, J=7.0 Hz), 2.48 (3H, s), 3.85 (1H, br), 3.86 (2H, t, J=6.5 Hz), 4.49 (2H, q, J=7.0 Hz), 5.22 (2H, s), 6.54 (1H, d, J=2.1 Hz), 6.72 (1H, d, J=2.1 Hz), 6.74 (1H, d, J=8.4 Hz), 7.36 (1H, dd, J=2.1 Hz, 8.4 Hz), 7.67 (1H, d, J=2.1 Hz).

MS: 435 (M−1).

### EXAMPLE 40

To a solution of ethyl 4-{[1-(2-chloro-4-ethoxybenzyl)-2-ethoxy-4-methyl-1H-benzimidazol-6-yl]oxy}butanoate (150 mg) in EtOH (1.5 ml) was added 1N NaOH (0.63 mL) at room temperature. The mixture was heated at 70° C. for 1 hour. After cooling, the reaction mixture was neutralized with 1N-HCl (pH 7). The resulting precipitates were collected by filtration and washed with water to give 4-{[1-(2-chloro-4-ethoxybenzyl)-2-ethoxy-4-methyl-1H-benzimidazol-6-yl]oxy}butanoic acid (81 mg) as white crystals.

NMR(200 MHz,DMSO-d₆,δ): 1.29 (3H, t, J=7.0 Hz), 1.33 (3H, t, J=7.0 Hz), 1.81-1.95 (2H, m), 2.34 (2H, t, J=7.2 Hz), 2.39 (3H, s), 3.30 (1H, br), 3.88 (2H, t, J=6.4 Hz), 4.02 (2H, t, J=7.0 Hz), 4.48 (2H, q, J=7.0 Hz), 5.15 (2H, s), 6.53 (1H, d, J=1.9 Hz), 6.66 (1H, d, J=2.1 Hz), 6.82-6.88 (2H, m), 7.05 (1H, d, J=1.9 Hz).

MS: 445 (M−1).

### EXAMPLE 41

To a suspension of ethyl 4-[(1-{[3-chloro-5-(trifluoromethyl)-2-pyridinyl]methyl}-2-ethoxy-4-methyl-1H-benzimidazol-6-yl)oxy]butanoate (168 mg) in EtOH (1.7 ml) was added 1N NaOH (0.69 mL) at room temperature. The mixture was heated at 70° C. for 20 minutes. After cooling to room temperature, the reaction mixture was neutralized with 1N-HCl (pH 7). The resulting precipitates were collected by filtration and washed with water to give 4-[(1-{[3-chloro-5-(trifluoromethyl)-2-pyridinyl]methyl}-2-ethoxy-4-methyl-1H-benzimidazol-6-yl)oxy]butanoic acid (129 mg) as white crystals.

NMR(200 MHz,DMSO-d₆,δ): 1.25 (3H, t, J=7.0 Hz), 1.85-1.90 (2H, m), 2.31 (2H, t, J=7.3 Hz), 2.39 (3H, s), 3.4 (1H, br), 3.86 (2H, t, J=6.4 Hz), 4.43 (2H, q, J=7.0 Hz), 5.48 (2H, s), 6.52 (1H, d, J=2.2 Hz), 6.74 (1H, d, J=2.2 Hz), 8.52 (1H, d, J=1.0 Hz), 8.80 (1H, d, J=1.0 Hz).

MS: 470 (M−1).

### EXAMPLE 42

To a suspension of ethyl 4-{[1-(2,4-dichlorobenzyl)-2-ethoxy-1H-benzimidazol-6-yl]oxy}butanoate (116 mg) in EtOH (1 ml) was added 1N NaOH (0.51 mL) at room temperature. The mixture was heated at 70° C. for 20 minutes. After cooling to room temperature, the reaction mixture was neutralized with 1N-HCl (pH 7). The resulting precipitates were collected by filtration and washed with water to give 4-{[1-(2,4-dichlorobenzyl)-2-ethoxy-1H-benzimidazol-6-yl]oxy}butanoic acid (106 mg) as white crystals.

NMR(200 MHz,DMSO-d₆,δ): 1.30 (3H, t, J=7.0 Hz), 1.86-1.94 (2H, m), 2.36 (2H, t, J=7.3 Hz), 3.91 (2H, t, J=6.4 Hz), 4.46 (2H, q, J=7.0 Hz), 5.26 (2H, s), 6.71 (1H, dd, J=2.4 Hz, 8.4 Hz), 6.80 (1H, d, J=8.4 Hz), 6.94 (1H, d, J=2.4 Hz), 7.30 (1H, d, J=8.4 Hz), 7.37 (1H, dd, J=2.1 Hz, 8.4 Hz), 7.69 (1H, d, J=2.1 Hz), 12.1 (1H, br).

### EXAMPLE 43

To a suspension of ethyl 4-{[1-(2,4-dichlorobenzyl)-4-methyl-2-(methylamino)-1H-benzimidazol-6-yl]oxy}butanoate (130 mg) in EtOH (1.3 ml) was added 1N NaOH (0.58 mL) at room temperature. The mixture was heated at 70° C. for 30 minutes. After cooling to room temperature, the reaction mixture was neutralized with 1N-HCl (pH 7). The resulting precipitates were collected by filtration and washed with water to give 4-{[1-(2,4-dichlorobenzyl)-4-methyl-2-(methylamino)-1H-benzimidazol-6-yl]oxy}butanoic acid (119 mg) as pale brown crystals.

NMR(200 MHz,DMSO-d₆,δ): 1.78-1.92 (2H, m), 2.33 (2H, t, J=7.2 Hz), 2.39 (3H, s), 2.88 (3H, d, J=4.0 Hz), 3.83 (2H, t, J=6.3 Hz), 5.32 (2H, s), 6.39 (1H, d, J=8.4 Hz), 6.47 (1H, s), 6.52 (1H, s), 6.76 (1H, br), 7.32 (1H, dd, J=2.1 Hz, 8.4 Hz), 7.70 (1H, d, J=2.1 Hz), 12.1 (1H, br).

MS: 422 (M+1).

### EXAMPLE 44

To a suspension of ethyl 4-{[1-(2,4-dichlorobenzyl)-2-(ethylthio)-4-methyl-1H-benzimidazol-6-yl]oxy}butanoate (261 mg) in EtOH (2.6 ml) was added 1N NaOH (1.0 mL) at room temperature. The mixture was heated at 70° C. for 30 minutes. After cooling to room temperature, the reaction mixture was neutralized with 1N-HCl (pH 7). The resulting precipitates were collected by filtration and washed with water to give 4-{[1-(2,4-dichlorobenzyl)-2-(ethylthio)-4-methyl-1H-benzimidazol-6-yl]oxy}butanoic acid (229 mg) as white crystals.

NMR(200 MHz,DMSO-d₆,δ): 1.30(3H, t, J=7.3 Hz), 1.87-1.94 (2H, m), 2.36 (2H, t, J=7.3 Hz), 2.49 (3H, s), 3.22 (2H, q, J=7.3 Hz), 3.91 (2H, t, J=6.4 Hz), 5.41 (2H, s), 6.44 (1H, d, J=8.4 Hz), 6.65 (1H, d, J=2.2 Hz), 6.89 (1H, d, J=2.1 Hz), 7.33 (1H, dd, J=2.1 Hz, 8.4 Hz), 7.72 (1H, d, J=2.2 Hz), 12.1 (1H, s).

MS: 453 (M+1).

### EXAMPLE 45

2-({1-[(3-Chloro-1,1′-biphenyl-4-yl)methyl]-2,4-dimethyl-1H-benzimidazol-6-yl}oxy)-2-methylpropanoic acid (106 mg) was synthesized from ethyl 2-({1-[(3-chloro-1,1′-biphenyl-4-yl)methyl]-2,4-dimethyl-1H-benzimidazol-6-yl}oxy)-2-methylpropanoate (145 mg) in a manner similar to that described in Example 51.

NMR(DMSO-d₆,δ): 1.39 (6H, s), 2.46 (3H, s), 2.48 (3H, s), 5.45 (2H, s), 6.55-6.70 (3H, m), 7.32-7.73 (6H, m), 7.82 (1H, d, J=1.0 Hz).

MS: 449 (M+).

### EXAMPLE 46

2-({1-[2-Chloro-4-(pentyloxy)benzyl]-2,4-dimethyl-1H-benzimidazol-6-yl}oxy)-2-methylpropanoic acid (106 mg) was synthesized from ethyl 2-({1-[2-chloro-4-(pentyloxy)benzyl]-2,4-dimethyl-1H-benzimidazol-6-yl}oxy)-2-methylpropanoate (136 mg) in a manner similar to that described in Example 51.

NMR(DMSO-d₆,δ): 0.87 (3H, t, J=6.0 Hz), 1.20-1.40 (4H, m), 1.41 (6H, s), 1.60-1.75 (2H, m), 2.44 (3H, s), 2.45 (3H, s), 3.95 (2H, t, J=6.0 Hz), 5.31 (2H, s), 6.50-6.63 (3H, m), 6.83 (1H, dd, J=8.0 Hz, 2.0 Hz), 7.10 (1H, d, J=2.0 Hz).

MS: 459 (M+).

### EXAMPLE 47

4-({1-[(3-Chloro-5-ethoxy-2-pyridinyl)methyl]-2,4-dimethyl-1H-benzimidazol-6-yl}oxy)butanoic acid (121 mg) was synthesized from ethyl 4-({1-[(3-chloro-5-ethoxy-2-pyridinyl)methyl]-2,4-dimethyl-1H-benzimidazol-6-yl}oxy)butanoate (183 mg) in a manner similar to that described in Example 51.

NMR(DMSO-d₆,δ): 1.30 (3H, t, J=6.0 Hz), 1.78-1.97 (2H, m), 2.30 (2H, d, J=6.0 Hz), 2.43 (6H, s), 3.88 (2H, t, J=6.0 Hz), 4.10 (2H, q, J=6.0 Hz), 5.47 (2H, s), 6.55 (1H, d, J=1.5 Hz), 6.72 (1H, d, J=1.5 Hz), 7.61 (1H, d, J=1.0 Hz), 8.11 (1H, d, J=1.0 Hz).

MS: 418 (M+).

### EXAMPLE 48

4-({1-[2-Chloro-4-(1,3-oxazol-2-yl)benzyl]-2,4-dimethyl-1H-benzimidazol-6-yl}oxy)butanoic acid (123 mg) was synthesized from ethyl 4-({1-[2-chloro-4-(1,3-oxazol-2-yl)benzyl]-2,4-dimethyl-1H-benzimidazol-6-yl}oxy)butanoate (149 mg) in a manner similar to that described in Example 51.

NMR(CDCl₃-CD₃OD,δ): 1.98-2.12 (2H, m), 2.48 (2H, t, J=6.0 Hz), 2.52 (3H, s), 2.60 (3H, s), 3.98 (2H, t, J=6.0 Hz), 5.40 (2H, s), 6.51 (1H, d, J=2.0 Hz), 6.57 (1H, d, J=8.0 Hz), 6.70 (1H, d, J=2.0 Hz), 7.40 (1H, s), 7.75-7.82 (2H, s), 8.15 (1H, d, J=1.0 Hz).

MS: 440 (M+).

### EXAMPLE 49

4-({1-[(2-Chloro-6-phenyl-3-pyridinyl)methyl]-2,4-dimethyl-1H-benzimidazol-6-yl}oxy)butanoic acid (295 mg) was synthesized from ethyl 4-({1-[(2-chloro-6-phenyl-3-pyridinyl)methyl]-2,4-dimethyl-1H-benzimidazol-6-yl}oxy)butanoate (356 mg) in a manner similar to that described in Example 51.

NMR(DMSO-d₆,δ): 1.80-1.99 (2H, m), 2.38 (2H, t, J=6.0 Hz), 2.42 (3H, s), 2.50 (3H, s), 3.90 (2H, t, J=6.0 Hz), 5.51 (2H, s), 6.63 (1H, d, J=1.5 Hz), 6.79-6.92 (2H, m), 7.40-7.58 (3H, m), 7.89 (1H, d, J=8.0 Hz), 7.96-8.08 (2H, m), 12.11 (1H, br s).

MS: 450 (M+).

### EXAMPLE 50

4-({1-[(3-Methoxy-1,1′-biphenyl-4-yl)methyl]-2,4-dimethyl-1H-benzimidazol-6-yl}oxy)butanoic acid (144 mg) was synthesized from ethyl 4-({1-[(3-methoxy-1,1′-biphenyl-4-yl)methyl]-2,4-dimethyl-1H-benzimidazol-6-yl}oxy)butanoate (175 mg) in a manner similar to that described in Example 51.

NMR(DMSO-d₆,δ): 1.80-2.00 (2H, m), 2.38 (2H, t, J=6.0 Hz), 2.46 (3H, s), 2.47 (3H, s), 3.93 (2H, t, J=6.0 Hz), 3.96 (3H, s), 5.33 (2H, s), 6.60 (1H, d, J=1.5 Hz), 6.65 (1H, d, J=8.0 Hz), 6.80 (1H, d, J=1.5 Hz), 7.12 (1H, dd, J=8.0 Hz, 1.5 Hz), 7.26-7.50 (4H, m), 7.66 (2H, d, J=8.0 Hz), 12.13 (1H, br s).

MS: 445 (M+).

### EXAMPLE 51

A mixture of ethyl 4-({1-[2-chloro-4-(pentyloxy)benzyl]-2-ethoxy-4-methyl-1H-benzimidazol-6-yl}oxy)butanoate (202 mg), 1N sodium hydroxide (NaOH, 0.8 mL) and ethanol (0.8 mL) was stirred at 80° C. for 2 hours. The reaction mixture was neutralized with 1N hydrochloric acid (HCl) to pH 4 under ice-water cooling. The precipitate was collected by vacuum filtration and washed with water to give 4-({1-[2-chloro-4-(pentyloxy)benzyl]-2-ethoxy-4-methyl-1H-benzimidazol-6-yl}oxy)butanoic acid (165 mg) as a white powder.

NMR(DMSO-d₆,δ): 0.87 (3H, t, J=6.0 Hz), 1.15-1.46 (4H, m), 1.56-1.97 (4H, m), 2.30 (2H, t, J=6.0 Hz), 2.40 (3H, s), 3.87 (2H, t, J=6.0 Hz), 3.93 (2H, t, J=6.0 Hz), 4.48 (2H, q, J=6.0 Hz), 5.15 (2H, s), 6.53 (1H, d, J=1.5 Hz), 6.66 (1H, d, J=1.5 Hz), 6.76-6.90 (2H, m), 7.05 (1H, d, J=1.5 Hz).

MS: 489 (M+).

### EXAMPLE 52

4-({1-[2-Chloro-4-(dimethylamino)benzyl]-2,4-dimethyl-1H-benzimidazol-6-yl}oxy)butanoic acid (80 mg) was synthesized from ethyl 4-({1-[2-chloro-4-(dimethylamino)benzyl]-2,4-dimethyl-1H-benzimidazol-6-yl}oxy)butanoate (165 mg) in a manner similar to that described in Example 67.

NMR(DMSO-d₆,δ): 1.8-2.0 (2H, m), 2.35 (2H, t, J=7 Hz), 2.40 (3H, s), 2.44 (3H, s), 2.85 (6H, s), 3.89 (2H, t, J=6 Hz), 5.30 (2H, s), 6.44 (1H, d, J=8 Hz), 6.54 (1H, d, J=2 Hz), 6.58 (1H, br s), 6.7-6.8 (2H, m), 11.8-12.5 (1H, br m).

MS: 414 (M−H).

### EXAMPLE 53

3-[({1-[2-Chloro-4-(pentyloxy)benzyl]-2,4-dimethyl-1H-benzimidazol-6-yl}oxy)methyl]benzoic acid (43 mg) was synthesized from methyl 3-[({1-[2-chloro-4-(pentyloxy)benzyl]-2,4-dimethyl-1H-benzimidazol-6-yl}oxy)methyl]benzoate (47 mg) in a manner similar to that described in Example 14.

NMR(DMSO-d₆,δ): 0.8-1.0 (3H, m), 1.2-1.5 (4H, m), 1.5-1.8 (2H, m), 2.40 (3H, s), 2.47 (3H, s), 3.94 (2H, t, J=6.4 Hz), 5.11 (2H, br s), 5.36 (2H, br s), 6.43 (1H, d, J=8.6 Hz), 6.72 (1H, d, J=2.0 Hz), 6.79 (1H, dd, J=2.5 Hz, 8.6 Hz), 6.88 (1H, d, J=2.0 Hz), 7.08 (1H, d, J=2.5 Hz), 7.48 (1H, t, J=7.7 Hz), 7.64 (1H, d, J=7.7 Hz), 7.87 (1H, d, J=7.7 Hz), 8.00 (1H, br s), 13.02 (1H, br s).

MS: 507 (M+1).

### EXAMPLE 54

To a mixture of 1-[2-chloro-4-(pentyloxy)benzyl]-2-methyl-1H-benzimidazol-6-ol (0.93 g) and N,N-dimethylformamide (2.5 mL) was added sodium hydride (60% dispersion in mineral oil; 11 mg). The mixture was stirred at ambient temperature for 5 minutes. To the mixture was added 2-benzofuran-1(3H)-one (107 mg) and stirring was continued overnight at 140° C. After cooling, the pH of the mixture was adjusted to 4 with 1 N hydrochloric acid. The mixture was partitioned between ethyl acetate and water. The organic layer was separated, washed with water (3 times) and brine, dried over anhydrous magnesium sulfate, and concentrated in vacuo. The residue was purified by column chromatography on silica gel (gradient elution; dichloromethane to 5% methanol in dichloromethane) to give 2-[({1-[2-chloro-4-(pentyloxy)benzyl]-2-methyl-1H-benzimidazol-6-yl}oxy)methyl]benzoic acid (24 mg).

NMR(DMSO-d₆,δ): 0.87 (3H, t, J=7.0 Hz), 1.2-1.4 (6H, m), 1.6-1.8 (2H, m), 2.42 (3H, s), 3.93 (2H, t, J=6.4 Hz), 5.37 (2H, s), 5.40 (2H, s), 6.55 (1H, d, J=8.6 Hz), 6.7-6.9 (1H, m), 6.98 (1H, d, J=2.1 Hz), 7.06 (1H, d, J=2.1 Hz), 7.3-7.7 (4H, m), 7.89 (1H, d, J=6.7 Hz).

MASS (API-ES, Nega): 491.2.

### EXAMPLE 55

2-[({1-[(3-Chloro-1,1′-biphenyl-4-yl)methyl]-2-ethoxy-1H-benzimidazol-6-yl}oxy)methyl]benzoic acid (84 mg) was synthesized from methyl 2-[({1-[(3-chloro-1,1′-biphenyl-4-yl)methyl]-2-ethoxy-1H-benzimidazol-6-yl}oxy)methyl]benzoate (120 mg) in a manner similar to that described in Example 56.

NMR(DMSO-d₆,δ): 1.35 (3H, t, J=7 Hz), 4.50 (2H, q, J=7 Hz), 5.30 (2H, s), 5.39 (2H, s), 6.77 (1H, dd, J=2 Hz, 8 Hz), 6.94 (1H, d, J=8 Hz), 6.98 (1H, d, J=2 Hz), 7.3-8.0 (12H, m), 12.7-13.4 (1H, br m).

MS: 511 (M−H).

### EXAMPLE 56

A mixture of methyl 2-[({1-[(3-chloro-1,1′-biphenyl-4-yl)methyl]-2,4-dimethyl-1H-benzimidazol-6-yl}oxy)methyl]benzoate (0.14 g), 1 N sodium hydroxide (1 mL) and ethanol (12 mL) was stirred at 80° C. for an hour. After cooling, the mixture was evaporated in vacuo and the residue was partitioned between ethyl acetate/tetrahydrofuran and brine. The organic layer was separated, dried over anhydrous sodium sulfate, and concentrated in vacuo. The resulting solid was suspended in ethyl acetate (10 mL), and the suspension was stirred at ambient temperature for half an hour. The precipitate was collected by filtration and dried in vacuo to give 2-[({1-[(3-chloro-1,1′-biphenyl-4-yl)methyl]-2,4-dimethyl-1H-benzimidazol-6-yl}oxy)methyl]benzoic acid (101 mg).

NMR(DMSO-d₆,δ): 2.43 (3H, s), 2.48 (3H, s), 5.42 (2H, s), 5.46 (2H, s), 6.55 (1H, d, J=8 Hz), 6.70 (1H, d, J=1 Hz), 6.87 (1H, d, J=2 Hz), 7.3-7.9 (11H, m).

MS: 495 (M−H).

### EXAMPLE 57

2-({[1-(2-Chloro-4-ethoxybenzyl)-2,4-dimethyl-1H-benzimidazol-6-yl]oxy)methyl)benzoic acid (0.38 g) was synthesized from methyl 2-({[1-(2-chloro-4-ethoxybenzyl)-2,4-dimethyl-1H-benzimidazol-6-yl]oxy}methyl)benzoate (510 mg) in a manner similar to that described in Example 56.

NMR(DMSO-d₆,δ): 1.29 (3H, t, J=7 Hz), 2.42 (3H, s), 2.47 (3H, s),4.03 (2H, q, J=7 Hz), 5.34 (2H, s), 5.37 (2H, s), 6.51 (1H, d, J=9 Hz), 6.69 (1H, d, J=1 Hz), 6.7-6.9 (2H, m), 7.04 (1H, d, J=2 Hz), 7.3-8.0 (4H, m), 12.7-13.4 (1H, br m).

MS: 463 (M−H).

### EXAMPLE 58

2-{[(1-{[3-Chloro-5-(trifluoromethyl)-2-pyridinyl]methyl}-2,4-dimethyl-1H-benzimidazol-6-yl)oxy]methyl}benzoic acid (98 mg) was synthesized from methyl 2-{[(1-{[3-chloro-5-(trifluoromethyl)-2-pyridinyl]methyl}-2,4-dimethyl-1H-benzimidazol-6-yl)oxy]methyl}benzoate (260 mg) in a manner similar to that described in Example 56.

NMR(DMSO-d₆,δ): 2.41 (3H, s), 2.45 (3H, s), 5.37 (2H, s), 5.67 (2H, s), 6.65 (1H, d, J=2 Hz), 6.78 (1H, d, J=2 Hz), 7.3-7.6 (3H, m), 7.87 (1H, dd, J=1 Hz, 8 Hz), 8.49 (1H, d, J=1 Hz), 8.74 (1H, s), 12.5-13.5 (1H, br m).

MS: 488 (M−H).

### EXAMPLE 59

2-{[(1-{2-Chloro-4-[(ethoxycarbonyl)(methyl)amino]benzyl}-2,4-dimethyl-1H-benzimidazol-6-yl)oxy]methyl}benzoic acid (98 mg) was synthesized from methyl 2-{[(1-{2-chloro-4-[(ethoxycarbonyl)(methyl)amino]benzyl}-2,4-dimethyl-1H-benzimidazol-6-yl)oxy]methyl}benzoate (200 mg) in a manner similar to that described in Example 56.

NMR(DMSO-d₆,δ): 1.14 (3H, t, J=7 Hz), 2.43 (3H, s), 2.47 (3H, s), 3.18 (3H, s), 4.06 (2H, q, J=Hz), 5.37 (2H, s), 5.42 (2H, s), 6.48 (1H, d, J=8 Hz), 6.69 (1H, d, J=1 Hz), 6.83 (1H, d, J=2 Hz), 7.16 (1H, dd, J=2 Hz, 8 Hz), 7.3-7.7 (4H, m), 7.8-8.0 (1H, m) 12.7-13.4 (1H, br m).

MS: 520 (M−H).

### EXAMPLE 60

2-[({1-[(3,5-Dichloro-2-pyridinyl)methyl]-2,4-dimethyl-1H-benzimidazol-6-yl}oxy)methyl]benzoic acid (0.37 g) was synthesized from methyl 2-[({1-[(3,5-dichloro-2-pyridinyl)methyl]-2,4-dimethyl-1H-benzimidazol-6-yl}oxy)methyl]benzoate (450 mg) in a manner similar to that described in Example 56.

NMR(DMSO-d₆,δ): 2.42 (3H, s), 2.44 (3H, s), 5.39 (2H, s), 5.53 (2H, s), 6.64 (1H, s), 6.74 (1H, s), 7.3-8.0 (4H, m), 8.23 (1H, d, J=2 Hz), 8.40 (1H, d, J=2 Hz).

MS: 455 (M−H).

### EXAMPLE 61

2-({[1-(2,4-Dichlorobenzyl)-2,4-dimethyl-1H-benzimidazol-6-yl]oxy}methyl)benzoic acid (0.49 g) was synthesized from methyl 2-({[1-(2,4-dichlorobenzyl)-2,4-dimethyl-1H-benzimidazol-6-yl]oxy}methyl)benzoate (570 mg) in a manner similar to that described in Example 56.

NMR(DMSO-d₆,δ): 2.41 (3H, s), 2.47 (3H, s), 5.37 (2H, s), 5.42 (2H, s), 6.47 (1H, d, J=8 Hz), 6.69 (1H, d, J=1 Hz), 6.79 (1H, d, J=2 Hz), 7.29 (1H, dd, J=2 Hz, 8 Hz), 7.4-7.6 (3H, m), 7.67 (1H, d, J=2 Hz), 7.8-8.0 (1H, m), 12.7-13.4 (1H, br m).

MS: 453 (M−H).

### EXAMPLE 62

2-[({1-[(2,6-Dichloro-3-pyridinyl)methyl]-2,4-dimethyl-1H-benzimidazol-6-yl}oxy)methyl]benzoic acid (0.41 g) was synthesized from methyl 2-[({1-[(2,6-dichloro-3-pyridinyl)methyl]-2,4-dimethyl-1H-benzimidazol-6-yl}oxy)methyl]benzoate (510 mg) in a manner similar to that described in Example 56.

NMR(DMSO-d₆,δ): 2.43 (3H, s), 2.48 (3H, s), 5.38 (2H, s), 5.43 (2H, s), 6.70 (1H, d, J=1 Hz), 6.7-7.0 (2H, m), 7.41 (1H, d, J=8 Hz), 7.4-8.0 (4H, m), 12.5-13.4 (1H, br m).

MS: 458, 456 (M+H).

### EXAMPLE 63

2-[({1-[(2-Chloro-6-phenyl-3-pyridinyl)methyl]-2,4-dimethyl-1H-benzimidazol-6-yl}oxy)methyl]benzoic acid (146 mg) was synthesized from methyl 2-[({1-[(2-chloro-6-phenyl-3-pyridinyl)methyl]-2,4-dimethyl-1H-benzimidazol-6-yl}oxy)methyl]benzoate (180 mg) in a manner similar to that described in Example 56.

NMR(DMSO-d₆,δ): 2.50 (6H, s), 5.39 (2H, s), 5.52 (2H, s), 6.75 (1H, s), 6.93 (1H, s), 6.98 (1H, d, J=8 Hz), 7.3-8.1 (9H, m), 12.7-13.4 (1H, br m).

MS: 498, 496 (M−H).

### EXAMPLE 64

2-[({1-[(3-Methoxy-1,1′-biphenyl-4-yl)methyl]-2,4-dimethyl-1H-benzimidazol-6-yl}oxy)methyl]benzoic acid (78 mg) was synthesized from methyl 2-[({1-[(3-methoxy-1,1′-biphenyl-4-yl)methyl]-2,4-dimethyl-1H-benzimidazol-6-yl}oxy)methyl]benzoate (120 mg) in a manner similar to that described in Example 56.

NMR(DMSO-d₆,δ): 2.4-2.6 (3H, s), 2.76 (3H, s), 3.89 (3H, s), 5.46 (2H, s), 5.48 (2H, s), 6.93 (1H, s), 7.11 (1H, s), 7.1-8.0 (12H, m), 12.7-13.4 (1H, br m).

MS: 491 (M−H).

### EXAMPLE 65

2-[({1-[2-Chloro-4-(1,3-oxazol-2-yl)benzyl]-2,4-dimethyl-1H-benzimidazol-6-yl}oxy)methyl]benzoic acid (76 mg) was synthesized from methyl 2-[({1-[2-chloro-4-(1,3-oxazol-2-yl)benzyl]-2,4-dimethyl-1H-benzimidazol-6-yl}oxy)methyl]benzoate (77 mg) in a manner similar to that described in Example 56.

NMR(DMSO-d₆,δ): 2.4-2.6 (6H, s), 5.40 (2H, s), 5.85 (2H, s), 6.77 (1H, d, J=8 Hz), 6.82 (1H, s), 6.88 (1H, s), 7.2-8.1 (6H, m), 8.02 (1H, d, J=8 Hz), 8.27 (1H, s), 12.5-13.5 (1H br m).

MS: 486 (M−H).

### EXAMPLE 66

2-[({1-[(3-Chloro-5-ethoxy-2-pyridinyl)methyl]-2,4-dimethyl-1H-benzimidazol-6-yl}oxy)methyl]benzoic acid (0.185 g) was synthesized from methyl 2-[({1-[(3-chloro-5-ethoxy-2-pyridinyl)methyl]-2,4-dimethyl-1H-benzimidazol-6-yl}oxy)methyl]benzoate (210 mg) in a manner similar to that described in Example 56.

NMR(DMSO-d₆,δ): 1.30 (3H, t, J=7 Hz), 2.44 (3H, s), 2.47 (3H, s), 4.09 (2H, q, J=7 Hz), 5.37 (2H, s), 5.44 (2H, s), 6.64 (1H, d, J=1 Hz), 6.73 (1H, d, J=2 Hz), 7.4-7.7 (4H, m), 7.91 (1H, d, J=1 Hz, 8 Hz), 8.08 (1H, d, J=2 Hz), 12.5-13.5 (1H, br m).

MS: 464 (M−H)

### EXAMPLE 67

A solution of methyl 2-[({1-[2-chloro-4-(dimethylamino)benzyl]-2,4-dimethyl-1H-benzimidazol-6-yl)oxy)methyl]benzoate (105 mg) in a solvent mixture of tetrahydrofuran (4 mL) and methanol (4 mL) was added 4 N sodium hydroxide (2 mL). The mixture was refluxed for half an hour, cooled to ambient temperature, and acidified with 1 N hydrochloric acid to afford the white precipitate. The suspension was stirred at ambient temperature for half an hour and filtered to give 2-[({1-[2-chloro-4-(dimethylamino)benzyl]-2,4-dimethyl-1H-benzimidazol-6-yl}oxy)methyl]benzoic acid (85 mg) as a white solid.

NMR(DMSO-d₆,δ): 2.49-2.51 (3H, s), 2.56 (3H, s), 2.87 (6H, s), 5.37 (2H, s), 5.40 (2H, s), 6.5-6.9 (5H, m), 7.3-8.0 (4H, m), 12.7-13.4 (1H, br m).

MS: 462 (M−H).

### EXAMPLE 68

2-({[1-[(3-Chloro-1,1′-biphenyl-4-yl)methyl]-4-methyl-2-(trifluoromethyl)-1H-benzimidazol-6-yl]oxy}methyl)benzoic acid (66 mg) was synthesized from methyl 2-({[1-[(3-chloro-1,1′-biphenyl-4-yl)methyl]-4-methyl-2-(trifluoromethyl)-1H -benzimidazol-6-yl]oxy}methyl)benzoate (90 mg) in a manner similar to that described in Example 56.

NMR(DMSO-d₆,δ): 2.58 (3H, s), 5.43 (2H, s), 5.72 (2H, s), 6.47 (1H, d, J=8 Hz), 6.95 (1H, d, J=1 Hz), 7.11 (1H, d, J=2 Hz), 7.4-7.9 (11H, m), 13.05 (1H, br s).

MS: 549 (M−H).

### EXAMPLE 69

2-[({1-[(3-Chloro-1,1′-biphenyl-4-yl)methyl]-2-ethyl-4-methyl-1H-benzimidazol-6-yl}oxy)methyl]benzoic acid (82 mg) was synthesized from methyl 2-[({1-[(3-chloro-1,1′-biphenyl-4-yl)methyl]-2-ethyl-4-methyl-1H-benzimidazol-6-yl)oxy)methyl]benzoate (110 mg) in a manner similar to that described in Example 56.

NMR(DMSO-d₆,δ): 1.26 (3H, t, J=7 Hz), 2.51 (3H, s), 2.78 (2H, q, J=7 Hz), 5.38 (2H, s), 5.49 (2H, s), 6.51 (1H, d, J=8 Hz), 6.71 (1H, d, J=1 Hz), 6.83 (1H, d, J=2 Hz), 7.3-7.9 (11H, m), 13.0 (1H, br s).

MS: 509 (M−H).

### EXAMPLE 70

2-[({1-[(3-Chloro-1,1′-biphenyl-4-yl)methyl]-2-ethoxy-4-methyl-1H-benzimidazol-6-yl)oxy)methyl]benzoic acid (135 mg) was synthesized from methyl 2-[({1-[(3-chloro-1,1′-biphenyl-4-yl)methyl]-2-ethoxy-4-methyl-1H-benzimidazol-6-yl)oxy)methyl]benzoate (150 mg) in a manner similar to that described in Example 56.

NMR(DMSO-d₆,δ): 1.35 (3H, t, J=7 Hz), 2.42 (3H, s), 4.52 (2H, q, J=7 Hz), 5.27 (2H, s), 5.37 (2H, s), 6.64 (1H, d, J=2 Hz), 6.78 (1H, d, J=2 Hz), 6.91 (1H, d, J=8 Hz), 7.4-7.9 (11H, m), 12.7-13.4 (1H, br m).

MS: 525 (M−H)

### EXAMPLE 71

2-{[(1-{[3-Chloro-5-(trifluoromethyl)-2-pyridinyl]methyl}-2-ethoxy-4-methyl-1H-benzimidazol-6-yl)oxy]methyl}benzoic acid (0.12 g) was synthesized from methyl 2-{[(1-{[3-chloro-5-(trifluoromethyl)-2-pyridinyl]methyl}-2-ethoxy-4-methyl-1H-benzimidazol-6-yl)oxy]methyl}benzoate (180 g) in a manner similar to that described in Example 56.

NMR(DMSO-d₆,δ): 1.26 (3H, t, J=7 Hz), 2.40 (3H, s), 3.77 (3H, s), 4.45 (2H, q, J=7 Hz), 5.35 (2H, s), 5.46 (2H, s), 6.60 (1H, d, J=2 Hz), 6.75 (1H, d, J=2 Hz), 7.3-8.0 (4H, m), 8.49 (1H, d, J=1 Hz), 8.76 (1H, d, J=1 Hz), 12.7-13.4 (1H, br m).

MS: 518 (M−H).

### EXAMPLE 72

2-({[1-[(3-Chloro-1,1′-biphenyl-4-yl)methyl]-2-(ethylthio)-4-methyl-1H-benzimidazol-6-yl]oxy}methyl)benzoic acid (117 mg) was synthesized from methyl 2-({[1-[(3-chloro-1,1′-biphenyl-4-yl)methyl]-2-(ethylthio)-4-methyl-1H-benzimidazol-6-yl]oxy}methyl)benzoate (125 mg) in a manner similar to that described in Example 56.

NMR(DMSO-d₆,δ): 1.33 (3H, t, J=7 Hz), 2.48-2.51 (3H, s), 3.26 (2H, q, J=7 Hz), 5.39 (2H, s), 5.45 (2H, s), 6.60 (1H, d, J=8 Hz), 6.73 (1H, d, J=1 Hz), 6.93 (1H, d, J=2 Hz), 7.4-7.9 (11H, m), 12.7-13.4 (1H, br m).

MS: 541 (M−H).

### EXAMPLE 73

2-({[1-(2,4-Dichlorobenzyl)-2-methyl-1H-benzimidazol-6-yl]oxy}methyl)benzoic acid (67 mg) was synthesized from methyl 2-({[1-(2,4-dichlorobenzyl)-2-methyl-1H-benzimidazol-6-yl]oxy}methyl)benzoate (144 mg) in a manner similar to that described in Example 56.

NMR(DMSO-d₆,δ): 2.41 (3H, s), 5.40 (2H, s), 5.45 (2H, s), 6.50 (1H, d, J=8 Hz), 6.84 (1H, dd, J=2 Hz, 9 Hz), 6.99 (1H, d, J=2 Hz), 7.3-7.6 (5H, m), 7.68 (1H, d, J=2 Hz), 7.8-8.0 (1H, m).

MS: 443, 441 (M+H).

### EXAMPLE 74

2-[({1-[(3-Chloro-1,1′-biphenyl-4-yl)methyl]-2-methyl-1H-benzimidazol-6-yl}oxy)methyl]benzoic acid (0.77 g) was synthesized from methyl 2-[({1-[(3-chloro-1,1′-biphenyl-4-yl)methyl]-2-methyl-1H-benzimidazol-6-yl}oxy)methyl]benzoate (950 mg) in a manner similar to that described in Example 56.

NMR(DMSO-d₆,δ): 2.45 (3H, s), 5.41 (2H, s), 5.52 (2H, s), 6.58 (1H, d, J=8 Hz), 6.86 (1H, dd, J=2 Hz, 8 Hz), 7.06 (1H, d, J=2 Hz), 7.3-7.9 (12H, m), 12.5-13.5 (1H, br m).

MS: 481 (M−H).

### EXAMPLE 75

2-{[(1-}[3-Chloro-5-(trifluoromethyl)-2-pyridinyl]methyl}-2-methyl-1H-benzimidazol-6-yl)oxy]methyl}benzoic acid (61 mg) was synthesized from methyl 2-{[(1-{[3-chloro-5-(trifluoromethyl)-2-pyridinyl]methyl}-2-methyl-1H-benzimidazol-6-yl)oxy]methyl}benzoate (110 mg) in a manner similar to that described in Example 56.

NMR(DMSO-d₆,δ): 2.41 (3H, s), 5.39 (2H, s), 5.70 (2H, s), 6.81 (1H, dd, J=2 Hz, 8 Hz), 7.00 (1H, d, J=2 Hz), 7.90 (1H, d, J=8 Hz), 7.3-7.9 (4H, m), 8.51 (1H, d, J=2 Hz), 8.75 (1H, d, J=1 Hz), 12.7-13.4 (1H, br m).

MS: 474 (M−H).

### EXAMPLE 76

Sodium hydride (60% in oil, 36 mg) was added in one portion to an ice-cooled suspension of [1-(2,4-dichlorobenzyl)-2,4-dimethyl-1H-benzimidazol-6-yl]methanol (100 mg) and 2-chloronicotinic acid (70.5 mg) in N,N-dimethylformamide (2 mL). The mixture was heated at 80° C. with stirring for 5 hours, and cooled to room temperature. The reaction was carefully quenched by addition of water, and the mixture was neutralized with 1N hydrochloric acid. The resulting precipitate was collected by filtration and purified over preparative thin layer chromatography (chloroform:methanol=10:1) to give 2-{[1-(2,4-dichlorobenzyl)-2,4-dimethyl-1H-benzimidazol-6-yl]methoxy}nicotinic acid (52 mg) as a powder.

¹H NMR (200 MHz, DMSO-d₆, δ/ppm): 2.47 (3H, s), 2.52 (3H, s), 5.45 (1H, d, J=3.1 Hz), 6.48 (1H, d, J=8.4 Hz), 7.06 (1H, dd, J=7.5 Hz, 4.5 Hz), 7.12 (1H, s), 7.2-7.4 (2H, m), 7.70 (1H, d, J=2.1 Hz), 8.09 (1H, dd, J=7.3 Hz, 2.0 Hz), 8.28 (1H, dd, J=4.9 Hz, 2.0 Hz), 12.6-13.3 (1H, br).

### EXAMPLE 77

An aqueous solution of sodium hydroxide (1N, 0.62 mL) was added to a suspension of ethyl 3-({[1-(2,4-dichlorobenzyl)-2,4-dimethyl-1H-benzimidazol-6-yl]methyl}amino)benzoate (60 mg) in 1,4-dioxane at room temperature, and the mixture was heated at 80° C. for 2 hours. It was cooled to room temperature and neutralized with 1N hydrochloric acid. The product was extracted with chloroform, and the organic layer was washed with water and brine and dried over sodium sulfate. The crude product was purified over preparative thin layer chromatography (chloroform:methanol=5:1) to give 3-({[1-(2,4-dichlorobenzyl)-2,4-dimethyl-1H-benzimidazol-6-yl]methyl}amino)benzoic acid (50 mg) as a powder.

¹H NMR (200 MHz, DMSO-d₆, δ/ppm): 2.45 (3H, s), 2.50 (3H, s), 4.27 (2H, d, J=5.7 Hz), 5.43 (2H, s), 6.3-6.5 (2H, m), 6.7-6.8 (1H, m), 7.01 (1H, s), 7.0-7.2 (2H, m), 7.15 (2H, br s), 7.27 (1H, dd, J=8.4 Hz, 2.1 Hz), 7.68 (1H, d, J=2.1 Hz), 12.5-12.7 (1H, br).

### EXAMPLE 78

3-[{[1-(2,4-Dichlorobenzyl)-2,4-dimethyl-1H-benzimidazol-6-yl]methyl}(methyl)amino]benzoic acid (119 mg) was synthesized from ethyl 3-[{[1-(2,4-dichlorobenzyl)-2,4-dimethyl-1H-benzimidazol-6-yl]methyl}(methyl)amino]benzoate (123 mg) in a manner similar to that described in Example 77.

¹H NMR (200 MHz, DMSO-d₆, δ/ppm): 2.46 (3H, s), 2.47 (3H, s), 4.58 (2H, s), 5.41 (2H, s), 6.52 (1H, d, J=8.4 Hz), 6.8-7.0 (3H, m), 7.1-7.3 (4H, m), 7.62 (1H, d, J=2.1 Hz), 12.5-12.9 (1H, br).

### EXAMPLE 79

Sodium triacetoxyborohydride (50 mg) and aqueous solution of formaldehyde (37%, 19.3 μL) were added to a solution of 2-({[1-(2,4-dichlorobenzyl)-2,4-dimethyl-1H-benzimidazol-6-yl]methyl}amino)benzoic acid (54 mg) in 1,2-dichloroethane (1.5 mL). The mixture was stirred at room temperature for 5 hours. It was diluted with methanol, and sodium borohydride (45 mg) was added. After stirring for 1 hour, the reaction was quenched by addition of water, and the organic materials were extracted with chloroform. The organic layer was washed with water, dried over sodium sulfate, and concentrated in vacuo. The crude product was purified over preparative thin layer chromatography (chloroform:methanol=10:1) to give 2-[{[1-(2,4-dichlorobenzyl)-2,4-dimethyl-1H-benzimidazol-6-yl]methyl}(methyl)amino]benzoic acid (21 mg) as a powder.

¹H NMR (200 MHz, DMSO-d₆, δ/ppm): 2.45 (3H, s), 2.46 (3H, s), 2.69 (3H, s), 4.27 (2H, s), 5.40 (2H, s), 6.38 (1H, d, J=8.4 Hz), 6.94 (1H, s), 7.01 (1H, s), 7.2-7.4 (2H, m), 7.57 (1H, t, J=7.4 Hz), 7.6-7.8 (2H, m), 7.86 (1H, dd, J=7.6 Hz, 0.9 Hz), 17.5-17.7 (1H, br).

### EXAMPLE 80

2-{[1-(2,4-Dichlorobenzyl)-2,4-dimethyl-1H-benzimidazol-6-yl]methoxy}benzoic acid (37 mg) was synthesized from methyl 2-{[1-(2,4-dichlorobenzyl)-2,4-dimethyl-1H-benzimidazol-6-yl]methoxy}benzoate (84 mg) in a manner similar to that described in Example 77.

¹H NMR (200 MHz, DMSO-d₆, δ/ppm): 2.47 (3H, s), 2.52 (3H, s), 5.17 (2H, s), 5.47 (2H, s), 6.47 (1H, d, J=8.3 Hz), 6.97 (1H, t, J=7.2 Hz), 7.1-7.2 (2H, m), 7.2-7.5 (3H, m), 7.60 (1H, dd, J=7.6 Hz, 1.6 Hz), 7.71 (1H, d, J=2.2 Hz), 12.2-13.0 (1H, br).

### EXAMPLE 81

3-{[1-(2,4-Dichlorobenzyl)-2,4-dimethyl-1H-benzimidazol-6-yl]methoxy}benzoic acid (82 mg) was synthesized from methyl 3-{[1-(2,4-dichlorobenzyl)-2,4-dimethyl-1H-benzimidazol-6-yl]methoxy}benzoate (117 mg) in a manner similar to that described in Example 77.

¹H NMR (200 MHz, DMSO-d₆, δ/ppm): 2.47 (3H, s), 2.53 (3H, s), 5.13 (2H, s), 5.49 (2H, s), 6.46 (1H, d, J=8.4 Hz), 7.09 (1H, s), 7.1-7.6 (6H, m), 7.71 (1H, d, J=2.1 Hz), 12.7-13.3 (1H, br).

### EXAMPLE 82

2-{[1-(2,4-Dichlorobenzyl)-2,4-dimethyl-1H-benzimidazol-6-yl]methoxy}isonicotinic acid (29 mg) was synthesized from ethyl 2-{[1-(2,4-dichlorobenzyl)-2,4-dimethyl-1H-benzimidazol-6-yl]methoxy}isonicotinate (52 mg) in a manner similar to that described in Example 77.

¹H NMR (400 MHz, DMSO-d₆, δ/ppm): 2.47 (3H, s), 2.52 (3H, s), 5.38 (2H, s), 5.49 (2H, s), 6.47 (1H, d, J=8.4 Hz), 7.09 (1H, s), 7.19 (1H, s), 7.2-7.3 (2H, m), 7.38 (1H, dd, J=5.2 Hz, 1.3 Hz), 7.70 (1H, d, J=2.1 Hz), 8.30 (1H, d, J=5.2 Hz), 13.4-13.9 (1H, br).

### EXAMPLE 83

6-{[1-(2,4-Dichlorobenzyl)-2,4-dimethyl-1H-benzimidazol-6-yl]methoxy}-2-pyridinecarboxylic acid (14 mg) was synthesized from methyl 6-{[1-(2,4-dichlorobenzyl)-2,4-dimethyl-1H-benzimidazol-6-yl]methoxy}-2-pyridinecarboxylate (36 mg) in a manner similar to that described in Example 77.

¹H NMR (400 MHz, DMSO-d₆, δ/ppm): 2.46 (3H, s), 2.52 (3H, s), 5.40 (2H, s), 5.48 (2H, s), 6.46 (1H, d, J=8.4 Hz), 7.03 (1H, d, J=8.3 Hz), 7.15 (1H, s), 7.29 (1H, dd, J=8.4 Hz, 2.0 Hz), 7.38 (1H, s), 7.65 (1H, d, J=7.2 Hz), 7.70 (1H, d, J=2.0 Hz), 7.85 (1H, t, J=7.8 Hz), 12.7-13.4 (1H, br).

### EXAMPLE 84

2-({1-[(3-Chloro-1,1′-biphenyl-4-yl)methyl]-2,4-dimethyl-1H-benzimidazol-6-yl}methoxy)benzoic acid (26 mg) was synthesized from methyl 2-({1-[(3-chloro-1,1′-biphenyl-4-yl)methyl]-2,4-dimethyl-1H-benzimidazol-6-yl}methoxy)benzoate (100 mg) in a manner similar to that described in Example 77.

¹H NMR (200 MHz, DMSO-d₆, δ/ppm): 2.5 (3H, s), 2.54 (3H, s), 5.18 (2H, s), 5.53 (2H, s), 6.53 (1H, d, J=8.1 Hz), 6.95 (1H, t, J=7.0 Hz), 7.1-7.2 (2H, m), 7.3-7.7 (9H, m), 7.82 (1H, d, J=1.8 Hz), 12.3-12.9 (1H, br).

### EXAMPLE 85

3-({1-[(3-Chloro-1,1′-biphenyl-4-yl)methyl]-2,4-dimethyl-1H-benzimidazol-6-yl}methoxy)benzoic acid (44 mg) was synthesized from methyl 3-({1-[(3-chloro-1,1′-biphenyl-4-yl)methyl]-2,4-dimethyl-1H-benzimidazol-6-yl}methoxy)benzoate (80 mg) in a manner similar to that described in Example 77.

¹H NMR (400 MHz, DMSO-d₆, δ/ppm): 2.51 (3H, s), 2.55 (3H, s), 5.14 (2H, s), 5.55 (2H, s), 6.53 (1H, d, J=8.1 Hz), 7.10 (1H, s), 7.2-7.3 (1H, m), 7.3-7.4 (3H, m), 7.4-7.6 (5H, m), 7.66 (2H, d, J=7.2 Hz), 7.83 (1H, d, J=1.8 Hz), 12.8-13.2 (1H, br).

### EXAMPLE 86

2-({1-[(3-Chloro-1,1′-biphenyl-4-yl)methyl]-2,4-dimethyl-1H-benzimidazol-6-yl}methoxy)nicotinic acid (71 mg) was synthesized from {1-[(3-chloro-1,1′-biphenyl-4-yl)methyl]-2,4-dimethyl-1H-benzimidazol-6-yl}methanol (200 mg) and 2-chloronicotinic acid (125 mg) in a manner similar to that described in Example 76.

¹H NMR (200 MHz, DMSO-d₆, δ/ppm): 2.5 (3H, s), 2.53 (3H, s), 5.45 (2H, s), 5.52 (2H, s), 6.55 (1H, d, J=8.1 Hz), 7.02 (1H, dd, J=7.5 Hz, 4.9 Hz), 7.13 (1H, s), 7.3-7.7 (7H, m), 7.82 (1H, d, J=1.8 Hz), 8.06 (1H, dd, J=7.4 Hz, 2.0 Hz), 8.27 (1H, dd, J=4.9 Hz, 2.0 Hz), 12.6-13.4 (1H, br).

### EXAMPLE 87

6-({1-[(3-Chloro-1,1′-biphenyl-4-yl)methyl]-2,4-dimethyl-1H-benzimidazol-6-yl}methoxy)-2-pyridinecarboxylic acid (72 mg) was synthesized from {1-[(3-chloro-1,1′-biphenyl-4-yl)methyl]-2,4-dimethyl-1H-benzimidazol-6-yl}methanol (100 mg) and 6-chloro-2-pyridinecarboxylic acid (62.7 mg) in a manner similar to that described in Example 76.

MS (ES+): 498 (M⁺+1).

### EXAMPLE 88

2-({1-[(3-Chloro-1,1′-biphenyl-4-yl)methyl]-2,4-dimethyl-1H-benzimidazol-6-yl}methoxy)isonicotinic acid (35 mg) was synthesized from {1-[(3-chloro-1,1′-biphenyl-4-yl)methyl]-2,4-dimethyl-1H-benzimidazol-6-yl}methanol (100 mg) and 2-chloroisonicotinic acid (62.7 mg) in a manner similar to that described in Example 76.

MS (ES+): 498 (M⁺+1).

### EXAMPLE 89

2-[({1-[(3-Chloro-1,1′-biphenyl-4-yl)methyl]-2,4-dimethyl-1H-benzimidazol-6-yl}methyl)amino]isonicotinic acid (65 mg) was synthesized from isopropyl 2-[({1-[(3-chloro-1,1′-biphenyl-4-yl)methyl]-2,4-dimethyl-1H-benzimidazol-6-yl}methyl)amino]isonicotinate (80 mg) in a manner similar to that described in Example 77.

¹H NMR (200 MHz, DMSO-d₆, δ/ppm): 2.5 (3H, s), 2.5 (3H, s), 4.51 (2H, d, J=5.7 Hz), 5.50 (2H, s), 6.51 (1H, d, J=8.1 Hz), 6.81 (1H, d, J=5.2 Hz), 6.99 (2H, s), 7.17 (1H, s), 7.28 (1H, t, J=5.5 Hz), 7.3-7.5 (4H, m), 7.63 (1H, s), 7.65 (1H, d, J=6.7 Hz), 7.80 (1H, d, J=1.5 Hz), 8.02 (1H, d, J=5.2 Hz), 10.0-10.9 (1H, br).

### EXAMPLE 90

6-[({1-[(3-Chloro-1,1′-biphenyl-4-yl)methyl]-2,4-dimethyl-1H-benzimidazol-6-yl}methyl)amino]-2-pyridinecarboxylic acid (45 mg) was synthesized from isopropyl 6-[({1-[(3-chloro-1,1′-biphenyl-4-yl)methyl]-2,4-dimethyl-1H-benzimidazol-6-yl}methyl)amino]-2-pyridinecarboxylate (60 mg) in a manner similar to that described in Example 77.

¹H NMR (200 MHz, DMSO-d₆, δ/ppm): 2.5 (3H, s), 2.5 (3H, s), 4.54 (2H, d, J=5.7 Hz), 5.50 (2H, s), 6.50 (1H, d, J=7.9 Hz), 6.66 (1H, d, J=8.3 Hz), 7.04 (1H, s), 7.14 (1H, d, J=7.1 Hz), 7.25 (1H, s), 7.3-7.6 (6H, m), 7.64 (1H, s), 7.65 (1H, d, J=6.7 Hz), 7.81 (1H, d, J=1.6 Hz), 11.8-12.6 (1H, br).

### EXAMPLE 91

2-{[1-(2-Chloro-4-ethoxybenzyl)-2,4-dimethyl-1H-benzimidazol-6-yl]methoxy}benzoic acid (70 mg) was synthesized from methyl 2-{[1-(2-chloro-4-ethoxybenzyl)-2,4-dimethyl-1H-benzimidazol-6-yl]methoxy}benzoate (139 mg) in a manner similar to that described in Example 77.

H NMR (200 MHz, DMSO-d₆, δ/ppm): 1.28 (3H, t, J=7.0 Hz), 2.46 (3H, s), 2.5 (3H, s), 3.99 (2H, q, J=7.0 Hz), 5.17 (2H, s), 5.40 (2H, s), 6.48 (1H, d, J=8.6 Hz), 6.79 (1H, dd, J=8.6 Hz, 2.5 Hz), 6.97 (1H, t, J=7.5 Hz), 7.0-7.2 (3H, m), 7.33 (1H, s), 7.4-7.5 (1H, m), 7.61 (1H, dd, J=7.6 Hz, 1.7 Hz), 12.3-12.9 (1H, br).

### EXAMPLE 92

3-{[1-(2-Chloro-4-ethoxybenzyl)-2,4-dimethyl-1H-benzimidazol-6-yl]methoxy}benzoic acid (46 mg) was synthesized from methyl 3-{[1-(2-chloro-4-ethoxybenzyl)-2,4-dimethyl-1H-benzimidazol-6-yl]methoxy}benzoate (80 mg) in a manner similar to that described in Example 77.

¹H NMR (400 MHz, DMSO-d₆, δ/ppm): 1.28 (3H, t, J=7.0 Hz), 2.48 (3H, s), 2.53 (3H, s), 4.00 (2H, q, J=7.0 Hz), 5.13 (2H, s), 5.41 (2H, s), 6.48 (1H, d, J=8.6 Hz), 6.79 (1H, dd, J=8.6 Hz, 2.5 Hz), 7.0-7.1 (2H, m), 7.2-7.3 (1H, m), 7.28 (1H, s), 7.38 (1H, t, J=7.9 Hz), 7.4-7.6 (2H, m), 12.8-13.1 (1H, br).

### EXAMPLE 93

2-[(1-{[3-Chloro-5-(trifluoromethyl)-2-pyridinyl]methyl}-2,4-dimethyl-1H-benzimidazol-6-yl)methoxy]isonicotinic acid (43 mg) was synthesized from ethyl 2-[(1-{[3-chloro-5-(trifluoromethyl)-2-pyridinyl]methyl}-2,4-dimethyl-1H-benzimidazol-6-yl)methoxy]isonicotinate (72 mg) in a manner similar to that described in Example 77.

¹H NMR (200 MHz, DMSO-d₆, δ/ppm): 2.47 (3H, s), 2.51 (3H, s), 5.37 (2H, s), 5.75 (2H, s), 7.05 (1H, s), 7.18 (1H, s), 7.28 (1H, s), 7.37 (1H, dd, J=7.1 Hz, 1.2 Hz), 8.29 (1H, d, J=5.3 Hz), 8.53 (1H, d, J=1.6 Hz), 8.74 (1H, s), 13.0-14.2 (1H, br).

### EXAMPLE 94

6-[(1-{[3-Chloro-5-(trifluoromethyl)-2-pyridinyl]methyl}-2,4-dimethyl-1H-benzimidazol-6-yl)methoxy]-2-pyridinecarboxylic acid (41 mg) was synthesized from methyl 6-[(1-{[3-chloro-5-(trifluoromethyl)-2-pyridinyl]methyl}-2,4-dimethyl-1H-benzimidazol-6-yl)methoxy]-2-pyridinecarboxylate (60 mg) in a manner similar to that described in Example 77.

¹H NMR (200 MHz, DMSO-d₆, δ/ppm): 2.46 (3H, s), 2.51 (3H, s), 5.39 (2H, s), 5.74 (2H, s), 7.02 (1H, d, J=8.6 Hz), 7.12 (1H, s), 7.35 (1H, d, J=6.1 Hz), 7.63 (1H, d, J=6.9 Hz), 7.84 (1H, t, J=7.8 Hz), 8.53 (1H, d, J=1.5 Hz), 8.72 (1H, d, J=1.5 Hz), 12.8-13.6 (1H, br).

### EXAMPLE 95

To a mixture of 6-bromo-1-[2-chloro-4-(pentyloxy)benzyl]-2,4-dimethyl-1H-benzimidazole (100 mg) and 3-(dihydroxyboryl)benzoic acid (50 mg) in 1,2-dimethoxyethane (DME) (2 ml) were added 2M-sodium carbonate (Na₂CO₃) (0.38 ml) and tetrakis(triphenylphosphine)palladium(0) {Pd(PPh₃)₄} (13 mg) at ambient temperature. The mixture was heated at 90° C. for 20 hours. After cooling, the reaction mixture was diluted with EtOAc (40 ml) and washed with brine (2×30 ml). The organic layer was dried over MgSO₄ and filtered. Evaporation gave a residue (150 mg) which was triturated with EtOAc—n-hexane to give 3-{1-[2-chloro-4-(pentyloxy)benzyl]-2,4-dimethyl-1H-benzimidazol-6-yl}benzoic acid (40 mg) as pale brown crystals.

NMR(DMSO-d₆,δ): 0.8-1.0 (3H, m), 1.2-1.4 (4H, m), 1.5-1.8 (2H, m), 2.48 (3H, s), 2.60 (3H, s), 3.93 (2H, t, J=6.4 Hz), 5.53 (2H, br s), 6.50 (1H, d, J=8.6 Hz), 6.81 (1H, dd, J=2.5 Hz, 8.6 Hz), 7.10 (1H, d, J=2.5 Hz), 7.34 (1H, br s), 7.4-7.7 (2H, m), 7.8-8.1 (2H, m), 8.1-8.3 (1H, m), 13.07 (1H, br s).

MS: 475 (M−1).

### EXAMPLE 96

To a mixture of 6-bromo-1-[2-chloro-4-(pentyloxy)benzyl]-2,4-dimethyl-1H-benzimidazole (150 mg) and 4-(dihydroxyboryl)benzoic acid (74 mg) in DME (3 ml) were added 2M-Na₂CO₃ (0.57 ml) and Pd(PPh₃)₄ (20 mg) at ambient temperature. The mixture was heated at 90° C. for 18 hours. After cooling, the reaction mixture was added water and the pH of the mixture was adjusted to around 3 with 1N-HCl. The aqueous phase was extracted with EtOAc (30 ml). The organic layer was washed with brine (2×30 ml), dried over MgSO₄, and filtered. Evaporation gave a residue (133 mg) which was triturated with EtOAc (1.5 ml)—n-hexane (0.5 ml) to give 4-{1-[2-chloro-4-(pentyloxy)benzyl]-2,4-dimethyl-1H-benzimidazol-6-yl}benzoic acid (56 mg) as white crystals.

NMR(DMSO-d₆,δ): 0.8-1.0 (3H, m), 1.2-1.5 (4H, m), 1.5-1.8 (2H, m), 2.48 (3H, s), 2.59 (3H, s), 3.93 (2H, t, J=6.4 Hz), 5.52 (2H, br s), 6.50 (1H, d, J=8.6 Hz), 6.82 (1H, dd, J=2.5 Hz, 8.6 Hz), 7.11 (1H, d, J=2.5 Hz), 7.39 (1H, br s), 7.62 (1H, br s), 7.78 (2H, d, J=8.4 Hz) 7.98 (2H, d, J=8.4 Hz), 12.91 (1H, br s).

MS: 477 (M+1).

### EXAMPLE 97

2-{1-[2-Chloro-4-(pentyloxy)benzyl]-2-methyl-1H-benzimidazol-6-yl}-4-methyl-1,3-oxazole-5-carboxylic acid (105 mg) was synthesized from ethyl 2-{1-[2-chloro-4-(pentyloxy)benzyl]-2-methyl-1H-benzimidazol-6-yl}-4-methyl-1,3-oxazole-5-carboxylate (112 mg) in a manner similar to that described in Example 14.

NMR(DMSO-d₆+NaOD,δ): 0.8-1.0 (3H, m), 1.2-1.5 (4H, m), 1.5-1.8 (2H, m), 2.35 (3H, s), 3.94 (2H, t, J=6.4 Hz), 6.53 (1H, d, J=8.6 Hz), 6.82 (1H, dd, J=2.5 Hz, 8.6 Hz), 7.12 (1H, d, J=2.5 Hz), 7.67 (1H, d, J=8.5 Hz), 7.85 (1H, dd, J=1.3 Hz, 8.5 Hz), 7.96 (1H, d, J=1.3 Hz).

MS: 466 (M−1).

### EXAMPLE 98

2-{1-[2-Chloro-4-(pentyloxy)benzyl]-2-methyl-1H-benzimidazol-6-yl}-4-methyl-1H-imidazole-5-carboxylic acid (101 mg) was synthesized from ethyl 2-{1-[2-chloro-4-(pentyloxy)benzyl]-2-methyl-1H-benzimidazol-6-yl}-4-methyl-1H-imidazole-5-carboxylate (140 mg) in a manner similar to that described in Example 14.

NMR(DMSO-d₆,δ): 0.87 (3H, t, J=6.9 Hz), 1.1-1.5 (4H, m), 1.5-1.8 (2H, m), 2.47 (3H, s), 3.94 (2H, t, J=6.4 Hz), 5.52 (2H, br s), 6.46 (1H, d, J=8.7 Hz), 6.82 (1H, dd, J=2.5 Hz, 8.7 Hz), 7.15 (1H, d, J=2.5 Hz), 7.72 (1H, d, J=8.5 Hz), 7.96 (1H, dd, J=1.1 Hz, 8.5 Hz), 8.16 (1H, br s).

MS: 465 (M−1).

### EXAMPLE 99

2-[1-(2,4-Dichlorobenzyl)-2,4-dimethyl-1H-benzimidazol-6-yl]-4-methyl-1,3-oxazole-5-carboxylic acid (194 mg) was synthesized from ethyl 2-[1-(2,4-dichlorobenzyl)-2,4-dimethyl-1H-benzimidazol-6-yl]-4-methyl-1,3-oxazole-5-carboxylate (240 mg) in a manner similar to that described in Example 14.

NMR(CD₃OD+NaOD,δ): 2.47 (3H, s), 2.58 (3H, s), 2.67 (3H, s), 5.59 (2H, br s), 6.49 (1H, d, J=8.4 Hz), 7.22 (1H, dd, J=2.0 Hz, 8.4 Hz), 7.59 (1H, d, J=2.0 Hz), 7.86 (1H, br s), 7.94 (1H, br s).

MS: 428 (M−1).

### EXAMPLE 100

2-[1-(2,4-Dichlorobenzyl)-2,4-dimethyl-1H-benzimidazol-6-yl]-4-methyl-1H-imidazole-5-carboxylic acid (114 mg) was synthesized from ethyl 2-[1-(2,4-dichlorobenzyl)-2,4-dimethyl-1H-benzimidazol-6-yl]-4-methyl-1H-imidazole-5-carboxylate (140 mg) in a manner similar to that described in Example 14.

NMR(DMSO-d₆,δ): 2.23 (3H, s), 2.43 (3H, s), 2.58 (3H, s), 5.52 (2H, br s), 6.36 (1H, d, J=8.4 Hz), 7.32 (1H, dd, J=2.1 Hz, 8.4 Hz), 7.6-8.1 (2H, m), 7.76 (1H, d, J=2.1 Hz), 12.60 (1H, br s).

MS: 427 (M−1).

### EXAMPLE 101

2-{1-[(3-Chloro-1,1′-biphenyl-4-yl)methyl]-2,4-dimethyl-1H-benzimidazol-6-yl}-4-methyl-1,3-oxazole-5-carboxylic acid (211 mg) was synthesized from ethyl 2-{1-[(3-chloro-1,1′-biphenyl-4-yl)methyl]-2,4-dimethyl-1H-benzimidazol-6-yl}-4-methyl-1,3-oxazole-5-carboxylate (270 mg) in a manner similar to that described in Example 14.

NMR(CD₃OD+NaOD,δ): 2.47 (3H, s), 2.60 (3H, s), 2.69 (3H, s), 5.65 (2H, br s), 6.57 (1H, d, J=8.1 Hz), 7.2-7.6 (6H, m), 7.76 (1H, d, J=1.6 Hz), 7.88 (1H, br s), 8.00 (1H, br s).

MS: 470 (M−1).

### EXAMPLE 102

2-{1-[(3-Chloro-1,1′-biphenyl-4-yl)methyl]-2,4-dimethyl-1H-benzimidazol-6-yl}-4-methyl-1H-imidazole-5-carboxylic acid (182 mg) was synthesized from ethyl 2-{1-[(3-chloro-1,1′-biphenyl-4-yl)methyl]-2,4-dimethyl-1H-benzimidazol-6-yl}-4-methyl-1H-imidazole-5-carboxylate (200 mg) in a manner similar to that described in Example 14.

NMR(DMSO-d₆,δ): 2.3-2.7 (9H), 5.58 (2H, br s), 6.41 (1H, d, J=8.1 Hz), 7.3-8.2 (10H, m), 12.61 (1H, br s).

MS: 469 (M−1).

### EXAMPLE 103

2-{1-[2-Chloro-4-(pentyloxy)benzyl]-2,4-dimethyl-1H-benzimidazol-6-yl}-4-methyl-1,3-oxazole-5-carboxylic acid (196 mg) was synthesized from ethyl 2-{1-[2-chloro-4-(pentyloxy)benzyl]-2,4-dimethyl-1H-benzimidazol-6-yl}-4-methyl-1,3-oxazole-5-carboxylate (250 mg) in a manner similar to that described in Example 14.

NMR(CD₃OD+NaOD,δ): 0.8-1.0 (3H, m), 1.2-1.5 (4H, m), 1.6-1.9 (2H, m), 2.47 (3H, s), 2.56 (3H, s), 2.67 (3H, s), 3.93 (2H, t, J=6.3 Hz), 5.52 (2H, br s), 6.49 (1H, d, J=8.6 Hz), 6.75 (1H, dd, J=2.5 Hz, 8.6 Hz), 7.05 (1H, d, J=2.5 Hz), 7.85 (1H, br s), 7.98 (1H, br s).

MS: 480 (M−1).

### EXAMPLE 104

2-{1-[2-Chloro-4-(pentyloxy)benzyl]-2,4-dimethyl-1H-benzimidazol-6-yl}-4-methyl-1H-imidazole-5-carboxylic acid (157 mg) was synthesized from ethyl 2-{1-[2-chloro-4-(pentyloxy)benzyl]-2,4-dimethyl-1H-benzimidazol-6-yl}-4-methyl-1H-imidazole-5-carboxylate (170 mg) in a manner similar to that described in Example 14.

NMR(DMSO-d₆,δ): 0.8-1.0 (3H, m), 1.2-1.5 (4H, m), 1.5-1.8 (2H, m), 2.44 (3H, s), 2.4-2.6 (3H, s), 2.58 (3H, s), 3.94 (2H, t, J=6.4 Hz), 5.47 (2H, br s), 6.38 (1H, d, J=8.6 Hz), 6.80 (1H, dd, J=2.2 Hz, 8.6 Hz), 7.14 (1H, d, J=2.2 Hz), 7.78 (1H, br s), 7.90 (1H, br s).

MS: 479 (M−1).

### EXAMPLE 105

3-[1-(2,4-Dichlorobenzyl)-2,4-dimethyl-1H-benzimidazol-6-yl]benzoic acid (74 mg) was synthesized from 6-bromo-1-(2,4-dichlorobenzyl)-2,4-dimethyl-1H-benzimidazole (143 mg) and 3-(dihydroxyboryl)benzoic acid (80 mg) in a manner similar to that described in Preparation Example 242.

¹H NMR (200 MHz, DMSO-d₆, δ/ppm): 2.46 (3H, s), 2.61 (3H, s), 5.61 (2H, s), 6.45 (1H, d, J=8.4 Hz), 7.32 (1H, dd, J=8 Hz, 2.1 Hz), 7.54 (1H, t, J=7.7 Hz), 7.63 (1H, s), 7.73 (1H, d, J=2.0 Hz), 7.89 (2H, m), 8.19 (1H, s), 12.9-13.2 (1H, br).

### EXAMPLE 106

3-(1-{[3-Chloro-5-(trifluoromethyl)-2-pyridinyl]methyl}-2,4-dimethyl-1H-benzimidazol-6-yl)benzoic acid (33 mg) was synthesized from 6-bromo-1-{[3-chloro-5-(trifluoromethyl)-2-pyridinyl]methyl}-2,4-dimethyl-1H-benzimidazole (183 mg) and 3-(dihydroxyboryl)benzoic acid (94 mg) in a manner similar to that described in Preparation Example 242.

¹H NMR (200 MHz, DMSO-d₆, δ/ppm): 2.45 (3H, s), 2.59 (3H, s), 5.87 (2H, s), 7.31 (1H, s), 7.53 (1H, t, J=7.8 Hz), 7.62 (1H, s), 7.84-7.91 (2H, m), 8.18 (1H, s), 8.55 (1H, s), 8.75 (1H, s), 12.6-13.5 (1H, br).

### EXAMPLE 107

3-[1-(4-Ethoxy-2-methylbenzyl)-2,4-dimethyl-1H-benzimidazol-6-yl]benzoic acid (84 mg) was synthesized from 6-bromo-1-(4-ethoxy-2-methylbenzyl)-2,4-dimethyl-1H-benzimidazole (180 mg) and 3-(dihydroxyboryl)benzoic acid (104 mg) in a manner similar to that described in Preparation Example 242.

¹H NMR (200 MHz, DMSO-d₆, δ/ppm): 1.26 (3H, t, J=6.9 Hz), 2.36 (3H, s), 2.44 (3H, s), 2.60 (3H, s), 3.93 (2H, q, J=6.9 Hz), 6.18 (1H, d, J=8.5 Hz), 6.59 (1H, dd, J=8.4 Hz, 2.6 Hz), 6.81 (1H, d, J=2.4 Hz), 7.32 (1H, s), 7.52 (1H, s), 7.53 (1H, t, J=7.8 Hz), 7.87 (1H, d, J=7.1 Hz), 8.16 (1H, s), 12.8-13.2 (1H, br).

### EXAMPLE 108

3-[1-(4-Ethoxy-2-methoxybenzyl)-2,4-dimethyl-1H-benzimidazol-6-yl]benzoic acid (33 mg) was synthesized from 6-bromo-1-(4-ethoxy-2-methoxybenzyl)-2,4-dimethyl-1H-benzimidazole (118 mg) and 3-(dihydroxyboryl)benzoic acid (65 mg) in a manner similar to that described in Preparation Example 242.

¹H NMR (200 MHz, DMSO-d₆, δ/ppm): 1.27 (3H, t, J=6.9 Hz), 2.54 (3H, s), 2.56 (3H, s), 3.81 (3H, s), 3.97 (2H, q, J=7.0 Hz), 5.34 (2H, s), 6.42 (1H, dd, J=8.1 Hz, 2.1 Hz), 6.76 (1H, d, J=8.5 Hz), 7.29 (1H, s), 7.55 (1H, t, J=7.6 Hz), 7.56 (1H, s), 7.88 (1H, d, J=7.3 Hz), 8.18 (1H, s), 12.9-13.3 (1H, br).

### EXAMPLE 109

3-[1-(2-Chloro-4-ethoxybenzyl)-2,4-dimethyl-1H-benzimidazol-6-yl]benzoic acid (58 mg) was synthesized from 6-bromo-1-(2-chloro-4-ethoxybenzyl)-2,4-dimethyl-1H-benzimidazole (180 mg) and 3-(dihydroxyboryl)benzoic acid (99 mg) in a manner similar to that described in Preparation Example 242.

¹H NMR (200 MHz, DMSO-d₆, δ/ppm): 1.27 (3H, t, J=6.9 Hz), 2.48 (3H, s), 2.59 (3H, s), 3.99 (2H, q, J=6.9 Hz), 5.53 (2H, s), 6.51 (1H, d, J=8.5 Hz), 6.82 (1H, dd, J=8.6 Hz, 2.4 Hz), 7.10 (1H, d, J=2.5 Hz), 7.34 (1H, s), 7.54 (1H, t, J=7.8 Hz), 7.58 (1H, s), 7.8-8.0 (2H, m), 8.17 (1H, s), 12.8-13.4 (1H, br).

### EXAMPLE 110

3-{1-[(3,5-Dichloro-2-pyridinyl)methyl]-2,4-dimethyl-1H-benzimidazol-6-yl}benzoic acid (47 mg) was synthesized from 6-bromo-1-[(3,5-dichloro-2-pyridinyl)methyl]-2,4-dimethyl-1H-benzimidazole (100 mg) and 3-(dihydroxyboryl)benzoic acid (56 mg) in a manner similar to that described in Preparation Example 242.

¹H NMR (200 MHz, DMSO-d₆, δ/ppm): 2.45 (3H, s), 2.58 (3H, s), 5.75 (2H, s), 7.30 (1H, s), 7.53 (1H, t, J=7.3 Hz), 7.59 (1H, s), 7.8-8.0 (2H, m), 8.18 (1H, s), 8.32 (1H, d, J=0.8 Hz), 8.43 (1H, d, J=2.0 Hz), 12.8-13.4 (1H, br).

### EXAMPLE 111

3-[1-(2-Chlorobenzyl)-2,4-dimethyl-1H-benzimidazol-6-yl]benzoic acid (54 mg) was synthesized from 6-bromo-1-(2-chlorobenzyl)-2,4-dimethyl-1H-benzimidazole (140 mg) and 3-(dihydroxyboryl)benzoic acid (86 mg) in a manner similar to that described in Preparation Example 242.

¹H NMR (200 MHz, DMSO-d₆, δ/ppm): 2.47 (3H, s), 2.61 (3H, s), 5.63 (2H, s), 6.49 (1H, dd, J=7.5 Hz, 1.5 Hz), 7.21 (1H, t, J=7.5 Hz), 7.32 (1H, t, J=7.6 Hz), 7.35 (1H, s), 7.5-7.7 (3H, m), 7.8-8.0 (2H, m), 8.18 (1H, s), 12.8-13.3 (1H, br).

### EXAMPLE 112

3-(1-{2-Chloro-4-[(ethoxycarbonyl)(methyl)amino]benzyl}-2,4-dimethyl-1H-benzimidazol-6-yl)benzoic acid (103 mg) was synthesized from ethyl 4-[(6-bromo-2,4-dimethyl-1H-benzimidazol-1-yl)methyl]-3-chlorophenyl(methyl)carbamate (180 mg) and 3-(dihydroxyboryl)benzoic acid (86 mg) in a manner similar to that described in Preparation Example 242.

¹H NMR (200 MHz, DMSO-d₆, δ/ppm): 2.5 (3H, s), 2.61 (3H, s), 3.18 (3H, s), 4.05 (2H, q, J=7.0 Hz), 5.61 (2H, s), 6.48 (1H, d, J=8.4 Hz), 7.18 (1H, dd, J=8.4 Hz, 2.2 Hz), 7.35 (1H, s), 7.5-7.6 (3H, m), 7.8-8.0 (2H, m), 8.18 (1H, s), 13.0-13.1 (1H, br).

### EXAMPLE 113

3-{1-[(2,6-Dichloro-3-pyridinyl)methyl]-2,4-dimethyl-1H-benzimidazol-6-yl}benzoic acid (148 mg) was synthesized from ethyl 3-{1-[(2,6-dichloro-3-pyridinyl)methyl]-2,4-dimethyl-1H-benzimidazol-6-yl}benzoate (190 mg) in a manner similar to that described in Example 134.

¹H NMR (200 MHz, DMSO-d₆, δ/ppm): 2.48 (3H, s), 2.61 (3H, s), 5.61 (2H, s), 6.87 (1H, d, J=8.1 Hz), 7.37 (1H, s), 7.44 (1H, d, J=8.1 Hz), 7.53 (1H, t, J=6.9 Hz), 7.67 (1H, s), 7.8-8.0 (2H, m), 8.20 (1H, s), 12.9-13.5 (1H, br).

### EXAMPLE 114

3-{2,4-Dimethyl-1-[(5-methyl-2-phenyl-1,3-oxazol-4-yl)methyl]-1H-benzimidazol-6-yl}benzoic acid (94 mg) was synthesized from ethyl 3-{2,4-dimethyl-1-[(5-methyl-2-phenyl-1,3-oxazol-4-yl)methyl]-1H-benzimidazol-6-yl}benzoate (160 mg) in a manner similar to that described in Example 134.

¹H NMR (200 MHz, DMSO-d₆, δ/ppm): 2.52 (3H, s), 2.55 (3H, s), 2.73 (3H, s), 5.44 (2H, s), 7.31 (1H, s), 7.4-7.5 (3H, m), 7.58 (1H, t, J=8.0 Hz), 7.77 (1H, s), 7.8-8.0 (4H, m), 8.28 (1H, s), 12.9-13.3 (1H, br).

MS (ES+): 438 (M⁺+1).

### EXAMPLE 115

3-{1-[(3-Chloro-1,1′-biphenyl-4-yl)methyl]-2,4-dimethyl-1H-benzimidazol-6-yl}benzoic acid (86 mg) was synthesized from ethyl 3-{1-[(3-chloro-1,1′-biphenyl-4-yl)methyl]-2,4-dimethyl-1H-benzimidazol-6-yl}benzoate (114 mg) in a manner similar to that described in Example 134.

¹H NMR (400 MHz, DMSO-d₆, δ/ppm): 2.51 (3H, s), 2.62 (3H, s), 5.67 (2H, s), 6.54 (1H, d, J=8.1 Hz), 7.3-7.4 (2H, m), 7.45 (2H, t, J=7.8 Hz), 7.53 (1H, d, J=7.3 Hz), 7.54 (1H, t, J=6.4 Hz), 7.6-7.7 (3H, m), 7.84 (1H, d, J=1.6 Hz), 7.87 (1H, d, J=8.0 Hz), 7.91 (1H, d, J=7.3 Hz), 8.20 (1H, t, J=1.6 Hz), 12.9-13.2 (1H, br).

### EXAMPLE 116

3-[2,4-Dimethyl-1-({4-methyl-2-[4-(trifluoromethyl)phenyl]-1,3-thiazol-5-yl}methyl)-1H-benzimidazol-6-yl]benzoic acid (113 mg) was synthesized from ethyl 3-[2,4-dimethyl-1-({4-methyl-2-[4-(trifluoromethyl)phenyl]-1,3-thiazol-5-yl}methyl)-1H-benzimidazol-6-yl]benzoate (129 mg) in a manner similar to that described in Example 134.

¹H NMR (400 MHz, DMSO-d₆, δ/ppm): 2.58 (3H, s), 2.60 (3H, s), 2.62 (3H, s), 5.83 (2H, s), 7.37 (1H, s), 7.60 (1H, t, J=7.7 Hz), 7.75 (2H, d, J=8.4 Hz), 7.80 (1H, s), 7.91 (1H, d, J=7.7 Hz), 7.97 (1H, d, J=7.7 Hz), 8.02 (2H, d, J=8.3 Hz), 8.27 (1H, s), 12.9-13.3 (1H, br).

### EXAMPLE 117

3-[1-(2-Chloro-4-ethoxybenzyl)-2-methyl-1H-benzimidazol-6-yl]benzoic acid (112 mg) was synthesized from ethyl 3-[1-(2-chloro-4-ethoxybenzyl)-2-methyl-1H-benzimidazol-6-yl]benzoate (158 mg) in a manner similar to that described in Example 134.

¹H NMR (200 MHz, DMSO-d₆, δ/ppm): 1.28 (3H, t, J=7.0 Hz), 2.48 (3H, s), 4.00 (2H, q, J=7.0 Hz), 5.56 (2H, s), 6.55 (1H, d, J=8.7 Hz), 6.82 (1H, dd, J=8.6 Hz, 2.4 Hz), 7.10 (1H, d, J=2.5 Hz), 7.51 (1H, d, J=7.2 Hz), 7.56 (1H, t, J=7.8 Hz), 7.66 (1H, d, J=8.4 Hz), 7.79 (1H, s), 7.8-8.0 (2H, m), 8.19 (1H, s), 12.8-13.2 (1H, br).

### EXAMPLE 118

3-[1-(2,4-Dichlorobenzyl)-2-methyl-1H-benzimidazol-6-yl]benzoic acid (78 mg) was synthesized from ethyl 3-[1-(2,4-dichlorobenzyl)-2-methyl-1H-benzimidazol-6-yl]benzoate (212 mg) in a manner similar to that described in Example 134.

¹H NMR (400 MHz, DMSO-d₆, δ/ppm): 2.46 (3H, s), 5.64 (2H, s), 6.49 (1H, d, J=8.4 Hz), 7.33 (1H, dd, J=8.4 Hz, 2.2 Hz), 7.53 (1H, dd, J=8.4 Hz, 0.9 Hz), 7.56 (1H, d, J=7.8 Hz), 7.68 (1H, d, J=8.4 Hz), 7.74 (1H, d, J=2.1 Hz), 7.83 (1H, d, J=1.3 Hz), 7.9-8.0 (2H, m), 8.20 (1H, t, J=1.6 Hz), 12.8-13.4 (br).

### EXAMPLE 119

3-(1-{[3-Chloro-5-(trifluoromethyl)-2-pyridinyl]methyl}-2-methyl-1H-benzimidazol-6-yl)benzoic acid (44 mg) was synthesized from ethyl 3-(1-{[3-chloro-5-(trifluoromethyl)-2-pyridinyl]methyl}-2-methyl-1H-benzimidazol-6-yl)benzoate (148 mg) in a manner similar to that described in Example 134.

¹H NMR (200 MHz, DMSO-d₆, δ/ppm: 2.45 (3H, s), 5.90 (2H, s), 7.48 (1H, dd, J=8.3 Hz, 1.5 Hz), 7.57 (1H, d, J=7.7 Hz), 7.64 (1H, d, J=8.3 Hz), 7.8-8.0 (3H, m), 8.19 (1H, s), 8.56 (1H, s), 8.76 (1H, s), 12.8-13.3 (1H, br).

### EXAMPLE 120

3-[1-(4-Ethoxy-2-methylbenzyl)-2-methyl-1H-benzimidazol-6-yl]benzoic acid (78 mg) was synthesized from ethyl 3-[1-(4-ethoxy-2-methylbenzyl)-2-methyl-1H-benzimidazol-6-yl]benzoate (92 mg) in a manner similar to that described in Example 134.

MS (ES+): 401 (M⁺+1).

### EXAMPLE 121

3-{1-[(2,6-Dichloro-3-pyridinyl)methyl]-2-methyl-1H-benzimidazol-6-yl}benzoic acid (156 mg) was synthesized from ethyl 3-{1-[(2,6-dichloro-3-pyridinyl)methyl]-2-methyl-1H-benzimidazol-6-yl}benzoate (208 mg) in a manner similar to that described in Example 134.

MS (ES+): 412 (M⁺+1).

### EXAMPLE 122

3-(1-{2-Chloro-4-[(ethoxycarbonyl)(methyl)amino]benzyl}-2-methyl-1H-benzimidazol-6-yl)benzoic acid (96 mg) was synthesized from ethyl 3-(1-{2-chloro-4-[(ethoxycarbonyl)(methyl)amino]benzyl}-2-methyl-1H-benzimidazol-6-yl)benzoate (177 mg) in a manner similar to that described in Example 134.

MS (ES+): 478 (M⁺+1).

### EXAMPLE 123

3-{1-[(3-Chloro-1,1′-biphenyl-4-yl)methyl]-2-methyl-1H-benzimidazol-6-yl}benzoic acid (128 mg) was synthesized from ethyl 3-{1-[(3-chloro-1,1′-biphenyl-4-yl)methyl]-2-methyl-1H-benzimidazol-6-yl}benzoate (150 mg) in a manner similar to that described in Example 134.

¹H NMR (200 MHz, DMSO-d₆, δ/ppm): 2.5 (3H, s), 5.70 (2H, s), 6.57 (1H, d, J=8.1 Hz), 7.3-7.8 (9H, m), 7.8-8.0 (4H, m), 8.21 (1H, s), 12.8-13.3 (1H, br).

### EXAMPLE 124

2-[1-(2,4-Dichlorobenzyl)-2,4-dimethyl-1H-benzimidazol-6-yl]isonicotinic acid (32 mg) was synthesized from methyl 2-[1-(2,4-dichlorobenzyl)-2,4-dimethyl-1H-benzimidazol-6-yl]isonicotinate (98 mg) in a manner similar to that described in Example 134.

¹H NMR (400 MHz, DMSO-d₆, δ/ppm): 2.47 (3H, s), 2.67 (3H, s), 5.63 (2H, s), 6.45 (1H, d, J=8.4 Hz), 7.32 (1H, dd, J=8.4 Hz, 2.1 Hz), 7.64 (1H, d, J=5.4 Hz), 7.74 (1H, d, J=2.1 Hz), 7.83 (1H, s), 8.01 (1H, s), 8.28 (1H, s), 8.68 (1H, d, J=4.8 Hz).

### EXAMPLE 125

6-[1-(2,4-Dichlorobenzyl)-2,4-dimethyl-1H-benzimidazol-6-yl]-2-pyridinecarboxylic acid (127 mg) was synthesized from 1-(2,4-dichlorobenzyl)-2,4-dimethyl-6-(4,4,5,5-tetramethyl-1,3,2-dioxaborolan-2-yl)-1H-benzimidazole (crude solution, Preparation Example 231) and 6-chloro-2-pyridinecarboxylic acid (90 mg) in a manner similar to that described in Preparation Example 242.

MS (ES+): 426 (M⁺+1).

### EXAMPLE 126

6-[1-(2,4-Dichlorobenzyl)-2,4-dimethyl-1H-benzimidazol-6-yl]nicotinic acid (41 mg) was synthesized from 1-(2,4-dichlorobenzyl)-2,4-dimethyl-6-(4,4,5,5-tetramethyl-1,3,2-dioxaborolan-2-yl)-1H-benzimidazole (crude solution, Preparation Example 231) and 6-chloronicotinic acid (90 mg) in a manner similar to that described in Preparation Example 242.

¹H NMR (400 MHz, DMSO-d₆, δ/ppm): 2.48 (3H, s), 2.62 (3H, s), 5.61 (2H, s), 6.42 (1H, d, J=8.4 Hz), 7.32 (1H, dd, J=8.4 Hz, 2.2 Hz), 7.76 (1H, d, J=2.1 Hz), 7.91 (1H, s), 8.0-8.1 (2H, m), 8.25 (1H, dd, J=8.4 Hz, 2.2 Hz), 9.07 (1H, dd, J=2.1 Hz, 0.5 Hz), 12-14 (1H, br).

### EXAMPLE 127

2-[1-(2-Chloro-4-ethoxybenzyl)-2,4-dimethyl-1H-benzimidazol-6-yl]isonicotinic acid (47 mg) was synthesized from ethyl 2-[1-(2-chloro-4-ethoxybenzyl)-2,4-dimethyl-1H-benzimidazol-6-yl]isonicotinate (82 mg) in a manner similar to that described in Example 134.

¹H NMR (200 MHz, DMSO-d₆, δ/ppm): 1.27 (3H, t, J=7.0 Hz), 2.49 (3H, s), 2.61 (3H, s), 4.00 (2H, q, J=7.0 Hz), 5.55 (2H, s), 6.47 (1H, d, J=8.6 Hz), 6.80 (1H, dd, J=8.6 Hz, 2.4 Hz), 7.11 (1H, d, J=2.4 Hz), 7.69 (1H, d, J=5.0 Hz), 7.83 (1H, s), 8.02 (1H, s), 8.32 (2H, s), 8.78 (1H, d, J=5.0 Hz), 13.2-14.3 (1H, br).

### EXAMPLE 128

6-[1-(2-Chloro-4-ethoxybenzyl)-2,4-dimethyl-1H-benzimidazol-6-yl]-2-pyridinecarboxylic acid (53 mg) was synthesized from ethyl 6-[1-(2-chloro-4-ethoxybenzyl)-2,4-dimethyl-1H-benzimidazol-6-yl]-2-pyridinecarboxylate (94 mg) in a manner similar to that described in Example 134.

MS (ES+): 436 (M⁺+1).

### EXAMPLE 129

6-[1-(2-Chloro-4-ethoxybenzyl)-2,4-dimethyl-1H-benzimidazol-6-yl]nicotinic acid (48 mg) was synthesized from methyl 6-[1-(2-chloro-4-ethoxybenzyl)-2,4-dimethyl-1H-benzimidazol-6-yl]nicotinate (55 mg) in a manner similar to that described in Example 134.

¹H NMR (200 MHz, DMSO-d₆, δ/ppm): 1.28 (3H, t, J=6.9 Hz), 2.52 (3H, s), 2.61 (3H, s), 4.00 (2H, q, J=7.0 Hz), 6.45 (1H, d, J=8.6 Hz), 6.81 (1H, dd, J=8.7 Hz, 2.5 Hz), 7.12 (1H, d, J=2.5 Hz), 7.89 (1H, s), 8.07 (1H, s), 8.09 (1H, d, J=8.5 Hz), 8.27 (1H, dd, J=8.3 Hz, 2.3 Hz), 9.08 (1H, d, J=1.8 Hz), 13.26 (1H, br s).

### EXAMPLE 130

5-[1-(2-Chloro-4-ethoxybenzyl)-2,4-dimethyl-1H-benzimidazol-6-yl]-2-thiophenecarboxylic acid (42 mg) was synthesized from ethyl 5-[1-(2-chloro-4-ethoxybenzyl)-2,4-dimethyl-1H-benzimidazol-6-yl]-2-thiophenecarboxylate (112 mg) in a manner similar to that described in Example 134.

¹H NMR (200 MHz, DMSO-d₆, δ/ppm): 1.28 (3H, t, J=6.9 Hz), 2.48 (3H, s), 2.55 (3H, s), 4.01 (2H, q, J=7.0 Hz), 5.50 (2H, s), 6.56 (1H, d, J=8.6 Hz), 6.83 (1H, dd, J=8.6 Hz, 2.5 Hz), 7.11 (1H, d, J=2.5 Hz), 7.36 (1H, s), 7.45 (1H, d, J=3.9 Hz), 7.61 (1H, s), 7.64 (1H, d, J=3.9 Hz), 12.5-13.8 (1H, br).

### EXAMPLE 131

2-(1-{[3-Chloro-5-(trifluoromethyl)-2-pyridinyl]methyl}-2,4-dimethyl-1H-benzimidazol-6-yl)isonicotinic acid (30 mg) was synthesized from ethyl 2-(1-{[3-chloro-5-(trifluoromethyl)-2-pyridinyl]methyl}-2,4-dimethyl-1H-benzimidazol-6-yl)isonicotinate (86 mg) in a manner similar to that described in Example 134.

¹H NMR (200 MHz, DMSO-d₆, δ/ppm): 2.46 (3H, S), 2.60 (3H, s), 5.90 (2H, s), 7.65 (1H, d, J=4.4 Hz), 7.80 (1H, s), 8.01 (1H, s), 8.38 (1H, s), 8.90 (1H, s), 8.71 (1H, d, J=4.5 Hz), 8.75 (1H, s), 12.8-14.0 (1H, br).

### EXAMPLE 132

6-(1-{[3-Chloro-5-(trifluoromethyl)-2-pyridinyl]methyl}-2,4-dimethyl-1H-benzimidazol-6-yl)-2-pyridinecarboxylic acid (20 mg) was synthesized from ethyl 6-(1-{[3-chloro-5-(trifluoromethyl)-2-pyridinyl]methyl}-2,4-dimethyl-1H-benzimidazol-6-yl)-2-pyridinecarboxylate (20 mg) in a manner similar to that described in Example 134.

MS (ES+): 461 (M⁺+1).

### EXAMPLE 133

4-[1-(2-Chloro-4-ethoxybenzyl)-2,4-dimethyl-1H-benzimidazol-6-yl]benzoic acid (85 mg) was synthesized from ethyl 4-[1-(2-chloro-4-ethoxybenzyl)-2,4-dimethyl-1H-benzimidazol-6-yl]benzoate (95 mg) in a manner similar to that described in Example 134.

¹H NMR (200 MHz, DMSO-d₆, δ/ppm): 1.28 (3H, t, J=6.9 Hz), 2.48 (3H, s), 2.59 (3H, s), 3.99 (2H, q, J=7.0 Hz), 5.52 (2H, s), 6.49 (1H, d, J=8.6 Hz), 6.82 (1H, dd, J=8.6 Hz, 2.5 Hz), 7.10 (1H, d, J=2.5 Hz), 7.39 (1H, s), 7.63 (1H, s), 7.78 (2H, d, J=8.5 Hz), 7.97 (2H, d, J=8.4 Hz), 12.4-13.2 (1H, br).

### EXAMPLE 134

An aqueous solution of sodium hydroxide (1N, 0.94 mL) was added to a suspension of ethyl 4-(1-{[3-chloro-5-(trifluoromethyl)-2-pyridinyl]methyl}-2,4-dimethyl-1H-benzimidazol-6-yl)benzoate (92 mg) in 1,4-dioxane at room temperature, and the mixture was heated at 80° C. for 30 minutes. It was cooled to room temperature and neutralized with 1N hydrochloric acid. The product was extracted with chloroform, and the organic layer was washed with water and brine and dried over sodium sulfate. The crude product was purified over preparative thin layer chromatography (chloroform:methanol=10:1) to give 4-(1-{[3-chloro-5-(trifluoromethyl)-2-pyridinyl]methyl}-2,4-dimethyl-1H-benzimidazol-6-yl)benzoic acid (52 mg) as a powder.

¹H NMR (200 MHz, DMSO-d₆, δ/ppm): 2.45 (3H, s), 2.59 (3H, s), 5.86 (2H, s), 7.37 (1H, s), 7.67 (1H, s), 7.77 (2H, d, J=8.5 Hz), 7.96 (2H, d,J=8.4 Hz), 8.56 (1H, s), 8.75 (1H, s).

### EXAMPLE 135

{3-[1-(2,4-Dichlorobenzyl)-2,4-dimethyl-1H-benzimidazol-6-yl]phenoxy}acetic acid (93 mg) was synthesized from ethyl {3-[1-(2,4-dichlorobenzyl)-2,4-dimethyl-1H-benzimidazol-6-yl]phenoxy}acetate (130 mg) in a manner similar to that described in Example 134.

¹H NMR (200 MHz, DMSO-d₆, δ/ppm): 2.46 (3H, s), 2.58 (3H, s), 4.72 (2H, s), 5.58 (2H, s), 6.48 (1H, d, J=8.3 Hz), 6.84 (1H, br d, J=7.6 Hz), 7.1-7.4 (5H, m), 7.56 (1H, s), 7.73 (1H, d, J=2.0 Hz).

### EXAMPLE 136

3-{1-[2-Chloro-4-(pentyloxy)benzyl]-2-ethoxy-4-methyl-1H-benzimidazol-6-yl}benzoic acid (117 mg) was synthesized from ethyl 3-{1-[2-chloro-4-(pentyloxy)benzyl]-2-ethoxy-4-methyl-1H-benzimidazol-6-yl}benzoate (150 mg) in a manner similar to that described in Example 134.

¹H NMR (200 MHz, DMSO-d₆, δ/ppm): 0.86 (3H, t, J=7.0 Hz), 1.2-1.5 (7H, m), 1.66 (2H, quint, J=6.6 Hz), 2.52 (3H, s), 3.93 (2H, t, J=6.4 Hz), 4.56 (2H, q, J=7.0 Hz), 5.30 (2H, s), 6.8-6.9 (2H, m), 7.06 (1H, d, J=1.6 Hz), 7.28 (1H, s), 7.45 (1H, s), 7.54 (1H, t, J=7.7 Hz), 7.86 (2H, d, J=7.9 Hz), 8.15 (1H, s), 12.7-13.5 (1H, br).

### EXAMPLE 137

A mixture of 6-bromo-1-[2-chloro-4-(pentyloxy)benzyl]-2-ethoxy-4-methyl-1H-benzimidazol (400 mg), 4-carboxyphenylboronic acid (214 mg), palladium(II) acetate (Pd(OAc)₂) (19.3 mg), triphenylphosphine (PPh₃) (90.1 mg), K₂CO₃ (356 mg), 1,2-dimethoxyethane (DME) (8 mL) and water (H₂O) (1.6 mL) was evacuated and backfield with nitrogen (N₂) three times, then degassed with nitrogen (N₂) for 10 minutes. The mixture was heated at reflux for 3 hours. After cooling to room temperature, the mixture was poured into water (20 mL). The phases were separated and the aqueous layer was extracted with 2×10 mL of ethyl acetate. The combined extracts were dried over Na₂SO₄ and concentrated under reduced pressure. Purification by chromatography on silica gel (CHCl₃/hexane 1/20) gave 4-{1-[2-chloro-4-(pentyloxy)benzyl]-2-ethoxy-4-methyl-1H-benzimidazol-6-yl}benzoic acid. Recrystallization from EtOAc-hexane afforded a colorless solid (48 mg).

¹H NMR (200 MHz, DMSO-d₆, δ): 0.86 (3H, t, J=7.0 Hz), 1.24-1.40 (7H, m), 1.62-1.69 (2H, m), 2.49 (3H, s), 3.93 (2H, t, J=6.4 Hz), 4.57 (2H, q, J=7.0 Hz), 5.30 (2H, s), 6.87 (2H, s), 7.07 (1H, s), 7.34 (1H, s), 7.49 (1H, s), 7.74 (2H, d, J=8.4 Hz), 7.97 (2H, d, J=8.4 Hz), 12.9(1H, br).

MS: 507 (M+1).

### PREPARATION EXAMPLE 247

To a mixture of N-(4-hydroxy-2-nitrophenyl)acetamide (4.0 g), ethyl 4-bromo-2,2-dimethylbutanoate (11.4 g) and DMF (20 ml) was added K₂CO₃ (7.1 g) at ambient temperature. After stirring for 5 hours at 80° C., the reaction mixture was diluted with EtOAc (100 ml) and washed with water (100 ml) and brine (2×100 ml). The organic layer was dried over MgSO₄ and filtered. Evaporation gave a residue which was chromatographed (silica gel (120 g) EtOAc/n-hexane=1/5-2/1), and triturated with n-hexane (10 ml) to give ethyl 4-[4-(acetylamino)-3-nitrophenoxy]-2,2-dimethylbutanoate (6.93 g) as pale brown crystals.

NMR(DMSO-d₆,δ): 1.16 (3H, t, J=7.2 Hz), 1.19 (6H, s), 1.99 (2H, t, J=6.7 Hz), 2.01 (3H, s), 3.9-4.2 (4H, m), 7.22 (1H, dd, J=2.9 Hz, 8.7 Hz), 7.3-7.5 (2H, m), 10.05 (1H, br s).

MS: 361 (M+Na).

### PREPARATION EXAMPLE 248

To a solution of ethyl 4-[4-(acetylamino)-3-nitrophenoxy]-2,2-dimethylbutanoate (6.8 g) in a mixture of EtOH (20 ml) and THF (14 ml) was added palladium on carbon (10%, 50% wet, 1.4 g) at ambient temperature, and the resultant mixture was hydrogenated under atmospheric pressure of hydrogen for 6 hours. The catalyst was removed by filtration. Evaporation gave a residue which was triturated with EtOAc (2.5 ml)—n-hexane (7 ml) to give ethyl 4-[4-(acetylamino)-3-aminophenoxy]-2,2-dimethylbutanoate (4.9 g) as crystals.

NMR(DMSO-d₆,δ): 1.1-1.3 (9H, m), 1.94 (2H, t, J=6.7 Hz), 1.98 (3H, s), 3.86 (2H, t, J=6.7 Hz), 4.06 (2H, q, J=7.1 Hz), 4.84 (2H, br s), 6.06 (1H, dd, J=2.7 Hz, 8.6 Hz), 6.23 (1H, d, J=2.7 Hz), 6.93 (1H, d, J=8.6 Hz), 8.96 (1H, br s).

MS: 331 (M+Na).

### PREPARATION EXAMPLE 249

A mixture of ethyl 4-[4-(acetylamino)-3-aminophenoxy]-2,2-dimethylbutanoate (200 mg), 2,4-dichloro-1-(chloromethyl)benzene (190 mg), K₂CO₃ (152 mg) and DMF (1 ml) was heated at 80° C. for 5 hours. After cooling, the reaction mixture was poured into water (30 ml) and extracted with EtOAc (3×30 ml). The combined organic layers were washed with brine (2×30 ml). The organic layer was dried over MgSO₄, filtered, and evaporated to give ethyl 4-{4-(acetylamino)-3-[(2,4-dichlorobenzyl)amino]phenoxy}-2,2-dimethylbutanoate (368 mg) as a crude oil.

### PREPARATION EXAMPLE 250

A mixture of ethyl 4-[4-(acetylamino)-3-aminophenoxy]-2,2-dimethylbutanoate (1.0 g), 1-(bromomethyl)-2-chloro-4-(pentyloxy)benzene (1.32 g), K₂CO₃ (672 mg) and DMF (5 ml) was heated at 80° C. for 2 hours. After cooling, the reaction mixture was poured into water (40 ml) and extracted with EtOAc (3×30ml). The combined organic layers were washed with brine (2×30ml). The organic layer was dried over MgSO₄, filtered, and evaporated to give ethyl 4-(4-(acetylamino)-3-{[2-chloro-4-(pentyloxy)benzyl]amino}phenoxy)-2,2-dimethylbutanoate (2.17 g) as a crude oil.

The following compound was obtained in a similar manner to that of Preparation Example 250.

### PREPARATION EXAMPLE 251

4-(acetylamino)-3-{[(3-chloro-1,1′-biphenyl-4-yl)methyl]amino}-5-methylphenyl acetate

NMR(DMSO-d₆,δ): 2.05 (3H, s), 2.08 (3H, s), 2.14 (3H, s), 4.38 (2H, d, J=6 Hz), 5.9-6.0 (2H, m), 6.1-6.2 (1H, m), 7.3-7.8 (8H, m), 8.97 (1H, s)

MS: 421 (M−H).

### PREPARATION EXAMPLE 252

A mixture of 5,6-dichloro-3-pyridinol (600 mg, 3.7 mmol), K₂CO₃ (1.52 g, 11 mmol), 1-iodopentane (1.24 g, 7.3 mmol) and DMF (6 mL) was stirred at room temperature for 3 hours. The mixture was diluted with EtOAc (20 mL) and washed with water (30 mL) and brine (2×30 mL). The organic layer was dried (Na₂SO₄) and filtered. The residue was purified by column chromatography (EtOAc/hexane 1/25) to give 2,3-dichloro-5-(pentyloxy)pyridine (580 mg) as a colorless oil.

NMR (CDCl₃, 200 MHz): 0.94 (3H, t, J=6.9 Hz), 1.29-1.50 (4H, m), 1.73-1.87 (2H, m), 3.99 (2H, t, J=6.4 Hz), 7.32 (1H, d, J=2.8 Hz), 7.99(1H, d, J=2.8 Hz).

MS: 234 (M+1).

### PREPARATION EXAMPLE 253

To a mixture (degassed under reduced pressure) of 2,3-dichloro-5-(pentyloxy)pyridine (860 mg), 1,3-bis(diphenylphosphino)propane (485 mg), triethylamine (1.54 mL) and EtOH (8.6 ml) was added palladium(II) acetate (264 mg) at ambient temperature. The mixture was stirred under atmospheric pressure of carbon monoxide at 70° C. for 28 hours. After cooling, the reaction mixture was evaporated and to the residue was added isopropyl ether (50 ml). The resulting precipitates were removed off by filtration and the filtrate was evaporated. The residue was purified by column chromatography (silica gel EtOAc/n-hexane=1/10) to give ethyl 3-chloro-5-(pentyloxy)-2-pyridinecarboxylate (675 mg) as a pale yellow oil.

NMR (CDCl₃, 200 MHz, δ): 0.94 (3H, t, J=7.0 Hz), 1.30-1.53 (7H, m), 1.76-1.89 (2H, m), 4.04 (2H, t, J=6.5 Hz), 4.46 (2H, q, J=7.0 Hz), 7.25 (1H, d, J=2.5 Hz), 8.27 (1H, d, J=2.5 Hz).

MS: 294(M+Na).

### PREPARATION EXAMPLE 254

To a solution of ethyl 3-chloro-5-(pentyloxy)-2-pyridinecarboxylate (850 mg) in EtOH (8.5 mL) was added sodium borohydride (473 mg) at 0° C. After stirring for 2 hours, the mixture was allowed to warm to room temperature, and stirred for 2 hours. The mixture was diluted with EtOAc (50 mL) and poured into water (50 mL). The phases were separated and the aqueous layer was extracted with 2×20 mL of EtOAc. The combined extracts were washed with NaHCO₃ (20 mL) and brine (20 mL), dried over Na₂SO₄, and concentrated. The residue was purified by column chromatography (silica gel, EtOAc/hexane=1/2) to give [3-chloro-5-(pentyloxy)-2-pyridinyl]methanol (700 mg) as a colorless oil.

NMR (CDCl₃, 200 MHz, δ): 0.94 (3H, t, J=6.9 Hz), 1.37-1.48 (4H, m), 1.74-1.88 (2H, m), 3.37-4.07 (3H, m), 4.73 (2H, d, J=4.7 Hz), 7.25 (1H, d, J=2.4 Hz), 8.17(1H, d, J=2.4 Hz).

MS: 252 (M+Na).

### PREPARATION EXAMPLE 255

To a solution of [3-chloro-5-(pentyloxy)-2-pyridinyl]methanol (0.6 g) in dichloromethane (7 mL) were added triethylamine (0.583 mL) and then methanesulfonyl chloride (283 uL) dropwise at 0° C. After stirring for 30 minutes, the reaction mixture was diluted with EtOAc (30 mL) and washed with NaHCO₃ (2×20 mL) and brine (30 mL). The organic layer was dried (Na₂SO₄), filtered and evaporated to give [3-chloro-5-(pentyloxy)-2-pyridinyl]methyl methanesulfonate (767 mg) as a dark red oil.

NMR (CDCl₃, 200 MHz, δ): 0.94 (3H, t, J=6.8 Hz), 1.37-1.48 (4H, m), 1.49-2.05 (2H, m), 3.08 (3H, s), 4.02 (2H, t, J=6.4 Hz), 5.41 (2H, s), 7.25 (1H, d, J=2.5 Hz), 8.22 (1H, d, J=2.5 Hz).

MS: 330 (M+Na).

### PREPARATION EXAMPLE 256

A mixture of ethyl 4-[4-(acetylamino)-3-aminophenoxy]-2,2-dimethylbutanoate (500 mg), [3-chloro-5-(trifluoromethyl)-2-pyridinyl]methyl methanesulfonate (564 mg), K₂CO₃ (269 mg), NaI (292 mg) and DMF (5 ml) was stirred at ambient temperature for 3 hours. To the reaction mixture was added water (30 ml) and the precipitates were collected by filtration and washed with water to give ethyl 4-[4-(acetylamino)-3-({[3-chloro-5-(trifluoromethyl)-2-pyridinyl]methyl}amino)phenoxy]-2,2-dimethylbutanoate as crude crystals. The crude crystals were triturated with EtOAc (3 ml)—n-hexane (3 ml) to give pure ethyl 4-[4-(acetylamino)-3-({[3-chloro-5-(trifluoromethyl)-2-pyridinyl]methyl}amino)phenoxy]-2,2-dimethylbutanoate (591 mg) as white crystals.

NMR(DMSO-d₆,δ): 1.13 (3H, t, J=7.1 Hz), 1.15 (6H, s), 1.92 (2H, t, J=6.6 Hz), 1.99 (3H, s), 3.88 (2H, t, J=6.6 Hz), 4.02 (2H, q, J=7.1 Hz), 4.55 (2H, d, J=5.4 Hz), 5.79 (1H, t, J=5.4 Hz), 6.0-6.2 (2H, m), 6.94 (1H, d, J=9.2 Hz), 8.50 (1H, d, J=1.5 Hz), 8.94 (1H, br s), 9.09 (1H, br s).

MS: 524 (M+Na).

The following compound was obtained in a similar manner to that of Example 139.

### PREPARATION EXAMPLE 257

1-[(3-chloro-1,1′-biphenyl-4-yl)methyl]-2,4-dimethyl-1H-benzimidazol-6-ol

NMR(DMSO-d₆,δ): 2.43 (3H, s), 2.45 (3H, s), 5.41 (2H, s), 6.4-6.5 (2H, m), 6.54 (1H, d, J=8 Hz), 7.3-7.7 (6H, m), 7.81 (1H, d, J=2 Hz), 9.04 (1H, s).

MS: 363 (M+H).

### EXAMPLE 138

To a solution of ethyl 4-{4-(acetylamino)-3-[(2,4-dichlorobenzyl)amino]phenoxy}-2,2-dimethylbutanoate (368 mg: crude) in EtOH (2 ml) was added H₂SO₄ (154 mg) at ambient temperature. After stirring for 12 hours at room temperature, the reaction mixture was heated at 80° C. for 3 hours. After cooling, the reaction mixture was alkalized with saturated NaHCO₃ (20 ml), and extracted with EtOAc (2×20 ml). The combined organic layers were washed with brine (20 ml), dried over MgSO₄, and filtered. Evaporation gave a residue (276 mg) which was chromatographed (silica gel (3 g) EtOAc/n-hexane=2/1) to give ethyl 4-{[1-(2,4-dichlorobenzyl)-2-methyl-1H-benzimidazol-6-yl]oxy}-2,2-dimethylbutanoate (141 mg) as an oil.

NMR(DMSO-d₆,δ): 1.10 (3H, t, J=7.1 Hz), 1.16 (6H, s), 1.94 (2H, t, J=6.7 Hz), 2.39 (3H, s), 3.92 (2H, t, J=6.7 Hz), 4.02 (2H, q, J=7.1 Hz), 5.48 (2H, br s), 6.47 (1H, d, J=8.3 Hz), 6.72 (1H, dd, J=2.2 Hz, 8.6 Hz), 6.94 (1H, d, J=2.2 Hz), 7.33 (1H, dd, J=2.1 Hz, 8.3 Hz), 7.43 (1H, d, J=8.6 Hz), 7.73 (1H, d, J=2.1 Hz).

MS: 449 (M+1).

### EXAMPLE 139

To a solution of ethyl 4-(4-(acetylamino)-3-{[2-chloro-4-(pentyloxy)benzyl]amino}phenoxy)-2,2-dimethylbutanoate (2.17 g: crude) in EtOH (10 ml) was added H₂SO₄ (820 mg) at ambient temperature. After stirring for 12 hours at room temperature, the reaction mixture was heated at 80° C. for 3 hours. After cooling, the reaction mixture was alkalized with saturated NaHCO₃ (30 ml), and extracted with EtOAc (2×30 ml). The combined organic layers were washed with brine (30 ml), dried over MgSO₄, and filtered. Evaporation gave a residue (1.94 g) which was chromatographed (silica gel (20 g) EtOAc/n-hexane=2/1) to give ethyl 4-({1-[2-chloro-4-(pentyloxy)benzyl]-2-methyl-1H-benzimidazol-6-yl}oxy)-2,2-dimethylbutanoate (876 mg) as an oil.

NMR(DMSO-d₆,δ): 0.8-1.0 (3H, m), 1.11 (3H, t, J=7.1 Hz), 1.16 (6H, s), 1.2-1.5 (4H, m), 1.6-1.8 (2H, m), 1.94 (2H, t, J=6.7 Hz), 2.40 (3H, s), 3.8-4.1 (6H, m), 5.40 (2H, br s), 6.52 (1H, d, J=8.6 Hz), 6.70 (1H, dd, J=2.3 Hz, 8.7 Hz), 6.82 (1H, dd, J=2.5 Hz, 8.6 Hz), 6.90 (1H, d, J=2.3 Hz), 7.10 (1H, d, J=2.5 Hz), 7.41 (1H, d, J=8.7 Hz).

MS: 501 (M+1).

### EXAMPLE 140

To a suspension of ethyl 4-[4-(acetylamino)-3-({[3-chloro-5-(trifluoromethyl)-2-pyridinyl]methyl}amino)phenoxy]-2,2-dimethylbutanoate (550 mg) in EtOH (2.75 ml) was added H₂SO₄ (215 mg) at ambient temperature. The reaction mixture was heated at 80° C. for 1 hour. After cooling, to the reaction mixture was added ice and the mixture was alkalized with 20%-NaOH (pH˜8), and the mixture was extracted with EtOAc (30 ml). The organic extract was washed with brine (30 ml), dried over MgSO₄, and filtered. Evaporation gave a residue which was triturated with n-hexane (5 ml) to give ethyl 4-[(1-{[3-chloro-5-(trifluoromethyl)-2-pyridinyl]methyl}-2-methyl-1H-benzimidazol-6-yl)oxy]-2,2-dimethylbutanoate (478 mg) as white crystals.

NMR(DMSO-d₆,δ): 1.10 (3H, t, J=7.1 Hz), 1.16 (6H, s), 1.94 (2H, t, J=6.6 Hz), 2.39 (3H, s), 3.91 (2H, t, J=6.6 Hz), 4.02 (2H, q, J=7.1 Hz), 5.73 (2H, br s), 6.68 (1H, dd, J=2.3 Hz, 8.6 Hz), 6.92 (1H, d, J=2.3 Hz), 7.38 (1H, d, J=8.6 Hz), 8.57 (1H, d, J=1.4 Hz), 8.78 (1H, d, J=0.9 Hz).

MS: 484 (M+1).

### EXAMPLE 141

To a 0° C. solution of 1-[(3-chloro-1,1′-biphenyl-4-yl)methyl]-2,4-dimethyl-1H-benzimidazol-6-ol (200 mg) in DMF (1.5 mL)-tetrahydrofuran (THF) (0.75 mL) was added sodium hydride (60% dispersion in mineral oil, 26.5 mg). After 1 hour at room temperature, the mixture was treated with a solution of methyl 2-(bromomethyl)nicotinate (165 mg) in DMF (0.5 mL). The resultant mixture was stirred for 4 hours at that temperature before the reaction was quenched by addition of water (20 mL). The mixture was extracted with EtOAc (2×20 mL). The combined organic layers ware washed with brine (1×25 mL), dried over Na₂SO₄, and concentrated. Purification by chromatography on silica gel (EtOAc/Hexane 2/1) gave methyl 2-[({1-[(3-chloro-1,1′-biphenyl-4-yl)methyl]-2,4-dimethyl-1H-benzimidazol-6-yl}oxy)methyl]nicotinate (223 mg) as a white solid.

NMR (DMSO-d₆, 200 MHz, δ): 8.66(1H, d, J=4.9 Hz), 8.13 (1H, d, 7.7 Hz), 7.84 (1H, S), 7.70-7.65 (2H, m), 7.54-7.41 (5H, m), 6.84 (1H, s), 6.63 (1H, s), 6.50 (1H, d, 8.1 Hz), 5.49 (2H, s) 5.33 (2H, s), 3.71 (3H, s), 2.48 (3H, s), 2.45 (3H, s).

MS: 512 (M+1).

The following compounds were obtained in a similar manner to that of Example 141.

### EXAMPLE 142

methyl 4-[({1-[(3-chloro-1,1′-biphenyl-4-yl)methyl]-2,4-dimethyl-1H-benzimidazol-6-yl}oxy)methyl]benzoate

NMR(DMSO-d₆,δ): 2.43 (3H, s), 2.49 (3H, s), 3.80 (3H, s), 5.14 (2H, s), 5.49 (2H, s), 6.47 (1H, d, J=8 Hz), 6.74 (1H, d, J=1 Hz), 6.94 (1H, d, J=2 Hz), 7.4-7.9 (1OH, m), 7.92 (1H, d, J=8 Hz).

MS: 511 (M+H).

### EXAMPLE 143

methyl 2-[({1-[(3-chloro-1,1′-biphenyl-4-yl)methyl]-2,4-dimethyl-1H-benzimidazol-6-yl}oxy)methyl]-6-methylbenzoate

NMR(DMSO-d₆,δ): 2.24 (3H, s), 2.44 (3H, s), 2.48 (3H, s), 3.66 (3H, s), 5.01 (2H, s), 5.50 (2H, s), 6.48 (1H, d, J=8 Hz), 6.61 (1H, d, J=1 Hz), 6.86 (1H, d, J=2 Hz), 7.2-7.7 (9H, m), 7.84 (1H, d, J=2 Hz).

MS: 525 (M+H).

### EXAMPLE 144

methyl 2-[({1-[(3-chloro-1,1′-biphenyl-4-yl)methyl]-2,4-dimethyl-1H-benzimidazol-6-yl}oxy)methyl]-6-nitrobenzoate

NMR(DMSO-d₆,δ): 2.44 (3H, s), 2.49 (3H, s), 3.72 (3H, s), 5.14 (2H, s), 5.50 (2H, s), 6.46 (1H, d, J=8 Hz), 6.65 (1H, d, J=1 Hz), 6.87 (1H, d, J=2 Hz), 7.4-8.1 (10H, m).

MS: 556 (M+H).

### EXAMPLE 145

methyl 3-chloro-2-[({1-[(3-chloro-1,1′-biphenyl-4-yl)methyl]-2,4-dimethyl-1H-benzimidazol-6-yl}oxy)methyl]benzoate

NMR(DMSO-d₆,δ): 2.44 (3H, s), 2.4-2.6 (3H, s), 3.67 (3H, s), 5.29 (2H, s), 5.52 (2H, s), 6.48 (1H, d, J=8 Hz), 6.62 (1H, d, J=1 Hz), 6.91 (1H, d, J=2-Hz), 7.3-7.7 (9H, m), 7.85 (1H, d, J=2 Hz).

MS: 545, 547 (M+H).

### EXAMPLE 146

methyl 4-chloro-2-[({1-[(3-chloro-1,1′-biphenyl-4-yl)methyl]-2,4-dimethyl-1H-benzimidazol-6-yl}oxy)methyl]benzoate

NMR(DMSO-d₆,δ): 2.45 (3H, s), 2.4-2.6 (3H, s), 3.77 (3H, s), 5.36 (2H, s), 5.49 (2H, s), 6.54 (1H, d, J=8 Hz), 6.73 (1H, br s), 6.85 (1H, d, J=2 Hz), 7.4-7.7 (8H, m), 7.80 (1H, d, J=2 Hz), 7.88 (1H, d, J=8 Hz).

MS: 545, 547 (M+H).

### EXAMPLE 147

ethyl 5-chloro-2-[({(1-[(3-chloro-1,1′-biphenyl-4-yl)methyl]-2,4-dimethyl-1H-benzimidazol-6-yl}oxy)methyl]benzoate

NMR(DMSO-d₆,δ): 2.45 (3H, s), 2.4-2.6 (3H, s), 3.76 (3H, s), 5.33 (2H, s), 5.49 (2H, s), 6.52 (1H, d, J=8 Hz), 6.69 (1H, d, J=2 Hz), 6.8-6.9 (1H, m), 7.4-8.0 (10H, m).

MS: 545, 547 (M+H).

### EXAMPLE 148

methyl 2-[({1-[(3-chloro-1,1′-biphenyl-4-yl)methyl]-2,4-dimethyl-1H-benzimidazol-6-yl}oxy)methyl]-6-fluorobenzoate

NMR(DMSO-d₆,δ): 2.44 (3H, s), 2.4-2.6 (3H, s), 3.70 (3H, s), 5.12 (2H, s), 5.50 (2H, s), 6.49 (1H, d, J=8 Hz), 6.63 (1H, d, J=1 Hz), 6.87 (1H, d, J=2 Hz), 7.3-7.6 (7H, m), 7.66 (1H, dd, J=1 Hz, J=8 Hz), 7.83 (1H, d, J=2 Hz).

MS: 529 (M+H)

### EXAMPLE 149

methyl 2-chloro-6-[({1-[(3-chloro-1,1′-biphenyl-4-yl)methyl]-2,4-dimethyl-1H-benzimidazol-6-yl}oxy)methyl]benzoate

NMR(DMSO-d₆,δ): 2.44 (3H, s), 2.4-2.6 (3H, s), 3.71 (3H, s), 5.04 (2H, s), 5.50 (2H, s), 6.48 (1H, d, J=8 Hz), 6.63 (1H, d, J=1 Hz), 6.87 (1H, d, J=2 Hz), 7.3-7.7 (9H, m), 7.84 (1H, d, J=2 Hz).

MS: 545, 547 (M+H).

### EXAMPLE 150

A mixture of ethyl 4-[(2,4-dimethyl-1H-benzimidazol-6-yl)oxy]butanoate (200 mg), K₂CO₃ (160 mg), [3-chloro-5-(pentyloxy)-2-pyridinyl]methyl methanesulfonate (334 mg) and DMF (4 mL) was heated at 80° C. for 3 hours. After cooling to room temperature, the reaction mixture was poured into aqueous NH₄Cl (20 mL), then extracted with EtOAc (20 mL×3). The combined organic layer was dried and concentrated. The residue was purified by column chromatography (EtOAc/hexane 1/4) to give ethyl 4-[(1-{[3-chloro-5-(pentyloxy)-2-pyridinyl]methyl}-2,4-dimethyl-1H-benzimidazol-6-yl)oxy]butanoate as a colorless oil.

NMR (CDCl₃, 200 MHz, δ): 0.91 (3H, t, J=6.9 Hz), 1.24 (3H, t, J=7.1 Hz), 1.26-1.50 (4H, m), 1.61-1.71 (2H, m), 1.65-1.85 (2H, m), 2.05-2.15 (2H, m), 2.50 (2H, t, J=7.3 Hz), 2.59 (3H, s), 2.65 (3H, s), 3.82-4.00 (4H, m), 4.13 (2H, q, J=7.1 Hz), 5.36 (2H, s), 6.63 (2H, s), 7.20 (1H, d, J=2.5 Hz), 8.08 (1H, d, J=2.5 Hz).

MS: 488 (M+1).

The following compounds were obtained in a similar manner to that of Example 150.

### EXAMPLE 151

ethyl 4-{[1-(2-bromo-4-ethoxybenzyl)-2,4-dimethyl-1H-benzimidazol-6-yl]oxy}butanoate

NMR(CDCl₃,δ): 1.24 (3H, t, J=7.1 Hz), 1.38 (3H, t, J=7.0 Hz), 2.05-2.11 (2H, m), 2.48-2.52 (5H, m), 2.63 (3H, s), 3.95-3.99 (4H, m), 4.13 (2H, q, J=7.1 Hz), 5.21 (2H, s), 6.31 (1H, d, J=8.6 Hz), 6.47 (1H, d, J=2.2 Hz), 6.64 (1H, dd, J=2.5 Hz and 8.6 Hz), 6.69 (1H, m), 7.17 (1H, d, J=2.5 Hz).

### EXAMPLE 152

ethyl 4-{[6-(4-ethoxy-4-oxobutoxy)-2,4-dimethyl-1H-benzimidazol-1-yl]methyl}-3-methoxybenzoate

NMR(CDCl₃,δ): 1.23 (3H, t, J=7.1 Hz), 1.37 (3H, t, J=7.1 Hz), 2.04-2.10 (2H, m), 2.49 (2H, t, J=7.3 Hz), 2.52 (3H, s), 2.63 (3H, s), 3.94 (2H, t, J=6.1 Hz), 3.98 (3H, s), 4.12 (2H, q, J=7.1 Hz), 4.36 (2H, q, J=7.1 Hz), 5.25 (2H, s), 6.46 (1H, d, J=2.1 Hz), 6.52 (1H, d, J=7.9 Hz), 6.68 (1H, J=1.6 Hz), 7.48 (1H, dd, J=1.2 Hz and 7.9 Hz), 7.58 (1H, d, J=1.2 Hz).

### EXAMPLE 153

ethyl 4-({1-[2-fluoro-4-(trifluoromethyl)benzyl]-2,4-dimethyl-1H-benzimidazol-6-yl}oxy)butanoate

NMR(CDCl₃,δ): 1.24 (3H, t, J=7.1 Hz), 2.07-2.10 (2H, m), 2.50 (2H, t, J=7.3 Hz), 2.55 (3H, s), 2.63 (3H, s), 3.97 (2H, t, J=6.1 Hz), 4.12 (2H, q, J=7.1 Hz), 5.34 (2H, s), 6.49 (1H, m), 6.70-6.71 (1H, m), 6.73-6.76 (1H, m), 7.27-7.28 (1H, m), 7.39-7.42 (1H, m).

### EXAMPLE 154

ethyl 4-({1-[2-chloro-4-(1H-pyrazol-1-yl)benzyl]-2,4-dimethyl-1H-benzimidazol-6-yl}oxy)butanoate

NMR(CDCl₃,δ): 1.23 (3H, t, J=7.1 Hz), 2.06-2.10 (2H, m), 2.49 (2H, t, J=7.2 Hz), 2.54 (3H, s), 2.65 (3H, s), 3.96 (2H, t, J=6.1 Hz), 4.12 (2H, q, J=7.1 Hz), 5.33 (2H, s), 6.46-6.48 (2H, m), 6.51 (1H, d, J=8.5 Hz), 6.71 (1H, m), 7.37 (1H, dd, J=2.2 Hz and 8.5 Hz), 7.71 (1H, m), 7.86 (1H, d, J=2.5 Hz), 7.91 (1H, d, J=2.2 Hz).

### EXAMPLE 155

ethyl 4-({1-[4-ethoxy-2-(trifluoromethyl)benzyl]-2,4-dimethyl-1H-benzimidazol-6-yl}oxy)butanoate

NMR(CDCl₃,δ): 1.24 (3H, t, J=7.1 Hz), 1.40 (3H, t, J=7.0 Hz), 2.06-2.10 (2H, m), 2.49 (3H, s), 2.49 (2H, t, J=7.1 Hz), 2.64 (3H, s), 3.95 (2H, t, J=6.1 Hz), 4.01 (2H, q, J=7.0 Hz), 4.12 (2H, q, J=7.1 Hz), 5.36 (2H, s), 6.40 (1H, d, J=8.6 Hz), 6.45 (1H, d, J=2.2 Hz), 6.69-6.70 (1H, m), 6.80 (1H, dd, J=2.6 Hz and 8.7 Hz), 7.25 (1H, d, J=2.6 Hz).

### EXAMPLE 156

ethyl 4-({1-[2-chloro-4-(2-pyridinyl)benzyl]-2,4-dimethyl-1H-benzimidazol-6-yl}oxy)butanoate

NMR(CDCl₃,δ): 1.22 (3H, t, J=7.2 Hz), 2.06-2.10 (2H, m), 2.49 (2H, t, J=7.3 Hz), 2.55 (3H, s), 2.65 (3H, s), 3.96 (2H, q, J=7.1 Hz), 4.11 (2H, q, J=7.1 Hz), 5.37 (2H, s), 6.49 (1H, d, J=2.1 Hz), 6.54 (1H, d, J=8.1 Hz), 6.71 (1H, d, J=1.4 Hz), 7.25-7.27 (1H, m), 7.65-7.67 (2H, m), 7.75 (1H, td, J=6.0 Hz and 1.8 Hz), 8.16 (1H, d, J=1.8 Hz), 8.67-8.69 (1H, m).

### EXAMPLE 157

ethyl 4-{[1-(2-chloro-4-ethylbenzyl)-2,4-dimethyl-1H-benzimidazol-6-yl]oxy}butanoate

NMR(CDCl₃,δ): 1.20 (3H, t, J=7.6 Hz), 1.24 (3H, t, J=7.1 Hz), 2.05-2.10 (2H, m), 2.50 (2H, t, J=7.2 Hz), 2.52 (3H, s), 2.59 (2H, q, J=7.6 Hz), 2.64 (3H, s), 3.95 (2H, t, J=6.1 Hz), 4.12 (2H, q, J=7.1 Hz), 5.28 (2H, s), 6.35 (1H, d, J=7.9 Hz), 6.47 (1H, d, J=2.1 Hz), 6.69 (1H, d, J=1.4 Hz), 6.90 (1H, d, J=7.9 Hz), 7.28 (1H, d, J=1.3 Hz).

### EXAMPLE 158

A mixture of methyl 2-{[(2,4-dimethyl-1H-benzimidazol-6-yl)oxy]methyl}benzoate (0.57 g), K₂CO₃ (0.508 g), 4-(bromomethyl)-3-chlorobenzonitrile (0.508 g), n-Bu₄NI (67.8 mg) and DMF (3.7mL) was stirred at ambient temperature for 2 days. The mixture was partitioned between EtOAc and H₂O, and the organic layer was separated, washed with brine, dried over MgSO₄, and concentrated in vacuo. The residue was purified by recrystallization from EtOH (5 mL) to give methyl 2-({[1-(2-chloro-4-cyanobenzyl)-2,4-dimethyl-1H-benzimidazol-6-yl]oxy}methyl)benzoate (0.32 g).

NMR(DMSO-d₆,δ): 2.40 (3H, s), 2.4-2.6 (3H, s), 3.77 (3H, s), 5.32 (2H, s), 5.52 (2H, s), 6.52-(1H, d, J=8 Hz), 6.69 (1H, br s), 6.77 (1H, d, J=2 Hz), 7.4-7.7 (4H, m), 7.86 (1H, d, J=7 Hz), 8.13 (1H, d, J=1 Hz).

MS: 460 (M+H).

The following compound was obtained in a similar manner to that of Example 158.

### EXAMPLE 159

methyl 2-({[1-(4-cyano-2-fluorobenzyl)-2,4-dimethyl-1H-benzimidazol-6-yl]oxy}methyl)benzoate

The following compounds were obtained in a similar manner to that of Preparation Example 153 and Preparation Example 154.

### EXAMPLE 160

ethyl 2-[({1-[2-chloro-4-(pentyloxy)benzyl]-2,4-dimethyl-1H-benzimidazol-6-yl}oxy)methyl]benzoate

NMR(CDCl₃,δ): 0.92 (3H, t, J=7.2 Hz), 1.34 (3H, t, J=7.1 Hz), 1.38 (4H, m), 1.75 (2H, m), 2.52 (3H, s), 2.64 (3H, s), 3.89 (2H, t, J=6.5 Hz), 4.33 (2H, q, J=7.1 Hz), 5.24 (2H, s), 5.44 (2H, s), 6.38 (1H, d, J=8.6 Hz), 6.58 (2H, m), 6.81 (1H, d, J=1.5 Hz), 6.96 (1H, d, J=2.5 Hz), 7.35 (1H, t, J=8.0 Hz), 7.51 (1H, dt, J=1.3 Hz, 7.5 Hz), 7.74 (1H, d, J=7.9 Hz), 8.00 (1H, dd, J=7.9 Hz, 1.1 Hz).

### EXAMPLE 161

ethyl 2-[({1-[(3-chloro-5-ethoxy-2-pyridinyl)methyl]-2,4-dimethyl-1H-benzimidazol-6-yl}oxy)methyl]-6-methylbenzoate

NMR(CDCl₃,δ): 1.25 (3H, t, J=7.1 Hz), 1.43 (3H, t, J=7.1 Hz), 2.39 (3H, s), 2.73 (3H, s), 2.97 (3H, br s), 4.06 (2H, q, J=7.1 Hz), 4.28 (2H, q, J=7.1 Hz), 5.09 (2H, s), 5.44 (2H, s), 6.75 (1H, s), 6.87 (1H, br s), 7.20 (1H, m), 7.25 (1H, m), 7.28 (2H, m), 8.02 (1H, d, J=2.5 Hz).

### EXAMPLE 162

ethyl 2-[({1-[2-chloro-4-(1,3-oxazol-2-yl)benzyl]-2,4-dimethyl-1H-benzimidazol-6-yl}oxy)methyl]-6-methylbenzoate

NMR(CDCl₃,δ): 1.22 (3H, t, J=7.1 Hz), 2.32 (3H, s), 2.58 (3H, s), 2.66 (3H, s), 4.25 (2H, q, J=7.2 Hz), 5.06 (2H, s), 5.35 (2H, s), 6.50 (2H, m), 6.79 (1H, s), 7.10 (1H, m), 7.25 (3H, m), 7.72 (2H, m), 8.15 (1H, d, J=1.6 Hz).

### EXAMPLE 163

ethyl 2-[({1-[2-fluoro-4-(trifluoromethyl)benzyl]-2,4-dimethyl-1H-benzimidazol-6-yl)oxy}methyl]benzoate

NMR(CDCl₃,δ): 1.35 (3H, t, J=7.1 Hz), 2.56 (3H, s), 2.64 (3H, s), 4.34 (2H, q, J=7.1 Hz), 5.32 (2H, s), 5.46 (2H, s), 6.57 (1H, d, J=2.1 Hz), 6.74 (1H, t, J=7.6 Hz), 6.83 (1H, d, J=1.5 Hz), 7.24 (1H, d, J=8.5 Hz), 7.36 (2H, m), 7.50 (1H, dt, J=1.1 Hz, 7.5 Hz), 7.72 (1H, d, J=7.8 Hz), 8.00 (1H, dd, J=7.8 Hz, 1.2 Hz).

### EXAMPLE 164

ethyl 2-[({1-[2-chloro-4-(2-pyridinyl)benzyl]-2,4-dimethyl-1H-benzimidazol-6-yl}oxy)methyl]benzoate

NMR(CDCl₃,δ): 1.32 (3H, t, J=7.2 Hz), 2.55 (3H, s), 2.66 (3H, s), 4.31 (2H, q, J=7.2 Hz), 5.37 (2H, s), 5.44 (2H, s), 6.55 (1H, d, J=8.1 Hz), 6.60 (1H, d, J=1.2 Hz), 6.83 (1H, d, J=1.5 Hz), 7.26 (1H, m), 7.30 (1H, m), 7.50 (1H, m), 7.65 (2H, m), 7.74 (2H, m), 7.97 (1H, dd, J=7.8 Hz, 1.2 Hz), 8.14 (1H, d, J=1.7 Hz), 8.68 (1H, d, J=4.2 Hz).

### EXAMPLE 165

ethyl 2-[({1-[2-chloro-4-(pentyloxy)benzyl]-2,4-dimethyl-1H-benzimidazol-6-yl}oxy)methyl]-6-methylbenzoate

NMR(CDCl₃,δ): 0.92 (3H, t, J=7.1 Hz), 1.22 (3H, t, J=7.1 Hz), 1.40 (4H, m), 1.76 (2H, m), 2.36 (3H, s), 2.57 (3H, s), 2.64 (3H, s), 3.90 (2H, t, J=6.5 Hz), 4.25 (2H, q, J=7.1 Hz), 5.06 (2H, s), 5.25 (2H, s), 6.37 (1H, d, J=8.7 Hz), 6.54 (1H, d, J=7.0 Hz), 6.59 (1H, dd, J=8.7 Hz, 2.5 Hz), 6.76 (1H, d, J=1.3 Hz), 6.97 (1H, d, J=2.5 Hz), 7.16 (1H, dd, J=6.2 Hz, 2.5 Hz), 7.27 (2H, m).

### EXAMPLE 166

ethyl 2-[({1-[(3,5-dichloro-2-pyridinyl)methyl]-2,4-dimethyl-1H-benzimidazol-6-yl}oxy)methyl]-6-methylbenzoate

NMR(CDCl₃,δ): 1.27 (3H, t, J=7.1 Hz), 2.37 (3H, s), 2.76 (3H, s), 3.01 (3H, s), 4.28 (2H, q, J=7.1 Hz), 5.10 (2H, s), 5.51 (2H, s), 6.66 (1H, d, J=1.9 Hz), 6.90 (1H, s), 7.19 (1H, d, J=7.3 Hz), 7.25 (1H, t, J=6.7 Hz), 7.29 (1H, d, J=7.5 Hz), 7.79 (1H, d, J=2.0 Hz), 8.24 (1H, d, J=2.1 Hz).

The following compounds were obtained in a similar manner to that of Preparation Example 249 and Example 138.

### EXAMPLE 167

ethyl 2-[({1-[2-chloro-4-(1,3-oxazol-2-yl)benzyl]-2-methyl-1H-benzimidazol-6-yl}oxy)methyl]benzoate

### EXAMPLE 168

ethyl 2-[({1-[(2-chloro-6-phenyl-3-pyridinyl)methyl]-2-methyl-1H-benzimidazol-6-yl}oxy)methyl]benzoate

### EXAMPLE 169

ethyl 2-[({1-[2-chloro-4-(1,3-oxazol-2-yl)benzyl]-2-methyl-1H-benzimidazol-6-yl}oxy)methyl]-6-methylbenzoate

### EXAMPLE 170

ethyl 2-[({1-[(3-chloro-1,1′-biphenyl-4-yl)methyl]-2-methyl-1H-benzimidazol-6-yl}oxy)methyl]-6-methylbenzoate

NMR(CDCl₃,δ): 1.20 (3H, t, J=7.2 Hz), 2.35 (3H, s), 2.55 (3H, s), 4.24 (2H, q, J=7.2 Hz), 5.09 (2H, s), 5.39 (2H, s), 6.49 (1H, d, J=8.1 Hz), 6.72 (1H, d, J=2.3 Hz), 6.93 (1H, dd, J=8.8 Hz, 2.3 Hz), 7.13 (1H, d, J=7.1 Hz), 7.24 (1H, m), 7.29 (2H, m), 7.37 (1H, m), 7.44 (2H, m), 7.53 (2H, m), 7.62 (1H, d, J=8.8 Hz), 7.67 (1H, d, J=1.8 Hz).

### EXAMPLE 171

ethyl 2-[({1-[(3,5-dichloro-2-pyridinyl)methyl]-2-methyl-1H-benzimidazol-6-yl}oxy)methyl]-6-methylbenzoate

NMR(CDCl₃,δ): 1.24 (3H, t, J=7.2 Hz), 2.38 (3H, s), 2.64 (3H, s), 4.26 (2H, q, J=7.2 Hz), 5.10 (2H, s), 5.42 (2H, s), 6.76 (1H, s), 6.90 (1H, d, J=9.0 Hz), 7.18 (1H, d, J=5.7 Hz), 7.29 (2H, m), 7.58 (1H, d, J=8.4 Hz), 7.74 (1H, s), 8.29 (1H, s).

### EXAMPLE 172

ethyl 2-[({1-[2-chloro-4-(pentyloxy)benzyl]-2-methyl-1H-benzimidazol-6-yl}oxy)methyl]-6-methylbenzoate

NMR(CDCl₃,δ): 0.93 (3H, t, J=7.1 Hz), 1.24 (3H, t, J=7.2 Hz), 1.40 (4H, m), 1.77 (2H, m), 2.37 (3H, s), 2.67 (3H, s), 3.92 (2H, t, J=6.5 Hz), 4.26 (2H, q, J=7.2 Hz), 5.10 (2H, s), 5.32 (2H, s), 6.45 (1H, d, J=8.7 Hz), 6.64 (1H, dd, J=8.6 Hz, 2.4 Hz), 6.77 (1H, d, J=1.9 Hz), 7.00 (2H, m), 7.19 (1H, m), 7.28 (2H, m), 7.70 (1H, d, J=8.8 Hz).

### EXAMPLE 173

A mixture of methyl 2-[({1-[(3-chloro-1,1′-biphenyl-4-yl)methyl]-2,4-dimethyl-1H-benzimidazol-6-yl}oxy)methyl]-6-nitrobenzoate (1.0 g), Fe (1.0 g), NH₄Cl (1.92 g), THF (20 mL), MeOH (20 mL) and H₂O (16 mL) was refluxed for 4 hours. After cooling, the mixture was diluted with MeOH and the insoluble materials were filtered off. The filtrate was evaporated in vacuo and the residue was partitioned between EtOAc/THF and H₂O. The organic layer was separated, washed with brine, dried over Na₂SO₄, and concentrated in vacuo to give methyl 2-amino-6-[({1-[(3-chloro-1,1′-biphenyl-4-yl)methyl]-2,4-dimethyl-1H-benzimidazol-6-yl}oxy)methyl]benzoate (0.34 g), which was used in the next step without further purification.

### EXAMPLE 174

To a solution of ethyl 4-{[1-(2-bromo-4-ethoxybenzyl)-2,4-dimethyl-1H-benzimidazol-6-yl]oxy}butanoate (0.19 g) in 1,4-dioxane (10 ml) was added tributyl(vinyl)tin (0.14 g), lithium chloride (0.047 g), and tetrakis (triphenylphsphine)palladium (0) (0.039 g). The solution was heated at 100° C. for 24 hours, and the solvent was removed in vacuo. The residue was partitioned between water and EtOAc. The organic layer was washed with-brine and 5% aqueous potassium fluoride solution, evaporated to dryness, diluted with acetonitrile, filtered, and concentrated in vacuo. The residue was purified using chromatography (silica gel, n-hexane/EtOAc=1/2) to yield ethyl 4-{[1-(4-ethoxy-2-vinylbenzyl)-2,4-dimethyl-1H-benzimidazol-6-yl]oxy}butanoate (0.14 g) as a yellow oil.

NMR(CDCl₃,δ): 1.23 (3H, t, J=7.1 Hz), 1.39 (3H, t, J=7.0 Hz), 2.04-2.08 (2H, m), 2.48 (3H, s), 2.49 (2H, t, J=7.3 Hz), 2.63 (3H, s), 3.94 (2H, t, J=6.1 Hz), 4.00 (2H, q, J=7.0 Hz), 4.12 (2H, q, J=7.1 Hz), 5.23 (2H, s), 5.45 (1H, dd, J=1.2 Hz and 11 Hz), 5.72 (1H, dd, J=1.2 Hz and 17 Hz), 6.36 (1H, d, J=0.6 Hz), 6.45 (1H, d, J=2.2 Hz), 6.62 (1H, dd, J=2.6 Hz and 8.6 Hz), 6.64-6.68 (1H, m), 6.95-6.99 (1H, m), 7.05 (1H, d, J=2.6 Hz).

### EXAMPLE 175

Ethyl 4-{[1-(4-ethoxy-2-vinylbenzyl)-2,4-dimethyl-1H-benzimidazol-6-yl]oxy}butanoate (0.14 g) was stirred in EtOH (5 ml) in the presence of 10% Pd/C. (14 mg) under H₂ ballon for 21 hours at room temperature. The mixture was filtered and evaporated to dryness to give ethyl 4-{[1-(4-ethoxy-2-ethylbenzyl)-2,4-dimethyl-1H-benzimidazol-6-yl]oxy}butanoate (0.14 g, 100%) as a colorless oil.

NMR(CDCl₃,δ): 1.23 (3H, t, J=7.1 Hz), 1.24 (3H, t, J=7.1 Hz), 1.40 (3H, t, J=7.0 Hz), 2.05-2.10 (2H, m), 2.49 (2H, t, J=7.2 Hz), 2.68-2.72 (8H, m), 3.95 (2H, t, J=6.1 Hz), 4.00 (2H, q, J=7.2 Hz), 4.13 (2H, q, J=7.2 Hz), 5.26 (2H, s), 6.36 (1H, d, J=8.5 Hz), 6.50 (1H, s), 6.58 (1H, dd, J=2.3 Hz and 8.5 Hz), 6.79 (1H, br s), 6.83 (1H, d, J=2.5 Hz).

### EXAMPLE 176

To a solution of ethyl 4-{[1-(2,4-dichlorobenzyl)-2-methyl-1H-benzimidazol-6-yl]oxy}-2,2-dimethylbutanoate (135 mg) in EtOH (1.4 ml) was added 2N-NaOH (0.6 ml) at ambient temperature. The mixture was heated at 80°.C for 4 hours. After cooling, the reaction mixture was acidified with 1N-HCl (pH 3-4). The precipitates were collected by filtration and washed with water and MeOH (small portion) to give-4-{[1-(2,4-dichlorobenzyl)-2-methyl-1H-benzimidazol-6-yl]oxy}-2,2-dimethylbutanoic acid (112 mg) as pale brown crystals.

NMR(DMSO-d₆,δ): 1.15 (6H, s), 1.92 (2H, t, J=7.0 Hz), 2.37 (3H, s), 3.93 (2H, t, J=7.0 Hz), 5.48 (2H, br s), 6.45 (1H, d, J=8.4 Hz), 6.75 (1H, dd, J=2.2 Hz, 8.6 Hz), 7.00 (1H, d, J=2.2 Hz), 7.34 (1H, dd, J=2.1 Hz, 8.4 Hz), 7.43 (1H, d, J=8.6 Hz), 7.73 (1H, d, J=2.1 Hz), 12.22 (1H, br s).

MS: 419 (M−1).

### EXAMPLE 177

To a solution of 4-({1-[2-chloro-4-(pentyloxy)benzyl]-2-methyl-1H-benzimidazol-6-yl}oxy)-2,2-dimethylbutanoate (800 mg) in EtOH (8 ml) was added 2N-NaOH (3.2 ml) at ambient temperature. The mixture was heated at 80° C. for 2 hours. After cooling, the reaction mixture was acidified with 1N-HCl (pH 3-4). The precipitates were collected by filtration and washed with water and MeOH (small portion) to give 4-({1-[2-chloro-4-(pentyloxy)benzyl]-2-methyl-1H-benzimidazol-6-yl}oxy)-2,2-dimethylbutanoic acid (616 mg) as pale brown crystals.

NMR(DMSO-d₆,δ): 0.8-1.0 (3H, m), 1.15 (6H, s), 1.2-1.5 (4H, m), 1.5-1.8 (2H, m), 1.92 (2H, t, J=6.9 Hz), 2.39 (3H, s), 3.8-4.0 (4H, m), 5.40 (2H, br s), 6.50 (1H, d, J=8.7 Hz), 6.73 (1H, dd, J=2.2 Hz, 8.7 Hz), 6.82 (1H, dd, J=2.5 Hz, 8.7 Hz), 6.96 (1H, d, J=2.2 Hz), 7.10 (1H, d, J=2.5 Hz), 7.41 (1H, d, J=8.7 Hz), 12.19 (1H, br s).

MS: 471 (M−1).

### EXAMPLE 178

To a solution of methyl 2-[({1-[(3-chloro-1,1′-biphenyl-4-yl)methyl]-2,4-dimethyl-1H-benzimidazol-6-yl}oxy)methyl]nicotinate(140 mg) in ethanol (1.4 ml) was added 1N NaOH (0.547 mL) at room temperature. The mixture was heated at 80° C. for 2 hours. After cooling, the reaction mixture was acidified with 1N-HCl (pH 5). The resulting precipitates were collected by filtration and washed with water to give 2-[({1-[(3-chloro-1,1′-biphenyl-4-yl)methyl]-2,4-dimethyl-1H-benzimidazol-6-yl}oxy)methyl]nicotinic acid (132 mg) as white crystals.

NMR(DMSO-d₆, 200 MHz, 8): 2.46 (3H, s), 2.48 (3H, s), 5.37 (2H, s), 5.50(2H, s), 6.52 (1H, d, J=8.1 Hz), 6.66 (1H, s), 6.89 (1H, s), 7.35-7.54 (5H, m), 7.66-7.69 (2H, m), 7.83 (1H, S), 8.16 (1H, d, J=7.7 Hz), 8.62 (1H, d, J=4.8 Hz), 13.5 (1H, br s).

MS: 496 (M−1).

The following compounds were obtained in a similar manner to that of Example 178.

### EXAMPLE 179

2-[({1-[2-chloro-4-(pentyloxy)benzyl]-2,4-dimethyl-1H-benzimidazol-6-yl}oxy)methyl]benzoic acid

NMR(DMSO-d₆,δ): 0.86 (3H, t, J=6.9 Hz), 1.33 (4H, m), 1.66 (2H, m), 2.45 (6H, s), 3.92 (2H, t, J=6.4 Hz), 5.36 (4H, s), 6.56 (1H, d, J=8.0 Hz), 6.71 (1H, s), 6.78 (2H, m), 7.05 (1H, d, J=2.2 Hz), 7.40 (1H, t, J=7.5 Hz), 7.52 (1H, t, J=7.6 Hz), 7.58 (1H, d, J=7.7 Hz), 7.89 (1H, d, J=7.7 Hz), 13.20 (1H, br s).

### EXAMPLE 180

2-[({1-[(2-chloro-6-phenyl-3-pyridinyl)methyl]-2-methyl-1H-benzimidazol-6-yl}oxy)methyl]benzoic acid

NMR(DMSO-d₆,δ): 2.54 (3H, s), 5.41 (2H, s), 5.58 (2H, s), 6.95 (1H, d, J=8.6 Hz), 7.07 (1H, d, J=7.7 Hz), 7.18 (1H, s), 7.35 (1H, t, J=7.5 Hz), 7.49 (4H, m), 7.55 (1H, d, J=8.7 Hz), 7.58 (1H, d, J=7.8 Hz), 7.86 (2H, m), 8.01 (2H, d, J=7.1 Hz), 13.01 (1H, br s).

### EXAMPLE 181

2-[({1-[2-chloro-4-(1,3-oxazol-2-yl)benzyl]-2-methyl-1H-benzimidazol-6-yl}oxy)methyl]benzoic acid

NMR(DMSO-d₆,δ): 2.43 (3H, s), 5.39 (2H, s), 5.53 (2H, s), 6.65 (1H, d, J=8.2 Hz), 6.84 (1H, dd, J=8.7 Hz, 2.3 Hz), 7.00 (1H, d, J=2.3 Hz), 7.32 (1H, t, J=7.5 Hz), 7.41 (1H, d, J=0.5 Hz), 7.47 (1H, d, J=8.7 Hz), 7.48 (1H, dt, J=1.2 Hz, 7.5 Hz), 7.56 (1H, d, J=7.6 Hz), 7.79 (1H, dd, J=8.2 Hz, 1.6 Hz), 7.85 (1H, dd, J=7.8 Hz, 1.1 Hz), 8.01 (1H, d, J=1.6 Hz), 8.25 (1H, s), 13.01 (1H, br s).

### EXAMPLE 182

2-[({1-[2-fluoro-4-(trifluoromethyl)benzyl]-2,4-dimethyl-1H-benzimidazol-6-yl}oxy)methyl]benzoic acid

NMR(DMSO-d₆,δ): 2.45 (6H, s), 5.38 (2H, s), 5.52 (2H, s), 6.68 (1H, s), 6.86 (1H, s), 6.94 (1H, t, J=7.9 Hz), 7.38 (1H, t, J=7.7 Hz), 7.46 (1H, d, J=8.1 Hz), 7.51 (1H, t, J=8.5 Hz), 7.58 (1H, d, J=7.8 Hz), 7.70 (1H, d, J=10.0 Hz), 7.88 (1H, d, J=7.8 Hz), 13.02 (1H, br s).

### EXAMPLE 183

2-[({1-[2-chloro-4-(2-pyridinyl)benzyl]-2,4-dimethyl-1H-benzimidazol-6-yl}oxy)methyl]benzoic acid

NMR(DMSO-d₆,δ): 2.48 (3H, s), 2.50 (3H, s), 5.38 (2H, s), 5.53 (2H, s), 6.67 (1H, d, J=7.6 Hz), 6.75 (1H, s), 6.89 (1H, s), 7.32 (1H, t, J=7.5 Hz), 7.38 (1H, dd, J=7.4 Hz, 4.8 Hz), 7.49 (1H, dt, J=1.1 Hz, 7.7 Hz), 7.57 (1H, d, J=7.6 Hz), 7.85 (1H, dd, J=7.7 Hz, 1.1 Hz), 7.89 (2H, m), 7.96 (1H, d, J=8.0 Hz), 8.20 (1H, d, J=1.7 Hz), 8.66 (1H, d, J=4.2 Hz), 13.00 (1H, br s).

### EXAMPLE 184

To a solution of ethyl 4-[(1-{[3-chloro-5-(pentyloxy)-2-pyridinyl]methyl}-2,4-dimethyl-1H-benzimidazol-6-yl)oxy]butanoate (220 mg) in EtOH (2.2 ml) was added 1N NaOH (0.9 mL) at room temperature. The mixture was heated at 80° C. for 1 hour. After cooling to room temperature, the reaction mixture was acidified with 1N-HCl (pH 4). The precipitates were collected by filtration and washed with water. Recrystallization from MeOH afforded 4-[(1-{[3-chloro-5-(pentyloxy)-2-pyridinyl]methyl)-2,4-dimethyl-1H-benzimidazol-6-yl)oxy]butanoic acid (101 mg) as white crystals.

NMR(DMSO-d₆, 200 MHz,δ): 0.87 (3H, t, J=6.7 Hz), 1.26-1.37 (4H, m), 1.61-1.71 (2H, m), 1.85-1.96 (2H, m), 2.35 (2H, t, J=7.1 Hz), 2.39 (3H, s), 2.51 (3H, s), 3.89 (2H, t, J=6.3 Hz), 4.03 (2H, t, J=6.5 Hz), 5.48 (2H, s), 6.55 (1H, d, J=2.1 Hz), 6.72 (1H, d, J=2.1 Hz), 7.63 (1H, d, J=2.5 Hz), 8.11 (1H, d, J=2.5 Hz), 12.3 (1H, br s).

MS: 460 (M+1).

The following compounds were obtained in a similar manner to that of Example 184.

### EXAMPLE 185

4-{[6-(3-carboxypropoxy)-2,4-dimethyl-1H-benzimidazol-1-yl]methyl}-3-methoxybenzoic acid

NMR(DMSO-d₆,δ): 1.85-1.93 (2H, m), 2.35 (2H, t, J=7.3 Hz), 2.48 (3H, s), 2.55 (3H, s), 3.90 (3H, s), 3.92 (2H, t, J=6.5 Hz), 5.45 (2H, s), 6.73 (1H, s), 6.83 (1H, br s), 6.92 (1H, s), 7.46 (1H, dd, J=1.1 Hz, 7.9 Hz), 7.53 (1H, d, J=1.3 Hz), 12.6 (2H, br s).

### EXAMPLE 186

4-({1-[2-fluoro-4-(trifluoromethyl)benzyl]-2,4-dimethyl-1H-benzimidazol-6-yl}oxy)butanoic acid

NMR(DMSO-d₆,δ): 1.86-1.92 (2H, m), 2.35 (2H, t, J=7.3 Hz), 2.42 (3H, s), 2.44 (3H, s), 3.90 (2H, t, J=6.4 Hz), 5.55 (2H, s), 6.60-6.61 (1H, m), 6.86 (1H, d, J=2.2 Hz), 6.90 (1H, t, J=7.7 Hz), 7.51 (1H, d, J=8.0 Hz), 7.74 (1H, d, J=9.2 Hz), 12.1 (1H, br s).

### EXAMPLE 187

4-({1-[2-chloro-4-(1H-pyrazol-1-yl)benzyl]-2,4-dimethyl-1H-benzimidazol-6-yl}oxy)butanoic acid

NMR(DMSO-d₆,δ): 1.86-1.91 (2H, m), 2.34 (2H, t, J=7.3 Hz), 2.40 (3H, s), 2.46 (3H, s), 3.89 (2H, t, J=6.4 Hz), 5.47 (2H, s), 6.54-6.61 (3H, m), 6.82 (1H, d, J=2.1 Hz), 7.71 (1H, dd, J=2.2 Hz, 8.5 Hz), 7.74 (1H, d, J=1.6 Hz), 8.03 (1H, d, J=2.3 Hz), 8.51 (1H, d, J=2.5 Hz), 12.1 (1H, br s).

### EXAMPLE 188

4-}[1-(2-bromo-4-ethoxybenzyl)-2,4-dimethyl-1H-benzimidazol-6-yl]oxy}butanoic acid

NMR(DMSO-d₆,δ): 1.27 (3H, t, J=7.0 Hz), 1.85-1.91 (2H, m), 2.34 (2H, t, J=7.3 Hz), 2.37 (3H, s), 2.45 (3H, s), 3.88 (2H, t, J=6.4 Hz), 3.99 (2H, q, J=7.0 Hz), 5.32 (2H, s), 6.33 (1H, d, J=8.7 Hz), 6.59 (1H, d, J=1.4 Hz), 6.74 (1H, d, J=2.2 Hz), 6.83 (1H, dd, J=2.6 Hz and 8.7 Hz), 7.24 (1H, d, J=2.6 Hz), 12.1 (1H, br s).

### EXAMPLE 189

4-({1-[4-ethoxy-2-(trifluoromethyl)benzyl]-2,4-dimethyl-1H-benzimidazol-6-yl}oxy)butanoic acid

NMR(DMSO-d₆,δ): 1.29 (3H, t, J=7.0 Hz), 1.85-1.91 (2H, m), 2.32-2.37 (5H, m), 2.46 (3H, s), 3.88 (2H, t, J=6.4 Hz), 4.04 (2H, q, J=7.0 Hz), 5.44 (2H, s), 6.34 (1H, d, J=8.7 Hz), 6.61-6.62 (1H, m), 6.74 (1H, d, J=2.2 Hz), 7.07 (1H, dd, J=2.6 Hz, 8.6 Hz), 7.27 (1H, d, J=2.6 Hz).

### EXAMPLE 190

4-({1-[2-chloro-4-(2-pyridinyl)benzyl]-2,4-dimethyl-1H-benzimidazol-6-yl}oxy)butanoic acid

NMR(DMSO-d₆,δ): 1.85-1.91 (2H, m), 2.33 (2H, t, J=7.3 Hz), 2.40 (3H, s), 2.47 (3H, s), 3.89 (2H, t, J=6.4 Hz), 5.51 (2H, s), 6.52 (1H, d, J=8.2 Hz), 6.61-6.62 (1H, m), 6.83 (1H, d, J=2.2 Hz), 7.36-7.39 (1H, m), 7.86-7.89 (1H, m), 7.91 (1H, dd, J=1.8 Hz, 8.2 Hz), 7.95-7.97 (1H, m), 8.23 (1H, d, J=1.8 Hz), 8.64-8.66 (1H, m), 12.0 (1H, br s).

### EXAMPLE 191

4-{[1-(4-ethoxy-2-ethylbenzyl)-2,4-dimethyl-1H-benzimidazol-6-yl]oxy}butanoic acid

NMR(DMSO-d₆,δ): 1.18 (3H, t, J=7.5 Hz), 1.27 (3H, t, J=7.0 Hz), 1.87-1.91 (2H, m), 2.33-2.36 (5H, m), 2.45 (3H, s), 2.69 (2H, q, J=7.5 Hz), 3.88 (2H, t, J=6.4 Hz), 3.94 (2H, q, J=7.0 Hz), 5.31 (2H, s), 6.16 (1H, d, J=8.5 Hz), 6.58-6.60 (2H, m), 6.72 (1H, d, J=2.1 Hz), 6.79 (1H, d, J=2.1 Hz).

### EXAMPLE 192

4-{[1-(2-chloro-4-ethylbenzyl)-2,4-dimethyl-1H-benzimidazol-6-yl]oxy}butanoic acid

NMR(DMSO-d₆,δ): 1.12 (3H, t, J=7.6 Hz), 1.85-1.90 (2H, m), 2.33 (2H, t, J=7.3 Hz), 2.37 (3H, s), 2.45 (3H, s), 2.55 (2H, q, J=7.6 Hz), 3.88 (2H, t, J=6.4 Hz), 5.40 (2H, s), 6.36 (1H, d, J=8.0 Hz), 6.59-6.60 (1H, m), 6.75 (1H, d, J=2.2 Hz), 7.05(1H, dd, J=1.5 Hz, 7.9 Hz), 7.37 (1H, d, J=1.6 Hz).

### EXAMPLE 193

To a mixture of methyl 4-[({1-[(3-chloro-1,1′-biphenyl-4-yl)methyl]-2,4-dimethyl-1H-benzimidazol-6-yl}oxy)methyl]benzoate (0.32 g), MeOH (6 mL) and THF (6 mL) was added 4 N NaOH (4 mL). After refluxing for an hour, the mixture was acidified with 1 N HCl in an ice-bath. The precipitate formed was collected and dried in vacuo to give 4-[({1-[(3-chloro-1,1′-biphenyl-4-yl)methyl]-2,4-dimethyl-1H-benzimidazol-6-yl}oxy)methyl]benzoic acid (0.29 g).

NMR(DMSO-d₆,δ): 2.43 (3H, s), 2.4-2.6 (3H, s), 5.13 (2H, s), 5.51 (2H, s), 6.51 (1H, d, J=8 Hz), 6.76 (1H, d, J=1 Hz), 6.98 (1H, d, J=2 Hz), 7.3-8.0 (10H, m), 12.8-13.2 (1H, br m).

MS: 495 (M−H).

The following compounds were obtained in a similar manner to that of Example 193.

### EXAMPLE 194

2-chloro-6-[({1-[(3-chloro-1,1′-biphenyl-4-yl)methyl]-2,4-dimethyl-1H-benzimidazol-6-yl}oxy)methyl]benzoic acid

NMR(DMSO-d₆,δ): 2.4-2.6 (6H, s), 5.06 (2H, s), 5.57 (2H, s), 6.58 (1H, d, J=8 Hz), 6.76 (1H, br s), 7.03 (1H, br s), 7.3-7.7 (9H, m), 7.85 (1H, d, J=2 Hz), 13.0-14.5 (1H, br m).

MS: 529, 531 (M−H).

### EXAMPLE 195

4-chloro-2-[({1-[(3-chloro-1,1′-biphenyl-4-yl)methyl]-2,4-dimethyl-1H-benzimidazol-6-yl}oxy)methyl]benzoic acid

NMR(DMSO-d₆,δ): 2.54 (3H, s), 2.61 (3H, s), 5.42 (2H, s), 5.61 (2H, s), 6.7-6.8 (1H, m), 6.91 (1H, br s), 7.00 (1H, br s), 7.4-7.8 (9H, m), 7.91 (1H, d, J=8 Hz), 13.1-13.5 (1H, br m).

MS: 531, 533 (M+H).

### EXAMPLE 196

5-chloro-2-[({1-[(3-chloro-1,1′-biphenyl-4-yl)methyl]-2,4-dimethyl-1H-benzimidazol-6-yl}oxy)methyl]benzoic acid

NMR(DMSO-d₆,δ): 2.45 (3H, s), 2.4-2.6 (3H, s), 5.36 (2H, s), 5.48 (2H, s), 6.54 (1H, d, J=8 Hz), 6.70 (1H, br s), 6.85 (1H, d, J=2 Hz), 7.3-7.9 (10H, m), 13.4-13.7 (1H, br m).

MS: 531, 533 (M+H).

### EXAMPLE 197

3-chloro-2-[({1-[(3-chloro-1,1′-biphenyl-4-yl)methyl]-2,4-dimethyl-1H-benzimidazol-6-yl}oxy)methyl]benzoic acid

NMR(DMSO-d₆,δ): 2.56 (3H, s), 2.70 (3H, s), 5.39 (2H, s), 5.73 (2H, s), 6.84 (1H, d, J=8 Hz), 6.94 (1H, br s), 7.28 (1H, br s), 7.4-7.8 (9H, m), 7.89 (1H, d, J=2 Hz), 13.0-13.7 (1H, br m).

MS: 531, 533 (M+H).

### EXAMPLE 198

2-[({1-[(3-chloro-1,1′-biphenyl-4-yl)methyl]-2,4-dimethyl-1H-benzimidazol-6-yl}oxy)methyl]-6-fluorobenzoic acid

NMR(DMSO-d₆,δ): 2.43 (3H, s), 2.4-2.6 (3H, s), 5.12 (2H, s), 5.51 (2H, s), 6.48 (1H, d, J=8 Hz), 6.66 (1H, d, J=1 Hz), 6.94 (1H, d, J=2 Hz), 7.2-7.6 (8H, m), 7.66 (1H, dd, J=1 Hz, J=8 Hz), 7.83 (1H, d, J=2 Hz), 13.3-14.0 (1H, br m).

MS: 513 (M−H).

### EXAMPLE 199

A mixture of methyl 2-[({1-[(3-chloro-1,1′-biphenyl-4-yl)methyl]-2,4-dimethyl-1H-benzimidazol-6-yl}oxy)methyl]-6-methylbenzoate (0.13 g), 50% NaOH (1 mL) and 2-ethoxyethanol (2 mL) was refluxed for an hour. After cooling, the reaction mixture was acidified with 1 N HCl in an ice-bath. The precipitate formed was collected and dried in vacuo to give 2-[({1-[(3-chloro-1,1′-biphenyl-4-yl)methyl]-2,4-dimethyl-1H-benzimidazol-6-yl}oxy)methyl]-6-methylbenzoic acid (0.13 g).

NMR(DMSO-d₆,δ): 2.29 (3H, s), 2.48 (3H, s), 2.58 (3H, s), 5.06 (2H, s), 5.61 (2H, s), 6.66 (1H, d, J=8 Hz), 6.80 (1H, br s), 7.07 (1H, br s), 7.2-7.7 (9H, m), 7.86 (1H, d, J=2 Hz), 13.0-13.5 (1H, br m).

MS: 511 (M+H).

The following compounds were obtained in a similar manner to that of Example 199.

### EXAMPLE 200

2-[({1-[(3-chloro-1,1′-biphenyl-4-yl)methyl]-2,4-dimethyl-1H-benzimidazol-6-yl}oxy)methyl]-6-nitrobenzoic acid

NMR(DMSO-d₆,δ): 2.53 (3H, s), 2.61 (3H, s), 5.19 (2H, s), 5.64 (2H, s), 6.69 (1H, d, J=8 Hz), 6.86 (1H, br s), 7.13 (1H, br s), 7.4-8.2 (10H, m), 14.0-15.0 (1H, br m).

MS: 540 (M−H).

### EXAMPLE 201

2-amino-6-[({1-[(3-chloro-1,1′-biphenyl-4-yl)methyl]-2,4-dimethyl-1H-benzimidazol-6-yl}oxy)methyl]benzoic acid

NMR(DMSO-d₆,δ): 2.4-2.5 (3H, s), 2.60 (3H, s), 5.17 (2H, s), 5.61 (2H, s), 6.6-7.1 (5H, m), 7.3-7.9 (8H, m), 8.3-8.6 (2H, br m).

MS: 512 (M−H).

### EXAMPLE 202

2-[({1-[2-chloro-4-(1,3-oxazol-2-yl)benzyl]-2-methyl-1H-benzimidazol-6-yl}oxy)methyl]-6-methylbenzoic acid

NMR(DMSO-d₆,δ): 2.27 (3H, s), 2.40 (3H, s), 5.04 (2H, s), 5.54 (2H, s), 6.55 (1H, d, J=8.1 Hz), 6.79 (1H, dd, J=8.7 Hz, 2.4 Hz), 7.09 (1H, d, J=2.3 Hz), 7.16 (1H, d, J=7.1 Hz), 7.24 (1H, t, J=7.4 Hz), 7.27 (1H, d, J=6.9 Hz), 7.40 (1H, d, J=1.6 Hz), 7.45 (1H, d, J=8.7 Hz), 7.79 (1H, dd, J=8.1 Hz, 1.6 Hz), 8.04 (1H, d, J=1.7 Hz), 8.24 (1H, d, J=0.4 Hz), 13.02 (1H, br s).

### EXAMPLE 203

2-[({1-[(3-chloro-1,1′-biphenyl-4-yl)methyl]-2-methyl-1H-benzimidazol-6-yl}oxy)methyl]-6-methylbenzoic acid

NMR(DMSO-d₆,δ): 2.28 (3H, s), 2.42 (3H, s), 5.05 (2H, s), 5.53 (2H, s), 6.49 (1H, d, J=8.2 Hz), 6.80 (1H, dd, J=8.7 Hz, 2.3 Hz), 7.12 (1H, dd, J=2.3 Hz), 7.19 (1H, d, J=7.4 Hz), 7.26 (1H, t, J=7.5 Hz), 7.30 (1H, d, J=7.4 Hz), 7.38 (1H, t, J=7.3 Hz), 7.46 (3H, m), 7.52 (1H, dd, J=8.1 Hz, 1.7 Hz), 7.66 (2H, m), 7.83 (1H, d, J=1.8 Hz), 13.14 (1H, br s).

### EXAMPLE 204

2-[({1-[(3,5-dichloro-2-pyridinyl)methyl]-2-methyl-1H-benzimidazol-6-yl}oxy)methyl]-6-methylbenzoic acid

NMR(DMSO-d₆,δ): 2.29 (3H, s), 2.38 (3H, s), 5.03 (2H, s), 5.59 (2H, s), 6.7.4 (1H, dd, J=8.7 Hz, 2.4 Hz), 7.00 (1H, d, J=2.3 Hz), 7.19 (1H, d, J=6.8 Hz), 7.27 (2H, m), 7.38 (1H, d, J=8.7 Hz), 8.30 (1H, d, J=2.1 Hz), 8.40 (1H, d, J=2.1 Hz), 13.20 (1H, br s).

### EXAMPLE 205

2-[({1-[2-chloro-4-(pentyloxy)benzyl]-2-methyl-1H-benzimidazol-6-yl}oxy)methyl]-6-methylbenzoic acid

NMR(DMSO-d₆,δ): 0.86 (3H, t, J=7.1 Hz), 1.32 (4H, m), 1.67 (2H, m), 2.30 (3H, s), 2.39 (3H, s), 3.93 (2H, t, J=6.5 Hz), 5.04 (2H, s), 5.38 (2H, s), 6.46 (1H, d, J=8.7 Hz), 6.78 (1H, dt, J=2.5 Hz, 9.0 Hz), 7.04 (1H, d, J=2.2 Hz), 7.09 (1H, d, J=2.5 Hz), 7.21 (1H, d, J=7.2 Hz), 7.29 (2H, m), 7.42 (1H, d, J=8.7 Hz), 13.14 (1H, br s).

### EXAMPLE 206

2-[({1-[2-chloro-4-(pentyloxy)benzyl]-2,4-dimethyl-1H-benzimidazol-6-yl)oxy}methyl]-6-methylbenzoic acid

NMR(DMSO-d₆, δ): 0.86 (3H, t, J=7.1 Hz), 1.33 (4H, m), 1.67 (2H, m), 2.30 (3H, s), 2.39 (3H, s), 2.45 (3H, s), 3.93 (2H, t, J=6.5 Hz), 5.01 (2H, s), 5.36 (2H, s), 6.43 (1H, d, J=8.6 Hz), 6.62 (1H, s), 6.79 (1H, dd, J=8.7 Hz, 2.5 Hz), 6.84 (1H, d, J=1.6 Hz), 7.09 (1H, d, J=3.5 Hz), 7.21 (1H, m), 7.29 (2H, m), 13.13 (1H, br s).

### EXAMPLE 207

2-[({1-[(3,5-dichloro-2-pyridinyl)methyl]-2,4-dimethyl-1H-benzimidazol-6-yl}oxy)methyl]-6-methylbenzoic acid

NMR(DMSO-d₆,δ): 2.30 (3H, s), 2.39 (3H, s) 2.43 (3H, s), 5.00 (2H, s), 5.56 (2H, s), 6.59 (1H, s), 6.79 (1H, s), 7.21 (1H, m), 7.28 (2H, m), 8.29 (1H, d, J=2.0 Hz), 8.39 (1H, d, J=2.0 Hz), 13.18 (1H, br s).

### EXAMPLE 208

2-[({1-[(3-chloro-5-ethoxy-2-pyridinyl)methyl]-2,4-dimethyl-1H-benzimidazol-6-yl}oxy)methyl]-6-methylbenzoic acid

NMR(DMSO-d₆,δ): 1.30 (3H, t, J=7.0 Hz), 2.30 (3H, s), 2.47 (3H, s), 2.53 (3H, s), 4.09 (2H, q, J=7.0 Hz), 5.04 (2H, s), 5.59 (2H, br s), 6.73 (1H, br s), 6.96 (1H, br s), 7.23 (1H, m), 7.30 (2H, m), 7.65 (1H, d, J=2.2 Hz), 8.07 (1H, d, J=2.4 Hz), 13.19 (1H, br s).

### EXAMPLE 209

2-[({1-[2-chloro-4-(1,3-oxazol-2-yl)benzyl]-2,4-dimethyl-1H-benzimidazol-6-yl}oxy)methyl]-6-methylbenzoic acid

NMR(DMSO-d₆,δ): 2.27 (3H, s), 2.40 (3H, s), 2.47 (3H, s), 5.01 (2H, s), 5.51 (2H, s), 6.53 (1H, d, J=8.2 Hz), 6.64 (1H, s), 6.89 (1H, d, J=2.0 Hz), 7.16 (1H, d, J=7.0 Hz), 7.26 (2H, m), 7.40 (1H, s), 7.79 (1H, dd, J=8.1 Hz, 1.4 Hz), 8.04 (1H, d, J=1.5 Hz), 8.25 (1H, s), 13.16 (1H, br s).

### EXAMPLE 210

A minture of methyl 2-({[1-(2-chloro-4-cyanobenzyl)-2,4-dimethyl-1H-benzimidazol-6-yl]oxy}methyl)benzoate (0.27 g), 4 N NaOH (293 μL) and 2-methoxyethanol (5.9 mL) was stirred at ambient temperature overnight. The mixture was diluted with H₂O (3 mL) and acidified with 1 N HCl. The precipitate formed was collected and suspended in a mixture of EtOH (3 mL) and H₂O (1 mL). The mixture was stirred at ambient temperature overnight and the precipitate was collected and dried in vacuo to give 2-({[1-(2-chloro-4-cyanobenzyl)-2,4-dimethyl-1H-benzimidazol-6-yl]oxy}methyl)benzoic acid (93 mg).

NMR(DMSO-d₆,δ): 2.40 (3H, s), 2.4-2.6 (3H, s), 5.36 (2H, s), 5.52 (2H, s), 6.52 (1H, d, J=8 Hz), 6.70 (1H, br s), 6.79 (1H, d, J=2 Hz), 7.3-8.0 (5H, m), 8.12 (1H, d, J=1 Hz), 12.6-13.4 (1H, br m),

MS: 446 (M+H).

The following compound was obtained in a similar manner to that of Example 210.

### EXAMPLE 211

2-({[1-(4-cyano-2-fluorobenzyl)-2,4-dimethyl-1H-benzimidazol-6-yl]oxy}methyl)benzoic acid

NMR(DMSO-d₆,δ): 2.45 (3H, s), 2.46 (3H, s), 5.38 (2H, s), 5.53 (2H, s), 6.69 (1H, br s), 6.84 (1H, d, J=2 Hz), 6.8-7.0 (1H, m), 7.3-8.0 (6H, m), 12.8-13.4 (1H, br m).

MS: 428 (M−H).

The following compounds were obtained in a similar manner to that of Example 151 and Example 184.

### EXAMPLE 212

4-({1-[2-(2,4-dichlorophenyl)ethyl]-2,4-dimethyl-1H-benzimidazol-6-yl}oxy)butanoic acid

NMR(DMSO-d₆,δ): 1.94 (2H, quint, J=6.7 Hz), 2.28 (3H, s), 2.40 (3H, s), 2.37-2.41 (2H, m), 3.10 (2H, t, J=7.0 Hz), 3.92 (2H, t, J=6.4 Hz), 4.31 (2H, t, J=6.9 Hz), 6.55 (1H, br s), 6.69 (1H, d, J=2.0 Hz), 7.20 (1H, d, J=8.2 Hz), 7.30 (1H, dd, J=8.3 Hz, 2.1 Hz), 7.57 (1H, d, J=2.1 Hz), 12.13 (1H, br s).

### EXAMPLE 213

4-({1-[2-(2,4-difluorophenyl)ethyl]-2,4-dimethyl-1H-benzimidazol-6-yl}oxy)butanoic acid

NMR(DMSO-d₆,δ): 1.93 (2H, quint, J=6.9 Hz), 2.24 (3H, s), 2.37-2.41 (5H, m), 3.00 (2H, t, J=6.9 Hz), 3.94 (2H, t, J=6.4 Hz), 4.29 (2H, t, J=6.9 Hz), 6.54 (1H, d, J=1.3 Hz), 6.71 (1H, d, J=2.0 Hz), 6.96 (1H, dt, J=2.4 Hz, 8.5 Hz), 7.15 (1H, dt, J=2.5 Hz, 9.8 Hz), 7.19-7.24 (1H, m), 12.13 (1H, br s).

### EXAMPLE 214

4-({1-[3-(2,4-dichlorophenyl)propyl]-2,4-dimethyl-1H-benzimidazol-6-yl}oxy)butanoic acid

NMR(DMSO-d₆,δ): 1.90-1.99 (4H, m), 2.35-2.41 (5H, m), 2.46 (3H, s), 2.69-2.74 (2H, m), 3.95 (2H, t, J=6.3 Hz), 4.15 (2H, t, J=7.3 Hz), 6.56 (1H, br s), 6.77 (1H, d, J=1.9 Hz), 7.31-7.39 (2H, m), 7.55 (1H, d, J=1.9 Hz), 12.12 (1H, br s).

The following compounds were obtained in a similar manner to that of Preparation Example 250.

### PREPARATION EXAMPLE 258

4-(acetylamino)-3-({[3-chloro-5-(methoxymethyl)-2-pyridinyl]methyl}amino)-5-methylphenyl acetate

NMR(DMSO-d₆,δ): 2.04 (3H, s), 2.08 (3H, s), 2.22 (3H, s), 3.31 (3H, s), 4.37 (2H, d, J=5 Hz), 4.47 (2H, s), 5.86 (1H, t, J=5 Hz), 6.24 (1H, d, J=2 Hz), 6.30 (1H, d, J=2 Hz), 7.91 (1H, d, J=1 Hz), 8.50 (1H, d, J=1 Hz), 9.04 (1H, s).

MS: 414 (M+Na).

### PREPARATION EXAMPLE 259

4-(acetylamino)-3-({[3-chloro-5-(ethoxymethyl)-2-pyridinyl]methyl}amino)-5-methylphenyl acetate

NMR(DMSO-d₆,δ): 1.15 (3H, t, J=7 Hz), 2.04 (3H, s), 2.08 (3H, s), 2.22 (3H, s), 3.50 (2H, q, J=7 Hz), 4.37 (2H, d, J=5 Hz), 4.51 (2H, s), 5.86 (1H, t, J=5 Hz), 6.25 (1H, d, J=2 Hz), 6.30 (1H, d, J=2 Hz), 7.89 (1H, d, J=1 Hz), 8.49 (1H, d, J=1 Hz), 9.04 (1H, s).

MS: 406 (M+H).

The following compounds were obtained in a similar manner to that of Example 139.

### PREPARATION EXAMPLE 260

1-{[3-chloro-5-(methoxymethyl)-2-pyridinyl]methyl}-2,4-dimethyl-1H-benzimidazol-6-ol

NMR(DMSO-d₆,δ): 2.38 (3H, s), 2.44 (3H, s), 3.28 (3H, s), 4.42 (2H, s), 5.49 (2H, s), 6.3-6.5 (2H, m), 7.91 (1H, d, J=2 Hz), 8.32 (1H, d, J=2 Hz), 8.90 (1H, s).

MS: 332 (M+H).

### PREPARATION EXAMPLE 261

1-{[3-chloro-5-(ethoxymethyl)-2-pyridinyl]methyl}-2,4-dimethyl-1H-benzimidazol-6-ol

NMR(DMSO-d₆,δ): 1.13 (3H, t, J=7 Hz), 2.39 (3H, s), 2.44 (3H, s), 3.47 (2H, q, J=7 Hz), 4.45 (2H, s), 5.48 (2H, s), 6.3-6.5 (2H, m), 7.91 (1H, d, J=2 Hz), 8.32 (1H, d, J=2 Hz), 8.90 (1H, s).

MS: 346 (M+H).

The following compounds were obtained in a similar manner to that of Example 141.

### EXAMPLE 215

methyl 2-{[(1-{[3-chloro-5-(ethoxymethyl)-2-pyridinyl]methyl}-2,4-dimethyl-1H-benzimidazol-6-yl)oxy]methyl}-6-methylbenzoate

NMR(DMSO-d₆,δ): 1.12 (3H, t, J=7 Hz), 2.26 (3H, s), 2.42 (3H, s), 2.44 (3H, s), 3.47 (2H, t, J=7 Hz), 3.68 (3H, s), 4.46 (2H, s), 4.99 (2H, s), 5.56 (2H, s), 6.56 (1H, d, J=2 Hz), 6.75 (1H, d, J=2 Hz), 7.3-7.5 (3H, m), 7.91 (1H, d, J=2 Hz), 8.28 (1H, d, J=2 Hz).

MS: 507 (M+H).

### EXAMPLE 216

methyl 2-{[(1-{[3-chloro-5-(methoxymethyl)-2-pyridinyl]methyl}-2,4-dimethyl-1H-benzimidazol-6-yl)oxy]methyl}benzoate

NMR(DMSO-d₆,δ): 2.44 (6H, s), 3.28 (3H, s), 3.79 (3H, s), 4.41 (2H, s), 5.32 (2H, s), 5.55 (2H, s), 6.63 (1H, d, J=2 Hz), 6.73 (1H, d, J=2 Hz), 7.4-7.6 (3H, m), 7.8-8.0 (2H, m), 8.28 (1H, d, J=2 Hz).

MS: 480 (M+H).

### EXAMPLE 217

methyl 2-{[(1-{[3-chloro-5-(methoxymethyl)-2-pyridinyl]methyl}-2,4-dimethyl-1H-benzimidazol-6-yl)oxy]methyl}-6-methylbenzoate

NMR(DMSO-d₆,δ): 2.26 (3H, s), 2.42 (3H, s), 2.44 (3H, s), 3.28 (3H, s), 3.68 (3H, s), 4.42 (2H, s), 4.99 (2H, s), 5.57 (2H, s), 6.56 (1H, d, J=2 Hz), 6.75 (1H, d, J=2 Hz), 7.2-7.4 (3H, m), 7.92 (1H, d, J=1 Hz), 8.28 (1H, d, J=1 Hz).

MS: 494 (M+H).

### EXAMPLE 218

methyl 2-{[(1-{[3-chloro-5-(ethoxymethyl)-2-pyridinyl]methyl}-2,4-dimethyl-1H-benzimidazol-6-yl)oxy]methyl}benzoate

NMR(DMSO-d₆,δ): 1.12 (3H, t, J=7 Hz), 2.45 (6H, s), 3.47 (2H, t, J=7 Hz), 3.79 (3H, s), 4.45 (2H, s), 5.32 (2H, s), 5.54 (2H, s), 6.64 (1H, d, J=2 Hz), 6.73 (1H, d, J=2 Hz), 7.5-7.7 (3H, m), 7.8-8.0 (2H, m), 8.28 (1H, d, J=2 Hz).

MS: 493 (M+H).

The following compounds were obtained in a similar manner to that of Example 193.

### EXAMPLE 219

2-{[(1-{[3-chloro-5-(ethoxymethyl)-2-pyridinyl]methyl}-2,4-dimethyl-1H-benzimidazol-6-yl)oxy]methyl}benzoic acid

NMR(DMSO-d₆,δ): 1.12 (3H, t, J=7 Hz), 2.45 (6H, s), 3.47 (2H, q, J=7 Hz), 4.45 (2H, s), 5.36 (2H, s), 5.54 (2H, s), 6.64 (1H, br s), 6.73 (1H, br s), 7.3-7.7 (3H, m), 7.8-8.0 (2H, m), 8.28 (1H, d, J=2 Hz), 12.5-13.5 (1H, br m).

MS: 480 (M+H).

### EXAMPLE 220

2-{[(1-{[3-chloro-5-(methoxymethyl)-2-pyridinyl]methyl}-2,4-dimethyl-1H-benzimidazol-6-yl)oxy]methyl}benzoic acid

NMR(DMSO-d₆,δ): 2.45 (6H, s), 3.28 (3H, s), 4.41 (2H, s), 5.36 (2H, s), 5.54 (2H, s), 6.65 (1H, d, J=2 Hz), 6.74 (1H, d, J=2 Hz), 7.3-7.7 (3H, m), 7.8-8.0 (2H, m), 8.28 (1H, d, J=2 Hz), 12.5-13.5 (1H, br m).

MS: 466 (M+H).

The following compounds were obtained in a similar manner to that of Example 199.

### EXAMPLE 221

2-{[(1-{[3-chloro-5-(ethoxymethyl)-2-pyridinyl]methyl}-2,4-dimethyl-1H-benzimidazol-6-yl)oxy]methyl}-6-methylbenzoic acid

NMR(DMSO-d₆,δ): 1.12 (3H, t, J=7 Hz), 2.30 (3H, s), 2.41 (3H, s), 2.44 (3H, s), 3.47 (2H, q, J=7 Hz), 4.45 (2H, s), 5.00 (2H, s), 5.57 (2H, s), 6.59 (1H, d, J=2 Hz), 6.80 (1H, d, J=2 Hz), 7.1-7.4 (3H, m), 7.90 (1H, d, J=2 Hz), 8.28 (1H, d, J=2 Hz), 12.8-13.6 (1H, br m).

MS: 494 (M+H).

### EXAMPLE 222

2-{[(1-{[3-chloro-5-(methoxymethyl)-2-pyridinyl]methyl}-2,4-dimethyl-1H-benzimidazol-6-yl)oxy]methyl}-6-methylbenzoic acid

NMR(DMSO-d₆, δ): 2.31 (3H, s), 2.41 (3H, s), 2.44 (3H, s), 3.28 (3H, s), 4.42 (2H, s), 5.00 (2H, s), 5.57 (2H, s), 6.59 (1H, d, J=2 Hz), 6.81 (1H, d, J=2 Hz), 7.2-7.4 (3H, m), 7.91 (1H, d, J=2 Hz), 8.28 (1H, d, J=2 Hz), 13.0-13.4 (1H, br m).

MS: 480 (M+H).

### Industrial Applicability

The benzimidazole compounds, pharmaceutically acceptable salts thereof and prodrugs thereof of the present invention are useful for the prophylaxis and treatment of, for example, impaired glucose tolerance disorder, diabetes (e.g., type II diabetes), gestational diabetes, diabetic complications (e.g., diabetic gangrene, diabetic arthropathy, diabetic osteopenia, diabetic glomerulosclerosis, diabetic nephropathy, diabetic dermatopathy, diabetic neuropathy, diabetic cataract, diabetic retinopathy and the like), insulin resistance syndrome (e.g., insulin receptor abnormality, Rabson-Mendenhall syndrome, leprechaunism, Kobberling-Dunnigan syndrome, Lawrence-Seip syndrome (lipoatrophy), Cushing syndrome, acromegaly and the like), polycystic ovary syndrome, hyperlipidemia, atherosclerosis, cardiovascular diseases (e.g., stenocardia, cardiac failure and the like), hyperglycemia (e.g., those characterized by abnormal saccharometabolism such as eating disorders), pancreatitis, osteoporosis, hyperuricemia, hypertension, inflammatory bowel diseases, skin disorders related to an anomaly of differentiation of epidermic cells, hypertension, Alzheimer's disease, Parkinson's disease, multiple sclerosis, stroke, traumatic brain and spinal cord injury, and the like. In addition, they, in combination with a retinoid, are useful for treating disease states caused by uncontrolled cell proliferation, including cancer, restenosis and atherosclerosis.

This application is based on patent application No. 2003902860 filed in Australia, the content of which is hereby incorporated by reference. The references cited herein, including patents and patent applications, are hereby incorporated in their entireties by reference, to the extent that they have been disclosed herein.

1. A compound represented by the formula: 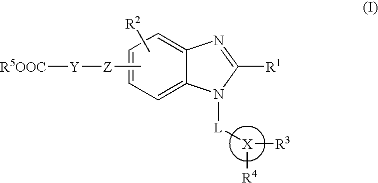
wherein
R¹ is lower alkyl, lower alkoxy, lower alkylthio, mono- or di(lower alkyl)amino or mono-, di- or trihalo(lower)alkyl,
R² is hydrogen or lower alkyl,
R³ is hydrogen, halogen, cyano, optionally esterified carboxy, lower alkoxy, lower alkyl optionally substituted by lower alkoxy, optionally substituted amino, mono-, di- or trihalo(lower)alkyl, optionally substituted aryl or heteroaryl,
R⁴ is halogen, lower alkyl, lower alkoxy, lower alkenyl, or mono-, di- or trihalo(lower)alkyl,
R⁵ is hydrogen or carboxy protective group,
L is lower alkylene,
ring X is benzene ring or heteroaryl ring,
Y is lower alkylene, optionally substituted phenylene or bivalent residue derived from optionally substituted heteroaryl, and
Z is bond, —O—, —CH₂O—, —OCH₂—, —N (R⁹)CH₂— or —CH₂N(R⁹)—, wherein R⁹ is hydrogen or lower alkyl,
provided that when Z is a bond, then Y is optionally substituted phenylene or bivalent residue derived from optionally substituted heteroaryl,
or a salt thereof or a prodrug thereof.

2. The compound of claim 1 wherein ring X is benzene ring, pyridine ring, oxazole ring or thiazole ring, Y is lower alkylene, phenylene optionally substituted by substituent(s) selected from the group consisting of lower alkyl, halogen, amino and nitro, or bivalent residue derived from pyridinyl, thiophenyl, imidazolyl or oxazolyl, each of which is optionally mono-substituted by lower alkyl, or a salt thereof or a prodrug thereof.

3. The compound of claim 2 wherein L is methylene, and Z is bond, —O—, —CH₂O— or —OCH₂—, or a salt thereof or a prodrug thereof.

4. The compound of claim 3 wherein Z is —O—, and Y is lower alkylene, or a salt thereof or a prodrug thereof.

5. The compound of claim 3 wherein Z is bond, —CH₂O— or —OCH₂— and Y is phenylene optionally substituted by substituent(s) selected from the group consisting of lower alkyl, halogen, amino and nitro, or bivalent residue derived from pyridinyl, thiophenyl, imidazolyl or oxazolyl, each of which is optionally mono-substituted by lower alkyl, or a salt thereof or a prodrug thereof.

6. The compound of claim 3, which is selected from the group consisting of
4-{[1-(2,4-dichlorobenzyl)-2,4-dimethyl-1H-benzimidazol-6-yl]oxy}butanoic acid,
4-[(1-{[3-chloro-5-(trifluoromethyl)-2-pyridinyl]methyl}-2,4-dimethyl-1H-denzimidazol-6-yl)oxy]butanoic acid,
4-({1-[(3,5-dichloro-2-pyridinyl)methyl]-2,4-dimethyl-1H-benzimidazol-6-yl}oxy)butanoic acid,
4-{[2-ethoxy-4-methyl-1-({4-methyl-2-[4-(trifluoromethyl)phenyl]-1,3-thiazol-5-yl}methyl)-1H-benzimidazol-6-yl]oxy}butanoic acid,
4-{[1-(2,4-dichlorobenzyl)-2-ethyl-4-methyl-1H-benzimidazol-6-yl]oxy}butanoic acid,
4-{[1-(2,4-dichlorobenzyl)-2-ethoxy-4-methyl-1H-benzimidazol-6-yl]oxy}butanoic acid,
2-[({1-[(3-chloro-1,1′-biphenyl-4-yl)methyl]-2,4-dimethyl-1H-benzimidazol-6-yl}oxy)methyl]benzoic acid,
2-{[(1-{[3-chloro-5-(trifluoromethyl)-2-pyridinyl]methyl}-2,4-dimethyl-1H-benzimidazol-6-yl)oxy]methyl}benzoic acid,
2-[({1-[(2-chloro-6-phenyl-3-pyridinyl)methyl]-2,4-dimethyl-1H-benzimidazol-6-yl}oxy)methyl]benzoic acid,
2-{[(1-{[3-chloro-5-(trifluoromethyl)-2-pyridinyl]methyl}-2-methyl-1H-benzimidazol-6-yl)oxy]methyl}benzoic acid,
4-{[1-(2,4-dichlorobenzyl)-2-methyl-1H-benzimidazol-6-yl]oxy}-2,2-dimethylbutanoic acid and
2-[({1-[(3-chloro-1,1′-biphenyl-4-yl)methyl]-2,4-dimethyl-1H-benzimidazol-6-yl}oxy)methyl]-6-methylbenzoic acid,
or a salt thereof or a prodrug thereof.

7. The compound of claim 1 or a salt thereof or a prodrug thereof for use as a medicament.

8. A pharmaceutical preparation comprising a compound of claim 1 or a salt thereof or a prodrug thereof as an active ingredient, in admixture with a pharmaceutically acceptable organic or inorganic excipient.

9. Use of a compound of claim 1 or a salt thereof or a prodrug thereof for preparing a blood lipid metabolism improver, a plasma lipid composition improver, a hypoglycemic agent, a hypoinsulinemic agent, an insulin resistance improver or an insulin sensitivity enhancer.

10. Use of a compound of claim 1 or a salt thereof or a prodrug thereof for preparing a medicament for the prophylaxis or treatment of impaired glucose tolerance disorder, diabetes, gestational diabetes, diabetic complications, insulin resistance syndrome, polycystic ovary syndrome, hyperlipidemia, atherosclerosis, cardiovascular diseases, hyperglycemia, pancreatitis, osteoporosis, hyperuricemia, hypertension, inflammatory bowel diseases, skin disorders related to an anomaly of differentiation of epidermic cells, hypertension, Alzheimer's disease, Parkinson's disease, multiple-sclerosis, stroke, traumatic brain or spinal cord injury.

11. A method for improving blood lipid metabolism, improving plasma lipid composition, lowering blood sugar, lowering blood insulin, improving insulin resistance or enhancing insulin sensitivity in a mammal, which comprises administering a pharmaceutically effective amount of a compound of claim 1 or a salt thereof or a prodrug thereof to the mammal.

12. A method for preventing or treating impaired glucose tolerance disorder, diabetes, gestational diabetes, diabetic complications, insulin resistance syndrome, polycystic ovary syndrome, hyperlipidemia, atherosclerosis, cardiovascular diseases, hyperglycemia, pancreatitis, osteoporosis, hyperuricemia, hypertension, inflammatory bowel diseases, skin disorders related to an anomaly of differentiation of epidermic cells, hypertension, Alzheimer's disease, Parkinson's disease, multiple sclerosis, stroke, traumatic brain or spinal cord injury in a mammal, which comprises administering a pharmaceutically effective amount of a compound of claim 1 or a salt thereof or a prodrug thereof to the mammal.

13. A production method of a compound represented by the formula: 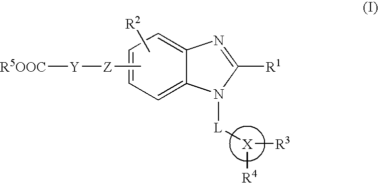
wherein
R¹ is lower alkyl, lower alkoxy, lower alkylthio, mono- or di(lower alkyl)amino or mono-, di- or trihalo(lower)alkyl,
R² is hydrogen or lower alkyl,
R³ is hydrogen, halogen, cyano, optionally esterified carboxy, lower alkoxy, lower alkyl optionally substituted by lower alkoxy, optionally substituted amino, mono-, di- or trihalo(lower)alkyl, optionally substituted aryl or heteroaryl,
R⁴ is halogen, lower alkyl, lower alkoxy, lower alkenyl, or mono-, di- or trihalo(lower)alkyl,
R⁵ is hydrogen or carboxy protective group,
L is lower alkylene,
ring X is benzene ring or heteroaryl ring,
Y is lower alkylene, optionally substituted phenylene or bivalent residue derived from optionally substituted heteroaryl, and
Z is bond, —O—, —CH₂O—, —OCH₂—, —N(R⁹)CH₂— or —CH₂N(R⁹)—, wherein R⁹ is hydrogen or lower alkyl,
provided that when Z is a bond, then Y is optionally substituted phenylene or bivalent residue derived from optionally substituted heteroaryl, or a salt thereof, which method comprises
(1) reacting a compound represented by the formula: 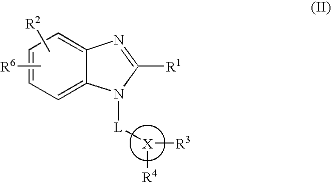
wherein R⁶ is hydroxyl, halogen or carboxy and other symbols are as defined above or a salt thereof, with a compound represented by the formula:
R⁵OOC—Y—Z—L₁ (III)
wherein L₁ is leaving group and other symbols are as defined above or a salt thereof, or
(2) reacting a compound represented by the formula: 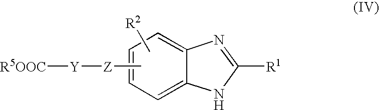
wherein each symbol is defined above or a salt thereof, with a compound represented by the formula: 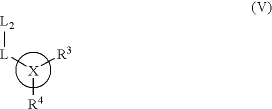
wherein L₂ is leaving group and other symbols are as defined above or a salt thereof.

14. A production method of a compound represented by the formula: 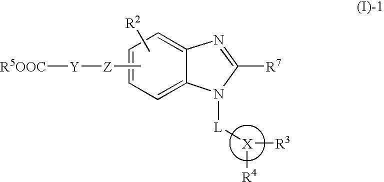
wherein
R² is hydrogen or lower alkyl,
R³ is hydrogen, halogen, cyano, optionally esterified carboxy, lower alkoxy, lower alkyl optionally substituted by lower alkoxy, optionally substituted amino, mono-, di- or trihalo(lower)alkyl, optionally substituted aryl or heteroaryl,
R⁴ is halogen, lower alkyl, lower alkoxy, lower alkenyl, or mono-, di- or trihalo(lower)alkyl,
R⁵ is hydrogen or carboxy protective group,
R⁷ is lower alkyl,
L is lower alkylene,
ring X is benzene ring or heteroaryl ring,
Y is lower alkylene, optionally substituted phenylene or bivalent residue derived from optionally substituted heteroaryl, and
Z is bond, —O—, —CH₂O—, —OCH₂—, —N(R⁹)CH₂— or —CH₂N(R⁹)—, wherein R⁹ is hydrogen or lower alkyl,
provided that when Z is a bond, then Y is optionally substituted phenylene or bivalent residue derived from optionally substituted heteroaryl, or a salt thereof, which method comprises dehydrating a compound represented by the formula: 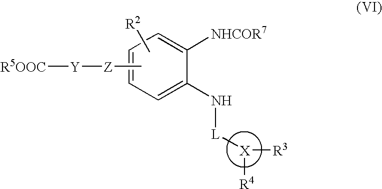
wherein each symbol is as defined above or a salt thereof.

15. A production method of a compound represented by the formula: 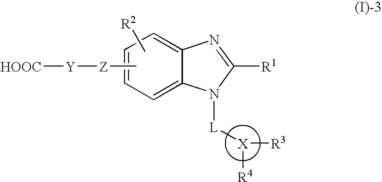
wherein
R¹ is lower alkyl, lower alkoxy, lower alkylthio, mono- or di(lower alkyl)amino or mono-, di- or trihalo(lower)alkyl,
R² is hydrogen or lower alkyl,
R³ is hydrogen, halogen, cyano, optionally esterified carboxy, lower alkoxy, lower alkyl optionally substituted by lower alkoxy, optionally substituted amino, mono-, di- or trihalo(lower)alkyl, optionally substituted aryl or heteroaryl,
R⁴ is halogen, lower alkyl, lower alkoxy, lower alkenyl, or mono-, di- or trihalo(lower)alkyl,
L is lower alkylene,
ring X is benzene ring or heteroaryl ring,
Y is lower alkylene, optionally substituted phenylene or bivalent residue derived from optionally substituted heteroaryl, and
Z is bond, —O—, —CH₂O—, —OCH₂—, —N(R⁹)CH₂— or —CH₂N(R⁹)—, wherein R⁹ is hydrogen or lower alkyl,
provided that when Z is a bond, then Y is optionally substituted phenylene or bivalent residue derived from optionally substituted heteroaryl, or a salt thereof, which method comprises hydrolyzing a compound represented by the formula: 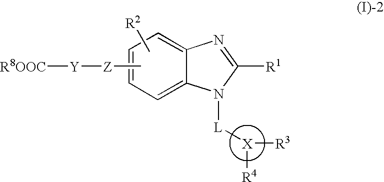
wherein R⁸ is carboxy protective group and other symbols are as defined above or a salt thereof.
